(19) United States
(12) Patent Application Publication
(10) Pub. No.: US 2010/0317515 A1
(43) Pub. Date: Dec. 16, 2010

(54) Azolylmethyloxiranes, use Thereof and Agents Containing the Same

(75) Inventors: Jochen Dietz, Karlsruhe (DE); Thomas Grote, Wachenheim (DE); Bernd Mueller, Frankenthal (DE); Jan Klaas Lohmann, Ludwigshafen (DE); Jens Renner, Bad Duerkheim (DE); Sarah Ulmschneider, Bad Duerkheim (DE); Alice Glaettli, Frankfurt (DE); Marianna Vrettou, Mannheim (DE)

Correspondence Address: BRINKS, HOFER, GILSON & LIONE, P.O. BOX 110285, RESEARCH TRIANGLE PARK, NC, 27709, US

(73) Assignee: BASF SE, Ludwigshafen (DE)

(21) Appl. No.: 12/808,793

(22) PCT Filed: Dec. 12, 2008
(86) PCT No.: PCT/EP2008/067394

§ 371 Date: Jun. 17, 2010

(30) Foreign Application Priority Data
Dec. 19, 2007 (EP) 07123700.2

(51) Int. Cl. A01C 1/06; C07D 405/06; C07F 9/28; A01N 43/653; C07D 303/36; C07D 303/08; C07D 303/14; A01P 3/00

(52) U.S. Cl. 504/100; 548/263.2; 548/264.2; 548/264.4; 548/118; 514/384; 549/513; 549/552; 548/268.8; 549/563; 549/559

(57) ABSTRACT

The present invention relates to triazolylmethyloxiranes of the formula I

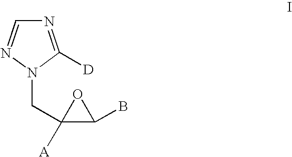

in which the variables A, B and D have the meanings described in the claims and the description.

The present invention relates to azolylmethyloxiranes of the formula I

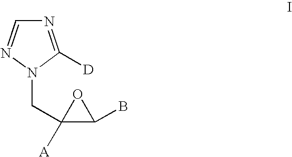

in which the variables have the following meanings:
A is phenyl which is substituted by an F and contains a further substituent L different from Br, where the phenyl may additionally contain one or two substituents L;
B is phenyl which is unsubstituted or substituted by one, two, three or four identical or different substituents L, where L is as defined below:
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-Cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino or di-C₁-C₈-alkylamino,
   A² is one of the groups mentioned for A₁ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
   A³,A⁴ independently of one another are hydrogen, C₁-C₈-alkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
   where the aliphatic and/or alicyclic and/or aromatic groups of the radical definitions of L for their part may carry one, two, three or four identical or different groups R^{L}:
   R^{L} is halogen, cyano, nitro, C₁-C₈-alkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D is S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
   R³ is C₁-C₈-alkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
is a group DI

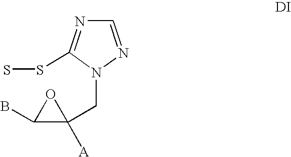

where A and B are as defined above;
is a group DII

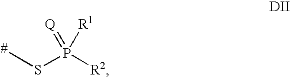

where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where M is as defined below:
M is an alkali metal cation, an equivalent of an alkaline earth metal cation, an equivalent of a copper, zinc, iron or nickel cation or an ammonium cation of the formula (E)

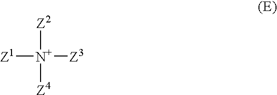

in which
   Z¹ and Z² independently are hydrogen or C₁-C₈-alkyl;
   Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and agriculturally acceptable salts thereof.

The compounds of the formula I can be present in the “thiol” form of the formula Ia or in the “thiono” form of the formula Ib.

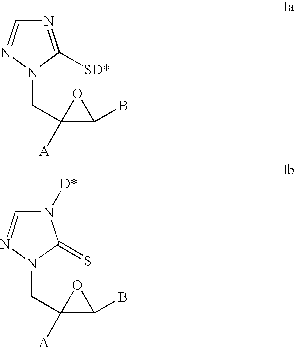

in which D* is as defined below:
R, where R has the meaning defined above;
a group DII*

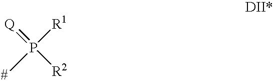

where # is the point of attachment to the sulfur atom in formula Ia or the azolyl ring in formula Ib and Q, R¹, and R² have the meaning defined above; or
a group M, where M has the meaning defined above,
   and where the other substituents have the meaning defined above.

However, for the sake of simplicity, in each case generally only the “thiol” form is shown here.

The invention furthermore relates to the preparation of the compounds I, to the intermediates for preparing the compounds I and to their preparation, and also to the use of the compounds according to the invention for controlling phytopathogenic fungi, and to compositions comprising them.

Triazolylmethyloxiranes having a substituted triazole group are known, for example, from WO 96/38440, WO 97/41107, WO 97/42178, WO 97/43269, WO 97/44331, WO 97/443332, WO 99/05149 and WO 99/21853.

However, in particular at low application rates, the fungicidal action of the compounds known from the prior art is sometimes unsatisfactory. Accordingly, it was an object of the present invention to provide novel compounds which preferably have improved properties, such as improved fungicidal action and/or better toxicological properties. Surprisingly, this object was achieved with the compounds of the formula I described here.

Owing to the basic character of their nitrogen atoms, the compounds I are capable of forming salts or adducts with inorganic or organic acids or with metal ions. This also applies to most of the precursors described herein of compounds I, the salts and adducts of which are also provided by the present invention.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid and other arylcarboxylic acids, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc and others. Particular preference is given to the metal ions of the elements of transition groups of the fourth period. The metals can be present in the various valencies that they can assume.

The compounds of the formula I according to the invention can be prepared by different routes analogously to processes known per se of the prior art (see, for example, the prior art cited at the outset and Pflanzenschutz-Nachrichten Bayer 57/2004, 2, pages 145-162). The compounds according to the invention can be prepared, for example, according to the syntheses shown in the schemes below.

The compounds according to the invention can be prepared in an advantageous manner from compounds of the formula II

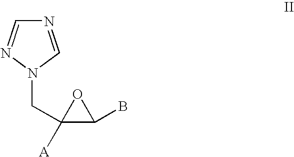

in which A and B are as defined herein by reaction with a strong base and sulfur powder. This results in the formation of compounds of the formula I in which D is SH (compounds I-1):

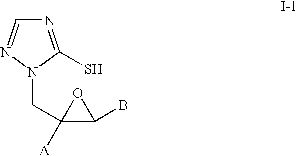

Suitable bases are all bases known to the person skilled in the art as being suitable for such reactions. Preference is given to using strong alkali metal bases such as, for example, n-butyllithium, lithium diisopropylamide, sodium hydride, sodium amide or potassium tert-butoxide. It may be preferred to carry out the reaction in the presence of an additive such as, for example, tetramethylethylenediamide (TMEDA).

Suitable solvents are all inert organic solvents customary for such reactions, where preferably ethers such as tetrahydrofuran, dioxane, diethyl ether and 1,2-dimethoxyethane or liquid ammonia or strongly polar solvents such as dimethyl sulfoxide may be used.

Sulfur is preferably used as a powder. For the hydrolysis, use is made of water, if appropriate in the presence of an organic or inorganic acid such as, for example, acetic acid, dilute sulfuric acid or dilute hydrochloric acid.

The reaction temperature is preferably between −70° C. and +20° C., in particular between −70° C. and 0° C. The reaction is generally carried out under atmospheric pressure.

In general, 1 to 3 equivalents, preferably 1 to 2.5 equivalents, of a strong base and then an equivalent amount or an excess of sulfur are employed per mole of the compound of the formula II. The reaction can be carried out under an atmosphere of protective gas such as, for example, under nitrogen or argon. Work-up is carried out according to procedures generally known to the person skilled in the art. Usually, the reaction mixture is extracted with a suitable organic solvent, and the residue is, if appropriate, purified by recrystallization and/or chromatography.

It is also possible to prepare compounds I by direct reaction with sulfur, preferably sulfur powder, without the use of a strong base such as butyllithium.

In an alternative method for preparing compounds I according to the invention from compounds II, compounds II are reacted with sulfur in the presence of an aprotic polar solvent, such as, for example, an amide (such as dimethylformamide (DMF)) or N-alkylpyrrolidone (such as N-octylpyrrolidone, N-dodecylpyrrolidone or N-methylpyrrolidone (NMP)). See also WO 99/19307, WO 97/06151, WO 97/05119 and WO 96/41804.

The reaction is generally carried out at temperatures in a range of from 140° C. to 160° C. The reaction components are usually employed in amounts such that about 6 to 15 mol of sulfur are used per mole of the compound II. Sulfur is generally used in the form of a powder. During the reaction, air is passed over the reaction mixture.

Moreover, the compounds according to the invention can be prepared in an advantageous manner from compounds of the formula II by reaction with disulfides or thiocyanogen:

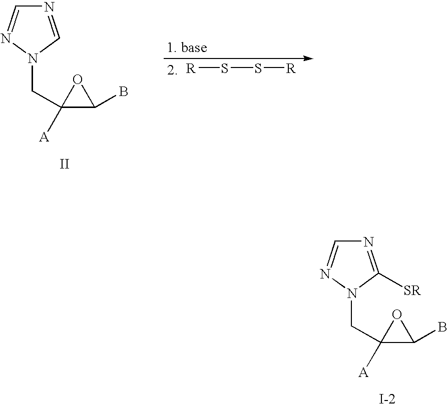

in which A and B are as described herein and R is C1-C8-alkyl, C1-C8-haloalkyl, C2-C8-alkenyl, C2-C8-haloalkenyl, C2-C8-alkynyl, C2-C8-haloalkynyl or CN.

Suitable bases are all bases known to the person skilled in the art as being suitable for such reactions. Preference is given to using strong alkali metal bases, such as, for example, n-butyllithium, lithium diisopropylamide, sodium hydride, sodium amide or potassium tert-butoxide. It may be preferred to carry out the reaction in the presence of an additive, such as, for example, tetramethylethylenediamine (TMEDA). The disulfides are commercially available or can be synthesized by known preparation processes. A specific disulfide is thiocyanogen NC—S—S—CN.

Suitable solvents are all inert organic solvents customary for such reactions, and preference is given to using ethers, such as tetrahydrofuran, dioxane, diethyl ether and 1,2-dimethoxyethane, or liquid ammonia or strongly polar solvents, such as dimethyl sulfoxide.

The reaction temperature is preferably from −70° C. to +20° C., in particular from −70° C. to 0° C. The reaction is generally carried out under atmospheric pressure.

In general, from 1 to 3 equivalents, preferably from 1 to 2.5 equivalents, of strong base and then an equivalent amount or an excess of disulfide are employed per mole of the compound of the formula II. The reaction can be carried out under an atmosphere of protective gas, such as, for example, under nitrogen or argon. Work-up is carried out by procedures known in a general manner to the person skilled in the art. Usually, the reaction mixture is extracted with a suitable organic solvent and the residue is, if appropriate, purified by recrystallization and/or chromatography.

By further reaction of compounds I-1 with R—X, where R is as defined herein at a different location and X is a leaving group such as, for example, halogen, such as Cl, Br or I, or trifluoro-C₁-C₆-alkylsulfonate, it is possible to prepare various compounds of the formula I according to the invention. To prepare compounds where D=SR where R=C₁-C₆-alkyl, preferably methyl or ethyl, a compound I-1 is reacted with the corresponding alkyl halide (see also WO 96/38440).

Compounds of the formula I in which D is S—C(═O)NA³A⁴ can be synthesized analogously to the process described in WO 99/21853.

Compounds of the formula I in which D is a group DII can be synthesized analogously to the process described in WO 99/05149.

Compounds of the formula I in which D is S—SO₂R⁴ can be synthesized analogously to the process described in WO 97/44332.

Compounds of the formula I in which D is S—CN can be synthesized analogously to the process described in WO 99/44331.

Compounds of the formula I in which D is a group DI can be synthesized analogously to the process described in WO 97/43269.

Compounds of the formula I in which D is a group S—C(═O)R³ where R³═C₁-C₈-alkyl, C₁-C₈-haloalkyl, C₁-C₈-alkoxy or C₁-C₈-haloalkoxy can be synthesized analogously to the process described in WO 97/42178.

Compounds of the formula I in which D is a group SM can be synthesized analogously to the process described in WO 97/41107.

The compounds of the formula II can be synthesized analogously to the prior art cited at the outset.

Some of the compounds of the formula II are described in the patent applications PCT/EP2007/056124, PCT/EP2007/055870 and PCT/EP200/055932. On the other hand, some of the compounds of the formula II are novel and these and their agriculturally compatible salts and adducts are likewise an object of the present invention. Compounds II also have fungicidal activity, and the present invention therefore also relates to the use of the compounds II and/or the salts and adducts thereof as fungicides.

Compounds of the formula III

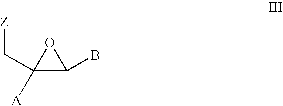

in which Z is a leaving group X (compounds III.1, see below) or OH (compounds III.2, see below) and A and B are as defined below are important starting compounds needed to obtain, finally, the compounds according to the invention.

Thus, compounds II can be prepared, for example, from compounds III.1

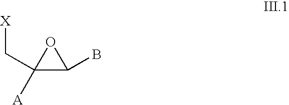

where X is a leaving group such as, for example, halogen (for example Cl or Br) or OSO₂R, where R is C₁-C₆-alkyl, C₁-C₆-haloalkyl, aryl or substituted aryl; OSO₂R is in particular a mesylate, triflate, phenyl or toluenesulfonate group. To obtain compounds of the formula II, compounds of the formula III.1 are reacted with 1,2,4-triazole and a base such as, for example, sodium hydride, for example in DMF. See also, for example, EP 0 421 125 A2.

Some of the compounds of the formula III.1 are novel. Accordingly, the invention also provides compounds of the formula III.1 in which A and B are as defined or as preferably defined for formula I and X is a leaving group, in particular halogen (for example Cl or Br) or OSO₂R where R is C₁-C₆-alkyl, C₁-C₆-haloalkyl, aryl or substituted aryl, except for the compounds anti-2-(2,4-difluorophenyl)-2-(chloromethyl)-3-(4-chlorophenyl)oxirane, anti-2-(2,4-difluorophenyl)-2-(chloromethyl)-3-(3-chlorophenyl)oxirane, anti-2-(2,4-difluorophenyl)-2-(chloromethyl)-3-(3,4-dichlorophenyl)oxirane, anti-2-(2,4-difluorophenyl)-2-(chloromethyl)-3-(2-fluorophenyl)oxirane, anti-2-(2,4-difluorophenyl)-2-(chloromethyl)-3-(3,4-difluorophenyl)oxirane, anti-2-(2,4-difluorophenyl)-2-(chloromethyl)-3-(4-methylphenyl)oxirane, anti-2-(2,4-difluorophenyl)-2-(chloromethyl)-3-(3,5-dichlorophenyl)oxirane, anti-2-(2,4-difluorophenyl)-2-(chloromethyl)-3-(3-methylphenyl)oxirane, anti-2-(2,4-difluorophenyl)-2-(chloromethyl)-3-(3,5-dimethylphenyl)oxirane, anti-2-(2,4-difluorophenyl)-2-(chloromethyl)-3-(3,5-difluorophenyl)oxirane, anti-2-(2,4-difluorophenyl)-2-(chloromethyl)-3-(2-trifluoromethylphenyl)oxirane, anti-2-(2,4-difluorophenyl)-2-(chloromethyl)-3-(2-chlorophenyl)oxirane, anti-2-(2,5-difluorophenyl)-2-(chloromethyl)-3-(2-chlorophenyl)oxirane, anti-2-(2,5-difluorophenyl)-2-(chloromethyl)-3-(4-chlorophenyl)oxirane, anti-2-(2,5-difluorophenyl)-2-(chloromethyl)-3-(2-fluorophenyl)oxirane, anti-2-(2,5-difluorophenyl)-2-(chloromethyl)-3-(2-methylphenyl)oxirane, anti-2-(2,5-difluorophenyl)-2-(chloromethyl)-3-(3-chlorophenyl)oxirane, anti-2-(3,4-difluorophenyl)-2-(chloromethyl)-3-(2-chlorophenyl)oxirane, anti-2-(3,4-difluorophenyl)-2-(chloromethyl)-3-(4-fluorophenyl)oxirane, anti-2-(3,4-difluorophenyl)-2-(chloromethyl)-3-(2-methylphenyl)oxirane, anti-2-(3,4-difluorophenyl)-2-(chloromethyl)-3-(2-fluorophenyl)oxirane, anti-2-(3,5-difluorophenyl)-2-(chloromethyl)-3-(2-chlorophenyl)oxirane, anti-2-(3,5-difluorophenyl)-2-(chloromethyl)-3-(2-methylphenyl)oxirane, anti-2-(3,5-difluorophenyl)-2-(chloromethyl)-3-(4-chlorophenyl)oxirane, anti-2-(2,4,5-trifluorophenyl)-2-(chloromethyl)-3-(4-fluorophenyl)oxirane, anti-2-(2,4,5-trifluorophenyl)-2-(chloromethyl)-3-(2-chlorophenyl)oxirane, anti-2-(2,4,5-trifluorophenyl)-2-(chloromethyl)-3-(2-methylphenyl)oxirane, anti-2-(3,4,5-trifluorophenyl)-2-(chloromethyl)-3-(4-fluorophenyl)oxirane, anti-2-(3,4,5-trifluorophenyl)-2-(chloromethyl)-3-(2-methylphenyl)oxirane, anti-2-(3,4,5-trifluorophenyl)-2-(chloromethyl)-3-(2-chlorophenyl)oxirane, anti-2-(3,4,5-trifluorophenyl)-2-(chloromethyl)-3-(2-fluorophenyl)oxirane, anti-2-(4-chloro-2-fluorophenyl)-2-(chloromethyl)-3-(2-chlorophenyl)oxirane, anti-2-(3-fluoro-4-methoxyphenyl)-2-(chloromethyl)-3-(2-chlorophenyl)oxirane, anti-2-(2-chloro-4-fluorophenyl)-2-(chloromethyl)-3-(2-fluorophenyl)oxirane, anti-2-(2-chloro-4-fluorophenyl)-2-(chloromethyl)-3-(4-fluorophenyl)oxirane, anti-2-(4-chloro-2-fluorophenyl)-2-(chloromethyl)-3-(4-chlorophenyl)oxirane, anti-2-(4-chloro-2-fluorophenyl)-2-(chloromethyl)-3-(4-methylphenyl)oxirane, anti-2-(4-chloro-2-fluorophenyl)-2-(chloromethyl)-3-(2-chlorophenyl)oxirane, anti-2-(2-fluoro-4-chlorophenyl)-2-(chloromethyl)-3-(3-chlorophenyl)oxirane, anti-2-(2-fluoro-4-chlorophenyl)-2-(chloromethyl)-3-(2-fluorophenyl)oxirane, anti-2-(2-fluoro-4-methoxyphenyl)-2-(chloromethyl)-3-(2-chlorophenyl)oxirane, anti-2-(2-fluoro-4-methoxyphenyl)-2-(chloromethyl)-3-(2-methylphenyl)oxirane, anti-2-(4-fluoro-2-methoxyphenyl)-2-(chloromethyl)-3-(2-methylphenyl)oxirane, anti-2-(4-fluoro-2-methoxyphenyl)-2-(chloromethyl)-3-(2-fluorophenyl)oxirane, anti-2-(4-fluoro-2-methoxyphenyl)-2-(chloromethyl)-3-(2-chlorophenyl)oxirane, cis-2-(4-methylphenylsulfonyloxymethyl)-2-(2,4-difluorophenyl)-3-(2-trifluoromethylphenyl)oxirane, cis-2-(4-methylphenylsulfonyloxymethyl)-2-(2,4-difluorophenyl)-3-(4-trifluoromethylphenyl)oxirane and cis-2-(methylsulfonyloxymethyl)-2-(2,4-difluorophenyl)-3-(2-methylphenyl)oxirane.

According to one embodiment of III.1, B is not ortho- or para-trifluoromethylphenyl when A is 2,4-difluorophenyl. According to a further embodiment from III.1, B is furthermore not ortho-methylphenyl when A is 2,4-difluorophenyl.

According to a further embodiment of III.1, B is not ortho- or para-trifluoromethylphenyl. According to a further specific embodiment, B is furthermore not ortho-methylphenyl.

According to a further embodiment of the compounds III.1, X is not CH₃SO₂O or 4-CH₃-phenyl-SO₂—O.

A and B have in particular the meanings as specified herein for formula I, taking into account the compounds which are excluded.

One way to prepare the compounds III.1 consists in converting the double bond in compounds of the formula IVa

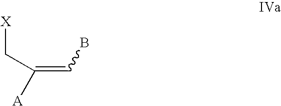

to the epoxide. X is as defined or preferably defined for formula III.1 and A and B are as defined or preferably defined for formula I. Suitable epoxidation methods are known to the person skilled in the art. It is possible, for example, to use hydrogen peroxide/maleic anhydride for this purpose.

In the formula IVa, the double bond may be present either in (E) or in (Z) configuration. This is indicated by the zigzag bond between B and the double bond. The present invention furthermore provides compounds of the formula IVa in which A and B are as defined or preferably defined for formula I, except for the compounds (Z)-1-[3-chloro-1-(4-chlorophenyl)prop-1-en-2-yl]-2,4-difluorobenzene, (Z)-1-[3-chloro-1-(3-chlorophenyl)prop-1-en-2-yl]-2,4-difluorobenzene, (Z)-1-[3-chloro-1-(3,4-dichlorophenyl)prop-1-en-2-yl]-2,4-difluorobenzene, (Z)-1-[3-chloro-1-(2-fluorophenyl)prop-1-en-2-yl]-2,4-difluorobenzene, (Z)-1-[3-chloro-1-(3,4-difluorophenyl)prop-1-en-2-yl]-2,4-difluorobenzene, (Z)-1-[3-chloro-1-(4-methylphenyl)prop-1-en-2-yl]-2,4-difluorobenzene, (Z)-1-[3-chloro-1-(3,5-dichlorophenyl)prop-1-en-2-yl]-2,4-difluorobenzene, (Z)-1-[3-chloro-1-(3-methylphenyl)prop-1-en-2-yl]-2,4-difluorobenzene, (Z)-1-[3-chloro-1-(3,5-dimethylphenyl)prop-1-en-2-yl]-2,4-difluorobenzene, (Z)-1-[3-chloro-1-(3,5-difluorophenyl)prop-1-en-2-yl]-2,4-difluorobenzene, (Z)-1-[3-chloro-1-(2-trifluoromethylphenyl)prop-1-en-2-yl]-2,4-difluorobenzene, (Z)-1-[3-chloro-1-(2-chlorophenyl)prop-1-en-2-yl]-2,4-difluorobenzene, (Z)-1-[3-chloro-1-(2-chlorophenyl)prop-1-en-2-yl]-2,5-difluorobenzene, (Z)-1-[3-chloro-1-(4-chlorophenyl)prop-1-en-2-yl]-2,5-difluorobenzene, (Z)-1-[3-chloro-1-(2-fluorophenyl)prop-1-en-2-yl]-2,5-difluorobenzene, (Z)-1-[3-chloro-1-(2-methylphenyl)prop-1-en-2-yl]-2,5-difluorobenzene, (Z)-1-[3-chloro-1-(3-chlorophenyl)prop-1-en-2-yl]-2,5-difluorobenzene, (Z)-1-[3-chloro-1-(2-chlorophenyl)prop-1-en-2-yl]-3,4-difluorobenzene, (Z)-1-[3-chloro-1-(4-fluorophenyl)prop-1-en-2-yl]-3,4-difluorobenzene, (Z)-1-[3-chloro-1-(2-methylphenyl)prop-1-en-2-yl]-3,4-difluorobenzene, (Z)-1-[3-chloro-1-(2-fluorophenyl)prop-1-en-2-yl]-3,4-difluorobenzene, (Z)-1-[3-chloro-1-(2-chlorophenyl)prop-1-en-2-yl]-3,5-difluorobenzene, (Z)-1-[3-chloro-1-(2-methylphenyl)prop-1-en-2-yl]-3,5-difluorobenzene, (Z)-1-[3-chloro-1-(4-chlorophenyl)prop-1-en-2-yl]-3,5-difluorobenzene, (Z)-1-[3-chloro-1-(4-fluorophenyl)prop-1-en-2-yl]-2,4,5-trifluorobenzene, (Z)-1-[3-chloro-1-(2-chlorophenyl)prop-1-en-2-yl]-2,4,5-trifluorobenzene, (Z)-1-[3-chloro-1-(2-methylphenyl)prop-1-en-2-yl]-2,4,5-trifluorobenzene, (Z)-1-[3-chloro-1-(4-fluorophenyl)prop-1-en-2-yl]-3,4,5-trifluorobenzene, (Z)-1-[3-chloro-1-(2-methylphenyl)prop-1-en-2-yl]-3,4,5-trifluorobenzene, (Z)-1-[3-chloro-1-(2-chlorophenyl)prop-1-en-2-yl]-3,4,5-trifluorobenzene, (Z)-1-[3-chloro-1-(2-fluorophenyl)prop-1-en-2-yl]-3,4,5-trifluorobenzene, (Z)-4-chloro-1-[3-chloro-1-(2-chlorophenyl)prop-1-en-2-yl]-2-fluorobenzene, (Z)-3-fluoro-1-[3-chloro-1-(2-chlorophenyl)prop-1-en-2-yl]-4-methoxybenzene, (Z)-2-chloro-1-[3-chloro-1-(2-fluorophenyl)prop-1-en-2-yl]-4-fluorobenzene, (Z)-4-chloro-1-[3-chloro-1-(4-chlorophenyl)prop-1-en-2-yl]-2-fluorobenzene, (Z)-2-chloro-1-[3-chloro-1-(4-fluorophenyl)prop-1-en-2-yl]-4-fluorobenzene, (Z)-2-chloro-1-[3-chloro-1-(2-chlorophenyl)prop-1-en-2-yl]-4-fluorobenzene, (Z)-4-chloro-1-[3-chloro-1-(4-methylphenyl)prop-1-en-2-yl]-2-fluorobenzene, (Z)-2-fluoro-1-[3-chloro-1-(3-chlorophenyl)prop-1-en-2-yl]-4-chlorobenzene, (Z)-2-fluoro-1-[3-chloro-1-(2-fluorophenyl)prop-1-en-2-yl]-4-chlorobenzene, (Z)-2-fluoro-1-[3-chloro-1-(2-chlorophenyl)prop-1-en-2-yl]-4-methoxybenzene, (Z)-2-fluoro-1-[3-chloro-1-(2-methylphenyl)prop-1-en-2-yl]-4-methoxybenzene, (Z)-4-fluoro-1-[3-chloro-1-(2-chlorophenyl)prop-1-en-2-yl]-2-methoxybenzene, (Z)-4-fluoro-1-[3-chloro-1-(2-fluorophenyl)prop-1-en-2-yl]-2-methoxybenzene and (Z)-4-fluoro-1-[3-chloro-1-(2-methylphenyl)prop-1-en-2-yl]-2-methoxybenzene. X has the meanings as described above for formula III.1.

According to one embodiment, B is not ortho- or para-trifluoromethylphenyl when A is 2,4,difluorophenyl. According to a further embodiment, B is not ortho-methylphenyl when A is 2,4-difluorophenyl.

According to a special embodiment, B is not ortho- or para-trifluoromethylphenyl. According to a further special embodiment, B is furthermore not ortho-methylphenyl.

A and B in IVa have in particular the meanings as specified herein for formula I, taking into account the compounds which are excluded.

Compounds IVa can be obtained from compounds IVc

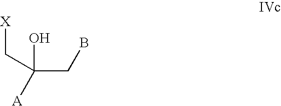

by reacting compounds IVc, for example, with acetic acid/H₂SO₄ in a suitable organic solvent such as, for example, an ether, such as Et₂O or dioxane, to form the double bond. Suitable methods are known to the person skilled in the art. X is as defined for formula III and A and B are as defined for formula I.

Some of the compounds of the formula IVc are novel. Accordingly, the invention also provides compounds of the formula IVc in which A and B are as defined or preferably defined for formula I, except for the compounds 1-chloro-2-(2,4-difluorophenyl)-3-(4-chlorophenyl)propan-2-ol, 1-chloro-2-(2,4-difluorophenyl)-3-(3-chlorophenyl)propan-2-ol, 1-chloro-2-(2,4-difluorophenyl)-3-(3,4-dichlorophenyl)propan-2-ol, 1-chloro-2-(2,4-difluorophenyl)-3-(2-fluorophenyl)propan-2-ol, 1-chloro-2-(2,4-difluorophenyl)-3-(3,4-difluorophenyl)propan-2-ol, 1-chloro-2-(2,4-difluorophenyl)-3-(4-methylphenyl)propan-2-ol, 1-chloro-2-(2,4-difluorophenyl)-3-(3,5-dichlorophenyl)propan-2-ol 1-chloro-2-(2,4-difluorophenyl)-3-(3-methylphenyl)propan-2-ol, 1-chloro-2-(2,4-difluorophenyl)-3-(3,5-dimethylphenyl)propan-2-ol, 1-chloro-2-(2,4-difluorophenyl)-3-(3,5-difluorophenyl)propan-2-ol, 1-chloro-2-(2,4-difluorophenyl)-3-(2-trifluoromethylphenyl)propan-2-ol, 1-chloro-2-(2,4-difluorophenyl)-3-(2-chlorophenyl)propan-2-ol, 1-chloro-2-(2,5-difluorophenyl)-3-(2-chlorophenyl)propan-2-ol, 1-chloro-2-(2,5-difluorophenyl)-3-(4-chlorophenyl)propan-2-ol, 1-chloro-2-(2,5-difluorophenyl)-3-(2-fluorophenyl)propan-2-ol, 1-chloro-2-(2,5-difluorophenyl)-3-(2-methylphenyl)propan-2-ol, 1-chloro-2-(2,5-difluorophenyl)-3-(3-chlorophenyl)propan-2-ol, 1-chloro-2-(3,4-difluorophenyl)-3-(2-chlorophenyl)propan-2-ol, 1-chloro-2-(3,4-difluorophenyl)-3-(4-fluorophenyl)propan-2-ol, 1-chloro-2-(3,4-difluorophenyl)-3-(2-methylphenyl)propan-2-ol, 1-chloro-2-(3,4-difluorophenyl)-3-(2-fluorophenyl)propan-2-ol, 1-chloro-2-(3,5-difluorophenyl)-3-(2-chlorophenyl)propan-2-ol, 1-chloro-2-(3,5-difluorophenyl)-3-(2-methylphenyl)propan-2-ol, 1-chloro-2-(3,5-difluorophenyl)-3-(4-chlorophenyl)propan-2-ol, 1-chloro-2-(2,4,5-trifluorophenyl)-3-(4-fluorophenyl)propan-2-ol, 1-chloro-2-(2,4,5-trifluorophenyl)-3-(2-chlorophenyl)propan-2-ol, 1-chloro-2-(2,4,5-trifluorophenyl)-3-(2-methylphenyl)propan-2-ol, 1-chloro-2-(3,4,5-trifluorophenyl)-3-(4-fluorophenyl)propan-2-ol, 1-chloro-2-(3,4,5-trifluorophenyl)-3-(2-methylphenyl)propan-2-ol, 1-chloro-2-(3,4,5-trifluorophenyl)-3-(2-chlorophenyl)propan-2-ol, 1-chloro-2-(3,4,5-trifluorophenyl)-3-(2-fluorophenyl)propan-2-ol, 1-chloro-2-(4-chloro-2-fluorophenyl)-3-(2-chlorophenyl)propan-2-ol, 1-chloro-2-(4-chloro-2-fluorophenyl)-3-(4-chlorophenyl)propan-2-ol, 1-chloro-2-(4-chloro-2-fluorophenyl)-3-(4-methylphenyl)propan-2-ol, 1-chloro-2-(3-fluoro-4-methoxyphenyl)-3-(2-chlorophenyl)propan-2-ol, 1-chloro-2-(2-chloro-4-fluorophenyl)-3-(2-chlorophenyl)propan-2-ol, 1-chloro-2-(2-chloro-4-fluorophenyl)-3-(2-fluorophenyl)propan-2-ol, 1-chloro-2-(2-chloro-4-fluorophenyl)-3-(4-fluorophenyl)propan-2-ol, 1-chloro-2-(2-fluoro-4-chlorophenyl)-3-(3-chlorophenyl)propan-2-ol, 1-chloro-2-(2-fluoro-4-chlorophenyl)-3-(2-fluorophenyl)propan-2-ol, 1-chloro-2-(2-fluoro-4-methoxyphenyl)-3-(2-chlorophenyl)propan-2-ol, 1-chloro-2-(2-fluoro-4-methoxyphenyl)-3-(2-methylphenyl)propan-2-ol, 1-chloro-2-(4-fluoro-2-methoxyphenyl)-3-(2-chlorophenyl)propan-2-ol, 1-chloro-2-(4-fluoro-2-methoxyphenyl)-3-(2-fluorophenyl)propan-2-ol and 1-chloro-2-(4-fluoro-2-methoxyphenyl)-3-(2-methylphenyl)propan-2-ol. X has the meanings as described above for formula III.1.

According to one embodiment, B is not ortho- or para-trifluoromethylphenyl, when A is 2,4-difluorophenyl. According to a further embodiment, B is not ortho-methylphenyl when A is 2,4-difluorophenyl. According to a specific embodiment, B is not ortho- or para-trifluoromethylphenyl. According to a further specific embodiment, B is furthermore not ortho-methylphenyl.

A and B in IVc have in particular the meanings as specified herein for formula I, taking into account the compounds which are excluded.

The compounds IVc can be obtained, for example, by a Grignard reaction according to the following scheme:

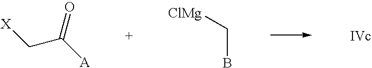

See also EP 409049.

Compounds of the formula III.1 can also be obtained from compounds of the formula III.2,

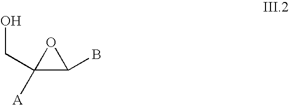

by introducing the leaving group X by methods known to the person skilled in the art. Thus, a compound of the formula III.2 is reacted, for example, with R—SO₂Y, where R is as defined for formula III.1 and Y is halogen, where R—SO₂Y is, for example, mesyl chloride, in the presence of a base (for example NEt₃) (see also EP386557). To obtain compounds III.1 in which X is halogen, the corresponding compound III.2 can be reacted with C(Hal)₄ (Hal=Br or Cl) with PPh₃, for example in CH₂Cl₂, to give a compound III.1. Alternatively, a compound III.2 can be reacted with SOCl₂/pyridine (see also WO 2005/056548).

Some of the compounds of the formula III.2 are novel. Accordingly, the present invention also provides compounds of the formula III.2 in which A and B are as defined or as preferably defined for formula I, except for the compounds 2-hydroxymethyl-2-(2,4-difluorophenyl)-3-(2-trifluoromethylphenyl)oxirane and 2-hydroxymethyl-2-(2,4-difluorophenyl)-3-(4-trifluoromethylphenyl)oxirane.

According to one embodiment, B is not ortho- or para-trifluoromethylphenyl when A is 2,4-difluorophenyl. According to a further embodiment, B is not ortho-methylphenyl when A is 2,4-difluorophenyl.

According to a specific embodiment, B is not ortho- or para-trifluoromethylphenyl. According to a further specific embodiment, B is furthermore not ortho-methylphenyl.

A and B in III.2 have in particular the meanings as specified herein for formula I and formula III.1, taking into account the compounds which are excluded.

Compounds of the formula III.2 can be obtained from α,β-disubstituted acroleins of the type of the formula V

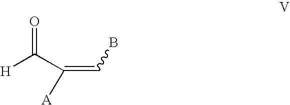

by initial epoxidation, for example with H₂O₂ in the presence of a base such as, for example, NaOH or by reaction with a peracid (for example MCPBA=m-chloroperoxybenzoic acid) or tert-butyl hydroperoxide). This gives compounds of the formula Va

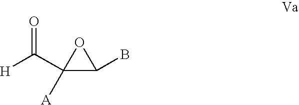

By reducing the aldehyde group in compounds Va, for example with NaBH₄ (see also EP 0 386 557A1), compounds III.2 are formed. Processes for epoxidation and reduction of the aldehyde group are well known to the person skilled in the art.

In formula V, the double bond can be present either in the (E) or in the (Z) configuration. This is indicated by the zig-zag bond between B and the double bond. Some of the compounds of the formula V are novel. Accordingly, the invention also provides compounds of the formula V in which A and B are as defined or as preferably defined for formula I, except for the compounds 2-(2,4-difluorophenyl)-3-(2-trifluoromethylphenyl)propenal and 2-(2,4-difluorophenyl)-3-(4-trifluoromethylphenyl)propenal.

According to one embodiment, B is not ortho-methylphenyl when A is 2,4-difluorophenyl. According to a further embodiment, B is not ortho- or para-trifluoromethylphenyl when A is 2,4-difluorophenyl.

According to a specific embodiment, B is not ortho- or para-trifluoromethylphenyl. According to a further specific embodiment, B is furthermore not ortho-methylphenyl.

A and B in V have in particular the meanings as specified herein for formula I, taking into account the compounds which are excluded.

Compounds Va in which A and B are as defined or as preferably defined as described herein for compounds of the formula I also form part of the subject matter of the present invention, except for the compounds in which A is 2,4-difluorophenyl and B is 2-trifluoromethyl or 4-trifluorophenyl.

According to one embodiment, B is not ortho-methylphenyl when A is 2,4-difluorophenyl.

According to a specific embodiment, B is not ortho- or para-trifluoromethylphenyl.

According to a further specific embodiment, B is not ortho-methylphenyl.

A and B in Va have in particular the meanings as specified herein for formula I, taking into account the compounds which are excluded.

The compounds V can be synthesized, for example, analogously to the procedure described in DE3601927, i.e. by reacting compounds of the type of the formula VI

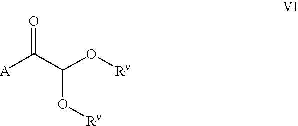

with appropriate phosphorus compounds of the Wittig or Horner-Emmons type with subsequent acidic cleavage of the acetal. In this case, R^{y} is in each case independently C₁-C₄-alkyl.

An alternative preparation of compounds V consists in the oxidation of compounds of the formula VII.

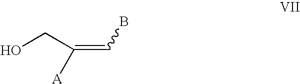

Suitable oxidizing agents and conditions are known to the person skilled in the art, for example a reaction according to Swern (Australian Journal of Chemistry, 57(6), 537-548; 2004), reactions with hypervalent iodine compounds (Organic Letters, 5(17), 2989-2992; 2003), with chromium compounds such as, for example, pyridinium dichromate (Tetrahedron, 45(1), 239-58; 1989) or with manganese oxides such as, for example, MnO₂ (Journal of the American Chemical Society, 107(13), 3963-71; 1985). The oxidation may also be carried out via a Dess-Martin oxidation in a solvent such as, for example, CH₂Cl₂.

In formula VII, the double bond may be present either in the (E) or in the (Z) configuration. This is indicated by the zig-zag bond between B and the double bond. Some compounds of the formula VII are novel. Accordingly, the invention furthermore provides compounds of the formula VII in which A and B are as defined or as preferably defined for formula I.

According to one embodiment of the compounds VII, B is not ortho- or para-trifluoromethylphenyl when A is 2,4-difluorophenyl. According to a specific embodiment, B is not ortho- or para-trifluoromethylphenyl.

A and B in VII have in particular the meanings as specified herein for formula I, taking into account the compounds which are excluded.

Starting with compounds VII, it is also possible to prepare compounds III.2 directly by epoxidation in the presence of a transition metal alkoxide such as, for example, V(O)(OR)₃ or Ti(OR)₄ (R═C₁-C₆-alkyl) and an oxidizing agent such as, for example, tBuOOH analogously to U.S. Pat. No. 5,399,708.

Compounds of the formula VII can be prepared from α,β-unsaturated acrylic esters of the formula VIII:

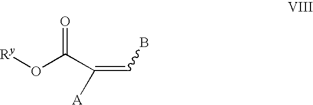

To this end, esters of the formula VIII are reduced to the alcohol VII. Suitable reducing procedures are well known to the person skilled in the art.

In formula VIII, the double bond may be present either in the (E) or in the (Z) configuration. This is indicated by the zig-zag bond between B and the double bond. Some compounds of the formula VIII are novel, and these compounds also form part of the subject matter of the present invention. In further preferred embodiments, A and B in the compounds VIII have the meanings as specified herein for formula I, taking into account the compounds which are excluded.

Compounds of the formula VIII can also be reduced in one step to the acrolein of the formula V, for example using metal hydrides such as, for example, diisobutylaluminum hydride at low temperatures. To this end, it is possible to use in particular aluminum hydrides, preferably lithium alanate (European Journal of Medicinal Chemistry, 40(6), 529-541; 2005) or dialkylaluminum hydrides such as, for example, DIBAL-H (Synlett, (18), 3182-3184; 2006).

The acrylic esters of the formula VIII are obtainable from glyoxalic esters of the formula IX by reaction with phosphorus compounds, for example of the Horner-Emmons type or Wittig compounds.

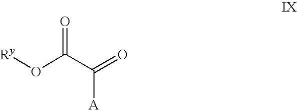

Such reactions are described, inter alia, Tetrahedron, 46(13-14), 4951-94; 1990, Tetrahedron Letters, 47(16), 2675-2678; 2006, Synthesis, (12), 1797-1802; 2003, WO9929645 or Synthetic Communications, 20(12), 1781-91; 1990.

The synthesis of glyoxalic esters is described, inter alia, in Journal of Organic Chemistry, 52(22), 5026-30; 1987 by reaction of Grignard compounds (starting with commercially available halogen compounds and magnesium) X¹MgA with oxalic esters of the formula

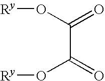

Suitable phosphorus compounds (of the Horner-Emmons type and Wittig type) can be prepared by known standard methods, for example from a compound of the type below:

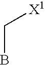

B is as defined above, X¹, is a leaving group such as, for example, a halide, preferably chlorine or bromine. The conversion of such halides into the desired Horner-Emmons or Wittig reagents can be carried out as described, for example, in Chemistry of Materials, 13(9), 3009-3017; 2001, European Journal of Organic Chemistry, (7), 1247-1257; 2005 or WO1992/05145.

The alkyl halides are either commercially available or can be prepared by standard methods, for example by halogenation of the corresponding methyl compound. Suitable halogenating agents for this reaction are N-bromosuccinimide (Chemistry-A European Journal, 12(21), 5632-5641; 2006) or N-chlorosuccinimide (Tetrahedron Letters, 47(37), 6607-6609; 2006).

According to one alternative, compounds of the formula V can also be prepared via an aldol synthesis according to the scheme below:

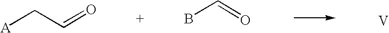

A further alternative of preparing compounds of the formula II consists in the epoxidation of a compound of the formula IVb.

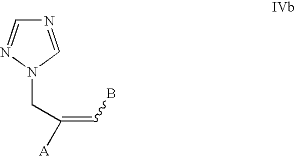

Suitable epoxidation methods are known to the person skilled in the art, see also the preparation of the compounds III.2 from compounds V.

In formula IVb, the double bond can be present either in the (E) or in the (Z) configuration. This is indicated by the zig-zag bond between B and the double bond. The present invention furthermore provides compounds of the formula IVb in which A and B are as defined or preferred for formula I.

According to one embodiment of the compounds IVb, B is not ortho- or para-trifluoromethylphenyl when A is 2,4-difluorophenyl. According to a specific embodiment, B is not ortho- or para-trifluoromethylphenyl.

According to a further embodiment of the compounds IVb, B is furthermore not ortho-methylphenyl when A is 2,4-difluorophenyl. According to a specific embodiment, B is not ortho-methylphenyl.

A and B in IVb have in particular the meanings as specified herein for formula I, taking into account the compounds which are excluded.

Compounds of the formula IVb can be obtained by reacting a compound of the formula IVa, as shown above, with 1,2,4-triazole and a base. The reaction conditions can be selected as described above for the preparation of compounds II starting with compounds III.

A further alternative of preparing compounds of the formula I consists in initially converting compounds of the formula III.1 (see above) with hydrazine into compounds of the formula IIIa.

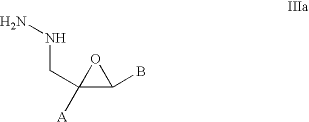

The present invention also provides compounds of the formula IIIa in which A and B are as defined or preferred for formula I.

Compounds of the formula IIIa can then be reacted with a thiocyanate YSCN in which Y is an alkali metal or ammonium, preferably sodium, potassium or ammonium, particularly preferably using NH₄SCN. This gives thiosemicarbazide compounds IIIb

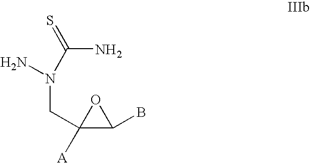

The triazolyl ring can be formed by reaction with formic acid, and what is obtained is the thione form of the corresponding compound of the formula I (D=SH) according to the invention, which, if appropriate, may be reacted further (see above). See also DE19744400 (WO99/18088).

The present invention furthermore provides compounds of the formula IIIb in which A and B are as defined or preferred for formula I.

According to a further alternative, compounds IIIa can be reacted with formaldehyde ((CH₂O)ₙ) and a thiocyanate (YSCN, see above), which gives compounds of the formula IIIc

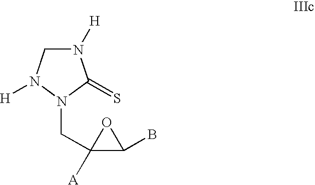

The triazolyl ring and thus the corresponding compound of the formula I is then formed by oxidation with, for example, iron(III) chloride in aqueous HCl (see also DE19961603 or WO 00/146158) or with oxygen in the presence of KOH and sulfur (see also WO 99/18087).

The present invention furthermore provides compounds of the formula IIIc in which A and B are as defined or preferred for formula I.

Yet another synthesis alternative consists in reacting a compound IIIa with a carbonyl compound (R^{x1}R^{x2})C═O (R^{x1} ═C₁-C₄-alkyl or phenyl, R^{x2}=hydrogen or C₁-C₄-alkyl or R^{x1} and R^{x2} together form a —(CH₂)₅— chain), for example using acetone ((CH₃)₂CO) and a thiocyanate YSCN in which Y is sodium, potassium or ammonium, to give compounds IIId,

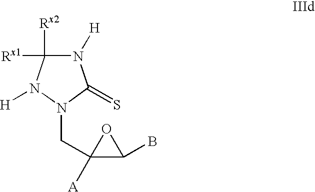

and then converting the compound formed in this manner with formic acid, if appropriate in the presence of a catalyst (for example HCl, H₂SO₄, p-toluenesulfonic acid, metal oxides such as, for example, amorphous TiO₂) into the corresponding triazole compound I. In this case, R^{x1} and R^{x2} are preferably both methyl (compounds IIId-1). See also DE19744401 and WO 99/18086.

The present invention furthermore provides compounds of the formula IIId in which A and B are as defined or preferred for formula I.

In some of the definitions of the symbols in the formulae given herein, collective terms are used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl
alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular by fluorine, chlorine or bromine;
alkadienyl: unsaturated straight-chain or branched hydrocarbon radicals having 4 to 6 or 4 to 8 carbon atoms and two double bonds in any position;
alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: alkynyl as defined above, where some or all of the hydrogen atoms in these groups are replaced by halogen atoms as described above under haloalkyl, in particular by fluorine, chlorine or bromine;
cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular by fluorine, chlorine or bromine;
cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;
halocycloalkenyl: cycloalkenyl as defined above, where some or all of the hydrogen atoms in these groups are replaced by halogen atoms as described above under haloalkyl, in particular by fluorine, chlorine or bromine;
alkoxy: an alkyl group as defined above which is attached via an oxygen, preferably having 1 to 8, more preferably 2 to 6, carbon atoms. Examples are: methoxy, ethoxy, npropoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.
alkylene: divalent unbranched chains of CH₂ groups. Preference is given to (C₁-C₆)alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄-CH₂;
a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which contains one or two heteroatoms from the group consisting of O, N and S as ring members;
a five- or six-membered saturated or partially unsaturated heterocycle which contains one, two, three or four heteroatoms from the group consisting of O, N and S as ring members: for example monocyclic saturated or partially unsaturated heterocycles which, in addition to carbon ring members, contain one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding-ylidene radicals;
a seven-membered saturated or partially unsaturated heterocycle which contains one, two, three or four heteroatoms from the group consisting of O, N and S as ring members: for example mono- and bicyclic heterocycles having 7 ring members which, in addition to carbon ring members, contain one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;
   a 5-, 6-, 7-, 8-, 9- or 10-membered aromatic heterocycle which contains 1, 2, 3 or 4 heteroatoms from the group consisting of O, N and S: in particular a five- or six-membered aromatic mono- or bicyclic heterocycle which contains one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
5-membered heteroaryl which contains one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may contain one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which contains one, two, three or four, preferably one, two or three, nitrogen atoms, where the heteroaryl may be attached via carbon or nitrogen, if present: 6-ring heteroaryl groups which, in addition to carbon atoms, may contain one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The novel compounds according to the invention contain chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both the pure enantiomers or diastereomers and mixtures thereof. This applies to the compounds according to the invention of the formula I and, if appropriate, correspondingly to their precursors. The scope of the present invention includes in particular the (R) and (S) isomers and the racemates of the compounds according to the invention, in particular of the formula I or II, which have centers of chirality. Suitable compounds according to the invention, in particular of the formula I or II, also include all possible stereoisomers (cis/trans isomers) and mixtures thereof.

The compounds according to the invention, in particular of the formula I or II, may be present in various crystal modifications which may differ in their biological activity. They are likewise provided by the present invention.

In the compounds according to the invention I, particular preference is given to the following meanings of the substituents, in each case on their own or in combination.

According to the invention, A is phenyl which is substituted by one F and contains a further substituent L different from Br, where the phenyl may additionally contain one or two substituents L selected independently of one another. According to a preferred embodiment, A is a group A-1

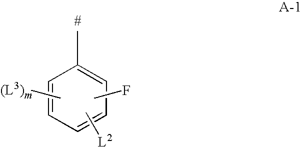

in which # is the point of attachment of the phenyl ring to the oxirane ring; and
L² is selected from the group consisting of F, Cl, NO₂, phenyl, halogenphenyl, phenoxy, halogenphenoxy, C₁-C₄-alkyl, C₁-C₄-haloalkyl, C₁-C₄-alkoxy, C₁-C₄-haloalkoxy and C₁-C₄-haloalkylthio;
L³ is independently selected from the group consisting of F, Cl, Br, NO₂, phenyl, halogenphenyl, phenoxy, halogenphenoxy, C₁-C₄-alkyl, C₁-C₄-haloalkyl, C₁-C₄-alkoxy, C₁-C₄-haloalkoxy and C₁-C₄-haloalkylthio; and
m is 0, 1 or 2.

In one embodiment, L² is selected from the group consisting of F, Cl, methyl, methoxy, CF₃, CHF₂, OCF₃, OCF₃ and OCHF₂. According to a more specific embodiment, L² is F or Cl.

In one embodiment, L³ is independently selected from the group consisting of F, Cl, methyl, methoxy, CF₃, CHF₂, OCF₃, OCF₃ or OCHF₂. According to a more specific embodiment, L³ is independently F or Cl.

According to a preferred embodiment, m=0. According to a further preferred embodiment, m=1.

In the formula A-1, the fluorine substituent is, according to a preferred embodiment, in the 4-position.

According to yet a further preferred embodiment, A is disubstituted phenyl which contains one F and a further substituent L selected from the group consisting of Cl, C₁-C₄-alkyl, C₁-C₄-haloalkyl and C₁-C₄-alkoxy, in particular selected from the group consisting of Cl, methyl, trifluoromethyl and methoxy. The second substituent L is specifically selected from the group consisting of methyl, methoxy and chlorine. According to one aspect thereof, one of the substituents is located in the 4-position of the phenyl ring.

According to a further embodiment, the phenyl ring A is substituted in the 2,4-position.

According to a further preferred embodiment of the invention, A is phenyl which is substituted by exactly two F. According to one aspect, A is 2,3-difluoro-substituted. According to a further aspect, A is 2,4-difluoro-substituted. According to yet a further aspect, A is 2,5-difluoro-substituted. According to yet a further aspect, A is 2,6-difluoro-substituted. According to yet a further aspect, A is 3,4-difluoro-substituted. According to yet a further aspect, A is 3,5-difluoro-substituted.

According to yet a further embodiment, A is phenyl which is substituted by exactly three F. According to one aspect, A is 2,3,4-trifluoro-substituted. According to a further aspect, A is 2,3,5-trifluoro-substituted. According to yet a further aspect, A is 2,3,6-trifluoro-substituted. According to yet a further aspect, A is 2,4,6-trifluoro-substituted. According to yet a further aspect, A is 3,4,5-trifluoro-substituted. According to yet a further aspect, A is 2,4,5-trifluoro-substituted.

According to one embodiment of the invention, B is unsubstituted phenyl.

According to a further embodiment, B is phenyl which contains one, two, three or four independently selected substituents L.

According to a further embodiment, the phenyl ring is monosubstituted by a substituent L, where according to a special aspect of this embodiment, L is located in the ortho-position to the point of attachment of the phenyl ring to the oxirane ring.

According to a further embodiment, B is phenyl which contains one, two or three independently selected substituents L as defined below. In a preferred embodiment, B is phenyl which is substituted by one, two or three halogen atoms.

According to a further embodiment of the invention, B is a phenyl ring which contains a substituent L in the ortho-position and furthermore has a further independently selected substituent L. According to one aspect, the phenyl ring is 2,3-disubstituted. According to a further aspect, the phenyl ring is 2,4-disubstituted. According to yet a further aspect, the phenyl ring is 2,5-disubstituted. According to yet a further aspect, the phenyl ring is 2,6-disubstituted.

According to a further embodiment of the invention, B is a phenyl ring which contains a substituent L in the ortho-position and furthermore contains two further independently selected substituents L. According to one aspect, the phenyl ring is 2,3,5-trisubstituted. According to a further aspect, the phenyl ring is 2,3,4-trisubstituted. According to yet a further aspect, the phenyl ring is 2,4,5-trisubstituted.

In a further embodiment of the invention, B is not ortho-methylphenyl.

In a further embodiment of the invention, B is not ortho- or para-trifluoromethylphenyl.

Unless indicated otherwise, L independently has the following preferred meanings:

According to one embodiment, L is independently selected from the group consisting of halogen, cyano, nitro, cyanato (OCN), C₁-C₄-alkoxy, C₁-C₄-haloalkoxy, C₃-C₆-cycloalkyl, C₃-C₆-halocycloalkyl, S-A¹, C(═O)A², C(═S)A², NA³A; where A¹, A², A³, A⁴ are as defined below:
A¹ is hydrogen, hydroxy, C₁-C₄-haloalkyl;
A² is one of the groups mentioned under A₁ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A³,A⁴ independently of one another are hydrogen, C₁-C₄-alkyl, C₁-C₄-haloalkyl;
where the aliphatic and/or alicyclic groups of the radical definitions of L for their part may carry one, two, three or four identical or different groups R^{L}:
R^{L} is halogen, cyano, nitro, C₁-C₄-alkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₈-alkylamino.

Furthermore preferably, L is independently selected from the group consisting of halogen, NO₂, amino, C₁-C₄-alkyl, C₁-C₄-alkoxy, C₁-C₄-haloalkoxy, C₄-alkylamino, di-C₁-C₄-alkylamino, thio and C₁-C₄-alkylthio.

Furthermore preferably, L is independently selected from the group consisting of halogen, C₁-C₄-alkyl, C₁-C₄-haloalkyl, C₁-C₄-alkoxy, C₁-C₄-haloalkoxy and C₁-C₄-haloalkylthio.

According to a further preferred embodiment, L is independently selected from the group consisting of F, Cl, Br, CH₃, C₂H₅, i-C₃H₇, t-C₄H₉, OCH₃, OC₂H₅, CF₃, CCl₃, CHF₂, CClF₂, OCF₃, OCHF₂ and SCF₃, in particular selected from the group consisting of F, Cl, CH₃, C₂H₅, OCH₃, OC₂H₅, CF₃, CHF₂, OCF₃, OCHF₂ and SCF₃. According to one aspect, L is independently selected from the group consisting of F, Cl, CH₃, OCH₃, CF₃, OCF₃ and OCHF₂. It may be preferred for L to be independently F or Cl.

In a preferred embodiment, the present invention relates to compounds of the formula I in which the variables have the following meanings:
A is phenyl which is substituted by one F and one further substituent L*, where the phenyl may additionally contain one or two substituents L, where L* is as defined below:
   L* fluorine, chlorine, iodine, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
      n is 0, 1 or 2;
      A¹ is hydrogen, hydroxyl, C₁-C₈-alkyl, C₁-C₈-haloalkyl, amino, C₁-C₈-alkylamino or di-C₁-C₈-alkylamino,
      A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
      A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
      where the aliphatic and/or alicyclic and/or aromatic groups of the radical definitions of L for their part may carry one, two, three or four identical or different groups R^{L}:
      R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
B is phenyl which is unsubstituted or substituted by one, two, three or four identical or different substituents L, where L is as defined below:
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-cycloalkenyloxy, hydroxyimino-C₁-C₈-alkyl, C₁-C₈-alkylene, oxy-C₂-C₄-alkylene, oxy-C₁-C₃-alkylenoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
      n is 0, 1 or 2;
      A¹ is hydrogen, hydroxyl, C₁-C₈-alkyl, C₁-C₈-haloalkyl, amino, C₁-C₈-alkylamino or di-C₁-C₈-alkylamino,
      A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
      A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
      where the aliphatic and/or alicyclic and/or aromatic groups of the radical definitions of L for their part may carry one, two, three or four identical or different groups R^{L}:
      R^{L} is halogen, cyano, nitro, C₁-C₈-alkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D is —S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³,
   R³ is C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
   a grouo DI

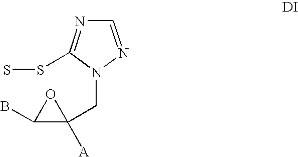

where A and B are as defined above;
a group DII

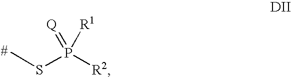

where # is the point of attachment to the triazolyl ring and Q, R¹, and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenylC₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂—, in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
a group SM, where M is as defined below:
   M is an alkali metal cation, an equivalent of an alkaline earth metal cation, an equivalent of a copper, zinc, iron or nickel cation or an ammonium cation of the formula (E)

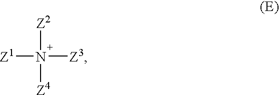

in which
Z¹ and Z² independently of one another are hydrogen or C₁-C₈-alkyl;
Z³ and Z⁴ independently of one another are hydrogen, C₁-C₈-alkyl, benzyl or phenyl; where the phenyl groups are in each case unsubstituted or substituted by one, two or three groups independently selected from the group consisting of halogen and C₁-C₄-alkyl;
   and agriculturally acceptable salts thereof.

In a preferred embodiment, the present invention relates to compounds of the formula I in which the variables have the meanings below:
A is phenyl which is substituted by one F and one further substituent L*, where the phenyl may additionally contain one or two substituents L, where L* is as defined below:
   L* is fluorine, chlorine, C₁-C₈-alkyl, C₁-C₈-alkylcarbonyloxy, C₃-C₈-cycloalkyl, C₃-C₈-cycloalkoxy,
B is phenyl which is unsubstituted or substituted by one, two, three or four identical or different substituents L, where L is as defined below:
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkylenoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
      n is 0, 1 or 2;
      A¹ is hydrogen, hydroxyl, C₁-C₈-alkyl, C₁-C₈-haloalkyl, amino, C₁-C₈-alkylamino or di-C₁-C₈-alkylamino,
      A² is one of the groups mentioned for A₁ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
      A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
      where the aliphatic and/or alicyclic and/or aromatic groups of the radical definitions of L for their part may carry one, two, three or four identical or different groups R^{L}:
      R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D is —S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
   R³ is C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
   a group DI

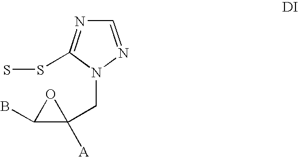

where A and B are as defined above;
a group DII

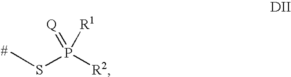

where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenylC₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂—, in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
a group SM, where M is as defined below:
M is an alkali metal cation, an equivalent of an alkaline earth metal cation, an equivalent of a copper, zinc, iron or nickel cation or an ammonium cation of the formula (E)

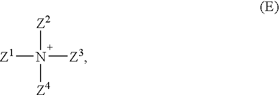

in which
Z¹, and Z² independently of one another are hydrogen or C₁-C₈-alkyl;
Z³ and Z⁴ independently of one another are hydrogen, C₁-C₈-alkyl, benzyl or phenyl; where the phenyl groups are in each case unsubstituted or substituted by one, two or three groups independently selected from the group consisting of halogen and C₁-C₄-alkyl;
   and agriculturally acceptable salts thereof.

In a further embodiment, B is phenyl which is unsubstituted or substituted by one, two or three substituents independently of one another selected from the group consisting of halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio and C₁-C₄-alkylthio.

The meanings described above of the variables A, B and L for compounds I apply, unless indicated otherwise, correspondingly to the precursors of the compounds according to the invention.

According to one embodiment of the invention, D is a group SR, where R is hydrogen (compounds I-1). According to a further embodiment, D is a group SR, where R is C₁-C₄-alkyl, in particular methyl or ethyl, preferably methyl.

According to a further embodiment of the invention, D is a group SR, where R is C(═O)R³ and R³ is NA³A⁴, where A³ and A⁴ independently of one another are hydrogen or C₁-C₈-alkyl.

According to a further embodiment of the invention, D is a group SR, where R is C(═O)R³ and R³ is hydrogen, C₁-C₄-alkyl, C₁-C₄-haloalkyl, C₁-C₄-alkoxy, C₁-C₄-haloalkoxy, phenyl or benzyl. According to a specific aspect thereof, R³ is hydrogen.

According to a further aspect thereof, R³ is C₁-C₄-alkyl, in particular methyl or ethyl, preferably methyl. According to yet a further aspect, R³ is C₁-C₄-haloalkyl, in particular trifluoromethyl. According to yet a further aspect, R³ is C₁-C₄-alkoxy, in particular methoxy or ethoxy.

According to a further embodiment of the invention, D is a group SR, where R is C(═O)R³ and R³ is (C₁-C₄)alkylamino, di(C₁-C₄)alkylamino or phenylamino. According to one aspect thereof, R³ is methylamino, dimethylamino, ethylamino, diethylamino or phenylamino.

According to a further embodiment of the invention, D is a group SR where R is CN.

According to a further embodiment of the invention, D is a group SR where R is SO₂R⁴ and R⁴ is C₁-C₄-alkyl, phenyl-C₁-C₄-alkyl or phenyl, where the phenyl groups are in each case unsubstituted or substituted by one, two or three groups independently selected from the group consisting of halogen and C₁-C₄-alkyl.

According to a further embodiment of the invention, D is a group SM where M is an alkali metal cation, an equivalent of an alkaline earth metal cation, an equivalent of a copper, zinc, iron or nickel cation or an ammonium cation of the formula (E)

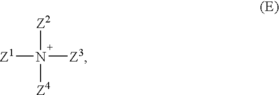

in which
Z¹ and Z² independently are hydrogen or C₁-C₄-alkyl; and
Z³ and Z⁴ independently are hydrogen, C₁-C₄-alkyl, benzyl or phenyl.

According to one embodiment, M is Na, 1/2 Cu, 1/3 Fe, HN(CH₃)₃, HN(C₂H₅)₃, N(CH₃)₄ or H₂N(C₃H₇)₂, in particular Na, 1/2 Cu, HN(CH₃)₃ or HN(C₂H₅)₃, especially Na, 1/2 Cu, HN(CH₃)₃ or HN(C₂H₅)₃.

According to a further embodiment of the invention, D is a group DI (compounds I-2) where A and B are independently as defined or preferred herein:

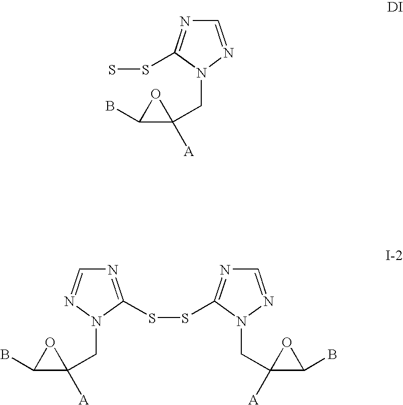

Preferably, both As and both Bs in the compounds 1-2 have the same meaning.

According to a further embodiment of the invention, D is a group DII where # is the point of attachment to the triazolyl ring and Q, R¹ and R² are as defined or preferred herein:

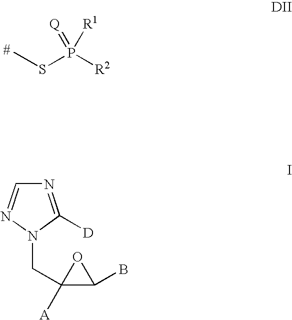

In particular with a view to their use, preference is given to the compounds I according to the invention compiled in Tables 1a to 93a and Tables 1b to 93b below. The groups mentioned for a substituent in the tables are furthermore per se, independently of the combination in which they are mentioned, a particularly preferred aspect of the substituent in question.

### Table 1a

Compounds I in which A is 2,3-difluorophenyl and the combination of B and D corresponds in each case to one row of Table A (compounds I.1aA-1 to I.1aA-2295)

### Table 2a

Compounds I in which A is 2,4-difluorophenyl and the combination of B and D corresponds in each case to one row of Table A (compounds I.2aA-1 to I.2aA-2295)

### Table 3a

Compounds I in which A is 2,5-difluorophenyl and the combination of B and D corresponds in each case to one row of Table A (compounds I.3aA-1 to I.3aA-2295)

### Table 4a

Compounds I in which A is 2,6-difluorophenyl and the combination of B and D corresponds in each case to one row of Table A (compounds I.4aA-1 to I.4aA-2295)

### Table 5a

Compounds I in which A is 3,4-difluorophenyl and the combination of B and D corresponds in each case to one row of Table A (compounds I.5aA-1 to I.5aA-2295)

### Table 6a

Compounds I in which A is 3,5-difluorophenyl and the combination of B and D corresponds in each case to one row of Table A (compounds I.6aA-1 to I.6aA-2295)

### Table 7a

Compounds I in which A is 2-fluoro-3-chlorophenyl and the combination of B and D corresponds in each case to one row of Table A (compounds I.7aA-1 to I.7aA-2295)

### Table 8a

Compounds I in which A is 2-fluoro-4-chlorophenyl and the combination of B and D corresponds in each case to one row of Table A (compounds I.8aA-1 to I.8aA-2295)

### Table 9a

Compounds I in which A is 2-fluoro-5-chlorophenyl and the combination of B and D corresponds in each case to one row of Table A (compounds I.9aA-1 to I.9aA-2295)

### Table 10a

Compounds I in which A is 2-fluoro-6-chlorophenyl I and the combination of B and D corresponds in each case to one row of Table A (compounds I.10aA-1 to I.10aA-2295)

### Table 11a

Compounds I in which A is 3-fluoro-4-chlorophenyl and the combination of B and D corresponds in each case to one row of Table A (compounds I.11aA-1 to I.11aA-2295)

### Table 12a

Compounds I in which A is 3-fluoro-5-chlorophenyl and the combination of B and D corresponds in each case to one row of Table A (compounds I.12aA-1 to I.12aA-2295)

### Table 13a

Compounds I in which A is 2-chloro-3-fluorophenyl and the combination of B and D corresponds in each case to one row of Table A (compounds I.13aA-1 to I.13aA-2295)

### Table 14a

Compounds I in which A is 2-chloro-4-fluorophenyl and the combination of B and D corresponds in each case to one row of Table A (compounds I.14aA-1 to I.14aA-2295)

### Table 15a

Compounds I in which A is 2-chloro-5-fluorophenyl and the combination of B and D corresponds in each case to one row of Table A (compounds I.15aA-1 to I.15aA-2295)

### Table 16a

Compounds I in which A is 3-chloro-4-fluorophenyl and the combination of B and D corresponds in each case to one row of Table A (compounds I.16aA-1 to I.16aA-2295)
   Table 17a Compounds I in which A is 2-methyl-3-fluorophenyl and the combination of B and D corresponds in each case to one row of Table A (compounds I.17aA-1 to I.17aA-2295)

### Table 18a

Compounds I in which A is 2-methyl-4-fluorophenyl and the combination of B and D corresponds in each case to one row of Table A (compounds I.18aA-1 to I.18aA-2295)

### Table 19a

Compounds I in which A is 2-methyl-5-fluorophenyl and the combination of B and D corresponds in each case to one row of Table A (compounds I.19aA-1 to I.19aA-2295)

### Table 20a

Compounds I in which A is 2-methyl-6-fluorophenyl and the combination of B and D corresponds in each case to one row of Table A (compounds I.20aA-1 to I.20aA-2295)

### Table 21a

Compounds I in which A is 3-methyl-4-fluorophenyl and the combination of B and D corresponds in each case to one row of Table A (compounds I.21aA-1 to I.21aA-2295)

### Table 22a

Compounds I in which A is 3-methyl-5-fluorophenyl and the combination of B and D corresponds in each case to one row of Table A (compounds I.22aA-1 to I.22aA-2295)

### Table 23a

Compounds I in which A is 2-fluoro-3-methylphenyl and the combination of B and D corresponds in each case to one row of Table A (compounds I.23aA-1 to I.23aA-2295)

### Table 24a

Compounds I in which A is 2-fluoro-4-methylphenyl and the combination of B and D corresponds in each case to one row of Table A (compounds I.24aA-1 to I.24aA-2295)

### Table 25a

Compounds I in which A is 2-fluoro-5-methylphenyl and the combination of B and D corresponds in each case to one row of Table A (compounds I.25aA-1 to I.25aA-2295)

### Table 26a

Compounds I in which A is 3-fluoro-4-methylphenyl and the combination of B and D corresponds in each case to one row of Table A (compounds I.26aA-1 to I.26aA-2295)

### Table 27a

Compounds I in which A is 2-ethyl-3-fluorophenyl and the combination of B and D corresponds in each case to one row of Table A (compounds I.27aA-1 to I.27aA-2295)

### Table 28a

Compounds I in which A is 2-ethyl-4-fluorophenyl and the combination of B and D corresponds in each case to one row of Table A (compounds I.28aA-1 to I.28aA-2295)

### Table 29a

Compounds I in which A is 2-ethyl-5-fluorophenyl and the combination of B and D corresponds in each case to one row of Table A (compounds I.29aA-1 to I.29aA-2295)

### Table 30a

Compounds I in which A is 2-ethyl-6-fluorophenyl and the combination of B and D corresponds in each case to one row of Table A (compounds I.30aA-1 to I.30aA-2295)

### Table 31a

Compounds I in which A is 3-ethyl-4-fluorophenyl and the combination of B and D corresponds in each case to one row of Table A (compounds I.31aA-1 to I.31aA-2295)

### Table 32a

Compounds I in which A is 3-ethyl-5-fluorophenyl and the combination of B and D corresponds in each case to one row of Table A (compounds I.32aA-1 to I.32aA-2295)

### Table 33a

Compounds I in which A is 2-fluoro-3-ethylphenyl and the combination of B and D corresponds in each case to one row of Table A (compounds I.33aA-1 to I.33aA-2295)

### Table 34a

Compounds I in which A is 2-fluoro-4-ethylphenyl and the combination of B and D corresponds in each case to one row of Table A (compounds I.34aA-1 to I.34aA-2295)

### Table 35a

Compounds I in which A is 2-fluoro-5-ethylphenyl and the combination of B and D corresponds in each case to one row of Table A (compounds I.35aA-1 to I.35aA-2295)

### Table 36a

Compounds I in which A is 3-fluoro-4-ethylphenyl and the combination of B and D corresponds in each case to one row of Table A (compounds I.36aA-1 to I.36aA-2295)

### Table 37a

Compounds I in which A is 2-methoxy-3-fluorophenyl and the combination of B and D corresponds in each case to one row of Table A (compounds I.37aA-1 to I.37aA-2295)

### Table 38a

Compounds I in which A is 2-methoxy-4-fluorophenyl and the combination of B and D corresponds in each case to one row of Table A (compounds I.38aA-1 to I.38aA-2295)

### Table 39a

Compounds I in which A is 2-methoxy-5-fluorophenyl and the combination of B and D corresponds in each case to one row of Table A (compounds I.39aA-1 to I.39aA-2295)

### Table 40a

Compounds I in which A is 2-methoxy-6-fluorophenyl and the combination of B and D corresponds in each case to one row of Table A (compounds I.40aA-1 to I.40aA-2295)

### Table 41a

Compounds I in which A is 3-methoxy-4-fluorophenyl and the combination of B and D corresponds in each case to one row of Table A (compounds I.41 aA-1 to I.41aA-2295)

### Table 42a

Compounds I in which A is 3-methoxy-5-fluorophenyl and the combination of B and D corresponds in each case to one row of Table A (compounds I.42aA-1 to I.42aA-2295)

### Table 43a

Compounds I in which A is 2-fluoro-3-methoxyphenyl and the combination of B and D corresponds in each case to one row of Table A (compounds I.43aA-1 to I.43aA-2295)

### Table 44a

Compounds I in which A is 2-fluoro-4-methoxyphenyl and the combination of B and D corresponds in each case to one row of Table A (compounds I.44aA-1 to I.44aA-2295)

### Table 45a

Compounds I in which A is 2-fluoro-5-methoxyphenyl and the combination of B and D corresponds in each case to one row of Table A (compounds I.45aA-1 to I.45aA-2295)

### Table 46a

Compounds I in which A is 3-fluoro-4-methoxyphenyl and the combination of B and D corresponds in each case to one row of Table A (compounds I.46aA-1 to I.46aA-2295)

### Table 47a

Compounds I in which A is 3-fluoro-5-methoxyphenyl and the combination of B and D corresponds in each case to one row of Table A (compounds I.47aA-1 to I.47aA-2295)

### Table 48a

Compounds I in which A is 2-(trifluoromethyl)-3-fluorophenyl and the combination of B and D corresponds in each case to one row of Table A (compounds I.48aA-1 to I.48aA-2295)

### Table 49a

Compounds I in which A is 2-(trifluoromethyl)-4-fluorophenyl and the combination of B and D corresponds in each case to one row of Table A (compounds I.49aA-1 to I.49aA-2295)

### Table 50a

Compounds I in which A is 2-(trifluoromethyl)-5-fluorophenyl and the combination of B and D corresponds in each case to one row of Table A (compounds I.50aA-1 to I.50aA-2295)

### Table 51a

Compounds I in which A is 2-(trifluoromethyl)-6-fluorophenyl and the combination of B and D corresponds in each case to one row of Table A (compounds I.51 aA-1 to I.51aA-2295)

### Table 52a

Compounds I in which A is 3-(trifluoromethyl)-4-fluorophenyl and the combination of B and D corresponds in each case to one row of Table A (compounds I.52aA-1 to I.52aA-2295)

### Table 53a

Compounds I in which A is 3-(trifluoromethyl)-5-fluorophenyl and the combination of B and D corresponds in each case to one row of Table A (compounds I.53aA-1 to I.53aA-2295)

### Table 54a

Compounds I in which A is 2-fluoro-3-(trifluoromethyl)phenyl and the combination of B and D corresponds in each case to one row of Table A (compounds I.54aA-1 to I.54aA-2295)

### Table 55a

Compounds I in which A is 2-fluoro-4-(trifluoromethyl)phenyl and the combination of B and D corresponds in each case to one row of Table A (compounds I.55aA-1 to I.55aA-2295)

### Table 56a

Compounds I in which A is 2-fluoro-5-(trifluoromethyl)phenyl and the combination of B and D corresponds in each case to one row of Table A (compounds I.56aA-1 to I.56aA-2295)

### Table 57a

Compounds I in which A is 3-fluoro-4-(trifluoromethyl)phenyl and the combination of B and D corresponds in each case to one row of Table A (compounds I.57aA-1 to I.57aA-2295)

### Table 58a

Compounds I in which A is 2-(trifluoromethoxy)-3-fluorophenyl and the combination of B and D corresponds in each case to one row of Table A (compounds I.58aA-1 to I.58aA-2295)

### Table 59a

Compounds I in which A is 2-(trifluoromethoxy)-4-fluorophenyl and the combination of B and D corresponds in each case to one row of Table A (compounds I.59aA-1 to I.59aA-2295)

### Table 60a

Compounds I in which A is 2-(trifluoromethoxy)-5-fluorophenyl and the combination of B and D corresponds in each case to one row of Table A (compounds I.60aA-1 to I.60aA-2295)

### Table 61a

Compounds I in which A is 2-(trifluoromethoxy)-6-fluorophenyl and the combination of B and D corresponds in each case to one row of Table A (compounds I.61aA-1 to I.61 aA-2295)

### Table 62a

Compounds I in which A is 3-(trifluoromethoxy)-4-fluorophenyl and the combination of B and D corresponds in each case to one row of Table A (compounds I.62aA-1 to I.62aA-2295)

### Table 63a

Compounds I in which A is 3-(trifluoromethoxy)-5-fluorophenyl and the combination of B and D corresponds in each case to one row of Table A (compounds I.63aA-1 to I.63aA-2295)

### Table 64a

Compounds I in which A is 2-fluoro-3-(trifluoromethoxy)phenyl and the combination of B and D corresponds in each case to one row of Table A (compounds I.64aA-1 to I.64aA-2295)

### Table 65a

Compounds I in which A is 2-fluoro-4-(trifluoromethoxy)phenyl and the combination of B and D corresponds in each case to one row of Table A (compounds I.65aA-1 to I.65aA-2295)

### Table 66a

Compounds I in which A is 2-fluoro-5-(trifluoromethoxy)phenyl and the combination of B and D corresponds in each case to one row of Table A (compounds I.66aA-1 to I.66aA-2295)

### Table 67a

Compounds I in which A is 3-fluoro-4-(trifluoromethoxy)phenyl and the combination of B and D corresponds in each case to one row of Table A (compounds I.67aA-1 to I.67aA-2295)

### Table 68a

Compounds I in which A is 2-(difluoromethoxy)-3-fluorophenyl and the combination of B and D corresponds in each case to one row of Table A (compounds I.68aA-1 to I.68aA-2295)

### Table 69a

Compounds I in which A is 2-(difluoromethoxy)-4-fluorophenyl and the combination of B and D corresponds in each case to one row of Table A (compounds I.69aA-1 to I.69aA-2295)

### Table 70a

Compounds I in which A is 2-(difluoromethoxy)-5-fluorophenyl and the combination of B and D corresponds in each case to one row of Table A (compounds I.70aA-1 to I.70aA-2295)

### Table 71a

Compounds I in which A is 2-(difluoromethoxy)-6-fluorophenyl and the combination of B and D corresponds in each case to one row of Table A (compounds I.71aA-1 to I.71aA-2295)

### Table 72a

Compounds I in which A is 3-(difluoromethoxy)-4-fluorophenyl and the combination of B and D corresponds in each case to one row of Table A (compounds I.72aA-1 to I.72aA-2295)

### Table 73a

Compounds I in which A is 3-(difluoromethoxy)-5-fluorophenyl and the combination of B and D corresponds in each case to one row of Table A (compounds I.73aA-1 to I.73aA-2295)

### Table 74a

Compounds I in which A is 2-fluoro-3-(difluoromethoxy)phenyl and the combination of B and D corresponds in each case to one row of Table A (compounds I.74aA-1 to I.74aA-2295)

### Table 75a

Compounds I in which A is 2-fluoro-4-(difluoromethoxy)phenyl and the combination of B and D corresponds in each case to one row of Table A (compounds I.75aA-1 to I.75aA-2295)

### Table 76a

Compounds I in which A is 2-fluoro-5-(difluoromethoxy)phenyl and the combination of B and D corresponds in each case to one row of Table A (compounds I.76aA-1 to I.76aA-2295)

### Table 77a

Compounds I in which A is 3-fluoro-4-(difluoromethoxy)phenyl and the combination of B and D corresponds in each case to one row of Table A (compounds I.77aA-1 to I.77aA-2295)

### Table 78a

Compounds I in which A is 2-(trifluoromethylthio)-3-fluorophenyl and the combination of B and D corresponds in each case to one row of Table A (compounds I.78aA-1 to I.78aA-2295)

### Table 79a

Compounds I in which A is 2-(trifluoromethylthio)-4-fluorophenyl and the combination of B and D corresponds in each case to one row of Table A (compounds I.79aA-1 to I.79aA-2295)

### Table 80a

Compounds I in which A is 2-(trifluoromethylthio)-5-fluorophenyl and the combination of B and D corresponds in each case to one row of Table A (compounds I.80aA-1 to I.80aA-2295)

### Table 81a

Compounds I in which A is 2-(trifluoromethylthio)-6-fluorophenyl and the combination of B and D corresponds in each case to one row of Table A (compounds I.81aA-1 to I.81aA-2295)

### Table 82a

Compounds I in which A is 3-(trifluoromethylthio)-4-fluorophenyl and the combination of B and D corresponds in each case to one row of Table A (compounds I.82aA-1 to I.82aA-2295)

### Table 83a

Compounds I in which A is 3-(trifluoromethylthio)-5-fluorophenyl and the combination of B and D corresponds in each case to one row of Table A (compounds I.83aA-1 to I.83aA-2295)

### Table 84a

Compounds I in which A is 2-fluoro-3-(trifluoromethylthio)phenyl and the combination of B and D corresponds in each case to one row of Table A (compounds I.84aA-1 to I.84aA-2295)

### Table 85a

Compounds I in which A is 2-fluoro-4-(trifluoromethylthio)phenyl and the combination of B and D corresponds in each case to one row of Table A (compounds I.85aA-1 to I.85aA-2295)

### Table 86a

Compounds I in which A is 2-fluoro-5-(trifluoromethylthio)phenyl and the combination of B and D corresponds in each case to one row of Table A (compounds I.86aA-1 to I.86aA-2295)

### Table 87a

Compounds I in which A is 3-fluoro-4-(trifluoromethylthio)phenyl and the combination of B and D corresponds in each case to one row of Table A (compounds I.87aA-1 to I.87aA-2295)

### Table 88a

Compounds I in which A is 2,3,4-trifluorophenyl and the combination of B and D corresponds in each case to one row of Table A (compounds I.88aA-1 to I.88aA-2295)

### Table 89a

Compounds I in which A is 2,3,5-trifluorophenyl and the combination of B and D corresponds in each case to one row of Table A (compounds I.89aA-1 to I.89aA-2295)

### Table 90a

Compounds I in which A is 2,3,6-trifluorophenyl and the combination of B and D corresponds in each case to one row of Table A (compounds I.90aA-1 to I.90aA-2295)

### Table 91a

Compounds I in which A is 2,4,5-trifluorophenyl and the combination of B and D corresponds in each case to one row of Table A (compounds I.91aA-1 to I.91aA-2295)

### Table 92a

Compounds I in which A is 2,4,6-trifluorophenyl and the combination of B and D corresponds in each case to one row of Table A (compounds I.92aA-1 to I.92aA-2295)

### Table 93a

Compounds I in which A is 3,4,5-trifluorophenyl and the combination of B and D corresponds in each case to one row of Table A (compounds I.93aA-1 to I.93aA-2295)

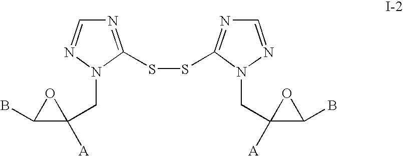

### Table 1b

Compounds I-2 in which both A are 2,3-difluorophenyl and B corresponds in each case to one row of Table B (compounds I-2.1bB-1 to I-2.1bB-255)

### Table 2b

Compounds I-2 in which both A are 2,4-difluorophenyl and B corresponds in each case to one row of Table B (compounds I-2.2bB-1 to I-2.2bB-255)

### Table 3b

Compounds I-2 in which both A are 2,5-difluorophenyl and B corresponds in each case to one row of Table B (compounds I-2.3bB-1 to I-2.3bB-255)

### Table 4b

Compounds I-2 in which both A are 2,6-difluorophenyl and B corresponds in each case to one row of Table B (compounds I-2.4bB-1 to I-2.4bB-255)

### Table 5b

Compounds I-2 in which both A are 3,4-difluorophenyl and B corresponds in each case to one row of Table B (compounds I-2.5bB-1 to I-2.5bB-255)

### Table 6b

Compounds I-2 in which both A are 3,5-difluorophenyl and B corresponds in each case to one row of Table B (compounds I-2.6bB-1 to I-2.6bB-255)

### Table 7b

Compounds I-2 in which both A are 2-fluoro-3-chlorophenyl and B corresponds in each case to one row of Table B (compounds I-2.7bB-1 to I-2.7bB-255)

### Table 8b

Compounds I-2 in which both A are 2-fluoro-4-chlorophenyl and B corresponds in each case to one row of Table B (compounds I-2.8bB-1 to I-2.8bB-255)

### Table 9b

Compounds I-2 in which both A are 2-fluoro-5-chlorophenyl and B corresponds in each case to one row of Table B (compounds I-2.9bB-1 to I-2.9bB-255)

### Table 10b

Compounds I-2 in which both A are 2-fluoro-6-chlorophenyl I and B corresponds in each case to one row of Table B (compounds I-2.10bB-1 to I-2.10bB-255)

### Table 11b

Compounds I-2 in which both A are 3-fluoro-4-chlorophenyl and B corresponds in each case to one row of Table B (compounds I-2.11bB-1 to I-2.11bB-255)

### Table 12b

Compounds I-2 in which both A are 3-fluoro-5-chlorophenyl and B corresponds in each case to one row of Table B (compounds I-2.12bB-1 to I-2.12bB-255)

### Table 13b

Compounds I-2 in which both A are 2-chloro-3-fluorophenyl and B corresponds in each case to one row of Table B (compounds I-2.13bB-1 to I-2.13bB-255)

### Table 14b

Compounds I-2 in which both A are 2-chloro-4-fluorophenyl and B corresponds in each case to one row of Table B (compounds I-2.14bB-1 to I-2.14bB-255)

### Table 15b

Compounds I-2 in which both A are 2-chloro-5-fluorophenyl and B corresponds in each case to one row of Table B (compounds I-2.15bB-1 to I-2.15bB-255)

### Table 16b

Compounds I-2 in which both A are 3-chloro-4-fluorophenyl and B corresponds in each case to one row of Table B (compounds I-2.16bB-1 to I-2.16bB-255)

### Table 17b

Compounds I-2 in which both A are 2-methyl-3-fluorophenyl and B corresponds in each case to one row of Table B (compounds I-2.17bB-1 to I-2.17bB-255)

### Table 18b

Compounds I-2 in which both A are 2-methyl-4-fluorophenyl and B corresponds in each case to one row of Table B (compounds I-2.18bB-1 to I-2.18bB-255)

### Table 19b

Compounds I-2 in which both A are 2-methyl-5-fluorophenyl and B corresponds in each case to one row of Table B (compounds I-2.19bB-1 to I-2.19bB-255)

### Table 20b

Compounds I-2 in which both A are 2-methyl-6-fluorophenyl and B corresponds in each case to one row of Table B (compounds I-2.20bB-1 to I-2.20bB-255)

### Table 21b

Compounds I-2 in which both A are 3-methyl-4-fluorophenyl and B corresponds in each case to one row of Table B (compounds I-2.21bB-1 to I-2.21bB-255)

### Table 22b

Compounds I-2 in which both A are 3-methyl-5-fluorophenyl and B corresponds in each case to one row of Table B (compounds I-2.22bB-1 to I-2.22bB-255)

### Table 23b

Compounds I-2 in which both A are 2-fluoro-3-methylphenyl and B corresponds in each case to one row of Table B (compounds I-2.23bB-1 to I-2.23bB-255)

### Table 24b

Compounds I-2 in which both A are 2-fluoro-4-methylphenyl and B corresponds in each case to one row of Table B (compounds I-2.24bB-1 to I-2.24bB-255)

### Table 25b

Compounds I-2 in which both A are 2-fluoro-5-methylphenyl and B corresponds in each case to one row of Table B (compounds I-2.25bB-1 to I-2.25bB-255)

### Table 26b

Compounds I-2 in which both A are 3-fluoro-4-methylphenyl and B corresponds in each case to one row of Table B (compounds I-2.26bB-1 to I-2.26bB-255)

### Table 27b

Compounds I-2 in which both A are 2-ethyl-3-fluorophenyl and B corresponds in each case to one row of Table B (compounds I-2.27bB-1 to I-2.27bB-255)

### Table 28b

Compounds I-2 in which both A are 2-ethyl-4-fluorophenyl and B corresponds in each case to one row of Table B (compounds I-2.28bB-1 to I-2.28bB-255)

### Table 29b

Compounds I-2 in which both A are 2-ethyl-5-fluorophenyl and B corresponds in each case to one row of Table B (compounds I-2.29bB-1 to I-2.29bB-255)

### Table 30b

Compounds I-2 in which both A are 2-ethyl-6-fluorophenyl and B corresponds in each case to one row of Table B (compounds I-2.30bB-1 to I-2.30bB-255)

### Table 31b

Compounds I-2 in which both A are 3-ethyl-4-fluorophenyl and B corresponds in each case to one row of Table B (compounds I-2.31bB-1 to I-2.31bB-255)

### Table 32b

Compounds I-2 in which both A are 3-ethyl-5-fluorophenyl and B corresponds in each case to one row of Table B (compounds I-2.32bB-1 to I-2.32bB-255)

### Table 33b

Compounds I-2 in which both A are 2-fluoro-3-ethylphenyl and B corresponds in each case to one row of Table B (compounds I-2.33bB-1 to I-2.33bB-255)

### Table 34b

Compounds I-2 in which both A are 2-fluoro-4-ethylphenyl and B corresponds in each case to one row of Table B (compounds I-2.34bB-1 to I-2.34bB-255)

### Table 35b

Compounds I-2 in which both A are 2-fluoro-5-ethylphenyl and B corresponds in each case to one row of Table B (compounds I-2.35bB-1 to I-2.35bB-255)

### Table 36b

Compounds I-2 in which both A are 3-fluoro-4-ethylphenyl and B corresponds in each case to one row of Table B (compounds I-2.36bB-1 to I-2.36bB-255)

### Table 37b

Compounds I-2 in which both A are 2-methoxy-3-fluorophenyl and B corresponds in each case to one row of Table B (compounds I-2.37bB-1 to I-2.37bB-255)

### Table 38b

Compounds I-2 in which both A are 2-methoxy-4-fluorophenyl and B corresponds in each case to one row of Table B (compounds I-2.38bB-1 to I-2.38bB-255)

### Table 39b

Compounds I-2 in which both A are 2-methoxy-5-fluorophenyl and B corresponds in each case to one row of Table B (compounds I-2.39bB-1 to I-2.39bB-255)

### Table 40b

Compounds I-2 in which both A are 2-methoxy-6-fluorophenyl and B corresponds in each case to one row of Table B (compounds I-2.40bB-1 to I-2.40bB-255)

### Table 41b

Compounds I-2 in which both A are 3-methoxy-4-fluorophenyl and B corresponds in each case to one row of Table B (compounds I-2.41 bB-1 to I-2.41 bB-255)

### Table 42b

Compounds I-2 in which both A are 3-methoxy-5-fluorophenyl and B corresponds in each case to one row of Table B (compounds I-2.42bB-1 to I-2.42bB-255)

### Table 43b

Compounds I-2 in which both A are 2-fluoro-3-methoxyphenyl and B corresponds in each case to one row of Table B (compounds I-2.43bB-1 to I-2.43bB-255)

### Table 44b

Compounds I-2 in which both A are 2-fluoro-4-methoxyphenyl and B corresponds in each case to one row of Table B (compounds I-2.44bB-1 to I-2.44bB-255)

### Table 45b

Compounds I-2 in which both A are 2-fluoro-5-methoxyphenyl and B corresponds in each case to one row of Table B (compounds I-2.45bB-1 to I-2.45bB-255)

### Table 46b

Compounds I-2 in which both A are 3-fluoro-4-methoxyphenyl and B corresponds in each case to one row of Table B (compounds I-2.46bB-1 to I-2.46bB-255)

### Table 47b

Compounds I-2 in which both A are 3-fluoro-5-methoxyphenyl and B corresponds in each case to one row of Table B (compounds I-2.47bB-1 to I-2.47bB-255)

### Table 48b

Compounds I-2 in which both A are 2-(trifluoromethyl)-3-fluorophenyl and B corresponds in each case to one row of Table B (compounds I-2.48bB-1 to I-2.48bB-255)

### Table 49b

Compounds I-2 in which both A are 2-(trifluoromethyl)-4-fluorophenyl and B corresponds in each case to one row of Table B (compounds I-2.49bB-1 to I-2.49bB-255)

### Table 50b

Compounds I-2 in which both A are 2-(trifluoromethyl)-5-fluorophenyl and B corresponds in each case to one row of Table B (compounds I-2.50bB-1 to I-2.50bB-255)

### Table 51b

Compounds I-2 in which both A are 2-(trifluoromethyl)-6-fluorophenyl and B corresponds in each case to one row of Table B (compounds I-2.51bB-1 to I-2.51bB-255)

### Table 52b

Compounds I-2 in which both A are 3-(trifluoromethyl)-4-fluorophenyl and B corresponds in each case to one row of Table B (compounds I-2.52bB-1 to I-2.52bB-255)

### Table 53b

Compounds I-2 in which both A are 3-(trifluoromethyl)-5-fluorophenyl and B corresponds in each case to one row of Table B (compounds I-2.53bB-1 to I-2.53bB-255)

### Table 54b

Compounds I-2 in which both A are 2-fluoro-3-(trifluoromethyl)phenyl and B corresponds in each case to one row of Table B (compounds I-2.54bB-1 to I-2.54bB-255)

### Table 55b

Compounds I-2 in which both A are 2-fluoro-4-(trifluoromethyl)phenyl and B corresponds in each case to one row of Table B (compounds I-2.55bB-1 to I-2.55bB-255)

### Table 56b

Compounds I-2 in which both A are 2-fluoro-5-(trifluoromethyl)phenyl and B corresponds in each case to one row of Table B (compounds I-2.56bB-1 to I-2.56bB-255)

### Table 57b

Compounds I-2 in which both A are 3-fluoro-4-(trifluoromethyl)phenyl and B corresponds in each case to one row of Table B (compounds I-2.57bB-1 to I-2.57bB-255)

### Table 58b

Compounds I-2 in which both A are 2-(trifluoromethoxy)-3-fluorophenyl and B corresponds in each case to one row of Table B (compounds I-2.58bB-1 to I-2.58bB-255)

### Table 59b

Compounds I-2 in which both A are 2-(trifluoromethoxy)-4-fluorophenyl and B corresponds in each case to one row of Table B (compounds I-2.59bB-1 to I-2.59bB-255)

### Table 60b

Compounds I-2 in which both A are 2-(trifluoromethoxy)-5-fluorophenyl and B corresponds in each case to one row of Table B (compounds I-2.60bB-1 to I-2.60bB-255)

### Table 61b

Compounds I-2 in which both A are 2-(trifluoromethoxy)-6-fluorophenyl and B corresponds in each case to one row of Table B (compounds I-2.61bB-1 to I-2.61bB-255)

### Table 62b

Compounds I-2 in which both A are 3-(trifluoromethoxy)-4-fluorophenyl and B corresponds in each case to one row of Table B (compounds I-2.62bB-1 to I-2.62bB-255)

### Table 63b

Compounds I-2 in which both A are 3-(trifluoromethoxy)-5-fluorophenyl and B corresponds in each case to one row of Table B (compounds I-2.63bB-1 to I-2.63bB-255)

### Table 64b

Compounds I-2 in which both A are 2-fluoro-3-(trifluoromethoxy)phenyl and B corresponds in each case to one row of Table B (compounds I-2.64bB-1 to I-2.64bB-255)

### Table 65b

Compounds I-2 in which both A are 2-fluoro-4-(trifluoromethoxy)phenyl and B corresponds in each case to one row of Table B (compounds I-2.65bB-1 to I-2.65bB-255)

### Table 66b

Compounds I-2 in which both A are 2-fluoro-5-(trifluoromethoxy)phenyl and B corresponds in each case to one row of Table B (compounds I-2.66bB-1 to I-2.66bB-255)

### Table 67b

Compounds I-2 in which both A are 3-fluoro-4-(trifluoromethoxy)phenyl and B corresponds in each case to one row of Table B (compounds I-2.67bB-1 to I-2.67bB-255)

### Table 68b

Compounds I-2 in which both A are 2-(difluoromethoxy)-3-fluorophenyl and B corresponds in each case to one row of Table B (compounds I-2.68bB-1 to I-2.68bB-255)

### Table 69b

Compounds I-2 in which both A are 2-(difluoromethoxy)-4-fluorophenyl and B corresponds in each case to one row of Table B (compounds I-2.69bB-1 to I-2.69bB-255)

### Table 70b

Compounds I-2 in which both A are 2-(difluoromethoxy)-5-fluorophenyl and B corresponds in each case to one row of Table B (compounds I-2.70bB-1 to I-2.70bB-255)

### Table 71b

Compounds I-2 in which both A are 2-(difluoromethoxy)-6-fluorophenyl and B corresponds in each case to one row of Table B (compounds I-2.71 bB-1 to I-2.71 bB-255)

### Table 72b

Compounds I-2 in which both A are 3-(difluoromethoxy)-4-fluorophenyl and B corresponds in each case to one row of Table B (compounds I-2.72bB-1 to I-2.72bB-255)

### Table 73b

Compounds I-2 in which both A are 3-(difluoromethoxy)-5-fluorophenyl and B corresponds in each case to one row of Table B (compounds I-2.73bB-1 to I-2.73bB-255)

### Table 74b

Compounds I-2 in which both A are 2-fluoro-3-(difluoromethoxy)phenyl and B corresponds in each case to one row of Table B (compounds I-2.74bB-1 to I-2.74bB-255)

### Table 75b

Compounds I-2 in which both A are 2-fluoro-4-(difluoromethoxy)phenyl and B corresponds in each case to one row of Table B (compounds I-2.75bB-1 to I-2.75bB-255)

### Table 76b

Compounds I-2 in which both A are 2-fluoro-5-(difluoromethoxy)phenyl and B corresponds in each case to one row of Table B (compounds I-2.76bB-1 to I-2.76bB-255)

### Table 77b

Compounds I-2 in which both A are 3-fluoro-4-(difluoromethoxy)phenyl and B corresponds in each case to one row of Table B (compounds I-2.77bB-1 to I-2.77bB-255)

### Table 78b

Compounds I-2 in which both A are 2-(trifluoromethylthio)-3-fluorophenyl and B corresponds in each case to one row of Table B (compounds I-2.78bB-1 to I-2.78bB-255)

### Table 79b

Compounds I-2 in which both A are 2-(trifluoromethylthio)-4-fluorophenyl and B corresponds in each case to one row of Table B (compounds I-2.79bB-1 to I-2.79bB-255)

### Table 80b

Compounds I-2 in which both A are 2-(trifluoromethylthio)-5-fluorophenyl and B corresponds in each case to one row of Table B (compounds I-2.80bB-1 to I-2.80bB-255)

### Table 81b

Compounds I-2 in which both A are 2-(trifluoromethylthio)-6-fluorophenyl and B corresponds in each case to one row of Table B (compounds I-2.81 bB-1 to I-2.81 bB-255)

### Table 82b

Compounds I-2 in which both A are 3-(trifluoromethylthio)-4-fluorophenyl and B corresponds in each case to one row of Table B (compounds I-2.82bB-1 to I-2.82bB-255)

### Table 83b

Compounds I-2 in which both A are 3-(trifluoromethylthio)-5-fluorophenyl and B corresponds in each case to one row of Table B (compounds I-2.83bB-1 to I-2.83bB-255)

### Table 84b

Compounds I-2 in which both A are 2-fluoro-3-(trifluoromethylthio)phenyl and B corresponds in each case to one row of Table B (compounds I-2.84bB-1 to I-2.84bB-255)

### Table 85b

Compounds I-2 in which both A are 2-fluoro-4-(trifluoromethylthio)phenyl and B corresponds in each case to one row of Table B (compounds I-2.85bB-1 to I-2.85bB-255)

### Table 86b

Compounds I-2 in which both A are 2-fluoro-5-(trifluoromethylthio)phenyl and B corresponds in each case to one row of Table B (compounds I-2.86bB-1 to I-2.86bB-255)

### Table 87b

Compounds I-2 in which both A are 3-fluoro-4-(trifluoromethylthio)phenyl and B corresponds in each case to one row of Table B (compounds I-2.87bB-1 to I-2.87bB-255)

### Table 88b

Compounds I-2 in which both A are 2,3,4-trifluorophenyl and B corresponds in each case to one row of Table B (compounds I-2.88bB-1 to I-2.88bB-255)

### Table 89b

Compounds I-2 in which both A are 2,3,5-trifluorophenyl and B corresponds in each case to one row of Table B (compounds I-2.89bB-1 to I-2.89bB-255)

### Table 90b

Compounds I-2 in which both A are 2,3,6-trifluorophenyl and B corresponds in each case to one row of Table B (compounds I-2.90bB-1 to I-2.90bB-255)

### Table 91b

Compounds I-2 in which both A are 2,4,5-trifluorophenyl and B corresponds in each case to one row of Table B (compounds I-2.91bB-1 to I-2.91bB-255)

### Table 92b

Compounds I-2 in which both A are 2,4,6-trifluorophenyl and B corresponds in each case to one row of Table B (compounds I-2.92bB-1 to I-2.92bB-255)

### Table 93b

Compounds I-2 in which both A are 3,4,5-trifluorophenyl and B corresponds in each case to one row of Table B (compounds I-2.93bB-1 to I-2.93bB-255)

TABLE A

| Row | B | D |
|---|---|---|
| A-1 | phenyl | SH |
| A-2 | 2-chlorophenyl | SH |
| A-3 | 3-chlorophenyl | SH |
| A-4 | 4-chlorophenyl | SH |
| A-5 | 2-fluorophenyl | SH |
| A-6 | 3-fluorophenyl | SH |
| A-7 | 4-fluorophenyl | SH |
| A-8 | 2-methylphenyl | SH |
| A-9 | 3-methylphenyl | SH |
| A-10 | 4-methylphenyl | SH |
| A-11 | 2-ethylphenyl | SH |
| A-12 | 3-ethylphenyl | SH |
| A-13 | 4-ethylphenyl | SH |
| A-14 | 2-methoxyphenyl | SH |
| A-15 | 3-methoxyphenyl | SH |
| A-16 | 4-methoxyphenyl | SH |
| A-17 | 2-trifluoromethylphenyl | SH |
| A-18 | 3-trifluoromethylphenyl | SH |
| A-19 | 4-trifluoromethylphenyl | SH |
| A-20 | 2-trifluoromethoxyphenyl | SH |
| A-21 | 3-trifluoromethoxyphenyl | SH |
| A-22 | 4-trifluoromethoxyphenyl | SH |
| A-23 | 2-difluoromethoxyphenyl | SH |
| A-24 | 3-difluoromethoxyphenyl | SH |
| A-25 | 4-difluoromethoxyphenyl | SH |
| A-26 | 2-trifluoromethylthiophenyl | SH |
| A-27 | 3-trifluoromethylthiophenyl | SH |
| A-28 | 4-trifluoromethylthiophenyl | SH |
| A-29 | 2,3-dichlorophenyl | SH |
| A-30 | 2,4-dichlorophenyl | SH |
| A-31 | 2,5-dichlorophenyl | SH |
| A-32 | 2,6-dichlorophenyl | SH |
| A-33 | 3,4-dichlorophenyl | SH |
| A-34 | 3,5-dichlorophenyl | SH |
| A-35 | 2,3-difluorophenyl | SH |
| A-36 | 2,4-difluorophenyl | SH |
| A-37 | 2,5-difluorophenyl | SH |
| A-38 | 2,6-difluorophenyl | SH |
| A-39 | 3,4-difluorophenyl | SH |
| A-40 | 3,5-difluorophenyl | SH |
| A-41 | 2,3-dimethylphenyl | SH |
| A-42 | 2,4-dimethylphenyl | SH |
| A-43 | 2,5-dimethylphenyl | SH |
| A-44 | 2,6-dimethylphenyl | SH |
| A-45 | 3,4-dimethylphenyl | SH |
| A-46 | 3,5-dimethylphenyl | SH |
| A-47 | 2,3-diethylphenyl | SH |
| A-48 | 2,4-diethylphenyl | SH |
| A-49 | 2,5-diethylphenyl | SH |
| A-50 | 2,6-diethylphenyl | SH |
| A-51 | 3,5-diethylphenyl | SH |
| A-52 | 3,4-diethylphenyl | SH |
| A-53 | 2,3-dimethoxyphenyl | SH |
| A-54 | 2,4-dimethoxyphenyl | SH |
| A-55 | 2,5-dimethoxyphenyl | SH |
| A-56 | 2,6-dimethoxyphenyl | SH |
| A-57 | 3,4-dimethoxyphenyl | SH |
| A-58 | 3,5-dimethoxyphenyl | SH |
| A-59 | 2,3-di(trifluoromethyl)phenyl | SH |
| A-60 | 2,4-di(trifluoromethyl)phenyl | SH |
| A-61 | 2,5-di(trifluoromethyl)phenyl | SH |
| A-62 | 2,6-di(trifluoromethyl)phenyl | SH |
| A-63 | 3,4-di(trifluoromethyl)phenyl | SH |
| A-64 | 3,5-di(trifluoromethyl)phenyl | SH |
| A-65 | 2,3-di(trifluoromethoxy)phenyl | SH |
| A-66 | 2,4-di(trifluoromethoxy)phenyl | SH |
| A-67 | 2,5-di(trifluoromethoxy)phenyl | SH |
| A-68 | 2,6-di(trifluoromethoxy)phenyl | SH |
| A-69 | 3,4-di(trifluoromethoxy)phenyl | SH |
| A-70 | 3,5-di(trifluoromethoxy)phenyl | SH |
| A-71 | 2,3-di(difluoromethoxy)phenyl | SH |
| A-72 | 2,4-di(difluoromethoxy)phenyl | SH |
| A-73 | 2,5-di(difluoromethoxy)phenyl | SH |
| A-74 | 2,6-di(difluoromethoxy)phenyl | SH |
| A-75 | 3,4-di(difluoromethoxy)phenyl | SH |
| A-76 | 3,5-di(difluoromethoxy)phenyl | SH |
| A-77 | 2,3-di(trifluoromethylthio)phenyl | SH |
| A-78 | 2,4-di(trifluoromethylthio)phenyl | SH |
| A-79 | 2,5-di(trifluoromethylthio)phenyl | SH |
| A-80 | 2,6-di(trifluoromethylthio)phenyl | SH |
| A-81 | 3,4-di(trifluoromethylthio)phenyl | SH |
| A-82 | 3,5-di(trifluoromethylthio)phenyl | SH |
| A-83 | 2-fluoro-3-chlorophenyl | SH |
| A-84 | 2-fluoro-4-chlorophenyl | SH |
| A-85 | 2-fluoro-5-chlorophenyl | SH |
| A-86 | 2-fluoro-6-chlorophenyl | SH |
| A-87 | 3-fluoro-4-chlorophenyl | SH |
| A-88 | 3-fluoro-5-chlorophenyl | SH |
| A-89 | 2-chloro-3-fluorophenyl | SH |
| A-90 | 2-chloro-4-fluorophenyl | SH |
| A-91 | 2-chloro-5-fluorophenyl | SH |
| A-92 | 3-chloro-4-fluorophenyl | SH |
| A-93 | 2-methyl-3-chlorophenyl | SH |
| A-94 | 2-methyl-4-chlorophenyl | SH |
| A-95 | 2-methyl-5-chlorophenyl | SH |
| A-96 | 2-methyl-6-chlorophenyl | SH |
| A-97 | 3-methyl-4-chlorophenyl | SH |
| A-98 | 3-methyl-5-chlorophenyl | SH |
| A-99 | 2-chloro-3-methylphenyl | SH |
| A-100 | 2-chloro-4-methylphenyl | SH |
| A-101 | 2-chloro-5-methylphenyl | SH |
| A-102 | 3-chloro-4-methylphenyl | SH |
| A-103 | 2-methyl-3-fluorophenyl | SH |
| A-104 | 2-methyl-4-fluorophenyl | SH |
| A-105 | 2-methyl-5-fluorophenyl | SH |
| A-106 | 2-methyl-6-fluorophenyl | SH |
| A-107 | 3-methyl-4-fluorophenyl | SH |
| A-108 | 3-methyl-5-fluorophenyl | SH |
| A-109 | 2-fluoro-3-methylphenyl | SH |
| A-110 | 2-fluoro-4-methylphenyl | SH |
| A-111 | 2-fluoro-5-methylphenyl | SH |
| A-112 | 3-fluoro-4-methylphenyl | SH |
| A-113 | 2-chloro-3-ethylphenyl | SH |
| A-114 | 2-chloro-4-ethylphenyl | SH |
| A-115 | 2-chloro-5-ethylphenyl | SH |
| A-116 | 3-chloro-4-ethylphenyl | SH |
| A-117 | 2-ethyl-3-chlorophenyl | SH |
| A-118 | 2-ethyl-4-chlorophenyl | SH |
| A-119 | 2-ethyl-5-chlorophenyl | SH |
| A-120 | 2-ethyl-6-chlorophenyl | SH |
| A-121 | 2-ethyl-3-fluorophenyl | SH |
| A-122 | 2-ethyl-4-fluorophenyl | SH |
| A-123 | 2-ethyl-5-fluorophenyl | SH |
| A-124 | 2-ethyl-6-fluorophenyl | SH |
| A-125 | 3-ethyl-4-fluorophenyl | SH |
| A-126 | 3-ethyl-5-fluorophenyl | SH |
| A-127 | 2-fluoro-3-ethylphenyl | SH |
| A-128 | 2-fluoro-4-ethylphenyl | SH |
| A-129 | 2-fluoro-5-ethylphenyl | SH |
| A-130 | 3-fluoro-4-ethylphenyl | SH |
| A-131 | 2-methoxy-3-chlorophenyl | SH |
| A-132 | 2-methoxy-4-chlorophenyl | SH |
| A-133 | 2-methoxy-5-chlorophenyl | SH |
| A-134 | 2-methoxy-6-chlorophenyl | SH |
| A-135 | 3-methoxy-4-chlorophenyl | SH |
| A-136 | 3-methoxy-5-chlorophenyl | SH |
| A-137 | 2-chloro-3-methoxyphenyl | SH |
| A-138 | 2-chloro-4-methoxyphenyl | SH |
| A-139 | 2-chloro-5-methoxyphenyl | SH |
| A-140 | 3-chloro-4-methoxyphenyl | SH |
| A-141 | 2-methoxy-3-fluorophenyl | SH |
| A-142 | 2-methoxy-4-fluorophenyl | SH |
| A-143 | 2-methoxy-5-fluorophenyl | SH |
| A-144 | 2-methoxy-6-fluorophenyl | SH |
| A-145 | 3-methoxy-4-fluorophenyl | SH |
| A-146 | 3-methoxy-5-fluorophenyl | SH |
| A-147 | 2-fluoro-3-methoxyphenyl | SH |
| A-148 | 2-fluoro-4-methoxyphenyl | SH |
| A-149 | 2-fluoro-5-methoxyphenyl | SH |
| A-150 | 3-fluoro-4-methoxyphenyl | SH |
| A-151 | 3-fluoro-5-methoxyphenyl | SH |
| A-152 | 2-(trifluoromethyl)-3-chlorophenyl | SH |
| A-153 | 2-(trifluoromethyl)-4-chlorophenyl | SH |
| A-154 | 2-(trifluoromethyl)-5-chlorophenyl | SH |
| A-155 | 2-(trifluoromethyl)-6-chlorophenyl | SH |
| A-156 | 3-(trifluoromethyl)-4-chlorophenyl | SH |
| A-157 | 3-(trifluoromethyl)-5-chlorophenyl | SH |
| A-158 | 2-chloro-3-(trifluoromethyl)phenyl | SH |
| A-159 | 2-chloro-4-(trifluoromethyl)phenyl | SH |
| A-160 | 2-chloro-5-(trifluoromethyl)phenyl | SH |
| A-161 | 3-chloro-4-(trifluoromethyl)phenyl | SH |
| A-162 | 2-(trifluoromethyl)-3-fluorophenyl | SH |
| A-163 | 2-(trifluoromethyl)-4-fluorophenyl | SH |
| A-164 | 2-(trifluoromethyl)-5-fluorophenyl | SH |
| A-165 | 2-(trifluoromethyl)-6-fluorophenyl | SH |
| A-166 | 3-(trifluoromethyl)-4-fluorophenyl | SH |
| A-167 | 3-(trifluoromethyl)-5-fluorophenyl | SH |
| A-168 | 2-fluoro-3-(trifluoromethyl)phenyl | SH |
| A-169 | 2-fluoro-4-(trifluoromethyl)phenyl | SH |
| A-170 | 2-fluoro-5-(trifluoromethyl)phenyl | SH |
| A-171 | 3-fluoro-4-(trifluoromethyl)phenyl | SH |
| A-172 | 2-(trifluoromethoxy)-3-chlorophenyl | SH |
| A-173 | 2-(trifluoromethoxy)-4-chlorophenyl | SH |
| A-174 | 2-(trifluoromethoxy)-5-chlorophenyl | SH |
| A-175 | 2-(trifluoromethoxy)-6-chlorophenyl | SH |
| A-176 | 3-(trifluoromethoxy)-4-chlorophenyl | SH |
| A-177 | 3-(trifluoromethoxy)-5-chlorophenyl | SH |
| A-178 | 2-chloro-3-(trifluoromethoxy)phenyl | SH |
| A-179 | 2-chloro-4-(trifluoromethoxy)phenyl | SH |
| A-180 | 2-chloro-5-(trifluoromethoxy)phenyl | SH |
| A-181 | 3-chloro-4-(trifluoromethoxy)phenyl | SH |
| A-182 | 2-(trifluoromethoxy)-3-fluorophenyl | SH |
| A-183 | 2-(trifluoromethoxy)-4-fluorophenyl | SH |
| A-184 | 2-(trifluoromethoxy)-5-fluorophenyl | SH |
| A-185 | 2-(trifluoromethoxy)-6-fluorophenyl | SH |
| A-186 | 3-(trifluoromethoxy)-4-fluorophenyl | SH |
| A-187 | 3-(trifluoromethoxy)-5-fluorophenyl | SH |
| A-188 | 2-fluoro-3-(trifluoromethoxy)phenyl | SH |
| A-189 | 2-fluoro-4-(trifluoromethoxy)phenyl | SH |
| A-190 | 2-fluoro-5-(trifluoromethoxy)phenyl | SH |
| A-191 | 3-fluoro-4-(trifluoromethoxy)phenyl | SH |
| A-192 | 2-(difluoromethoxy)-3-chlorophenyl | SH |
| A-193 | 2-(difluoromethoxy)-4-chlorophenyl | SH |
| A-194 | 2-(difluoromethoxy)-5-chlorophenyl | SH |
| A-195 | 2-(difluoromethoxy)-6-chlorophenyl | SH |
| A-196 | 3-(difluoromethoxy)-4-chlorophenyl | SH |
| A-197 | 3-(difluoromethoxy)-5-chlorophenyl | SH |
| A-198 | 2-chloro-3-(difluoromethoxy)phenyl | SH |
| A-199 | 2-chloro-4-(difluoromethoxy)phenyl | SH |
| A-200 | 2-chloro-5-(difluoromethoxy)phenyl | SH |
| A-201 | 3-chloro-4-(difluoromethoxy)phenyl | SH |
| A-202 | 2-(difluoromethoxy)-3-fluorophenyl | SH |
| A-203 | 2-(difluoromethoxy)-4-fluorophenyl | SH |
| A-204 | 2-(difluoromethoxy)-5-fluorophenyl | SH |
| A-205 | 2-(difluoromethoxy)-6-fluorophenyl | SH |
| A-206 | 3-(difluoromethoxy)-4-fluorophenyl | SH |
| A-207 | 3-(difluoromethoxy)-5-fluorophenyl | SH |
| A-208 | 2-fluoro-3-(difluoromethoxy)phenyl | SH |
| A-209 | 2-fluoro-4-(difluoromethoxy)phenyl | SH |
| A-210 | 2-fluoro-5-(difluoromethoxy)phenyl | SH |
| A-211 | 3-fluoro-4-(difluoromethoxy)phenyl | SH |
| A-212 | 2-(trifluoromethylthio)-3-chlorophenyl | SH |
| A-213 | 2-(trifluoromethylthio)-4-chlorophenyl | SH |
| A-214 | 2-(trifluoromethylthio)-5-chlorophenyl | SH |
| A-215 | 2-(trifluoromethylthio)-6-chlorophenyl | SH |
| A-216 | 3-(trifluoromethylthio)-4-chlorophenyl | SH |
| A-217 | 3-(trifluoromethylthio)-5-chlorophenyl | SH |
| A-218 | 2-chloro-3-(trifluoromethylthio)phenyl | SH |
| A-219 | 2-chloro-4-(trifluoromethylthio)phenyl | SH |
| A-220 | 2-chloro-5-(trifluoromethylthio)phenyl | SH |
| A-221 | 3-chloro-4-(trifluoromethylthio)phenyl | SH |
| A-222 | 2-(trifluoromethylthio)-3-fluorophenyl | SH |
| A-223 | 2-(trifluoromethylthio)-4-fluorophenyl | SH |
| A-224 | 2-(trifluoromethylthio)-5-fluorophenyl | SH |
| A-225 | 2-(trifluoromethylthio)-6-fluorophenyl | SH |
| A-226 | 3-(trifluoromethylthio)-4-fluorophenyl | SH |
| A-227 | 3-(trifluoromethylthio)-5-fluorophenyl | SH |
| A-228 | 2-fluoro-3-(trifluoromethylthio)phenyl | SH |
| A-229 | 2-fluoro-4-(trifluoromethylthio)phenyl | SH |
| A-230 | 2-fluoro-5-(trifluoromethylthio)phenyl | SH |
| A-231 | 3-fluoro-4-(trifluoromethylthio)phenyl | SH |
| A-232 | 2,3,4-trichlorophenyl | SH |
| A-233 | 2,3,5-trichlorophenyl | SH |
| A-234 | 2,3,6-trichlorophenyl | SH |
| A-235 | 2,4,5-trichlorophenyl | SH |
| A-236 | 2,4,6-trichlorophenyl | SH |
| A-237 | 3,4,5-trichlorophenyl | SH |
| A-238 | 2,3,4-trifluorophenyl | SH |
| A-239 | 2,3,5-trifluorophenyl | SH |
| A-240 | 2,3,6-trifluorophenyl | SH |
| A-241 | 2,4,5-trifluorophenyl | SH |
| A-242 | 2,4,6-trifluorophenyl | SH |
| A-243 | 3,4,5-trifluorophenyl | SH |
| A-244 | 2,3,4-trimethylphenyl | SH |
| A-245 | 2,3,5-trimethylphenyl | SH |
| A-246 | 2,3,6-trimethylphenyl | SH |
| A-247 | 2,4,5-trimethylphenyl | SH |
| A-248 | 2,4,6-trimethylphenyl | SH |
| A-249 | 3,4,5-trimethylphenyl | SH |
| A-250 | 2,3,4-trimethoxyphenyl | SH |
| A-251 | 2,3,5-trimethoxyphenyl | SH |
| A-252 | 2,3,6-trimethoxyphenyl | SH |
| A-253 | 2,4,5-trimethoxyphenyl | SH |
| A-254 | 2,4,6-trimethoxyphenyl | SH |
| A-255 | 3,4,5-trimethoxyphenyl | SH |
| A-256 | phenyl | SCH₃ |
| A-257 | 2-chlorophenyl | SCH₃ |
| A-258 | 3-chlorophenyl | SCH₃ |
| A-259 | 4-chlorophenyl | SCH₃ |
| A-260 | 2-fluorophenyl | SCH₃ |
| A-261 | 3-fluorophenyl | SCH₃ |
| A-262 | 4-fluorophenyl | SCH₃ |
| A-263 | 2-methylphenyl | SCH₃ |
| A-264 | 3-methylphenyl | SCH₃ |
| A-265 | 4-methylphenyl | SCH₃ |
| A-266 | 2-ethylphenyl | SCH₃ |
| A-267 | 3-ethylphenyl | SCH₃ |
| A-268 | 4-ethylphenyl | SCH₃ |
| A-269 | 2-methoxyphenyl | SCH₃ |
| A-270 | 3-methoxyphenyl | SCH₃ |
| A-271 | 4-methoxyphenyl | SCH₃ |
| A-272 | 2-trifluoromethylphenyl | SCH₃ |
| A-273 | 3-trifluoromethylphenyl | SCH₃ |
| A-274 | 4-trifluoromethylphenyl | SCH₃ |
| A-275 | 2-trifluoromethoxyphenyl | SCH₃ |
| A-276 | 3-trifluoromethoxyphenyl | SCH₃ |
| A-277 | 4-trifluoromethoxyphenyl | SCH₃ |
| A-278 | 2-difluoromethoxyphenyl | SCH₃ |
| A-279 | 3-difluoromethoxyphenyl | SCH₃ |
| A-280 | 4-difluoromethoxyphenyl | SCH₃ |
| A-281 | 2-trifluoromethylthiophenyl | SCH₃ |
| A-282 | 3-trifluoromethylthiophenyl | SCH₃ |
| A-283 | 4-trifluoromethylthiophenyl | SCH₃ |
| A-284 | 2,3-dichlorophenyl | SCH₃ |
| A-285 | 2,4-dichlorophenyl | SCH₃ |
| A-286 | 2,5-dichlorophenyl | SCH₃ |
| A-287 | 2,6-dichlorophenyl | SCH₃ |
| A-288 | 3,4-dichlorophenyl | SCH₃ |
| A-289 | 3,5-dichlorophenyl | SCH₃ |
| A-290 | 2,3-difluorophenyl | SCH₃ |
| A-291 | 2,4-difluorophenyl | SCH₃ |
| A-292 | 2,5-difluorophenyl | SCH₃ |
| A-293 | 2,6-difluorophenyl | SCH₃ |
| A-294 | 3,4-difluorophenyl | SCH₃ |
| A-295 | 3,5-difluorophenyl | SCH₃ |
| A-296 | 2,3-dimethylphenyl | SCH₃ |
| A-297 | 2,4-dimethylphenyl | SCH₃ |
| A-298 | 2,5-dimethylphenyl | SCH₃ |
| A-299 | 2,6-dimethylphenyl | SCH₃ |
| A-300 | 3,4-dimethylphenyl | SCH₃ |
| A-301 | 3,5-dimethylphenyl | SCH₃ |
| A-302 | 2,3-diethylphenyl | SCH₃ |
| A-303 | 2,4-diethylphenyl | SCH₃ |
| A-304 | 2,5-diethylphenyl | SCH₃ |
| A-305 | 2,6-diethylphenyl | SCH₃ |
| A-306 | 3,5-diethylphenyl | SCH₃ |
| A-307 | 3,4-diethylphenyl | SCH₃ |
| A-308 | 2,3-dimethoxyphenyl | SCH₃ |
| A-309 | 2,4-dimethoxyphenyl | SCH₃ |
| A-310 | 2,5-dimethoxyphenyl | SCH₃ |
| A-311 | 2,6-dimethoxyphenyl | SCH₃ |
| A-312 | 3,4-dimethoxyphenyl | SCH₃ |
| A-313 | 3,5-dimethoxyphenyl | SCH₃ |
| A-314 | 2,3-di(trifluoromethyl)phenyl | SCH₃ |
| A-315 | 2,4-di(trifluoromethyl)phenyl | SCH₃ |
| A-316 | 2,5-di(trifluoromethyl)phenyl | SCH₃ |
| A-317 | 2,6-di(trifluoromethyl)phenyl | SCH₃ |
| A-318 | 3,4-di(trifluoromethyl)phenyl | SCH₃ |
| A-319 | 3,5-di(trifluoromethyl)phenyl | SCH₃ |
| A-320 | 2,3-di(trifluoromethoxy)phenyl | SCH₃ |
| A-321 | 2,4-di(trifluoromethoxy)phenyl | SCH₃ |
| A-322 | 2,5-di(trifluoromethoxy)phenyl | SCH₃ |
| A-323 | 2,6-di(trifluoromethoxy)phenyl | SCH₃ |
| A-324 | 3,4-di(trifluoromethoxy)phenyl | SCH₃ |
| A-325 | 3,5-di(trifluoromethoxy)phenyl | SCH₃ |
| A-326 | 2,3-di(difluoromethoxy)phenyl | SCH₃ |
| A-327 | 2,4-di(difluoromethoxy)phenyl | SCH₃ |
| A-328 | 2,5-di(difluoromethoxy)phenyl | SCH₃ |
| A-329 | 2,6-di(difluoromethoxy)phenyl | SCH₃ |
| A-330 | 3,4-di(difluoromethoxy)phenyl | SCH₃ |
| A-331 | 3,5-di(difluoromethoxy)phenyl | SCH₃ |
| A-332 | 2,3-di(trifluoromethylthio)phenyl | SCH₃ |
| A-333 | 2,4-di(trifluoromethylthio)phenyl | SCH₃ |
| A-334 | 2,5-di(trifluoromethylthio)phenyl | SCH₃ |
| A-335 | 2,6-di(trifluoromethylthio)phenyl | SCH₃ |
| A-336 | 3,4-di(trifluoromethylthio)phenyl | SCH₃ |
| A-337 | 3,5-di(trifluoromethylthio)phenyl | SCH₃ |
| A-338 | 2-fluoro-3-chlorophenyl | SCH₃ |
| A-339 | 2-fluoro-4-chlorophenyl | SCH₃ |
| A-340 | 2-fluoro-5-chlorophenyl | SCH₃ |
| A-341 | 2-fluoro-6-chlorophenyl | SCH₃ |
| A-342 | 3-fluoro-4-chlorophenyl | SCH₃ |
| A-343 | 3-fluoro-5-chlorophenyl | SCH₃ |
| A-344 | 2-chloro-3-fluorophenyl | SCH₃ |
| A-345 | 2-chloro-4-fluorophenyl | SCH₃ |
| A-346 | 2-chloro-5-fluorophenyl | SCH₃ |
| A-347 | 3-chloro-4-fluorophenyl | SCH₃ |
| A-348 | 2-methyl-3-chlorophenyl | SCH₃ |
| A-349 | 2-methyl-4-chlorophenyl | SCH₃ |
| A-350 | 2-methyl-5-chlorophenyl | SCH₃ |
| A-351 | 2-methyl-6-chlorophenyl | SCH₃ |
| A-352 | 3-methyl-4-chlorophenyl | SCH₃ |
| A-353 | 3-methyl-5-chlorophenyl | SCH₃ |
| A-354 | 2-chloro-3-methylphenyl | SCH₃ |
| A-355 | 2-chloro-4-methylphenyl | SCH₃ |
| A-356 | 2-chloro-5-methylphenyl | SCH₃ |
| A-357 | 3-chloro-4-methylphenyl | SCH₃ |
| A-358 | 2-methyl-3-fluorophenyl | SCH₃ |
| A-359 | 2-methyl-4-fluorophenyl | SCH₃ |
| A-360 | 2-methyl-5-fluorophenyl | SCH₃ |
| A-361 | 2-methyl-6-fluorophenyl | SCH₃ |
| A-362 | 3-methyl-4-fluorophenyl | SCH₃ |
| A-363 | 3-methyl-5-fluorophenyl | SCH₃ |
| A-364 | 2-fluoro-3-methylphenyl | SCH₃ |
| A-365 | 2-fluoro-4-methylphenyl | SCH₃ |
| A-366 | 2-fluoro-5-methylphenyl | SCH₃ |
| A-367 | 3-fluoro-4-methylphenyl | SCH₃ |
| A-368 | 2-chloro-3-ethylphenyl | SCH₃ |
| A-369 | 2-chloro-4-ethylphenyl | SCH₃ |
| A-370 | 2-chloro-5-ethylphenyl | SCH₃ |
| A-371 | 3-chloro-4-ethylphenyl | SCH₃ |
| A-372 | 2-ethyl-3-chlorophenyl | SCH₃ |
| A-373 | 2-ethyl-4-chlorophenyl | SCH₃ |
| A-374 | 2-ethyl-5-chlorophenyl | SCH₃ |
| A-375 | 2-ethyl-6-chlorophenyl | SCH₃ |
| A-376 | 2-ethyl-3-fluorophenyl | SCH₃ |
| A-377 | 2-ethyl-4-fluorophenyl | SCH₃ |
| A-378 | 2-ethyl-5-fluorophenyl | SCH₃ |
| A-379 | 2-ethyl-6-fluorophenyl | SCH₃ |
| A-380 | 3-ethyl-4-fluorophenyl | SCH₃ |
| A-381 | 3-ethyl-5-fluorophenyl | SCH₃ |
| A-382 | 2-fluoro-3-ethylphenyl | SCH₃ |
| A-383 | 2-fluoro-4-ethylphenyl | SCH₃ |
| A-384 | 2-fluoro-5-ethylphenyl | SCH₃ |
| A-385 | 3-fluoro-4-ethylphenyl | SCH₃ |
| A-386 | 2-methoxy-3-chlorophenyl | SCH₃ |
| A-387 | 2-methoxy-4-chlorophenyl | SCH₃ |
| A-388 | 2-methoxy-5-chlorophenyl | SCH₃ |
| A-389 | 2-methoxy-6-chlorophenyl | SCH₃ |
| A-390 | 3-methoxy-4-chlorophenyl | SCH₃ |
| A-391 | 3-methoxy-5-chlorophenyl | SCH₃ |
| A-392 | 2-chloro-3-methoxyphenyl | SCH₃ |
| A-393 | 2-chloro-4-methoxyphenyl | SCH₃ |
| A-394 | 2-chloro-5-methoxyphenyl | SCH₃ |
| A-395 | 3-chloro-4-methoxyphenyl | SCH₃ |
| A-396 | 2-methoxy-3-fluorophenyl | SCH₃ |
| A-397 | 2-methoxy-4-fluorophenyl | SCH₃ |
| A-398 | 2-methoxy-5-fluorophenyl | SCH₃ |
| A-399 | 2-methoxy-6-fluorophenyl | SCH₃ |
| A-400 | 3-methoxy-4-fluorophenyl | SCH₃ |
| A-401 | 3-methoxy-5-fluorophenyl | SCH₃ |
| A-402 | 2-fluoro-3-methoxyphenyl | SCH₃ |
| A-403 | 2-fluoro-4-methoxyphenyl | SCH₃ |
| A-404 | 2-fluoro-5-methoxyphenyl | SCH₃ |
| A-405 | 3-fluoro-4-methoxyphenyl | SCH₃ |
| A-406 | 3-fluoro-5-methoxyphenyl | SCH₃ |
| A-407 | 2-(trifluoromethyl)-3-chlorophenyl | SCH₃ |
| A-408 | 2-(trifluoromethyl)-4-chlorophenyl | SCH₃ |
| A-409 | 2-(trifluoromethyl)-5-chlorophenyl | SCH₃ |
| A-410 | 2-(trifluoromethyl)-6-chlorophenyl | SCH₃ |
| A-411 | 3-(trifluoromethyl)-4-chlorophenyl | SCH₃ |
| A-412 | 3-(trifluoromethyl)-5-chlorophenyl | SCH₃ |
| A-413 | 2-chloro-3-(trifluoromethyl)phenyl | SCH₃ |
| A-414 | 2-chloro-4-(trifluoromethyl)phenyl | SCH₃ |
| A-415 | 2-chloro-5-(trifluoromethyl)phenyl | SCH₃ |
| A-416 | 3-chloro-4-(trifluoromethyl)phenyl | SCH₃ |
| A-417 | 2-(trifluoromethyl)-3-fluorophenyl | SCH₃ |
| A-418 | 2-(trifluoromethyl)-4-fluorophenyl | SCH₃ |
| A-419 | 2-(trifluoromethyl)-5-fluorophenyl | SCH₃ |
| A-420 | 2-(trifluoromethyl)-6-fluorophenyl | SCH₃ |
| A-421 | 3-(trifluoromethyl)-4-fluorophenyl | SCH₃ |
| A-422 | 3-(trifluoromethyl)-5-fluorophenyl | SCH₃ |
| A-423 | 2-fluoro-3-(trifluoromethyl)phenyl | SCH₃ |
| A-424 | 2-fluoro-4-(trifluoromethyl)phenyl | SCH₃ |
| A-425 | 2-fluoro-5-(trifluoromethyl)phenyl | SCH₃ |
| A-426 | 3-fluoro-4-(trifluoromethyl)phenyl | SCH₃ |
| A-427 | 2-(trifluoromethoxy)-3-chlorophenyl | SCH₃ |
| A-428 | 2-(trifluoromethoxy)-4-chlorophenyl | SCH₃ |
| A-429 | 2-(trifluoromethoxy)-5-chlorophenyl | SCH₃ |
| A-430 | 2-(trifluoromethoxy)-6-chlorophenyl | SCH₃ |
| A-431 | 3-(trifluoromethoxy)-4-chlorophenyl | SCH₃ |
| A-432 | 3-(trifluoromethoxy)-5-chlorophenyl | SCH₃ |
| A-433 | 2-chloro-3-(trifluoromethoxy)phenyl | SCH₃ |
| A-434 | 2-chloro-4-(trifluoromethoxy)phenyl | SCH₃ |
| A-435 | 2-chloro-5-(trifluoromethoxy)phenyl | SCH₃ |
| A-436 | 3-chloro-4-(trifluoromethoxy)phenyl | SCH₃ |
| A-437 | 2-(trifluoromethoxy)-3-fluorophenyl | SCH₃ |
| A-438 | 2-(trifluoromethoxy)-4-fluorophenyl | SCH₃ |
| A-439 | 2-(trifluoromethoxy)-5-fluorophenyl | SCH₃ |
| A-440 | 2-(trifluoromethoxy)-6-fluorophenyl | SCH₃ |
| A-441 | 3-(trifluoromethoxy)-4-fluorophenyl | SCH₃ |
| A-442 | 3-(trifluoromethoxy)-5-fluorophenyl | SCH₃ |
| A-443 | 2-fluoro-3-(trifluoromethoxy)phenyl | SCH₃ |
| A-444 | 2-fluoro-4-(trifluoromethoxy)phenyl | SCH₃ |
| A-445 | 2-fluoro-5-(trifluoromethoxy)phenyl | SCH₃ |
| A-446 | 3-fluoro-4-(trifluoromethoxy)phenyl | SCH₃ |
| A-447 | 2-(difluoromethoxy)-3-chlorophenyl | SCH₃ |
| A-448 | 2-(difluoromethoxy)-4-chlorophenyl | SCH₃ |
| A-449 | 2-(difluoromethoxy)-5-chlorophenyl | SCH₃ |
| A-450 | 2-(difluoromethoxy)-6-chlorophenyl | SCH₃ |
| A-451 | 3-(difluoromethoxy)-4-chlorophenyl | SCH₃ |
| A-452 | 3-(difluoromethoxy)-5-chlorophenyl | SCH₃ |
| A-453 | 2-chloro-3-(difluoromethoxy)phenyl | SCH₃ |
| A-454 | 2-chloro-4-(difluoromethoxy)phenyl | SCH₃ |
| A-455 | 2-chloro-5-(difluoromethoxy)phenyl | SCH₃ |
| A-456 | 3-chloro-4-(difluoromethoxy)phenyl | SCH₃ |
| A-457 | 2-(difluoromethoxy)-3-fluorophenyl | SCH₃ |
| A-458 | 2-(difluoromethoxy)-4-fluorophenyl | SCH₃ |
| A-459 | 2-(difluoromethoxy)-5-fluorophenyl | SCH₃ |
| A-460 | 2-(difluoromethoxy)-6-fluorophenyl | SCH₃ |
| A-461 | 3-(difluoromethoxy)-4-fluorophenyl | SCH₃ |
| A-462 | 3-(difluoromethoxy)-5-fluorophenyl | SCH₃ |
| A-463 | 2-fluoro-3-(difluoromethoxy)phenyl | SCH₃ |
| A-464 | 2-fluoro-4-(difluoromethoxy)phenyl | SCH₃ |
| A-465 | 2-fluoro-5-(difluoromethoxy)phenyl | SCH₃ |
| A-466 | 3-fluoro-4-(difluoromethoxy)phenyl | SCH₃ |
| A-467 | 2-(trifluoromethylthio)-3-chlorophenyl | SCH₃ |
| A-468 | 2-(trifluoromethylthio)-4-chlorophenyl | SCH₃ |
| A-469 | 2-(trifluoromethylthio)-5-chlorophenyl | SCH₃ |
| A-470 | 2-(trifluoromethylthio)-6-chlorophenyl | SCH₃ |
| A-471 | 3-(trifluoromethylthio)-4-chlorophenyl | SCH₃ |
| A-472 | 3-(trifluoromethylthio)-5-chlorophenyl | SCH₃ |
| A-473 | 2-chloro-3-(trifluoromethylthio)phenyl | SCH₃ |
| A-474 | 2-chloro-4-(trifluoromethylthio)phenyl | SCH₃ |
| A-475 | 2-chloro-5-(trifluoromethylthio)phenyl | SCH₃ |
| A-476 | 3-chloro-4-(trifluoromethylthio)phenyl | SCH₃ |
| A-477 | 2-(trifluoromethylthio)-3-fluorophenyl | SCH₃ |
| A-478 | 2-(trifluoromethylthio)-4-fluorophenyl | SCH₃ |
| A-479 | 2-(trifluoromethylthio)-5-fluorophenyl | SCH₃ |
| A-480 | 2-(trifluoromethylthio)-6-fluorophenyl | SCH₃ |
| A-481 | 3-(trifluoromethylthio)-4-fluorophenyl | SCH₃ |
| A-482 | 3-(trifluoromethylthio)-5-fluorophenyl | SCH₃ |
| A-483 | 2-fluoro-3-(trifluoromethylthio)phenyl | SCH₃ |
| A-484 | 2-fluoro-4-(trifluoromethylthio)phenyl | SCH₃ |
| A-485 | 2-fluoro-5-(trifluoromethylthio)phenyl | SCH₃ |
| A-486 | 3-fluoro-4-(trifluoromethylthio)phenyl | SCH₃ |
| A-487 | 2,3,4-trichlorophenyl | SCH₃ |
| A-488 | 2,3,5-trichlorophenyl | SCH₃ |
| A-489 | 2,3,6-trichlorophenyl | SCH₃ |
| A-490 | 2,4,5-trichlorophenyl | SCH₃ |
| A-491 | 2,4,6-trichlorophenyl | SCH₃ |
| A-492 | 3,4,5-trichlorophenyl | SCH₃ |
| A-493 | 2,3,4-trifluorophenyl | SCH₃ |
| A-494 | 2,3,5-trifluorophenyl | SCH₃ |
| A-495 | 2,3,6-trifluorophenyl | SCH₃ |
| A-496 | 2,4,5-trifluorophenyl | SCH₃ |
| A-497 | 2,4,6-trifluorophenyl | SCH₃ |
| A-498 | 3,4,5-trifluorophenyl | SCH₃ |
| A-499 | 2,3,4-trimethylphenyl | SCH₃ |
| A-500 | 2,3,5-trimethylphenyl | SCH₃ |
| A-501 | 2,3,6-trimethylphenyl | SCH₃ |
| A-502 | 2,4,5-trimethylphenyl | SCH₃ |
| A-503 | 2,4,6-trimethylphenyl | SCH₃ |
| A-504 | 3,4,5-trimethylphenyl | SCH₃ |
| A-505 | 2,3,4-trimethoxyphenyl | SCH₃ |
| A-506 | 2,3,5-trimethoxyphenyl | SCH₃ |
| A-507 | 2,3,6-trimethoxyphenyl | SCH₃ |
| A-508 | 2,4,5-trimethoxyphenyl | SCH₃ |
| A-509 | 2,4,6-trimethoxyphenyl | SCH₃ |
| A-510 | 3,4,5-trimethoxyphenyl | SCH₃ |
| A-511 | phenyl | SC₂H₅ |
| A-512 | 2-chlorophenyl | SC₂H₅ |
| A-513 | 3-chlorophenyl | SC₂H₅ |
| A-514 | 4-chlorophenyl | SC₂H₅ |
| A-515 | 2-fluorophenyl | SC₂H₅ |
| A-516 | 3-fluorophenyl | SC₂H₅ |
| A-517 | 4-fluorophenyl | SC₂H₅ |
| A-518 | 2-methylphenyl | SC₂H₅ |
| A-519 | 3-methylphenyl | SC₂H₅ |
| A-520 | 4-methylphenyl | SC₂H₅ |
| A-521 | 2-ethylphenyl | SC₂H₅ |
| A-522 | 3-ethylphenyl | SC₂H₅ |
| A-523 | 4-ethylphenyl | SC₂H₅ |
| A-524 | 2-methoxyphenyl | SC₂H₅ |
| A-525 | 3-methoxyphenyl | SC₂H₅ |
| A-526 | 4-methoxyphenyl | SC₂H₅ |
| A-527 | 2-trifluoromethylphenyl | SC₂H₅ |
| A-528 | 3-trifluoromethylphenyl | SC₂H₅ |
| A-529 | 4-trifluoromethylphenyl | SC₂H₅ |
| A-530 | 2-trifluoromethoxyphenyl | SC₂H₅ |
| A-531 | 3-trifluoromethoxyphenyl | SC₂H₅ |
| A-532 | 4-trifluoromethoxyphenyl | SC₂H₅ |
| A-533 | 2-difluoromethoxyphenyl | SC₂H₅ |
| A-534 | 3-difluoromethoxyphenyl | SC₂H₅ |
| A-535 | 4-difluoromethoxyphenyl | SC₂H₅ |
| A-536 | 2-trifluoromethylthiophenyl | SC₂H₅ |
| A-537 | 3-trifluoromethylthiophenyl | SC₂H₅ |
| A-538 | 4-trifluoromethylthiophenyl | SC₂H₅ |
| A-539 | 2,3-dichlorophenyl | SC₂H₅ |
| A-540 | 2,4-dichlorophenyl | SC₂H₅ |
| A-541 | 2,5-dichlorophenyl | SC₂H₅ |
| A-542 | 2,6-dichlorophenyl | SC₂H₅ |
| A-543 | 3,4-dichlorophenyl | SC₂H₅ |
| A-544 | 3,5-dichlorophenyl | SC₂H₅ |
| A-545 | 2,3-difluorophenyl | SC₂H₅ |
| A-546 | 2,4-difluorophenyl | SC₂H₅ |
| A-547 | 2,5-difluorophenyl | SC₂H₅ |
| A-548 | 2,6-difluorophenyl | SC₂H₅ |
| A-549 | 3,4-difluorophenyl | SC₂H₅ |
| A-550 | 3,5-difluorophenyl | SC₂H₅ |
| A-551 | 2,3-dimethylphenyl | SC₂H₅ |
| A-552 | 2,4-dimethylphenyl | SC₂H₅ |
| A-553 | 2,5-dimethylphenyl | SC₂H₅ |
| A-554 | 2,6-dimethylphenyl | SC₂H₅ |
| A-555 | 3,4-dimethylphenyl | SC₂H₅ |
| A-556 | 3,5-dimethylphenyl | SC₂H₅ |
| A-557 | 2,3-diethylphenyl | SC₂H₅ |
| A-558 | 2,4-diethylphenyl | SC₂H₅ |
| A-559 | 2,5-diethylphenyl | SC₂H₅ |
| A-560 | 2,6-diethylphenyl | SC₂H₅ |
| A-561 | 3,5-diethylphenyl | SC₂H₅ |
| A-562 | 3,4-diethylphenyl | SC₂H₅ |
| A-563 | 2,3-dimethoxyphenyl | SC₂H₅ |
| A-564 | 2,4-dimethoxyphenyl | SC₂H₅ |
| A-565 | 2,5-dimethoxyphenyl | SC₂H₅ |
| A-566 | 2,6-dimethoxyphenyl | SC₂H₅ |
| A-567 | 3,4-dimethoxyphenyl | SC₂H₅ |
| A-568 | 3,5-dimethoxyphenyl | SC₂H₅ |
| A-569 | 2,3-di(trifluoromethyl)phenyl | SC₂H₅ |
| A-570 | 2,4-di(trifluoromethyl)phenyl | SC₂H₅ |
| A-571 | 2,5-di(trifluoromethyl)phenyl | SC₂H₅ |
| A-572 | 2,6-di(trifluoromethyl)phenyl | SC₂H₅ |
| A-573 | 3,4-di(trifluoromethyl)phenyl | SC₂H₅ |
| A-574 | 3,5-di(trifluoromethyl)phenyl | SC₂H₅ |
| A-575 | 2,3-di(trifluoromethoxy)phenyl | SC₂H₅ |
| A-576 | 2,4-di(trifluoromethoxy)phenyl | SC₂H₅ |
| A-577 | 2,5-di(trifluoromethoxy)phenyl | SC₂H₅ |
| A-578 | 2,6-di(trifluoromethoxy)phenyl | SC₂H₅ |
| A-579 | 3,4-di(trifluoromethoxy)phenyl | SC₂H₅ |
| A-580 | 3,5-di(trifluoromethoxy)phenyl | SC₂H₅ |
| A-581 | 2,3-di(difluoromethoxy)phenyl | SC₂H₅ |
| A-582 | 2,4-di(difluoromethoxy)phenyl | SC₂H₅ |
| A-583 | 2,5-di(difluoromethoxy)phenyl | SC₂H₅ |
| A-584 | 2,6-di(difluoromethoxy)phenyl | SC₂H₅ |
| A-585 | 3,4-di(difluoromethoxy)phenyl | SC₂H₅ |
| A-586 | 3,5-di(difluoromethoxy)phenyl | SC₂H₅ |
| A-587 | 2,3-di(trifluoromethylthio)phenyl | SC₂H₅ |
| A-588 | 2,4-di(trifluoromethylthio)phenyl | SC₂H₅ |
| A-589 | 2,5-di(trifluoromethylthio)phenyl | SC₂H₅ |
| A-590 | 2,6-di(trifluoromethylthio)phenyl | SC₂H₅ |
| A-591 | 3,4-di(trifluoromethylthio)phenyl | SC₂H₅ |
| A-592 | 3,5-di(trifluoromethylthio)phenyl | SC₂H₅ |
| A-593 | 2-fluoro-3-chlorophenyl | SC₂H₅ |
| A-594 | 2-fluoro-4-chlorophenyl | SC₂H₅ |
| A-595 | 2-fluoro-5-chlorophenyl | SC₂H₅ |
| A-596 | 2-fluoro-6-chlorophenyl | SC₂H₅ |
| A-597 | 3-fluoro-4-chlorophenyl | SC₂H₅ |
| A-598 | 3-fluoro-5-chlorophenyl | SC₂H₅ |
| A-599 | 2-chloro-3-fluorophenyl | SC₂H₅ |
| A-600 | 2-chloro-4-fluorophenyl | SC₂H₅ |
| A-601 | 2-chloro-5-fluorophenyl | SC₂H₅ |
| A-602 | 3-chloro-4-fluorophenyl | SC₂H₅ |
| A-603 | 2-methyl-3-chlorophenyl | SC₂H₅ |
| A-604 | 2-methyl-4-chlorophenyl | SC₂H₅ |
| A-605 | 2-methyl-5-chlorophenyl | SC₂H₅ |
| A-606 | 2-methyl-6-chlorophenyl | SC₂H₅ |
| A-607 | 3-methyl-4-chlorophenyl | SC₂H₅ |
| A-608 | 3-methyl-5-chlorophenyl | SC₂H₅ |
| A-609 | 2-chloro-3-methylphenyl | SC₂H₅ |
| A-610 | 2-chloro-4-methylphenyl | SC₂H₅ |
| A-611 | 2-chloro-5-methylphenyl | SC₂H₅ |
| A-612 | 3-chloro-4-methylphenyl | SC₂H₅ |
| A-613 | 2-methyl-3-fluorophenyl | SC₂H₅ |
| A-614 | 2-methyl-4-fluorophenyl | SC₂H₅ |
| A-615 | 2-methyl-5-fluorophenyl | SC₂H₅ |
| A-616 | 2-methyl-6-fluorophenyl | SC₂H₅ |
| A-617 | 3-methyl-4-fluorophenyl | SC₂H₅ |
| A-618 | 3-methyl-5-fluorophenyl | SC₂H₅ |
| A-619 | 2-fluoro-3-methylphenyl | SC₂H₅ |
| A-620 | 2-fluoro-4-methylphenyl | SC₂H₅ |
| A-621 | 2-fluoro-5-methylphenyl | SC₂H₅ |
| A-622 | 3-fluoro-4-methylphenyl | SC₂H₅ |
| A-623 | 2-chloro-3-ethylphenyl | SC₂H₅ |
| A-624 | 2-chloro-4-ethylphenyl | SC₂H₅ |
| A-625 | 2-chloro-5-ethylphenyl | SC₂H₅ |
| A-626 | 3-chloro-4-ethylphenyl | SC₂H₅ |
| A-627 | 2-ethyl-3-chlorophenyl | SC₂H₅ |
| A-628 | 2-ethyl-4-chlorophenyl | SC₂H₅ |
| A-629 | 2-ethyl-5-chlorophenyl | SC₂H₅ |
| A-630 | 2-ethyl-6-chlorophenyl | SC₂H₅ |
| A-631 | 2-ethyl-3-fluorophenyl | SC₂H₅ |
| A-632 | 2-ethyl-4-fluorophenyl | SC₂H₅ |
| A-633 | 2-ethyl-5-fluorophenyl | SC₂H₅ |
| A-634 | 2-ethyl-6-fluorophenyl | SC₂H₅ |
| A-635 | 3-ethyl-4-fluorophenyl | SC₂H₅ |
| A-636 | 3-ethyl-5-fluorophenyl | SC₂H₅ |
| A-637 | 2-fluoro-3-ethylphenyl | SC₂H₅ |
| A-638 | 2-fluoro-4-ethylphenyl | SC₂H₅ |
| A-639 | 2-fluoro-5-ethylphenyl | SC₂H₅ |
| A-640 | 3-fluoro-4-ethylphenyl | SC₂H₅ |
| A-641 | 2-methoxy-3-chlorophenyl | SC₂H₅ |
| A-642 | 2-methoxy-4-chlorophenyl | SC₂H₅ |
| A-643 | 2-methoxy-5-chlorophenyl | SC₂H₅ |
| A-644 | 2-methoxy-6-chlorophenyl | SC₂H₅ |
| A-645 | 3-methoxy-4-chlorophenyl | SC₂H₅ |
| A-646 | 3-methoxy-5-chlorophenyl | SC₂H₅ |
| A-647 | 2-chloro-3-methoxyphenyl | SC₂H₅ |
| A-648 | 2-chloro-4-methoxyphenyl | SC₂H₅ |
| A-649 | 2-chloro-5-methoxyphenyl | SC₂H₅ |
| A-650 | 3-chloro-4-methoxyphenyl | SC₂H₅ |
| A-651 | 2-methoxy-3-fluorophenyl | SC₂H₅ |
| A-652 | 2-methoxy-4-fluorophenyl | SC₂H₅ |
| A-653 | 2-methoxy-5-fluorophenyl | SC₂H₅ |
| A-654 | 2-methoxy-6-fluorophenyl | SC₂H₅ |
| A-655 | 3-methoxy-4-fluorophenyl | SC₂H₅ |
| A-656 | 3-methoxy-5-fluorophenyl | SC₂H₅ |
| A-657 | 2-fluoro-3-methoxyphenyl | SC₂H₅ |
| A-658 | 2-fluoro-4-methoxyphenyl | SC₂H₅ |
| A-659 | 2-fluoro-5-methoxyphenyl | SC₂H₅ |
| A-660 | 3-fluoro-4-methoxyphenyl | SC₂H₅ |
| A-661 | 3-fluoro-5-methoxyphenyl | SC₂H₅ |
| A-662 | 2-(trifluoromethyl)-3-chlorophenyl | SC₂H₅ |
| A-663 | 2-(trifluoromethyl)-4-chlorophenyl | SC₂H₅ |
| A-664 | 2-(trifluoromethyl)-5-chlorophenyl | SC₂H₅ |
| A-665 | 2-(trifluoromethyl)-6-chlorophenyl | SC₂H₅ |
| A-666 | 3-(trifluoromethyl)-4-chlorophenyl | SC₂H₅ |
| A-667 | 3-(trifluoromethyl)-5-chlorophenyl | SC₂H₅ |
| A-668 | 2-chloro-3-(trifluoromethyl)phenyl | SC₂H₅ |
| A-669 | 2-chloro-4-(trifluoromethyl)phenyl | SC₂H₅ |
| A-670 | 2-chloro-5-(trifluoromethyl)phenyl | SC₂H₅ |
| A-671 | 3-chloro-4-(trifluoromethyl)phenyl | SC₂H₅ |
| A-672 | 2-(trifluoromethyl)-3-fluorophenyl | SC₂H₅ |
| A-673 | 2-(trifluoromethyl)-4-fluorophenyl | SC₂H₅ |
| A-674 | 2-(trifluoromethyl)-5-fluorophenyl | SC₂H₅ |
| A-675 | 2-(trifluoromethyl)-6-fluorophenyl | SC₂H₅ |
| A-676 | 3-(trifluoromethyl)-4-fluorophenyl | SC₂H₅ |
| A-677 | 3-(trifluoromethyl)-5-fluorophenyl | SC₂H₅ |
| A-678 | 2-fluoro-3-(trifluoromethyl)phenyl | SC₂H₅ |
| A-679 | 2-fluoro-4-(trifluoromethyl)phenyl | SC₂H₅ |
| A-680 | 2-fluoro-5-(trifluoromethyl)phenyl | SC₂H₅ |
| A-681 | 3-fluoro-4-(trifluoromethyl)phenyl | SC₂H₅ |
| A-682 | 2-(trifluoromethoxy)-3-chlorophenyl | SC₂H₅ |
| A-683 | 2-(trifluoromethoxy)-4-chlorophenyl | SC₂H₅ |
| A-684 | 2-(trifluoromethoxy)-5-chlorophenyl | SC₂H₅ |
| A-685 | 2-(trifluoromethoxy)-6-chlorophenyl | SC₂H₅ |
| A-686 | 3-(trifluoromethoxy)-4-chlorophenyl | SC₂H₅ |
| A-687 | 3-(trifluoromethoxy)-5-chlorophenyl | SC₂H₅ |
| A-688 | 2-chloro-3-(trifluoromethoxy)phenyl | SC₂H₅ |
| A-689 | 2-chloro-4-(trifluoromethoxy)phenyl | SC₂H₅ |
| A-690 | 2-chloro-5-(trifluoromethoxy)phenyl | SC₂H₅ |
| A-691 | 3-chloro-4-(trifluoromethoxy)phenyl | SC₂H₅ |
| A-692 | 2-(trifluoromethoxy)-3-fluorophenyl | SC₂H₅ |
| A-693 | 2-(trifluoromethoxy)-4-fluorophenyl | SC₂H₅ |
| A-694 | 2-(trifluoromethoxy)-5-fluorophenyl | SC₂H₅ |
| A-695 | 2-(trifluoromethoxy)-6-fluorophenyl | SC₂H₅ |
| A-696 | 3-(trifluoromethoxy)-4-fluorophenyl | SC₂H₅ |
| A-697 | 3-(trifluoromethoxy)-5-fluorophenyl | SC₂H₅ |
| A-698 | 2-fluoro-3-(trifluoromethoxy)phenyl | SC₂H₅ |
| A-699 | 2-fluoro-4-(trifluoromethoxy)phenyl | SC₂H₅ |
| A-700 | 2-fluoro-5-(trifluoromethoxy)phenyl | SC₂H₅ |
| A-701 | 3-fluoro-4-(trifluoromethoxy)phenyl | SC₂H₅ |
| A-702 | 2-(difluoromethoxy)-3-chlorophenyl | SC₂H₅ |
| A-703 | 2-(difluoromethoxy)-4-chlorophenyl | SC₂H₅ |
| A-704 | 2-(difluoromethoxy)-5-chlorophenyl | SC₂H₅ |
| A-705 | 2-(difluoromethoxy)-6-chlorophenyl | SC₂H₅ |
| A-706 | 3-(difluoromethoxy)-4-chlorophenyl | SC₂H₅ |
| A-707 | 3-(difluoromethoxy)-5-chlorophenyl | SC₂H₅ |
| A-708 | 2-chloro-3-(difluoromethoxy)phenyl | SC₂H₅ |
| A-709 | 2-chloro-4-(difluoromethoxy)phenyl | SC₂H₅ |
| A-710 | 2-chloro-5-(difluoromethoxy)phenyl | SC₂H₅ |
| A-711 | 3-chloro-4-(difluoromethoxy)phenyl | SC₂H₅ |
| A-712 | 2-(difluoromethoxy)-3-fluorophenyl | SC₂H₅ |
| A-713 | 2-(difluoromethoxy)-4-fluorophenyl | SC₂H₅ |
| A-714 | 2-(difluoromethoxy)-5-fluorophenyl | SC₂H₅ |
| A-715 | 2-(difluoromethoxy)-6-fluorophenyl | SC₂H₅ |
| A-716 | 3-(difluoromethoxy)-4-fluorophenyl | SC₂H₅ |
| A-717 | 3-(difluoromethoxy)-5-fluorophenyl | SC₂H₅ |
| A-718 | 2-fluoro-3-(difluoromethoxy)phenyl | SC₂H₅ |
| A-719 | 2-fluoro-4-(difluoromethoxy)phenyl | SC₂H₅ |
| A-720 | 2-fluoro-5-(difluoromethoxy)phenyl | SC₂H₅ |
| A-721 | 3-fluoro-4-(difluoromethoxy)phenyl | SC₂H₅ |
| A-722 | 2-(trifluoromethylthio)-3-chlorophenyl | SC₂H₅ |
| A-723 | 2-(trifluoromethylthio)-4-chlorophenyl | SC₂H₅ |
| A-724 | 2-(trifluoromethylthio)-5-chlorophenyl | SC₂H₅ |
| A-725 | 2-(trifluoromethylthio)-6-chlorophenyl | SC₂H₅ |
| A-726 | 3-(trifluoromethylthio)-4-chlorophenyl | SC₂H₅ |
| A-727 | 3-(trifluoromethylthio)-5-chlorophenyl | SC₂H₅ |
| A-728 | 2-chloro-3-(trifluoromethylthio)phenyl | SC₂H₅ |
| A-729 | 2-chloro-4-(trifluoromethylthio)phenyl | SC₂H₅ |
| A-730 | 2-chloro-5-(trifluoromethylthio)phenyl | SC₂H₅ |
| A-731 | 3-chloro-4-(trifluoromethylthio)phenyl | SC₂H₅ |
| A-732 | 2-(trifluoromethylthio)-3-fluorophenyl | SC₂H₅ |
| A-733 | 2-(trifluoromethylthio)-4-fluorophenyl | SC₂H₅ |
| A-734 | 2-(trifluoromethylthio)-5-fluorophenyl | SC₂H₅ |
| A-735 | 2-(trifluoromethylthio)-6-fluorophenyl | SC₂H₅ |
| A-736 | 3-(trifluoromethylthio)-4-fluorophenyl | SC₂H₅ |
| A-737 | 3-(trifluoromethylthio)-5-fluorophenyl | SC₂H₅ |
| A-738 | 2-fluoro-3-(trifluoromethylthio)phenyl | SC₂H₅ |
| A-739 | 2-fluoro-4-(trifluoromethylthio)phenyl | SC₂H₅ |
| A-740 | 2-fluoro-5-(trifluoromethylthio)phenyl | SC₂H₅ |
| A-741 | 3-fluoro-4-(trifluoromethylthio)phenyl | SC₂H₅ |
| A-742 | 2,3,4-trichlorophenyl | SC₂H₅ |
| A-743 | 2,3,5-trichlorophenyl | SC₂H₅ |
| A-744 | 2,3,6-trichlorophenyl | SC₂H₅ |
| A-745 | 2,4,5-trichlorophenyl | SC₂H₅ |
| A-746 | 2,4,6-trichlorophenyl | SC₂H₅ |
| A-747 | 3,4,5-trichlorophenyl | SC₂H₅ |
| A-748 | 2,3,4-trifluorophenyl | SC₂H₅ |
| A-749 | 2,3,5-trifluorophenyl | SC₂H₅ |
| A-750 | 2,3,6-trifluorophenyl | SC₂H₅ |
| A-751 | 2,4,5-trifluorophenyl | SC₂H₅ |
| A-752 | 2,4,6-trifluorophenyl | SC₂H₅ |
| A-753 | 3,4,5-trifluorophenyl | SC₂H₅ |
| A-754 | 2,3,4-trimethylphenyl | SC₂H₅ |
| A-755 | 2,3,5-trimethylphenyl | SC₂H₅ |
| A-756 | 2,3,6-trimethylphenyl | SC₂H₅ |
| A-757 | 2,4,5-trimethylphenyl | SC₂H₅ |
| A-758 | 2,4,6-trimethylphenyl | SC₂H₅ |
| A-759 | 3,4,5-trimethylphenyl | SC₂H₅ |
| A-760 | 2,3,4-trimethoxyphenyl | SC₂H₅ |
| A-761 | 2,3,5-trimethoxyphenyl | SC₂H₅ |
| A-762 | 2,3,6-trimethoxyphenyl | SC₂H₅ |
| A-763 | 2,4,5-trimethoxyphenyl | SC₂H₅ |
| A-764 | 2,4,6-trimethoxyphenyl | SC₂H₅ |
| A-765 | 3,4,5-trimethoxyphenyl | SC₂H₅ |
| A-766 | phenyl | SNa |
| A-767 | 2-chlorophenyl | SNa |
| A-768 | 3-chlorophenyl | SNa |
| A-769 | 4-chlorophenyl | SNa |
| A-770 | 2-fluorophenyl | SNa |
| A-771 | 3-fluorophenyl | SNa |
| A-772 | 4-fluorophenyl | SNa |
| A-773 | 2-methylphenyl | SNa |
| A-774 | 3-methylphenyl | SNa |
| A-775 | 4-methylphenyl | SNa |
| A-776 | 2-ethylphenyl | SNa |
| A-777 | 3-ethylphenyl | SNa |
| A-778 | 4-ethylphenyl | SNa |
| A-779 | 2-methoxyphenyl | SNa |
| A-780 | 3-methoxyphenyl | SNa |
| A-781 | 4-methoxyphenyl | SNa |
| A-782 | 2-trifluoromethylphenyl | SNa |
| A-783 | 3-trifluoromethylphenyl | SNa |
| A-784 | 4-trifluoromethylphenyl | SNa |
| A-785 | 2-trifluoromethoxyphenyl | SNa |
| A-786 | 3-trifluoromethoxyphenyl | SNa |
| A-787 | 4-trifluoromethoxyphenyl | SNa |
| A-788 | 2-difluoromethoxyphenyl | SNa |
| A-789 | 3-difluoromethoxyphenyl | SNa |
| A-790 | 4-difluoromethoxyphenyl | SNa |
| A-791 | 2-trifluoromethylthiophenyl | SNa |
| A-792 | 3-trifluoromethylthiophenyl | SNa |
| A-793 | 4-trifluoromethylthiophenyl | SNa |
| A-794 | 2,3-dichlorophenyl | SNa |
| A-795 | 2,4-dichlorophenyl | SNa |
| A-796 | 2,5-dichlorophenyl | SNa |
| A-797 | 2,6-dichlorophenyl | SNa |
| A-798 | 3,4-dichlorophenyl | SNa |
| A-799 | 3,5-dichlorophenyl | SNa |
| A-800 | 2,3-difluorophenyl | SNa |
| A-801 | 2,4-difluorophenyl | SNa |
| A-802 | 2,5-difluorophenyl | SNa |
| A-803 | 2,6-difluorophenyl | SNa |
| A-804 | 3,4-difluorophenyl | SNa |
| A-805 | 3,5-difluorophenyl | SNa |
| A-806 | 2,3-dimethylphenyl | SNa |
| A-807 | 2,4-dimethylphenyl | SNa |
| A-808 | 2,5-dimethylphenyl | SNa |
| A-809 | 2,6-dimethylphenyl | SNa |
| A-810 | 3,4-dimethylphenyl | SNa |
| A-811 | 3,5-dimethylphenyl | SNa |
| A-812 | 2,3-diethylphenyl | SNa |
| A-813 | 2,4-diethylphenyl | SNa |
| A-814 | 2,5-diethylphenyl | SNa |
| A-815 | 2,6-diethylphenyl | SNa |
| A-816 | 3,5-diethylphenyl | SNa |
| A-817 | 3,4-diethylphenyl | SNa |
| A-818 | 2,3-dimethoxyphenyl | SNa |
| A-819 | 2,4-dimethoxyphenyl | SNa |
| A-820 | 2,5-dimethoxyphenyl | SNa |
| A-821 | 2,6-dimethoxyphenyl | SNa |
| A-822 | 3,4-dimethoxyphenyl | SNa |
| A-823 | 3,5-dimethoxyphenyl | SNa |
| A-824 | 2,3-di(trifluoromethyl)phenyl | SNa |
| A-825 | 2,4-di(trifluoromethyl)phenyl | SNa |
| A-826 | 2,5-di(trifluoromethyl)phenyl | SNa |
| A-827 | 2,6-di(trifluoromethyl)phenyl | SNa |
| A-828 | 3,4-di(trifluoromethyl)phenyl | SNa |
| A-829 | 3,5-di(trifluoromethyl)phenyl | SNa |
| A-830 | 2,3-di(trifluoromethoxy)phenyl | SNa |
| A-831 | 2,4-di(trifluoromethoxy)phenyl | SNa |
| A-832 | 2,5-di(trifluoromethoxy)phenyl | SNa |
| A-833 | 2,6-di(trifluoromethoxy)phenyl | SNa |
| A-834 | 3,4-di(trifluoromethoxy)phenyl | SNa |
| A-835 | 3,5-di(trifluoromethoxy)phenyl | SNa |
| A-836 | 2,3-di(difluoromethoxy)phenyl | SNa |
| A-837 | 2,4-di(difluoromethoxy)phenyl | SNa |
| A-838 | 2,5-di(difluoromethoxy)phenyl | SNa |
| A-839 | 2,6-di(difluoromethoxy)phenyl | SNa |
| A-840 | 3,4-di(difluoromethoxy)phenyl | SNa |
| A-841 | 3,5-di(difluoromethoxy)phenyl | SNa |
| A-842 | 2,3-di(trifluoromethylthio)phenyl | SNa |
| A-843 | 2,4-di(trifluoromethylthio)phenyl | SNa |
| A-844 | 2,5-di(trifluoromethylthio)phenyl | SNa |
| A-845 | 2,6-di(trifluoromethylthio)phenyl | SNa |
| A-846 | 3,4-di(trifluoromethylthio)phenyl | SNa |
| A-847 | 3,5-di(trifluoromethylthio)phenyl | SNa |
| A-848 | 2-fluoro-3-chlorophenyl | SNa |
| A-849 | 2-fluoro-4-chlorophenyl | SNa |
| A-850 | 2-fluoro-5-chlorophenyl | SNa |
| A-851 | 2-fluoro-6-chlorophenyl | SNa |
| A-852 | 3-fluoro-4-chlorophenyl | SNa |
| A-853 | 3-fluoro-5-chlorophenyl | SNa |
| A-854 | 2-chloro-3-fluorophenyl | SNa |
| A-855 | 2-chloro-4-fluorophenyl | SNa |
| A-856 | 2-chloro-5-fluorophenyl | SNa |
| A-857 | 3-chloro-4-fluorophenyl | SNa |
| A-858 | 2-methyl-3-chlorophenyl | SNa |
| A-859 | 2-methyl-4-chlorophenyl | SNa |
| A-860 | 2-methyl-5-chlorophenyl | SNa |
| A-861 | 2-methyl-6-chlorophenyl | SNa |
| A-862 | 3-methyl-4-chlorophenyl | SNa |
| A-863 | 3-methyl-5-chlorophenyl | SNa |
| A-864 | 2-chloro-3-methylphenyl | SNa |
| A-865 | 2-chloro-4-methylphenyl | SNa |
| A-866 | 2-chloro-5-methylphenyl | SNa |
| A-867 | 3-chloro-4-methylphenyl | SNa |
| A-868 | 2-methyl-3-fluorophenyl | SNa |
| A-869 | 2-methyl-4-fluorophenyl | SNa |
| A-870 | 2-methyl-5-fluorophenyl | SNa |
| A-871 | 2-methyl-6-fluorophenyl | SNa |
| A-872 | 3-methyl-4-fluorophenyl | SNa |
| A-873 | 3-methyl-5-fluorophenyl | SNa |
| A-874 | 2-fluoro-3-methylphenyl | SNa |
| A-875 | 2-fluoro-4-methylphenyl | SNa |
| A-876 | 2-fluoro-5-methylphenyl | SNa |
| A-877 | 3-fluoro-4-methylphenyl | SNa |
| A-878 | 2-chloro-3-ethylphenyl | SNa |
| A-879 | 2-chloro-4-ethylphenyl | SNa |
| A-880 | 2-chloro-5-ethylphenyl | SNa |
| A-881 | 3-chloro-4-ethylphenyl | SNa |
| A-882 | 2-ethyl-3-chlorophenyl | SNa |
| A-883 | 2-ethyl-4-chlorophenyl | SNa |
| A-884 | 2-ethyl-5-chlorophenyl | SNa |
| A-885 | 2-ethyl-6-chlorophenyl | SNa |
| A-886 | 2-ethyl-3-fluorophenyl | SNa |
| A-887 | 2-ethyl-4-fluorophenyl | SNa |
| A-888 | 2-ethyl-5-fluorophenyl | SNa |
| A-889 | 2-ethyl-6-fluorophenyl | SNa |
| A-890 | 3-ethyl-4-fluorophenyl | SNa |
| A-891 | 3-ethyl-5-fluorophenyl | SNa |
| A-892 | 2-fluoro-3-ethylphenyl | SNa |
| A-893 | 2-fluoro-4-ethylphenyl | SNa |
| A-894 | 2-fluoro-5-ethylphenyl | SNa |
| A-895 | 3-fluoro-4-ethylphenyl | SNa |
| A-896 | 2-methoxy-3-chlorophenyl | SNa |
| A-897 | 2-methoxy-4-chlorophenyl | SNa |
| A-898 | 2-methoxy-5-chlorophenyl | SNa |
| A-899 | 2-methoxy-6-chlorophenyl | SNa |
| A-900 | 3-methoxy-4-chlorophenyl | SNa |
| A-901 | 3-methoxy-5-chlorophenyl | SNa |
| A-902 | 2-chloro-3-methoxyphenyl | SNa |
| A-903 | 2-chloro-4-methoxyphenyl | SNa |
| A-904 | 2-chloro-5-methoxyphenyl | SNa |
| A-905 | 3-chloro-4-methoxyphenyl | SNa |
| A-906 | 2-methoxy-3-fluorophenyl | SNa |
| A-907 | 2-methoxy-4-fluorophenyl | SNa |
| A-908 | 2-methoxy-5-fluorophenyl | SNa |
| A-909 | 2-methoxy-6-fluorophenyl | SNa |
| A-910 | 3-methoxy-4-fluorophenyl | SNa |
| A-911 | 3-methoxy-5-fluorophenyl | SNa |
| A-912 | 2-fluoro-3-methoxyphenyl | SNa |
| A-913 | 2-fluoro-4-methoxyphenyl | SNa |
| A-914 | 2-fluoro-5-methoxyphenyl | SNa |
| A-915 | 3-fluoro-4-methoxyphenyl | SNa |
| A-916 | 3-fluoro-5-methoxyphenyl | SNa |
| A-917 | 2-(trifluoromethyl)-3-chlorophenyl | SNa |
| A-918 | 2-(trifluoromethyl)-4-chlorophenyl | SNa |
| A-919 | 2-(trifluoromethyl)-5-chlorophenyl | SNa |
| A-920 | 2-(trifluoromethyl)-6-chlorophenyl | SNa |
| A-921 | 3-(trifluoromethyl)-4-chlorophenyl | SNa |
| A-922 | 3-(trifluoromethyl)-5-chlorophenyl | SNa |
| A-923 | 2-chloro-3-(trifluoromethyl)phenyl | SNa |
| A-924 | 2-chloro-4-(trifluoromethyl)phenyl | SNa |
| A-925 | 2-chloro-5-(trifluoromethyl)phenyl | SNa |
| A-926 | 3-chloro-4-(trifluoromethyl)phenyl | SNa |
| A-927 | 2-(trifluoromethyl)-3-fluorophenyl | SNa |
| A-928 | 2-(trifluoromethyl)-4-fluorophenyl | SNa |
| A-929 | 2-(trifluoromethyl)-5-fluorophenyl | SNa |
| A-930 | 2-(trifluoromethyl)-6-fluorophenyl | SNa |
| A-931 | 3-(trifluoromethyl)-4-fluorophenyl | SNa |
| A-932 | 3-(trifluoromethyl)-5-fluorophenyl | SNa |
| A-933 | 2-fluoro-3-(trifluoromethyl)phenyl | SNa |
| A-934 | 2-fluoro-4-(trifluoromethyl)phenyl | SNa |
| A-935 | 2-fluoro-5-(trifluoromethyl)phenyl | SNa |
| A-936 | 3-fluoro-4-(trifluoromethyl)phenyl | SNa |
| A-937 | 2-(trifluoromethoxy)-3-chlorophenyl | SNa |
| A-938 | 2-(trifluoromethoxy)-4-chlorophenyl | SNa |
| A-939 | 2-(trifluoromethoxy)-5-chlorophenyl | SNa |
| A-940 | 2-(trifluoromethoxy)-6-chlorophenyl | SNa |
| A-941 | 3-(trifluoromethoxy)-4-chlorophenyl | SNa |
| A-942 | 3-(trifluoromethoxy)-5-chlorophenyl | SNa |
| A-943 | 2-chloro-3-(trifluoromethoxy)phenyl | SNa |
| A-944 | 2-chloro-4-(trifluoromethoxy)phenyl | SNa |
| A-945 | 2-chloro-5-(trifluoromethoxy)phenyl | SNa |
| A-946 | 3-chloro-4-(trifluoromethoxy)phenyl | SNa |
| A-947 | 2-(trifluoromethoxy)-3-fluorophenyl | SNa |
| A-948 | 2-(trifluoromethoxy)-4-fluorophenyl | SNa |
| A-949 | 2-(trifluoromethoxy)-5-fluorophenyl | SNa |
| A-950 | 2-(trifluoromethoxy)-6-fluorophenyl | SNa |
| A-951 | 3-(trifluoromethoxy)-4-fluorophenyl | SNa |
| A-952 | 3-(trifluoromethoxy)-5-fluorophenyl | SNa |
| A-953 | 2-fluoro-3-(trifluoromethoxy)phenyl | SNa |
| A-954 | 2-fluoro-4-(trifluoromethoxy)phenyl | SNa |
| A-955 | 2-fluoro-5-(trifluoromethoxy)phenyl | SNa |
| A-956 | 3-fluoro-4-(trifluoromethoxy)phenyl | SNa |
| A-957 | 2-(difluoromethoxy)-3-chlorophenyl | SNa |
| A-958 | 2-(difluoromethoxy)-4-chlorophenyl | SNa |
| A-959 | 2-(difluoromethoxy)-5-chlorophenyl | SNa |
| A-960 | 2-(difluoromethoxy)-6-chlorophenyl | SNa |
| A-961 | 3-(difluoromethoxy)-4-chlorophenyl | SNa |
| A-962 | 3-(difluoromethoxy)-5-chlorophenyl | SNa |
| A-963 | 2-chloro-3-(difluoromethoxy)phenyl | SNa |
| A-964 | 2-chloro-4-(difluoromethoxy)phenyl | SNa |
| A-965 | 2-chloro-5-(difluoromethoxy)phenyl | SNa |
| A-966 | 3-chloro-4-(difluoromethoxy)phenyl | SNa |
| A-967 | 2-(difluoromethoxy)-3-fluorophenyl | SNa |
| A-968 | 2-(difluoromethoxy)-4-fluorophenyl | SNa |
| A-969 | 2-(difluoromethoxy)-5-fluorophenyl | SNa |
| A-970 | 2-(difluoromethoxy)-6-fluorophenyl | SNa |
| A-971 | 3-(difluoromethoxy)-4-fluorophenyl | SNa |
| A-972 | 3-(difluoromethoxy)-5-fluorophenyl | SNa |
| A-973 | 2-fluoro-3-(difluoromethoxy)phenyl | SNa |
| A-974 | 2-fluoro-4-(difluoromethoxy)phenyl | SNa |
| A-975 | 2-fluoro-5-(difluoromethoxy)phenyl | SNa |
| A-976 | 3-fluoro-4-(difluoromethoxy)phenyl | SNa |
| A-977 | 2-(trifluoromethylthio)-3-chlorophenyl | SNa |
| A-978 | 2-(trifluoromethylthio)-4-chlorophenyl | SNa |
| A-979 | 2-(trifluoromethylthio)-5-chlorophenyl | SNa |
| A-980 | 2-(trifluoromethylthio)-6-chlorophenyl | SNa |
| A-981 | 3-(trifluoromethylthio)-4-chlorophenyl | SNa |
| A-982 | 3-(trifluoromethylthio)-5-chlorophenyl | SNa |
| A-983 | 2-chloro-3-(trifluoromethylthio)phenyl | SNa |
| A-984 | 2-chloro-4-(trifluoromethylthio)phenyl | SNa |
| A-985 | 2-chloro-5-(trifluoromethylthio)phenyl | SNa |
| A-986 | 3-chloro-4-(trifluoromethylthio)phenyl | SNa |
| A-987 | 2-(trifluoromethylthio)-3-fluorophenyl | SNa |
| A-988 | 2-(trifluoromethylthio)-4-fluorophenyl | SNa |
| A-989 | 2-(trifluoromethylthio)-5-fluorophenyl | SNa |
| A-990 | 2-(trifluoromethylthio)-6-fluorophenyl | SNa |
| A-991 | 3-(trifluoromethylthio)-4-fluorophenyl | SNa |
| A-992 | 3-(trifluoromethylthio)-5-fluorophenyl | SNa |
| A-993 | 2-fluoro-3-(trifluoromethylthio)phenyl | SNa |
| A-994 | 2-fluoro-4-(trifluoromethylthio)phenyl | SNa |
| A-995 | 2-fluoro-5-(trifluoromethylthio)phenyl | SNa |
| A-996 | 3-fluoro-4-(trifluoromethylthio)phenyl | SNa |
| A-997 | 2,3,4-trichlorophenyl | SNa |
| A-998 | 2,3,5-trichlorophenyl | SNa |
| A-999 | 2,3,6-trichlorophenyl | SNa |
| A-1000 | 2,4,5-trichlorophenyl | SNa |
| A-1001 | 2,4,6-trichlorophenyl | SNa |
| A-1002 | 3,4,5-trichlorophenyl | SNa |
| A-1003 | 2,3,4-trifluorophenyl | SNa |
| A-1004 | 2,3,5-trifluorophenyl | SNa |
| A-1005 | 2,3,6-trifluorophenyl | SNa |
| A-1006 | 2,4,5-trifluorophenyl | SNa |
| A-1007 | 2,4,6-trifluorophenyl | SNa |
| A-1008 | 3,4,5-trifluorophenyl | SNa |
| A-1009 | 2,3,4-trimethylphenyl | SNa |
| A-1010 | 2,3,5-trimethylphenyl | SNa |
| A-1011 | 2,3,6-trimethylphenyl | SNa |
| A-1012 | 2,4,5-trimethylphenyl | SNa |
| A-1013 | 2,4,6-trimethylphenyl | SNa |
| A-1014 | 3,4,5-trimethylphenyl | SNa |
| A-1015 | 2,3,4-trimethoxyphenyl | SNa |
| A-1016 | 2,3,5-trimethoxyphenyl | SNa |
| A-1017 | 2,3,6-trimethoxyphenyl | SNa |
| A-1018 | 2,4,5-trimethoxyphenyl | SNa |
| A-1019 | 2,4,6-trimethoxyphenyl | SNa |
| A-1020 | 3,4,5-trimethoxyphenyl | SNa |
| A-1021 | phenyl | S(½Cu) |
| A-1022 | 2-chlorophenyl | S(½Cu) |
| A-1023 | 3-chlorophenyl | S(½Cu) |
| A-1024 | 4-chlorophenyl | S(½Cu) |
| A-1025 | 2-fluorophenyl | S(½Cu) |
| A-1026 | 3-fluorophenyl | S(½Cu) |
| A-1027 | 4-fluorophenyl | S(½Cu) |
| A-1028 | 2-methylphenyl | S(½Cu) |
| A-1029 | 3-methylphenyl | S(½Cu) |
| A-1030 | 4-methylphenyl | S(½Cu) |
| A-1031 | 2-ethylphenyl | S(½Cu) |
| A-1032 | 3-ethylphenyl | S(½Cu) |
| A-1033 | 4-ethylphenyl | S(½Cu) |
| A-1034 | 2-methoxyphenyl | S(½Cu) |
| A-1035 | 3-methoxyphenyl | S(½Cu) |
| A-1036 | 4-methoxyphenyl | S(½Cu) |
| A-1037 | 2-trifluoromethylphenyl | S(½Cu) |
| A-1038 | 3-trifluoromethylphenyl | S(½Cu) |
| A-1039 | 4-trifluoromethylphenyl | S(½Cu) |
| A-1040 | 2-trifluoromethoxyphenyl | S(½Cu) |
| A-1041 | 3-trifluoromethoxyphenyl | S(½Cu) |
| A-1042 | 4-trifluoromethoxyphenyl | S(½Cu) |
| A-1043 | 2-difluoromethoxyphenyl | S(½Cu) |
| A-1044 | 3-difluoromethoxyphenyl | S(½Cu) |
| A-1045 | 4-difluoromethoxyphenyl | S(½Cu) |
| A-1046 | 2-trifluoromethylthiophenyl | S(½Cu) |
| A-1047 | 3-trifluoromethylthiophenyl | S(½Cu) |
| A-1048 | 4-trifluoromethylthiophenyl | S(½Cu) |
| A-1049 | 2,3-dichlorophenyl | S(½Cu) |
| A-1050 | 2,4-dichlorophenyl | S(½Cu) |
| A-1051 | 2,5-dichlorophenyl | S(½Cu) |
| A-1052 | 2,6-dichlorophenyl | S(½Cu) |
| A-1053 | 3,4-dichlorophenyl | S(½Cu) |
| A-1054 | 3,5-dichlorophenyl | S(½Cu) |
| A-1055 | 2,3-difluorophenyl | S(½Cu) |
| A-1056 | 2,4-difluorophenyl | S(½Cu) |
| A-1057 | 2,5-difluorophenyl | S(½Cu) |
| A-1058 | 2,6-difluorophenyl | S(½Cu) |
| A-1059 | 3,4-difluorophenyl | S(½Cu) |
| A-1060 | 3,5-difluorophenyl | S(½Cu) |
| A-1061 | 2,3-dimethylphenyl | S(½Cu) |
| A-1062 | 2,4-dimethylphenyl | S(½Cu) |
| A-1063 | 2,5-dimethylphenyl | S(½Cu) |
| A-1064 | 2,6-dimethylphenyl | S(½Cu) |
| A-1065 | 3,4-dimethylphenyl | S(½Cu) |
| A-1066 | 3,5-dimethylphenyl | S(½Cu) |
| A-1067 | 2,3-diethylphenyl | S(½Cu) |
| A-1068 | 2,4-diethylphenyl | S(½Cu) |
| A-1069 | 2,5-diethylphenyl | S(½Cu) |
| A-1070 | 2,6-diethylphenyl | S(½Cu) |
| A-1071 | 3,5-diethylphenyl | S(½Cu) |
| A-1072 | 3,4-diethylphenyl | S(½Cu) |
| A-1073 | 2,3-dimethoxyphenyl | S(½Cu) |
| A-1074 | 2,4-dimethoxyphenyl | S(½Cu) |
| A-1075 | 2,5-dimethoxyphenyl | S(½Cu) |
| A-1076 | 2,6-dimethoxyphenyl | S(½Cu) |
| A-1077 | 3,4-dimethoxyphenyl | S(½Cu) |
| A-1078 | 3,5-dimethoxyphenyl | S(½Cu) |
| A-1079 | 2,3-di(trifluoromethyl)phenyl | S(½Cu) |
| A-1080 | 2,4-di(trifluoromethyl)phenyl | S(½Cu) |
| A-1081 | 2,5-di(trifluoromethyl)phenyl | S(½Cu) |
| A-1082 | 2,6-di(trifluoromethyl)phenyl | S(½Cu) |
| A-1083 | 3,4-di(trifluoromethyl)phenyl | S(½Cu) |
| A-1084 | 3,5-di(trifluoromethyl)phenyl | S(½Cu) |
| A-1085 | 2,3-di(trifluoromethoxy)phenyl | S(½Cu) |
| A-1086 | 2,4-di(trifluoromethoxy)phenyl | S(½Cu) |
| A-1087 | 2,5-di(trifluoromethoxy)phenyl | S(½Cu) |
| A-1088 | 2,6-di(trifluoromethoxy)phenyl | S(½Cu) |
| A-1089 | 3,4-di(trifluoromethoxy)phenyl | S(½Cu) |
| A-1090 | 3,5-di(trifluoromethoxy)phenyl | S(½Cu) |
| A-1091 | 2,3-di(difluoromethoxy)phenyl | S(½Cu) |
| A-1092 | 2,4-di(difluoromethoxy)phenyl | S(½Cu) |
| A-1093 | 2,5-di(difluoromethoxy)phenyl | S(½Cu) |
| A-1094 | 2,6-di(difluoromethoxy)phenyl | S(½Cu) |
| A-1095 | 3,4-di(difluoromethoxy)phenyl | S(½Cu) |
| A-1096 | 3,5-di(difluoromethoxy)phenyl | S(½Cu) |
| A-1097 | 2,3-di(trifluoromethylthio)phenyl | S(½Cu) |
| A-1098 | 2,4-di(trifluoromethylthio)phenyl | S(½Cu) |
| A-1099 | 2,5-di(trifluoromethylthio)phenyl | S(½Cu) |
| A-1100 | 2,6-di(trifluoromethylthio)phenyl | S(½Cu) |
| A-1101 | 3,4-di(trifluoromethylthio)phenyl | S(½Cu) |
| A-1102 | 3,5-di(trifluoromethylthio)phenyl | S(½Cu) |
| A-1103 | 2-fluoro-3-chlorophenyl | S(½Cu) |
| A-1104 | 2-fluoro-4-chlorophenyl | S(½Cu) |
| A-1105 | 2-fluoro-5-chlorophenyl | S(½Cu) |
| A-1106 | 2-fluoro-6-chlorophenyl | S(½Cu) |
| A-1107 | 3-fluoro-4-chlorophenyl | S(½Cu) |
| A-1108 | 3-fluoro-5-chlorophenyl | S(½Cu) |
| A-1109 | 2-chloro-3-fluorophenyl | S(½Cu) |
| A-1110 | 2-chloro-4-fluorophenyl | S(½Cu) |
| A-1111 | 2-chloro-5-fluorophenyl | S(½Cu) |
| A-1112 | 3-chloro-4-fluorophenyl | S(½Cu) |
| A-1113 | 2-methyl-3-chlorophenyl | S(½Cu) |
| A-1114 | 2-methyl-4-chlorophenyl | S(½Cu) |
| A-1115 | 2-methyl-5-chlorophenyl | S(½Cu) |
| A-1116 | 2-methyl-6-chlorophenyl | S(½Cu) |
| A-1117 | 3-methyl-4-chlorophenyl | S(½Cu) |
| A-1118 | 3-methyl-5-chlorophenyl | S(½Cu) |
| A-1119 | 2-chloro-3-methylphenyl | S(½Cu) |
| A-1120 | 2-chloro-4-methylphenyl | S(½Cu) |
| A-1121 | 2-chloro-5-methylphenyl | S(½Cu) |
| A-1122 | 3-chloro-4-methylphenyl | S(½Cu) |
| A-1123 | 2-methyl-3-fluorophenyl | S(½Cu) |
| A-1124 | 2-methyl-4-fluorophenyl | S(½Cu) |
| A-1125 | 2-methyl-5-fluorophenyl | S(½Cu) |
| A-1126 | 2-methyl-6-fluorophenyl | S(½Cu) |
| A-1127 | 3-methyl-4-fluorophenyl | S(½Cu) |
| A-1128 | 3-methyl-5-fluorophenyl | S(½Cu) |
| A-1129 | 2-fluoro-3-methylphenyl | S(½Cu) |
| A-1130 | 2-fluoro-4-methylphenyl | S(½Cu) |
| A-1131 | 2-fluoro-5-methylphenyl | S(½Cu) |
| A-1132 | 3-fluoro-4-methylphenyl | S(½Cu) |
| A-1133 | 2-chloro-3-ethylphenyl | S(½Cu) |
| A-1134 | 2-chloro-4-ethylphenyl | S(½Cu) |
| A-1135 | 2-chloro-5-ethylphenyl | S(½Cu) |
| A-1136 | 3-chloro-4-ethylphenyl | S(½Cu) |
| A-1137 | 2-ethyl-3-chlorophenyl | S(½Cu) |
| A-1138 | 2-ethyl-4-chlorophenyl | S(½Cu) |
| A-1139 | 2-ethyl-5-chlorophenyl | S(½Cu) |
| A-1140 | 2-ethyl-6-chlorophenyl | S(½Cu) |
| A-1141 | 2-ethyl-3-fluorophenyl | S(½Cu) |
| A-1142 | 2-ethyl-4-fluorophenyl | S(½Cu) |
| A-1143 | 2-ethyl-5-fluorophenyl | S(½Cu) |
| A-1144 | 2-ethyl-6-fluorophenyl | S(½Cu) |
| A-1145 | 3-ethyl-4-fluorophenyl | S(½Cu) |
| A-1146 | 3-ethyl-5-fluorophenyl | S(½Cu) |
| A-1147 | 2-fluoro-3-ethylphenyl | S(½Cu) |
| A-1148 | 2-fluoro-4-ethylphenyl | S(½Cu) |
| A-1149 | 2-fluoro-5-ethylphenyl | S(½Cu) |
| A-1150 | 3-fluoro-4-ethylphenyl | S(½Cu) |
| A-1151 | 2-methoxy-3-chlorophenyl | S(½Cu) |
| A-1152 | 2-methoxy-4-chlorophenyl | S(½Cu) |
| A-1153 | 2-methoxy-5-chlorophenyl | S(½Cu) |
| A-1154 | 2-methoxy-6-chlorophenyl | S(½Cu) |
| A-1155 | 3-methoxy-4-chlorophenyl | S(½Cu) |
| A-1156 | 3-methoxy-5-chlorophenyl | S(½Cu) |
| A-1157 | 2-chloro-3-methoxyphenyl | S(½Cu) |
| A-1158 | 2-chloro-4-methoxyphenyl | S(½Cu) |
| A-1159 | 2-chloro-5-methoxyphenyl | S(½Cu) |
| A-1160 | 3-chloro-4-methoxyphenyl | S(½Cu) |
| A-1161 | 2-methoxy-3-fluorophenyl | S(½Cu) |
| A-1162 | 2-methoxy-4-fluorophenyl | S(½Cu) |
| A-1163 | 2-methoxy-5-fluorophenyl | S(½Cu) |
| A-1164 | 2-methoxy-6-fluorophenyl | S(½Cu) |
| A-1165 | 3-methoxy-4-fluorophenyl | S(½Cu) |
| A-1166 | 3-methoxy-5-fluorophenyl | S(½Cu) |
| A-1167 | 2-fluoro-3-methoxyphenyl | S(½Cu) |
| A-1168 | 2-fluoro-4-methoxyphenyl | S(½Cu) |
| A-1169 | 2-fluoro-5-methoxyphenyl | S(½Cu) |
| A-1170 | 3-fluoro-4-methoxyphenyl | S(½Cu) |
| A-1171 | 3-fluoro-5-methoxyphenyl | S(½Cu) |
| A-1172 | 2-(trifluoromethyl)-3-chlorophenyl | S(½Cu) |
| A-1173 | 2-(trifluoromethyl)-4-chlorophenyl | S(½Cu) |
| A-1174 | 2-(trifluoromethyl)-5-chlorophenyl | S(½Cu) |
| A-1175 | 2-(trifluoromethyl)-6-chlorophenyl | S(½Cu) |
| A-1176 | 3-(trifluoromethyl)-4-chlorophenyl | S(½Cu) |
| A-1177 | 3-(trifluoromethyl)-5-chlorophenyl | S(½Cu) |
| A-1178 | 2-chloro-3-(trifluoromethyl)phenyl | S(½Cu) |
| A-1179 | 2-chloro-4-(trifluoromethyl)phenyl | S(½Cu) |
| A-1180 | 2-chloro-5-(trifluoromethyl)phenyl | S(½Cu) |
| A-1181 | 3-chloro-4-(trifluoromethyl)phenyl | S(½Cu) |
| A-1182 | 2-(trifluoromethyl)-3-fluorophenyl | S(½Cu) |
| A-1183 | 2-(trifluoromethyl)-4-fluorophenyl | S(½Cu) |
| A-1184 | 2-(trifluoromethyl)-5-fluorophenyl | S(½Cu) |
| A-1185 | 2-(trifluoromethyl)-6-fluorophenyl | S(½Cu) |
| A-1186 | 3-(trifluoromethyl)-4-fluorophenyl | S(½Cu) |
| A-1187 | 3-(trifluoromethyl)-5-fluorophenyl | S(½Cu) |
| A-1188 | 2-fluoro-3-(trifluoromethyl)phenyl | S(½Cu) |
| A-1189 | 2-fluoro-4-(trifluoromethyl)phenyl | S(½Cu) |
| A-1190 | 2-fluoro-5-(trifluoromethyl)phenyl | S(½Cu) |
| A-1191 | 3-fluoro-4-(trifluoromethyl)phenyl | S(½Cu) |
| A-1192 | 2-(trifluoromethoxy)-3-chlorophenyl | S(½Cu) |
| A-1193 | 2-(trifluoromethoxy)-4-chlorophenyl | S(½Cu) |
| A-1194 | 2-(trifluoromethoxy)-5-chlorophenyl | S(½Cu) |
| A-1195 | 2-(trifluoromethoxy)-6-chlorophenyl | S(½Cu) |
| A-1196 | 3-(trifluoromethoxy)-4-chlorophenyl | S(½Cu) |
| A-1197 | 3-(trifluoromethoxy)-5-chlorophenyl | S(½Cu) |
| A-1198 | 2-chloro-3-(trifluoromethoxy)phenyl | S(½Cu) |
| A-1199 | 2-chloro-4-(trifluoromethoxy)phenyl | S(½Cu) |
| A-1200 | 2-chloro-5-(trifluoromethoxy)phenyl | S(½Cu) |
| A-1201 | 3-chloro-4-(trifluoromethoxy)phenyl | S(½Cu) |
| A-1202 | 2-(trifluoromethoxy)-3-fluorophenyl | S(½Cu) |
| A-1203 | 2-(trifluoromethoxy)-4-fluorophenyl | S(½Cu) |
| A-1204 | 2-(trifluoromethoxy)-5-fluorophenyl | S(½Cu) |
| A-1205 | 2-(trifluoromethoxy)-6-fluorophenyl | S(½Cu) |
| A-1206 | 3-(trifluoromethoxy)-4-fluorophenyl | S(½Cu) |
| A-1207 | 3-(trifluoromethoxy)-5-fluorophenyl | S(½Cu) |
| A-1208 | 2-fluoro-3-(trifluoromethoxy)phenyl | S(½Cu) |
| A-1209 | 2-fluoro-4-(trifluoromethoxy)phenyl | S(½Cu) |
| A-1210 | 2-fluoro-5-(trifluoromethoxy)phenyl | S(½Cu) |
| A-1211 | 3-fluoro-4-(trifluoromethoxy)phenyl | S(½Cu) |
| A-1212 | 2-(difluoromethoxy)-3-chlorophenyl | S(½Cu) |
| A-1213 | 2-(difluoromethoxy)-4-chlorophenyl | S(½Cu) |
| A-1214 | 2-(difluoromethoxy)-5-chlorophenyl | S(½Cu) |
| A-1215 | 2-(difluoromethoxy)-6-chlorophenyl | S(½Cu) |
| A-1216 | 3-(difluoromethoxy)-4-chlorophenyl | S(½Cu) |
| A-1217 | 3-(difluoromethoxy)-5-chlorophenyl | S(½Cu) |
| A-1218 | 2-chloro-3-(difluoromethoxy)phenyl | S(½Cu) |
| A-1219 | 2-chloro-4-(difluoromethoxy)phenyl | S(½Cu) |
| A-1220 | 2-chloro-5-(difluoromethoxy)phenyl | S(½Cu) |
| A-1221 | 3-chloro-4-(difluoromethoxy)phenyl | S(½Cu) |
| A-1222 | 2-(difluoromethoxy)-3-fluorophenyl | S(½Cu) |
| A-1223 | 2-(difluoromethoxy)-4-fluorophenyl | S(½Cu) |
| A-1224 | 2-(difluoromethoxy)-5-fluorophenyl | S(½Cu) |
| A-1225 | 2-(difluoromethoxy)-6-fluorophenyl | S(½Cu) |
| A-1226 | 3-(difluoromethoxy)-4-fluorophenyl | S(½Cu) |
| A-1227 | 3-(difluoromethoxy)-5-fluorophenyl | S(½Cu) |
| A-1228 | 2-fluoro-3-(difluoromethoxy)phenyl | S(½Cu) |
| A-1229 | 2-fluoro-4-(difluoromethoxy)phenyl | S(½Cu) |
| A-1230 | 2-fluoro-5-(difluoromethoxy)phenyl | S(½Cu) |
| A-1231 | 3-fluoro-4-(difluoromethoxy)phenyl | S(½Cu) |
| A-1232 | 2-(trifluoromethylthio)-3-chlorophenyl | S(½Cu) |
| A-1233 | 2-(trifluoromethylthio)-4-chlorophenyl | S(½Cu) |
| A-1234 | 2-(trifluoromethylthio)-5-chlorophenyl | S(½Cu) |
| A-1235 | 2-(trifluoromethylthio)-6-chlorophenyl | S(½Cu) |
| A-1236 | 3-(trifluoromethylthio)-4-chlorophenyl | S(½Cu) |
| A-1237 | 3-(trifluoromethylthio)-5-chlorophenyl | S(½Cu) |
| A-1238 | 2-chloro-3-(trifluoromethylthio)phenyl | S(½Cu) |
| A-1239 | 2-chloro-4-(trifluoromethylthio)phenyl | S(½Cu) |
| A-1240 | 2-chloro-5-(trifluoromethylthio)phenyl | S(½Cu) |
| A-1241 | 3-chloro-4-(trifluoromethylthio)phenyl | S(½Cu) |
| A-1242 | 2-(trifluoromethylthio)-3-fluorophenyl | S(½Cu) |
| A-1243 | 2-(trifluoromethylthio)-4-fluorophenyl | S(½Cu) |
| A-1244 | 2-(trifluoromethylthio)-5-fluorophenyl | S(½Cu) |
| A-1245 | 2-(trifluoromethylthio)-6-fluorophenyl | S(½Cu) |
| A-1246 | 3-(trifluoromethylthio)-4-fluorophenyl | S(½Cu) |
| A-1247 | 3-(trifluoromethylthio)-5-fluorophenyl | S(½Cu) |
| A-1248 | 2-fluoro-3-(trifluoromethylthio)phenyl | S(½Cu) |
| A-1249 | 2-fluoro-4-(trifluoromethylthio)phenyl | S(½Cu) |
| A-1250 | 2-fluoro-5-(trifluoromethylthio)phenyl | S(½Cu) |
| A-1251 | 3-fluoro-4-(trifluoromethylthio)phenyl | S(½Cu) |
| A-1252 | 2,3,4-trichlorophenyl | S(½Cu) |
| A-1253 | 2,3,5-trichlorophenyl | S(½Cu) |
| A-1254 | 2,3,6-trichlorophenyl | S(½Cu) |
| A-1255 | 2,4,5-trichlorophenyl | S(½Cu) |
| A-1256 | 2,4,6-trichlorophenyl | S(½Cu) |
| A-1257 | 3,4,5-trichlorophenyl | S(½Cu) |
| A-1258 | 2,3,4-trifluorophenyl | S(½Cu) |
| A-1259 | 2,3,5-trifluorophenyl | S(½Cu) |
| A-1260 | 2,3,6-trifluorophenyl | S(½Cu) |
| A-1261 | 2,4,5-trifluorophenyl | S(½Cu) |
| A-1262 | 2,4,6-trifluorophenyl | S(½Cu) |
| A-1263 | 3,4,5-trifluorophenyl | S(½Cu) |
| A-1264 | 2,3,4-trimethylphenyl | S(½Cu) |
| A-1265 | 2,3,5-trimethylphenyl | S(½Cu) |
| A-1266 | 2,3,6-trimethylphenyl | S(½Cu) |
| A-1267 | 2,4,5-trimethylphenyl | S(½Cu) |
| A-1268 | 2,4,6-trimethylphenyl | S(½Cu) |
| A-1269 | 3,4,5-trimethylphenyl | S(½Cu) |
| A-1270 | 2,3,4-trimethoxyphenyl | S(½Cu) |
| A-1271 | 2,3,5-trimethoxyphenyl | S(½Cu) |
| A-1272 | 2,3,6-trimethoxyphenyl | S(½Cu) |
| A-1273 | 2,4,5-trimethoxyphenyl | S(½Cu) |
| A-1274 | 2,4,6-trimethoxyphenyl | S(½Cu) |
| A-1275 | 3,4,5-trimethoxyphenyl | S(½Cu) |
| A-1276 | phenyl | S(HNEt₃) |
| A-1277 | 2-chlorophenyl | S(HNEt₃) |
| A-1278 | 3-chlorophenyl | S(HNEt₃) |
| A-1279 | 4-chlorophenyl | S(HNEt₃) |
| A-1280 | 2-fluorophenyl | S(HNEt₃) |
| A-1281 | 3-fluorophenyl | S(HNEt₃) |
| A-1282 | 4-fluorophenyl | S(HNEt₃) |
| A-1283 | 2-methylphenyl | S(HNEt₃) |
| A-1284 | 3-methylphenyl | S(HNEt₃) |
| A-1285 | 4-methylphenyl | S(HNEt₃) |
| A-1286 | 2-ethylphenyl | S(HNEt₃) |
| A-1287 | 3-ethylphenyl | S(HNEt₃) |
| A-1288 | 4-ethylphenyl | S(HNEt₃) |
| A-1289 | 2-methoxyphenyl | S(HNEt₃) |
| A-1290 | 3-methoxyphenyl | S(HNEt₃) |
| A-1291 | 4-methoxyphenyl | S(HNEt₃) |
| A-1292 | 2-trifluoromethylphenyl | S(HNEt₃) |
| A-1293 | 3-trifluoromethylphenyl | S(HNEt₃) |
| A-1294 | 4-trifluoromethylphenyl | S(HNEt₃) |
| A-1295 | 2-trifluoromethoxyphenyl | S(HNEt₃) |
| A-1296 | 3-trifluoromethoxyphenyl | S(HNEt₃) |
| A-1297 | 4-trifluoromethoxyphenyl | S(HNEt₃) |
| A-1298 | 2-difluoromethoxyphenyl | S(HNEt₃) |
| A-1299 | 3-difluoromethoxyphenyl | S(HNEt₃) |
| A-1300 | 4-difluoromethoxyphenyl | S(HNEt₃) |
| A-1301 | 2-trifluoromethylthiophenyl | S(HNEt₃) |
| A-1302 | 3-trifluoromethylthiophenyl | S(HNEt₃) |
| A-1303 | 4-trifluoromethylthiophenyl | S(HNEt₃) |
| A-1304 | 2,3-dichlorophenyl | S(HNEt₃) |
| A-1305 | 2,4-dichlorophenyl | S(HNEt₃) |
| A-1306 | 2,5-dichlorophenyl | S(HNEt₃) |
| A-1307 | 2,6-dichlorophenyl | S(HNEt₃) |
| A-1308 | 3,4-dichlorophenyl | S(HNEt₃) |
| A-1309 | 3,5-dichlorophenyl | S(HNEt₃) |
| A-1310 | 2,3-difluorophenyl | S(HNEt₃) |
| A-1311 | 2,4-difluorophenyl | S(HNEt₃) |
| A-1312 | 2,5-difluorophenyl | S(HNEt₃) |
| A-1313 | 2,6-difluorophenyl | S(HNEt₃) |
| A-1314 | 3,4-difluorophenyl | S(HNEt₃) |
| A-1315 | 3,5-difluorophenyl | S(HNEt₃) |
| A-1316 | 2,3-dimethylphenyl | S(HNEt₃) |
| A-1317 | 2,4-dimethylphenyl | S(HNEt₃) |
| A-1318 | 2,5-dimethylphenyl | S(HNEt₃) |
| A-1319 | 2,6-dimethylphenyl | S(HNEt₃) |
| A-1320 | 3,4-dimethylphenyl | S(HNEt₃) |
| A-1321 | 3,5-dimethylphenyl | S(HNEt₃) |
| A-1322 | 2,3-diethylphenyl | S(HNEt₃) |
| A-1323 | 2,4-diethylphenyl | S(HNEt₃) |
| A-1324 | 2,5-diethylphenyl | S(HNEt₃) |
| A-1325 | 2,6-diethylphenyl | S(HNEt₃) |
| A-1326 | 3,5-diethylphenyl | S(HNEt₃) |
| A-1327 | 3,4-diethylphenyl | S(HNEt₃) |
| A-1328 | 2,3-dimethoxyphenyl | S(HNEt₃) |
| A-1329 | 2,4-dimethoxyphenyl | S(HNEt₃) |
| A-1330 | 2,5-dimethoxyphenyl | S(HNEt₃) |
| A-1331 | 2,6-dimethoxyphenyl | S(HNEt₃) |
| A-1332 | 3,4-dimethoxyphenyl | S(HNEt₃) |
| A-1333 | 3,5-dimethoxyphenyl | S(HNEt₃) |
| A-1334 | 2,3-di(trifluoromethyl)phenyl | S(HNEt₃) |
| A-1335 | 2,4-di(trifluoromethyl)phenyl | S(HNEt₃) |
| A-1336 | 2,5-di(trifluoromethyl)phenyl | S(HNEt₃) |
| A-1337 | 2,6-di(trifluoromethyl)phenyl | S(HNEt₃) |
| A-1338 | 3,4-di(trifluoromethyl)phenyl | S(HNEt₃) |
| A-1339 | 3,5-di(trifluoromethyl)phenyl | S(HNEt₃) |
| A-1340 | 2,3-di(trifluoromethoxy)phenyl | S(HNEt₃) |
| A-1341 | 2,4-di(trifluoromethoxy)phenyl | S(HNEt₃) |
| A-1342 | 2,5-di(trifluoromethoxy)phenyl | S(HNEt₃) |
| A-1343 | 2,6-di(trifluoromethoxy)phenyl | S(HNEt₃) |
| A-1344 | 3,4-di(trifluoromethoxy)phenyl | S(HNEt₃) |
| A-1345 | 3,5-di(trifluoromethoxy)phenyl | S(HNEt₃) |
| A-1346 | 2,3-di(difluoromethoxy)phenyl | S(HNEt₃) |
| A-1347 | 2,4-di(difluoromethoxy)phenyl | S(HNEt₃) |
| A-1348 | 2,5-di(difluoromethoxy)phenyl | S(HNEt₃) |
| A-1349 | 2,6-di(difluoromethoxy)phenyl | S(HNEt₃) |
| A-1350 | 3,4-di(difluoromethoxy)phenyl | S(HNEt₃) |
| A-1351 | 3,5-di(difluoromethoxy)phenyl | S(HNEt₃) |
| A-1352 | 2,3-di(trifluoromethylthio)phenyl | S(HNEt₃) |
| A-1353 | 2,4-di(trifluoromethylthio)phenyl | S(HNEt₃) |
| A-1354 | 2,5-di(trifluoromethylthio)phenyl | S(HNEt₃) |
| A-1355 | 2,6-di(trifluoromethylthio)phenyl | S(HNEt₃) |
| A-1356 | 3,4-di(trifluoromethylthio)phenyl | S(HNEt₃) |
| A-1357 | 3,5-di(trifluoromethylthio)phenyl | S(HNEt₃) |
| A-1358 | 2-fluoro-3-chlorophenyl | S(HNEt₃) |
| A-1359 | 2-fluoro-4-chlorophenyl | S(HNEt₃) |
| A-1360 | 2-fluoro-5-chlorophenyl | S(HNEt₃) |
| A-1361 | 2-fluoro-6-chlorophenyl | S(HNEt₃) |
| A-1362 | 3-fluoro-4-chlorophenyl | S(HNEt₃) |
| A-1363 | 3-fluoro-5-chlorophenyl | S(HNEt₃) |
| A-1364 | 2-chloro-3-fluorophenyl | S(HNEt₃) |
| A-1365 | 2-chloro-4-fluorophenyl | S(HNEt₃) |
| A-1366 | 2-chloro-5-fluorophenyl | S(HNEt₃) |
| A-1367 | 3-chloro-4-fluorophenyl | S(HNEt₃) |
| A-1368 | 2-methyl-3-chlorophenyl | S(HNEt₃) |
| A-1369 | 2-methyl-4-chlorophenyl | S(HNEt₃) |
| A-1370 | 2-methyl-5-chlorophenyl | S(HNEt₃) |
| A-1371 | 2-methyl-6-chlorophenyl | S(HNEt₃) |
| A-1372 | 3-methyl-4-chlorophenyl | S(HNEt₃) |
| A-1373 | 3-methyl-5-chlorophenyl | S(HNEt₃) |
| A-1374 | 2-chloro-3-methylphenyl | S(HNEt₃) |
| A-1375 | 2-chloro-4-methylphenyl | S(HNEt₃) |
| A-1376 | 2-chloro-5-methylphenyl | S(HNEt₃) |
| A-1377 | 3-chloro-4-methylphenyl | S(HNEt₃) |
| A-1378 | 2-methyl-3-fluorophenyl | S(HNEt₃) |
| A-1379 | 2-methyl-4-fluorophenyl | S(HNEt₃) |
| A-1380 | 2-methyl-5-fluorophenyl | S(HNEt₃) |
| A-1381 | 2-methyl-6-fluorophenyl | S(HNEt₃) |
| A-1382 | 3-methyl-4-fluorophenyl | S(HNEt₃) |
| A-1383 | 3-methyl-5-fluorophenyl | S(HNEt₃) |
| A-1384 | 2-fluoro-3-methylphenyl | S(HNEt₃) |
| A-1385 | 2-fluoro-4-methylphenyl | S(HNEt₃) |
| A-1386 | 2-fluoro-5-methylphenyl | S(HNEt₃) |
| A-1387 | 3-fluoro-4-methylphenyl | S(HNEt₃) |
| A-1388 | 2-chloro-3-ethylphenyl | S(HNEt₃) |
| A-1389 | 2-chloro-4-ethylphenyl | S(HNEt₃) |
| A-1390 | 2-chloro-5-ethylphenyl | S(HNEt₃) |
| A-1391 | 3-chloro-4-ethylphenyl | S(HNEt₃) |
| A-1392 | 2-ethyl-3-chlorophenyl | S(HNEt₃) |
| A-1393 | 2-ethyl-4-chlorophenyl | S(HNEt₃) |
| A-1394 | 2-ethyl-5-chlorophenyl | S(HNEt₃) |
| A-1395 | 2-ethyl-6-chlorophenyl | S(HNEt₃) |
| A-1396 | 2-ethyl-3-fluorophenyl | S(HNEt₃) |
| A-1397 | 2-ethyl-4-fluorophenyl | S(HNEt₃) |
| A-1398 | 2-ethyl-5-fluorophenyl | S(HNEt₃) |
| A-1399 | 2-ethyl-6-fluorophenyl | S(HNEt₃) |
| A-1400 | 3-ethyl-4-fluorophenyl | S(HNEt₃) |
| A-1401 | 3-ethyl-5-fluorophenyl | S(HNEt₃) |
| A-1402 | 2-fluoro-3-ethylphenyl | S(HNEt₃) |
| A-1403 | 2-fluoro-4-ethylphenyl | S(HNEt₃) |
| A-1404 | 2-fluoro-5-ethylphenyl | S(HNEt₃) |
| A-1405 | 3-fluoro-4-ethylphenyl | S(HNEt₃) |
| A-1406 | 2-methoxy-3-chlorophenyl | S(HNEt₃) |
| A-1407 | 2-methoxy-4-chlorophenyl | S(HNEt₃) |
| A-1408 | 2-methoxy-5-chlorophenyl | S(HNEt₃) |
| A-1409 | 2-methoxy-6-chlorophenyl | S(HNEt₃) |
| A-1410 | 3-methoxy-4-chlorophenyl | S(HNEt₃) |
| A-1411 | 3-methoxy-5-chlorophenyl | S(HNEt₃) |
| A-1412 | 2-chloro-3-methoxyphenyl | S(HNEt₃) |
| A-1413 | 2-chloro-4-methoxyphenyl | S(HNEt₃) |
| A-1414 | 2-chloro-5-methoxyphenyl | S(HNEt₃) |
| A-1415 | 3-chloro-4-methoxyphenyl | S(HNEt₃) |
| A-1416 | 2-methoxy-3-fluorophenyl | S(HNEt₃) |
| A-1417 | 2-methoxy-4-fluorophenyl | S(HNEt₃) |
| A-1418 | 2-methoxy-5-fluorophenyl | S(HNEt₃) |
| A-1419 | 2-methoxy-6-fluorophenyl | S(HNEt₃) |
| A-1420 | 3-methoxy-4-fluorophenyl | S(HNEt₃) |
| A-1421 | 3-methoxy-5-fluorophenyl | S(HNEt₃) |
| A-1422 | 2-fluoro-3-methoxyphenyl | S(HNEt₃) |
| A-1423 | 2-fluoro-4-methoxyphenyl | S(HNEt₃) |
| A-1424 | 2-fluoro-5-methoxyphenyl | S(HNEt₃) |
| A-1425 | 3-fluoro-4-methoxyphenyl | S(HNEt₃) |
| A-1426 | 3-fluoro-5-methoxyphenyl | S(HNEt₃) |
| A-1427 | 2-(trifluoromethyl)-3-chlorophenyl | S(HNEt₃) |
| A-1428 | 2-(trifluoromethyl)-4-chlorophenyl | S(HNEt₃) |
| A-1429 | 2-(trifluoromethyl)-5-chlorophenyl | S(HNEt₃) |
| A-1430 | 2-(trifluoromethyl)-6-chlorophenyl | S(HNEt₃) |
| A-1431 | 3-(trifluoromethyl)-4-chlorophenyl | S(HNEt₃) |
| A-1432 | 3-(trifluoromethyl)-5-chlorophenyl | S(HNEt₃) |
| A-1433 | 2-chloro-3-(trifluoromethyl)phenyl | S(HNEt₃) |
| A-1434 | 2-chloro-4-(trifluoromethyl)phenyl | S(HNEt₃) |
| A-1435 | 2-chloro-5-(trifluoromethyl)phenyl | S(HNEt₃) |
| A-1436 | 3-chloro-4-(trifluoromethyl)phenyl | S(HNEt₃) |
| A-1437 | 2-(trifluoromethyl)-3-fluorophenyl | S(HNEt₃) |
| A-1438 | 2-(trifluoromethyl)-4-fluorophenyl | S(HNEt₃) |
| A-1439 | 2-(trifluoromethyl)-5-fluorophenyl | S(HNEt₃) |
| A-1440 | 2-(trifluoromethyl)-6-fluorophenyl | S(HNEt₃) |
| A-1441 | 3-(trifluoromethyl)-4-fluorophenyl | S(HNEt₃) |
| A-1442 | 3-(trifluoromethyl)-5-fluorophenyl | S(HNEt₃) |
| A-1443 | 2-fluoro-3-(trifluoromethyl)phenyl | S(HNEt₃) |
| A-1444 | 2-fluoro-4-(trifluoromethyl)phenyl | S(HNEt₃) |
| A-1445 | 2-fluoro-5-(trifluoromethyl)phenyl | S(HNEt₃) |
| A-1446 | 3-fluoro-4-(trifluoromethyl)phenyl | S(HNEt₃) |
| A-1447 | 2-(trifluoromethoxy)-3-chlorophenyl | S(HNEt₃) |
| A-1448 | 2-(trifluoromethoxy)-4-chlorophenyl | S(HNEt₃) |
| A-1449 | 2-(trifluoromethoxy)-5-chlorophenyl | S(HNEt₃) |
| A-1450 | 2-(trifluoromethoxy)-6-chlorophenyl | S(HNEt₃) |
| A-1451 | 3-(trifluoromethoxy)-4-chlorophenyl | S(HNEt₃) |
| A-1452 | 3-(trifluoromethoxy)-5-chlorophenyl | S(HNEt₃) |
| A-1453 | 2-chloro-3-(trifluoromethoxy)phenyl | S(HNEt₃) |
| A-1454 | 2-chloro-4-(trifluoromethoxy)phenyl | S(HNEt₃) |
| A-1455 | 2-chloro-5-(trifluoromethoxy)phenyl | S(HNEt₃) |
| A-1456 | 3-chloro-4-(trifluoromethoxy)phenyl | S(HNEt₃) |
| A-1457 | 2-(trifluoromethoxy)-3-fluorophenyl | S(HNEt₃) |
| A-1458 | 2-(trifluoromethoxy)-4-fluorophenyl | S(HNEt₃) |
| A-1459 | 2-(trifluoromethoxy)-5-fluorophenyl | S(HNEt₃) |
| A-1460 | 2-(trifluoromethoxy)-6-fluorophenyl | S(HNEt₃) |
| A-1461 | 3-(trifluoromethoxy)-4-fluorophenyl | S(HNEt₃) |
| A-1462 | 3-(trifluoromethoxy)-5-fluorophenyl | S(HNEt₃) |
| A-1463 | 2-fluoro-3-(trifluoromethoxy)phenyl | S(HNEt₃) |
| A-1464 | 2-fluoro-4-(trifluoromethoxy)phenyl | S(HNEt₃) |
| A-1465 | 2-fluoro-5-(trifluoromethoxy)phenyl | S(HNEt₃) |
| A-1466 | 3-fluoro-4-(trifluoromethoxy)phenyl | S(HNEt₃) |
| A-1467 | 2-(difluoromethoxy)-3-chlorophenyl | S(HNEt₃) |
| A-1468 | 2-(difluoromethoxy)-4-chlorophenyl | S(HNEt₃) |
| A-1469 | 2-(difluoromethoxy)-5-chlorophenyl | S(HNEt₃) |
| A-1470 | 2-(difluoromethoxy)-6-chlorophenyl | S(HNEt₃) |
| A-1471 | 3-(difluoromethoxy)-4-chlorophenyl | S(HNEt₃) |
| A-1472 | 3-(difluoromethoxy)-5-chlorophenyl | S(HNEt₃) |
| A-1473 | 2-chloro-3-(difluoromethoxy)phenyl | S(HNEt₃) |
| A-1474 | 2-chloro-4-(difluoromethoxy)phenyl | S(HNEt₃) |
| A-1475 | 2-chloro-5-(difluoromethoxy)phenyl | S(HNEt₃) |
| A-1476 | 3-chloro-4-(difluoromethoxy)phenyl | S(HNEt₃) |
| A-1477 | 2-(difluoromethoxy)-3-fluorophenyl | S(HNEt₃) |
| A-1478 | 2-(difluoromethoxy)-4-fluorophenyl | S(HNEt₃) |
| A-1479 | 2-(difluoromethoxy)-5-fluorophenyl | S(HNEt₃) |
| A-1480 | 2-(difluoromethoxy)-6-fluorophenyl | S(HNEt₃) |
| A-1481 | 3-(difluoromethoxy)-4-fluorophenyl | S(HNEt₃) |
| A-1482 | 3-(difluoromethoxy)-5-fluorophenyl | S(HNEt₃) |
| A-1483 | 2-fluoro-3-(difluoromethoxy)phenyl | S(HNEt₃) |
| A-1484 | 2-fluoro-4-(difluoromethoxy)phenyl | S(HNEt₃) |
| A-1485 | 2-fluoro-5-(difluoromethoxy)phenyl | S(HNEt₃) |
| A-1486 | 3-fluoro-4-(difluoromethoxy)phenyl | S(HNEt₃) |
| A-1487 | 2-(trifluoromethylthio)-3-chlorophenyl | S(HNEt₃) |
| A-1488 | 2-(trifluoromethylthio)-4-chlorophenyl | S(HNEt₃) |
| A-1489 | 2-(trifluoromethylthio)-5-chlorophenyl | S(HNEt₃) |
| A-1490 | 2-(trifluoromethylthio)-6-chlorophenyl | S(HNEt₃) |
| A-1491 | 3-(trifluoromethylthio)-4-chlorophenyl | S(HNEt₃) |
| A-1492 | 3-(trifluoromethylthio)-5-chlorophenyl | S(HNEt₃) |
| A-1493 | 2-chloro-3-(trifluoromethylthio)phenyl | S(HNEt₃) |
| A-1494 | 2-chloro-4-(trifluoromethylthio)phenyl | S(HNEt₃) |
| A-1495 | 2-chloro-5-(trifluoromethylthio)phenyl | S(HNEt₃) |
| A-1496 | 3-chloro-4-(trifluoromethylthio)phenyl | S(HNEt₃) |
| A-1497 | 2-(trifluoromethylthio)-3-fluorophenyl | S(HNEt₃) |
| A-1498 | 2-(trifluoromethylthio)-4-fluorophenyl | S(HNEt₃) |
| A-1499 | 2-(trifluoromethylthio)-5-fluorophenyl | S(HNEt₃) |
| A-1500 | 2-(trifluoromethylthio)-6-fluorophenyl | S(HNEt₃) |
| A-1501 | 3-(trifluoromethylthio)-4-fluorophenyl | S(HNEt₃) |
| A-1502 | 3-(trifluoromethylthio)-5-fluorophenyl | S(HNEt₃) |
| A-1503 | 2-fluoro-3-(trifluoromethylthio)phenyl | S(HNEt₃) |
| A-1504 | 2-fluoro-4-(trifluoromethylthio)phenyl | S(HNEt₃) |
| A-1505 | 2-fluoro-5-(trifluoromethylthio)phenyl | S(HNEt₃) |
| A-1506 | 3-fluoro-4-(trifluoromethylthio)phenyl | S(HNEt₃) |
| A-1507 | 2,3,4-trichlorophenyl | S(HNEt₃) |
| A-1508 | 2,3,5-trichlorophenyl | S(HNEt₃) |
| A-1509 | 2,3,6-trichlorophenyl | S(HNEt₃) |
| A-1510 | 2,4,5-trichlorophenyl | S(HNEt₃) |
| A-1511 | 2,4,6-trichlorophenyl | S(HNEt₃) |
| A-1512 | 3,4,5-trichlorophenyl | S(HNEt₃) |
| A-1513 | 2,3,4-trifluorophenyl | S(HNEt₃) |
| A-1514 | 2,3,5-trifluorophenyl | S(HNEt₃) |
| A-1515 | 2,3,6-trifluorophenyl | S(HNEt₃) |
| A-1516 | 2,4,5-trifluorophenyl | S(HNEt₃) |
| A-1517 | 2,4,6-trifluorophenyl | S(HNEt₃) |
| A-1518 | 3,4,5-trifluorophenyl | S(HNEt₃) |
| A-1519 | 2,3,4-trimethylphenyl | S(HNEt₃) |
| A-1520 | 2,3,5-trimethylphenyl | S(HNEt₃) |
| A-1521 | 2,3,6-trimethylphenyl | S(HNEt₃) |
| A-1522 | 2,4,5-trimethylphenyl | S(HNEt₃) |
| A-1523 | 2,4,6-trimethylphenyl | S(HNEt₃) |
| A-1524 | 3,4,5-trimethylphenyl | S(HNEt₃) |
| A-1525 | 2,3,4-trimethoxyphenyl | S(HNEt₃) |
| A-1526 | 2,3,5-trimethoxyphenyl | S(HNEt₃) |
| A-1527 | 2,3,6-trimethoxyphenyl | S(HNEt₃) |
| A-1528 | 2,4,5-trimethoxyphenyl | S(HNEt₃) |
| A-1529 | 2,4,6-trimethoxyphenyl | S(HNEt₃) |
| A-1530 | 3,4,5-trimethoxyphenyl | S(HNEt₃) |
| A-1531 | phenyl | SCN |
| A-1532 | 2-chlorophenyl | SCN |
| A-1533 | 3-chlorophenyl | SCN |
| A-1534 | 4-chlorophenyl | SCN |
| A-1535 | 2-fluorophenyl | SCN |
| A-1536 | 3-fluorophenyl | SCN |
| A-1537 | 4-fluorophenyl | SCN |
| A-1538 | 2-methylphenyl | SCN |
| A-1539 | 3-methylphenyl | SCN |
| A-1540 | 4-methylphenyl | SCN |
| A-1541 | 2-ethylphenyl | SCN |
| A-1542 | 3-ethylphenyl | SCN |
| A-1543 | 4-ethylphenyl | SCN |
| A-1544 | 2-methoxyphenyl | SCN |
| A-1545 | 3-methoxyphenyl | SCN |
| A-1546 | 4-methoxyphenyl | SCN |
| A-1547 | 2-trifluoromethylphenyl | SCN |
| A-1548 | 3-trifluoromethylphenyl | SCN |
| A-1549 | 4-trifluoromethylphenyl | SCN |
| A-1550 | 2-trifluoromethoxyphenyl | SCN |
| A-1551 | 3-trifluoromethoxyphenyl | SCN |
| A-1552 | 4-trifluoromethoxyphenyl | SCN |
| A-1553 | 2-difluoromethoxyphenyl | SCN |
| A-1554 | 3-difluoromethoxyphenyl | SCN |
| A-1555 | 4-difluoromethoxyphenyl | SCN |
| A-1556 | 2-trifluoromethylthiophenyl | SCN |
| A-1557 | 3-trifluoromethylthiophenyl | SCN |
| A-1558 | 4-trifluoromethylthiophenyl | SCN |
| A-1559 | 2,3-dichlorophenyl | SCN |
| A-1560 | 2,4-dichlorophenyl | SCN |
| A-1561 | 2,5-dichlorophenyl | SCN |
| A-1562 | 2,6-dichlorophenyl | SCN |
| A-1563 | 3,4-dichlorophenyl | SCN |
| A-1564 | 3,5-dichlorophenyl | SCN |
| A-1565 | 2,3-difluorophenyl | SCN |
| A-1566 | 2,4-difluorophenyl | SCN |
| A-1567 | 2,5-difluorophenyl | SCN |
| A-1568 | 2,6-difluorophenyl | SCN |
| A-1569 | 3,4-difluorophenyl | SCN |
| A-1570 | 3,5-difluorophenyl | SCN |
| A-1571 | 2,3-dimethylphenyl | SCN |
| A-1572 | 2,4-dimethylphenyl | SCN |
| A-1573 | 2,5-dimethylphenyl | SCN |
| A-1574 | 2,6-dimethylphenyl | SCN |
| A-1575 | 3,4-dimethylphenyl | SCN |
| A-1576 | 3,5-dimethylphenyl | SCN |
| A-1577 | 2,3-diethylphenyl | SCN |
| A-1578 | 2,4-diethylphenyl | SCN |
| A-1579 | 2,5-diethylphenyl | SCN |
| A-1580 | 2,6-diethylphenyl | SCN |
| A-1581 | 3,5-diethylphenyl | SCN |
| A-1582 | 3,4-diethylphenyl | SCN |
| A-1583 | 2,3-dimethoxyphenyl | SCN |
| A-1584 | 2,4-dimethoxyphenyl | SCN |
| A-1585 | 2,5-dimethoxyphenyl | SCN |
| A-1586 | 2,6-dimethoxyphenyl | SCN |
| A-1587 | 3,4-dimethoxyphenyl | SCN |
| A-1588 | 3,5-dimethoxyphenyl | SCN |
| A-1589 | 2,3-di(trifluoromethyl)phenyl | SCN |
| A-1590 | 2,4-di(trifluoromethyl)phenyl | SCN |
| A-1591 | 2,5-di(trifluoromethyl)phenyl | SCN |
| A-1592 | 2,6-di(trifluoromethyl)phenyl | SCN |
| A-1593 | 3,4-di(trifluoromethyl)phenyl | SCN |
| A-1594 | 3,5-di(trifluoromethyl)phenyl | SCN |
| A-1595 | 2,3-di(trifluoromethoxy)phenyl | SCN |
| A-1596 | 2,4-di(trifluoromethoxy)phenyl | SCN |
| A-1597 | 2,5-di(trifluoromethoxy)phenyl | SCN |
| A-1598 | 2,6-di(trifluoromethoxy)phenyl | SCN |
| A-1599 | 3,4-di(trifluoromethoxy)phenyl | SCN |
| A-1600 | 3,5-di(trifluoromethoxy)phenyl | SCN |
| A-1601 | 2,3-di(difluoromethoxy)phenyl | SCN |
| A-1602 | 2,4-di(difluoromethoxy)phenyl | SCN |
| A-1603 | 2,5-di(difluoromethoxy)phenyl | SCN |
| A-1604 | 2,6-di(difluoromethoxy)phenyl | SCN |
| A-1605 | 3,4-di(difluoromethoxy)phenyl | SCN |
| A-1606 | 3,5-di(difluoromethoxy)phenyl | SCN |
| A-1607 | 2,3-di(trifluoromethylthio)phenyl | SCN |
| A-1608 | 2,4-di(trifluoromethylthio)phenyl | SCN |
| A-1609 | 2,5-di(trifluoromethylthio)phenyl | SCN |
| A-1610 | 2,6-di(trifluoromethylthio)phenyl | SCN |
| A-1611 | 3,4-di(trifluoromethylthio)phenyl | SCN |
| A-1612 | 3,5-di(trifluoromethylthio)phenyl | SCN |
| A-1613 | 2-fluoro-3-chlorophenyl | SCN |
| A-1614 | 2-fluoro-4-chlorophenyl | SCN |
| A-1615 | 2-fluoro-5-chlorophenyl | SCN |
| A-1616 | 2-fluoro-6-chlorophenyl | SCN |
| A-1617 | 3-fluoro-4-chlorophenyl | SCN |
| A-1618 | 3-fluoro-5-chlorophenyl | SCN |
| A-1619 | 2-chloro-3-fluorophenyl | SCN |
| A-1620 | 2-chloro-4-fluorophenyl | SCN |
| A-1621 | 2-chloro-5-fluorophenyl | SCN |
| A-1622 | 3-chloro-4-fluorophenyl | SCN |
| A-1623 | 2-methyl-3-chlorophenyl | SCN |
| A-1624 | 2-methyl-4-chlorophenyl | SCN |
| A-1625 | 2-methyl-5-chlorophenyl | SCN |
| A-1626 | 2-methyl-6-chlorophenyl | SCN |
| A-1627 | 3-methyl-4-chlorophenyl | SCN |
| A-1628 | 3-methyl-5-chlorophenyl | SCN |
| A-1629 | 2-chloro-3-methylphenyl | SCN |
| A-1630 | 2-chloro-4-methylphenyl | SCN |
| A-1631 | 2-chloro-5-methylphenyl | SCN |
| A-1632 | 3-chloro-4-methylphenyl | SCN |
| A-1633 | 2-methyl-3-fluorophenyl | SCN |
| A-1634 | 2-methyl-4-fluorophenyl | SCN |
| A-1635 | 2-methyl-5-fluorophenyl | SCN |
| A-1636 | 2-methyl-6-fluorophenyl | SCN |
| A-1637 | 3-methyl-4-fluorophenyl | SCN |
| A-1638 | 3-methyl-5-fluorophenyl | SCN |
| A-1639 | 2-fluoro-3-methylphenyl | SCN |
| A-1640 | 2-fluoro-4-methylphenyl | SCN |
| A-1641 | 2-fluoro-5-methylphenyl | SCN |
| A-1642 | 3-fluoro-4-methylphenyl | SCN |
| A-1643 | 2-chloro-3-ethylphenyl | SCN |
| A-1644 | 2-chloro-4-ethylphenyl | SCN |
| A-1645 | 2-chloro-5-ethylphenyl | SCN |
| A-1646 | 3-chloro-4-ethylphenyl | SCN |
| A-1647 | 2-ethyl-3-chlorophenyl | SCN |
| A-1648 | 2-ethyl-4-chlorophenyl | SCN |
| A-1649 | 2-ethyl-5-chlorophenyl | SCN |
| A-1650 | 2-ethyl-6-chlorophenyl | SCN |
| A-1651 | 2-ethyl-3-fluorophenyl | SCN |
| A-1652 | 2-ethyl-4-fluorophenyl | SCN |
| A-1653 | 2-ethyl-5-fluorophenyl | SCN |
| A-1654 | 2-ethyl-6-fluorophenyl | SCN |
| A-1655 | 3-ethyl-4-fluorophenyl | SCN |
| A-1656 | 3-ethyl-5-fluorophenyl | SCN |
| A-1657 | 2-fluoro-3-ethylphenyl | SCN |
| A-1658 | 2-fluoro-4-ethylphenyl | SCN |
| A-1659 | 2-fluoro-5-ethylphenyl | SCN |
| A-1660 | 3-fluoro-4-ethylphenyl | SCN |
| A-1661 | 2-methoxy-3-chlorophenyl | SCN |
| A-1662 | 2-methoxy-4-chlorophenyl | SCN |
| A-1663 | 2-methoxy-5-chlorophenyl | SCN |
| A-1664 | 2-methoxy-6-chlorophenyl | SCN |
| A-1665 | 3-methoxy-4-chlorophenyl | SCN |
| A-1666 | 3-methoxy-5-chlorophenyl | SCN |
| A-1667 | 2-chloro-3-methoxyphenyl | SCN |
| A-1668 | 2-chloro-4-methoxyphenyl | SCN |
| A-1669 | 2-chloro-5-methoxyphenyl | SCN |
| A-1670 | 3-chloro-4-methoxyphenyl | SCN |
| A-1671 | 2-methoxy-3-fluorophenyl | SCN |
| A-1672 | 2-methoxy-4-fluorophenyl | SCN |
| A-1673 | 2-methoxy-5-fluorophenyl | SCN |
| A-1674 | 2-methoxy-6-fluorophenyl | SCN |
| A-1675 | 3-methoxy-4-fluorophenyl | SCN |
| A-1676 | 3-methoxy-5-fluorophenyl | SCN |
| A-1677 | 2-fluoro-3-methoxyphenyl | SCN |
| A-1678 | 2-fluoro-4-methoxyphenyl | SCN |
| A-1679 | 2-fluoro-5-methoxyphenyl | SCN |
| A-1680 | 3-fluoro-4-methoxyphenyl | SCN |
| A-1681 | 3-fluoro-5-methoxyphenyl | SCN |
| A-1682 | 2-(trifluoromethyl)-3-chlorophenyl | SCN |
| A-1683 | 2-(trifluoromethyl)-4-chlorophenyl | SCN |
| A-1684 | 2-(trifluoromethyl)-5-chlorophenyl | SCN |
| A-1685 | 2-(trifluoromethyl)-6-chlorophenyl | SCN |
| A-1686 | 3-(trifluoromethyl)-4-chlorophenyl | SCN |
| A-1687 | 3-(trifluoromethyl)-5-chlorophenyl | SCN |
| A-1688 | 2-chloro-3-(trifluoromethyl)phenyl | SCN |
| A-1689 | 2-chloro-4-(trifluoromethyl)phenyl | SCN |
| A-1690 | 2-chloro-5-(trifluoromethyl)phenyl | SCN |
| A-1691 | 3-chloro-4-(trifluoromethyl)phenyl | SCN |
| A-1692 | 2-(trifluoromethyl)-3-fluorophenyl | SCN |
| A-1693 | 2-(trifluoromethyl)-4-fluorophenyl | SCN |
| A-1694 | 2-(trifluoromethyl)-5-fluorophenyl | SCN |
| A-1695 | 2-(trifluoromethyl)-6-fluorophenyl | SCN |
| A-1696 | 3-(trifluoromethyl)-4-fluorophenyl | SCN |
| A-1697 | 3-(trifluoromethyl)-5-fluorophenyl | SCN |
| A-1698 | 2-fluoro-3-(trifluoromethyl)phenyl | SCN |
| A-1699 | 2-fluoro-4-(trifluoromethyl)phenyl | SCN |
| A-1700 | 2-fluoro-5-(trifluoromethyl)phenyl | SCN |
| A-1701 | 3-fluoro-4-(trifluoromethyl)phenyl | SCN |
| A-1702 | 2-(trifluoromethoxy)-3-chlorophenyl | SCN |
| A-1703 | 2-(trifluoromethoxy)-4-chlorophenyl | SCN |
| A-1704 | 2-(trifluoromethoxy)-5-chlorophenyl | SCN |
| A-1705 | 2-(trifluoromethoxy)-6-chlorophenyl | SCN |
| A-1706 | 3-(trifluoromethoxy)-4-chlorophenyl | SCN |
| A-1707 | 3-(trifluoromethoxy)-5-chlorophenyl | SCN |
| A-1708 | 2-chloro-3-(trifluoromethoxy)phenyl | SCN |
| A-1709 | 2-chloro-4-(trifluoromethoxy)phenyl | SCN |
| A-1710 | 2-chloro-5-(trifluoromethoxy)phenyl | SCN |
| A-1711 | 3-chloro-4-(trifluoromethoxy)phenyl | SCN |
| A-1712 | 2-(trifluoromethoxy)-3-fluorophenyl | SCN |
| A-1713 | 2-(trifluoromethoxy)-4-fluorophenyl | SCN |
| A-1714 | 2-(trifluoromethoxy)-5-fluorophenyl | SCN |
| A-1715 | 2-(trifluoromethoxy)-6-fluorophenyl | SCN |
| A-1716 | 3-(trifluoromethoxy)-4-fluorophenyl | SCN |
| A-1717 | 3-(trifluoromethoxy)-5-fluorophenyl | SCN |
| A-1718 | 2-fluoro-3-(trifluoromethoxy)phenyl | SCN |
| A-1719 | 2-fluoro-4-(trifluoromethoxy)phenyl | SCN |
| A-1720 | 2-fluoro-5-(trifluoromethoxy)phenyl | SCN |
| A-1721 | 3-fluoro-4-(trifluoromethoxy)phenyl | SCN |
| A-1722 | 2-(difluoromethoxy)-3-chlorophenyl | SCN |
| A-1723 | 2-(difluoromethoxy)-4-chlorophenyl | SCN |
| A-1724 | 2-(difluoromethoxy)-5-chlorophenyl | SCN |
| A-1725 | 2-(difluoromethoxy)-6-chlorophenyl | SCN |
| A-1726 | 3-(difluoromethoxy)-4-chlorophenyl | SCN |
| A-1727 | 3-(difluoromethoxy)-5-chlorophenyl | SCN |
| A-1728 | 2-chloro-3-(difluoromethoxy)phenyl | SCN |
| A-1729 | 2-chloro-4-(difluoromethoxy)phenyl | SCN |
| A-1730 | 2-chloro-5-(difluoromethoxy)phenyl | SCN |
| A-1731 | 3-chloro-4-(difluoromethoxy)phenyl | SCN |
| A-1732 | 2-(difluoromethoxy)-3-fluorophenyl | SCN |
| A-1733 | 2-(difluoromethoxy)-4-fluorophenyl | SCN |
| A-1734 | 2-(difluoromethoxy)-5-fluorophenyl | SCN |
| A-1735 | 2-(difluoromethoxy)-6-fluorophenyl | SCN |
| A-1736 | 3-(difluoromethoxy)-4-fluorophenyl | SCN |
| A-1737 | 3-(difluoromethoxy)-5-fluorophenyl | SCN |
| A-1738 | 2-fluoro-3-(difluoromethoxy)phenyl | SCN |
| A-1739 | 2-fluoro-4-(difluoromethoxy)phenyl | SCN |
| A-1740 | 2-fluoro-5-(difluoromethoxy)phenyl | SCN |
| A-1741 | 3-fluoro-4-(difluoromethoxy)phenyl | SCN |
| A-1742 | 2-(trifluoromethylthio)-3-chlorophenyl | SCN |
| A-1743 | 2-(trifluoromethylthio)-4-chlorophenyl | SCN |
| A-1744 | 2-(trifluoromethylthio)-5-chlorophenyl | SCN |
| A-1745 | 2-(trifluoromethylthio)-6-chlorophenyl | SCN |
| A-1746 | 3-(trifluoromethylthio)-4-chlorophenyl | SCN |
| A-1747 | 3-(trifluoromethylthio)-5-chlorophenyl | SCN |
| A-1748 | 2-chloro-3-(trifluoromethylthio)phenyl | SCN |
| A-1749 | 2-chloro-4-(trifluoromethylthio)phenyl | SCN |
| A-1750 | 2-chloro-5-(trifluoromethylthio)phenyl | SCN |
| A-1751 | 3-chloro-4-(trifluoromethylthio)phenyl | SCN |
| A-1752 | 2-(trifluoromethylthio)-3-fluorophenyl | SCN |
| A-1753 | 2-(trifluoromethylthio)-4-fluorophenyl | SCN |
| A-1754 | 2-(trifluoromethylthio)-5-fluorophenyl | SCN |
| A-1755 | 2-(trifluoromethylthio)-6-fluorophenyl | SCN |
| A-1756 | 3-(trifluoromethylthio)-4-fluorophenyl | SCN |
| A-1757 | 3-(trifluoromethylthio)-5-fluorophenyl | SCN |
| A-1758 | 2-fluoro-3-(trifluoromethylthio)phenyl | SCN |
| A-1759 | 2-fluoro-4-(trifluoromethylthio)phenyl | SCN |
| A-1760 | 2-fluoro-5-(trifluoromethylthio)phenyl | SCN |
| A-1761 | 3-fluoro-4-(trifluoromethylthio)phenyl | SCN |
| A-1762 | 2,3,4-trichlorophenyl | SCN |
| A-1763 | 2,3,5-trichlorophenyl | SCN |
| A-1764 | 2,3,6-trichlorophenyl | SCN |
| A-1765 | 2,4,5-trichlorophenyl | SCN |
| A-1766 | 2,4,6-trichlorophenyl | SCN |
| A-1767 | 3,4,5-trichlorophenyl | SCN |
| A-1768 | 2,3,4-trifluorophenyl | SCN |
| A-1769 | 2,3,5-trifluorophenyl | SCN |
| A-1770 | 2,3,6-trifluorophenyl | SCN |
| A-1771 | 2,4,5-trifluorophenyl | SCN |
| A-1772 | 2,4,6-trifluorophenyl | SCN |
| A-1773 | 3,4,5-trifluorophenyl | SCN |
| A-1774 | 2,3,4-trimethylphenyl | SCN |
| A-1775 | 2,3,5-trimethylphenyl | SCN |
| A-1776 | 2,3,6-trimethylphenyl | SCN |
| A-1777 | 2,4,5-trimethylphenyl | SCN |
| A-1778 | 2,4,6-trimethylphenyl | SCN |
| A-1779 | 3,4,5-trimethylphenyl | SCN |
| A-1780 | 2,3,4-trimethoxyphenyl | SCN |
| A-1781 | 2,3,5-trimethoxyphenyl | SCN |
| A-1782 | 2,3,6-trimethoxyphenyl | SCN |
| A-1783 | 2,4,5-trimethoxyphenyl | SCN |
| A-1784 | 2,4,6-trimethoxyphenyl | SCN |
| A-1785 | 3,4,5-trimethoxyphenyl | SCN |
| A-1786 | phenyl | S(C═O)CH₃ |
| A-1787 | 2-chlorophenyl | S(C═O)CH₃ |
| A-1788 | 3-chlorophenyl | S(C═O)CH₃ |
| A-1789 | 4-chlorophenyl | S(C═O)CH₃ |
| A-1790 | 2-fluorophenyl | S(C═O)CH₃ |
| A-1791 | 3-fluorophenyl | S(C═O)CH₃ |
| A-1792 | 4-fluorophenyl | S(C═O)CH₃ |
| A-1793 | 2-methylphenyl | S(C═O)CH₃ |
| A-1794 | 3-methylphenyl | S(C═O)CH₃ |
| A-1795 | 4-methylphenyl | S(C═O)CH₃ |
| A-1796 | 2-ethylphenyl | S(C═O)CH₃ |
| A-1797 | 3-ethylphenyl | S(C═O)CH₃ |
| A-1798 | 4-ethylphenyl | S(C═O)CH₃ |
| A-1799 | 2-methoxyphenyl | S(C═O)CH₃ |
| A-1800 | 3-methoxyphenyl | S(C═O)CH₃ |
| A-1801 | 4-methoxyphenyl | S(C═O)CH₃ |
| A-1802 | 2-trifluoromethylphenyl | S(C═O)CH₃ |
| A-1803 | 3-trifluoromethylphenyl | S(C═O)CH₃ |
| A-1804 | 4-trifluoromethylphenyl | S(C═O)CH₃ |
| A-1805 | 2-trifluoromethoxyphenyl | S(C═O)CH₃ |
| A-1806 | 3-trifluoromethoxyphenyl | S(C═O)CH₃ |
| A-1807 | 4-trifluoromethoxyphenyl | S(C═O)CH₃ |
| A-1808 | 2-difluoromethoxyphenyl | S(C═O)CH₃ |
| A-1809 | 3-difluoromethoxyphenyl | S(C═O)CH₃ |
| A-1810 | 4-difluoromethoxyphenyl | S(C═O)CH₃ |
| A-1811 | 2-trifluoromethylthiophenyl | S(C═O)CH₃ |
| A-1812 | 3-trifluoromethylthiophenyl | S(C═O)CH₃ |
| A-1813 | 4-trifluoromethylthiophenyl | S(C═O)CH₃ |
| A-1814 | 2,3-dichlorophenyl | S(C═O)CH₃ |
| A-1815 | 2,4-dichlorophenyl | S(C═O)CH₃ |
| A-1816 | 2,5-dichlorophenyl | S(C═O)CH₃ |
| A-1817 | 2,6-dichlorophenyl | S(C═O)CH₃ |
| A-1818 | 3,4-dichlorophenyl | S(C═O)CH₃ |
| A-1819 | 3,5-dichlorophenyl | S(C═O)CH₃ |
| A-1820 | 2,3-difluorophenyl | S(C═O)CH₃ |
| A-1821 | 2,4-difluorophenyl | S(C═O)CH₃ |
| A-1822 | 2,5-difluorophenyl | S(C═O)CH₃ |
| A-1823 | 2,6-difluorophenyl | S(C═O)CH₃ |
| A-1824 | 3,4-difluorophenyl | S(C═O)CH₃ |
| A-1825 | 3,5-difluorophenyl | S(C═O)CH₃ |
| A-1826 | 2,3-dimethylphenyl | S(C═O)CH₃ |
| A-1827 | 2,4-dimethylphenyl | S(C═O)CH₃ |
| A-1828 | 2,5-dimethylphenyl | S(C═O)CH₃ |
| A-1829 | 2,6-dimethylphenyl | S(C═O)CH₃ |
| A-1830 | 3,4-dimethylphenyl | S(C═O)CH₃ |
| A-1831 | 3,5-dimethylphenyl | S(C═O)CH₃ |
| A-1832 | 2,3-diethylphenyl | S(C═O)CH₃ |
| A-1833 | 2,4-diethylphenyl | S(C═O)CH₃ |
| A-1834 | 2,5-diethylphenyl | S(C═O)CH₃ |
| A-1835 | 2,6-diethylphenyl | S(C═O)CH₃ |
| A-1836 | 3,5-diethylphenyl | S(C═O)CH₃ |
| A-1837 | 3,4-diethylphenyl | S(C═O)CH₃ |
| A-1838 | 2,3-dimethoxyphenyl | S(C═O)CH₃ |
| A-1839 | 2,4-dimethoxyphenyl | S(C═O)CH₃ |
| A-1840 | 2,5-dimethoxyphenyl | S(C═O)CH₃ |
| A-1841 | 2,6-dimethoxyphenyl | S(C═O)CH₃ |
| A-1842 | 3,4-dimethoxyphenyl | S(C═O)CH₃ |
| A-1843 | 3,5-dimethoxyphenyl | S(C═O)CH₃ |
| A-1844 | 2,3-di(trifluoromethyl)phenyl | S(C═O)CH₃ |
| A-1845 | 2,4-di(trifluoromethyl)phenyl | S(C═O)CH₃ |
| A-1846 | 2,5-di(trifluoromethyl)phenyl | S(C═O)CH₃ |
| A-1847 | 2,6-di(trifluoromethyl)phenyl | S(C═O)CH₃ |
| A-1848 | 3,4-di(trifluoromethyl)phenyl | S(C═O)CH₃ |
| A-1849 | 3,5-di(trifluoromethyl)phenyl | S(C═O)CH₃ |
| A-1850 | 2,3-di(trifluoromethoxy)phenyl | S(C═O)CH₃ |
| A-1851 | 2,4-di(trifluoromethoxy)phenyl | S(C═O)CH₃ |
| A-1852 | 2,5-di(trifluoromethoxy)phenyl | S(C═O)CH₃ |
| A-1853 | 2,6-di(trifluoromethoxy)phenyl | S(C═O)CH₃ |
| A-1854 | 3,4-di(trifluoromethoxy)phenyl | S(C═O)CH₃ |
| A-1855 | 3,5-di(trifluoromethoxy)phenyl | S(C═O)CH₃ |
| A-1856 | 2,3-di(difluoromethoxy)phenyl | S(C═O)CH₃ |
| A-1857 | 2,4-di(difluoromethoxy)phenyl | S(C═O)CH₃ |
| A-1858 | 2,5-di(difluoromethoxy)phenyl | S(C═O)CH₃ |
| A-1859 | 2,6-di(difluoromethoxy)phenyl | S(C═O)CH₃ |
| A-1860 | 3,4-di(difluoromethoxy)phenyl | S(C═O)CH₃ |
| A-1861 | 3,5-di(difluoromethoxy)phenyl | S(C═O)CH₃ |
| A-1862 | 2,3-di(trifluoromethylthio)phenyl | S(C═O)CH₃ |
| A-1863 | 2,4-di(trifluoromethylthio)phenyl | S(C═O)CH₃ |
| A-1864 | 2,5-di(trifluoromethylthio)phenyl | S(C═O)CH₃ |
| A-1865 | 2,6-di(trifluoromethylthio)phenyl | S(C═O)CH₃ |
| A-1866 | 3,4-di(trifluoromethylthio)phenyl | S(C═O)CH₃ |
| A-1867 | 3,5-di(trifluoromethylthio)phenyl | S(C═O)CH₃ |
| A-1868 | 2-fluoro-3-chlorophenyl | S(C═O)CH₃ |
| A-1869 | 2-fluoro-4-chlorophenyl | S(C═O)CH₃ |
| A-1870 | 2-fluoro-5-chlorophenyl | S(C═O)CH₃ |
| A-1871 | 2-fluoro-6-chlorophenyl | S(C═O)CH₃ |
| A-1872 | 3-fluoro-4-chlorophenyl | S(C═O)CH₃ |
| A-1873 | 3-fluoro-5-chlorophenyl | S(C═O)CH₃ |
| A-1874 | 2-chloro-3-fluorophenyl | S(C═O)CH₃ |
| A-1875 | 2-chloro-4-fluorophenyl | S(C═O)CH₃ |
| A-1876 | 2-chloro-5-fluorophenyl | S(C═O)CH₃ |
| A-1877 | 3-chloro-4-fluorophenyl | S(C═O)CH₃ |
| A-1878 | 2-methyl-3-chlorophenyl | S(C═O)CH₃ |
| A-1879 | 2-methyl-4-chlorophenyl | S(C═O)CH₃ |
| A-1880 | 2-methyl-5-chlorophenyl | S(C═O)CH₃ |
| A-1881 | 2-methyl-6-chlorophenyl | S(C═O)CH₃ |
| A-1882 | 3-methyl-4-chlorophenyl | S(C═O)CH₃ |
| A-1883 | 3-methyl-5-chlorophenyl | S(C═O)CH₃ |
| A-1884 | 2-chloro-3-methylphenyl | S(C═O)CH₃ |
| A-1885 | 2-chloro-4-methylphenyl | S(C═O)CH₃ |
| A-1886 | 2-chloro-5-methylphenyl | S(C═O)CH₃ |
| A-1887 | 3-chloro-4-methylphenyl | S(C═O)CH₃ |
| A-1888 | 2-methyl-3-fluorophenyl | S(C═O)CH₃ |
| A-1889 | 2-methyl-4-fluorophenyl | S(C═O)CH₃ |
| A-1890 | 2-methyl-5-fluorophenyl | S(C═O)CH₃ |
| A-1891 | 2-methyl-6-fluorophenyl | S(C═O)CH₃ |
| A-1892 | 3-methyl-4-fluorophenyl | S(C═O)CH₃ |
| A-1893 | 3-methyl-5-fluorophenyl | S(C═O)CH₃ |
| A-1894 | 2-fluoro-3-methylphenyl | S(C═O)CH₃ |
| A-1895 | 2-fluoro-4-methylphenyl | S(C═O)CH₃ |
| A-1896 | 2-fluoro-5-methylphenyl | S(C═O)CH₃ |
| A-1897 | 3-fluoro-4-methylphenyl | S(C═O)CH₃ |
| A-1898 | 2-chloro-3-ethylphenyl | S(C═O)CH₃ |
| A-1899 | 2-chloro-4-ethylphenyl | S(C═O)CH₃ |
| A-1900 | 2-chloro-5-ethylphenyl | S(C═O)CH₃ |
| A-1901 | 3-chloro-4-ethylphenyl | S(C═O)CH₃ |
| A-1902 | 2-ethyl-3-chlorophenyl | S(C═O)CH₃ |
| A-1903 | 2-ethyl-4-chlorophenyl | S(C═O)CH₃ |
| A-1904 | 2-ethyl-5-chlorophenyl | S(C═O)CH₃ |
| A-1905 | 2-ethyl-6-chlorophenyl | S(C═O)CH₃ |
| A-1906 | 2-ethyl-3-fluorophenyl | S(C═O)CH₃ |
| A-1907 | 2-ethyl-4-fluorophenyl | S(C═O)CH₃ |
| A-1908 | 2-ethyl-5-fluorophenyl | S(C═O)CH₃ |
| A-1909 | 2-ethyl-6-fluorophenyl | S(C═O)CH₃ |
| A-1910 | 3-ethyl-4-fluorophenyl | S(C═O)CH₃ |
| A-1911 | 3-ethyl-5-fluorophenyl | S(C═O)CH₃ |
| A-1912 | 2-fluoro-3-ethylphenyl | S(C═O)CH₃ |
| A-1913 | 2-fluoro-4-ethylphenyl | S(C═O)CH₃ |
| A-1914 | 2-fluoro-5-ethylphenyl | S(C═O)CH₃ |
| A-1915 | 3-fluoro-4-ethylphenyl | S(C═O)CH₃ |
| A-1916 | 2-methoxy-3-chlorophenyl | S(C═O)CH₃ |
| A-1917 | 2-methoxy-4-chlorophenyl | S(C═O)CH₃ |
| A-1918 | 2-methoxy-5-chlorophenyl | S(C═O)CH₃ |
| A-1919 | 2-methoxy-6-chlorophenyl | S(C═O)CH₃ |
| A-1920 | 3-methoxy-4-chlorophenyl | S(C═O)CH₃ |
| A-1921 | 3-methoxy-5-chlorophenyl | S(C═O)CH₃ |
| A-1922 | 2-chloro-3-methoxyphenyl | S(C═O)CH₃ |
| A-1923 | 2-chloro-4-methoxyphenyl | S(C═O)CH₃ |
| A-1924 | 2-chloro-5-methoxyphenyl | S(C═O)CH₃ |
| A-1925 | 3-chloro-4-methoxyphenyl | S(C═O)CH₃ |
| A-1926 | 2-methoxy-3-fluorophenyl | S(C═O)CH₃ |
| A-1927 | 2-methoxy-4-fluorophenyl | S(C═O)CH₃ |
| A-1928 | 2-methoxy-5-fluorophenyl | S(C═O)CH₃ |
| A-1929 | 2-methoxy-6-fluorophenyl | S(C═O)CH₃ |
| A-1930 | 3-methoxy-4-fluorophenyl | S(C═O)CH₃ |
| A-1931 | 3-methoxy-5-fluorophenyl | S(C═O)CH₃ |
| A-1932 | 2-fluoro-3-methoxyphenyl | S(C═O)CH₃ |
| A-1933 | 2-fluoro-4-methoxyphenyl | S(C═O)CH₃ |
| A-1934 | 2-fluoro-5-methoxyphenyl | S(C═O)CH₃ |
| A-1935 | 3-fluoro-4-methoxyphenyl | S(C═O)CH₃ |
| A-1936 | 3-fluoro-5-methoxyphenyl | S(C═O)CH₃ |
| A-1937 | 2-(trifluoromethyl)-3-chlorophenyl | S(C═O)CH₃ |
| A-1938 | 2-(trifluoromethyl)-4-chlorophenyl | S(C═O)CH₃ |
| A-1939 | 2-(trifluoromethyl)-5-chlorophenyl | S(C═O)CH₃ |
| A-1940 | 2-(trifluoromethyl)-6-chlorophenyl | S(C═O)CH₃ |
| A-1941 | 3-(trifluoromethyl)-4-chlorophenyl | S(C═O)CH₃ |
| A-1942 | 3-(trifluoromethyl)-5-chlorophenyl | S(C═O)CH₃ |
| A-1943 | 2-chloro-3-(trifluoromethyl)phenyl | S(C═O)CH₃ |
| A-1944 | 2-chloro-4-(trifluoromethyl)phenyl | S(C═O)CH₃ |
| A-1945 | 2-chloro-5-(trifluoromethyl)phenyl | S(C═O)CH₃ |
| A-1946 | 3-chloro-4-(trifluoromethyl)phenyl | S(C═O)CH₃ |
| A-1947 | 2-(trifluoromethyl)-3-fluorophenyl | S(C═O)CH₃ |
| A-1948 | 2-(trifluoromethyl)-4-fluorophenyl | S(C═O)CH₃ |
| A-1949 | 2-(trifluoromethyl)-5-fluorophenyl | S(C═O)CH₃ |
| A-1950 | 2-(trifluoromethyl)-6-fluorophenyl | S(C═O)CH₃ |
| A-1951 | 3-(trifluoromethyl)-4-fluorophenyl | S(C═O)CH₃ |
| A-1952 | 3-(trifluoromethyl)-5-fluorophenyl | S(C═O)CH₃ |
| A-1953 | 2-fluoro-3-(trifluoromethyl)phenyl | S(C═O)CH₃ |
| A-1954 | 2-fluoro-4-(trifluoromethyl)phenyl | S(C═O)CH₃ |
| A-1955 | 2-fluoro-5-(trifluoromethyl)phenyl | S(C═O)CH₃ |
| A-1956 | 3-fluoro-4-(trifluoromethyl)phenyl | S(C═O)CH₃ |
| A-1957 | 2-(trifluoromethoxy)-3-chlorophenyl | S(C═O)CH₃ |
| A-1958 | 2-(trifluoromethoxy)-4-chlorophenyl | S(C═O)CH₃ |
| A-1959 | 2-(trifluoromethoxy)-5-chlorophenyl | S(C═O)CH₃ |
| A-1960 | 2-(trifluoromethoxy)-6-chlorophenyl | S(C═O)CH₃ |
| A-1961 | 3-(trifluoromethoxy)-4-chlorophenyl | S(C═O)CH₃ |
| A-1962 | 3-(trifluoromethoxy)-5-chlorophenyl | S(C═O)CH₃ |
| A-1963 | 2-chloro-3-(trifluoromethoxy)phenyl | S(C═O)CH₃ |
| A-1964 | 2-chloro-4-(trifluoromethoxy)phenyl | S(C═O)CH₃ |
| A-1965 | 2-chloro-5-(trifluoromethoxy)phenyl | S(C═O)CH₃ |
| A-1966 | 3-chloro-4-(trifluoromethoxy)phenyl | S(C═O)CH₃ |
| A-1967 | 2-(trifluoromethoxy)-3-fluorophenyl | S(C═O)CH₃ |
| A-1968 | 2-(trifluoromethoxy)-4-fluorophenyl | S(C═O)CH₃ |
| A-1969 | 2-(trifluoromethoxy)-5-fluorophenyl | S(C═O)CH₃ |
| A-1970 | 2-(trifluoromethoxy)-6-fluorophenyl | S(C═O)CH₃ |
| A-1971 | 3-(trifluoromethoxy)-4-fluorophenyl | S(C═O)CH₃ |
| A-1972 | 3-(trifluoromethoxy)-5-fluorophenyl | S(C═O)CH₃ |
| A-1973 | 2-fluoro-3-(trifluoromethoxy)phenyl | S(C═O)CH₃ |
| A-1974 | 2-fluoro-4-(trifluoromethoxy)phenyl | S(C═O)CH₃ |
| A-1975 | 2-fluoro-5-(trifluoromethoxy)phenyl | S(C═O)CH₃ |
| A-1976 | 3-fluoro-4-(trifluoromethoxy)phenyl | S(C═O)CH₃ |
| A-1977 | 2-(difluoromethoxy)-3-chlorophenyl | S(C═O)CH₃ |
| A-1978 | 2-(difluoromethoxy)-4-chlorophenyl | S(C═O)CH₃ |
| A-1979 | 2-(difluoromethoxy)-5-chlorophenyl | S(C═O)CH₃ |
| A-1980 | 2-(difluoromethoxy)-6-chlorophenyl | S(C═O)CH₃ |
| A-1981 | 3-(difluoromethoxy)-4-chlorophenyl | S(C═O)CH₃ |
| A-1982 | 3-(difluoromethoxy)-5-chlorophenyl | S(C═O)CH₃ |
| A-1983 | 2-chloro-3-(difluoromethoxy)phenyl | S(C═O)CH₃ |
| A-1984 | 2-chloro-4-(difluoromethoxy)phenyl | S(C═O)CH₃ |
| A-1985 | 2-chloro-5-(difluoromethoxy)phenyl | S(C═O)CH₃ |
| A-1986 | 3-chloro-4-(difluoromethoxy)phenyl | S(C═O)CH₃ |
| A-1987 | 2-(difluoromethoxy)-3-fluorophenyl | S(C═O)CH₃ |
| A-1988 | 2-(difluoromethoxy)-4-fluorophenyl | S(C═O)CH₃ |
| A-1989 | 2-(difluoromethoxy)-5-fluorophenyl | S(C═O)CH₃ |
| A-1990 | 2-(difluoromethoxy)-6-fluorophenyl | S(C═O)CH₃ |
| A-1991 | 3-(difluoromethoxy)-4-fluorophenyl | S(C═O)CH₃ |
| A-1992 | 3-(difluoromethoxy)-5-fluorophenyl | S(C═O)CH₃ |
| A-1993 | 2-fluoro-3-(difluoromethoxy)phenyl | S(C═O)CH₃ |
| A-1994 | 2-fluoro-4-(difluoromethoxy)phenyl | S(C═O)CH₃ |
| A-1995 | 2-fluoro-5-(difluoromethoxy)phenyl | S(C═O)CH₃ |
| A-1996 | 3-fluoro-4-(difluoromethoxy)phenyl | S(C═O)CH₃ |
| A-1997 | 2-(trifluoromethylthio)-3-chlorophenyl | S(C═O)CH₃ |
| A-1998 | 2-(trifluoromethylthio)-4-chlorophenyl | S(C═O)CH₃ |
| A-1999 | 2-(trifluoromethylthio)-5-chlorophenyl | S(C═O)CH₃ |
| A-2000 | 2-(trifluoromethylthio)-6-chlorophenyl | S(C═O)CH₃ |
| A-2001 | 3-(trifluoromethylthio)-4-chlorophenyl | S(C═O)CH₃ |
| A-2002 | 3-(trifluoromethylthio)-5-chlorophenyl | S(C═O)CH₃ |
| A-2003 | 2-chloro-3-(trifluoromethylthio)phenyl | S(C═O)CH₃ |
| A-2004 | 2-chloro-4-(trifluoromethylthio)phenyl | S(C═O)CH₃ |
| A-2005 | 2-chloro-5-(trifluoromethylthio)phenyl | S(C═O)CH₃ |
| A-2006 | 3-chloro-4-(trifluoromethylthio)phenyl | S(C═O)CH₃ |
| A-2007 | 2-(trifluoromethylthio)-3-fluorophenyl | S(C═O)CH₃ |
| A-2008 | 2-(trifluoromethylthio)-4-fluorophenyl | S(C═O)CH₃ |
| A-2009 | 2-(trifluoromethylthio)-5-fluorophenyl | S(C═O)CH₃ |
| A-2010 | 2-(trifluoromethylthio)-6-fluorophenyl | S(C═O)CH₃ |
| A-2011 | 3-(trifluoromethylthio)-4-fluorophenyl | S(C═O)CH₃ |
| A-2012 | 3-(trifluoromethylthio)-5-fluorophenyl | S(C═O)CH₃ |
| A-2013 | 2-fluoro-3-(trifluoromethylthio)phenyl | S(C═O)CH₃ |
| A-2014 | 2-fluoro-4-(trifluoromethylthio)phenyl | S(C═O)CH₃ |
| A-2015 | 2-fluoro-5-(trifluoromethylthio)phenyl | S(C═O)CH₃ |
| A-2016 | 3-fluoro-4-(trifluoromethylthio)phenyl | S(C═O)CH₃ |
| A-2017 | 2,3,4-trichlorophenyl | S(C═O)CH₃ |
| A-2018 | 2,3,5-trichlorophenyl | S(C═O)CH₃ |
| A-2019 | 2,3,6-trichlorophenyl | S(C═O)CH₃ |
| A-2020 | 2,4,5-trichlorophenyl | S(C═O)CH₃ |
| A-2021 | 2,4,6-trichlorophenyl | S(C═O)CH₃ |
| A-2022 | 3,4,5-trichlorophenyl | S(C═O)CH₃ |
| A-2023 | 2,3,4-trifluorophenyl | S(C═O)CH₃ |
| A-2024 | 2,3,5-trifluorophenyl | S(C═O)CH₃ |
| A-2025 | 2,3,6-trifluorophenyl | S(C═O)CH₃ |
| A-2026 | 2,4,5-trifluorophenyl | S(C═O)CH₃ |
| A-2027 | 2,4,6-trifluorophenyl | S(C═O)CH₃ |
| A-2028 | 3,4,5-trifluorophenyl | S(C═O)CH₃ |
| A-2029 | 2,3,4-trimethylphenyl | S(C═O)CH₃ |
| A-2030 | 2,3,5-trimethylphenyl | S(C═O)CH₃ |
| A-2031 | 2,3,6-trimethylphenyl | S(C═O)CH₃ |
| A-2032 | 2,4,5-trimethylphenyl | S(C═O)CH₃ |
| A-2033 | 2,4,6-trimethylphenyl | S(C═O)CH₃ |
| A-2034 | 3,4,5-trimethylphenyl | S(C═O)CH₃ |
| A-2035 | 2,3,4-trimethoxyphenyl | S(C═O)CH₃ |
| A-2036 | 2,3,5-trimethoxyphenyl | S(C═O)CH₃ |
| A-2037 | 2,3,6-trimethoxyphenyl | S(C═O)CH₃ |
| A-2038 | 2,4,5-trimethoxyphenyl | S(C═O)CH₃ |
| A-2039 | 2,4,6-trimethoxyphenyl | S(C═O)CH₃ |
| A-2040 | 3,4,5-trimethoxyphenyl | S(C═O)CH₃ |
| A-2041 | phenyl | S(C═O)OCH₃ |
| A-2042 | 2-chlorophenyl | S(C═O)OCH₃ |
| A-2043 | 3-chlorophenyl | S(C═O)OCH₃ |
| A-2044 | 4-chlorophenyl | S(C═O)OCH₃ |
| A-2045 | 2-fluorophenyl | S(C═O)OCH₃ |
| A-2046 | 3-fluorophenyl | S(C═O)OCH₃ |
| A-2047 | 4-fluorophenyl | S(C═O)OCH₃ |
| A-2048 | 2-methylphenyl | S(C═O)OCH₃ |
| A-2049 | 3-methylphenyl | S(C═O)OCH₃ |
| A-2050 | 4-methylphenyl | S(C═O)OCH₃ |
| A-2051 | 2-ethylphenyl | S(C═O)OCH₃ |
| A-2052 | 3-ethylphenyl | S(C═O)OCH₃ |
| A-2053 | 4-ethylphenyl | S(C═O)OCH₃ |
| A-2054 | 2-methoxyphenyl | S(C═O)OCH₃ |
| A-2055 | 3-methoxyphenyl | S(C═O)OCH₃ |
| A-2056 | 4-methoxyphenyl | S(C═O)OCH₃ |
| A-2057 | 2-trifluoromethylphenyl | S(C═O)OCH₃ |
| A-2058 | 3-trifluoromethylphenyl | S(C═O)OCH₃ |
| A-2059 | 4-trifluoromethylphenyl | S(C═O)OCH₃ |
| A-2060 | 2-trifluoromethoxyphenyl | S(C═O)OCH₃ |
| A-2061 | 3-trifluoromethoxyphenyl | S(C═O)OCH₃ |
| A-2062 | 4-trifluoromethoxyphenyl | S(C═O)OCH₃ |
| A-2063 | 2-difluoromethoxyphenyl | S(C═O)OCH₃ |
| A-2064 | 3-difluoromethoxyphenyl | S(C═O)OCH₃ |
| A-2065 | 4-difluoromethoxyphenyl | S(C═O)OCH₃ |
| A-2066 | 2-trifluoromethylthiophenyl | S(C═O)OCH₃ |
| A-2067 | 3-trifluoromethylthiophenyl | S(C═O)OCH₃ |
| A-2068 | 4-trifluoromethylthiophenyl | S(C═O)OCH₃ |
| A-2069 | 2,3-dichlorophenyl | S(C═O)OCH₃ |
| A-2070 | 2,4-dichlorophenyl | S(C═O)OCH₃ |
| A-2071 | 2,5-dichlorophenyl | S(C═O)OCH₃ |
| A-2072 | 2,6-dichlorophenyl | S(C═O)OCH₃ |
| A-2073 | 3,4-dichlorophenyl | S(C═O)OCH₃ |
| A-2074 | 3,5-dichlorophenyl | S(C═O)OCH₃ |
| A-2075 | 2,3-difluorophenyl | S(C═O)OCH₃ |
| A-2076 | 2,4-difluorophenyl | S(C═O)OCH₃ |
| A-2077 | 2,5-difluorophenyl | S(C═O)OCH₃ |
| A-2078 | 2,6-difluorophenyl | S(C═O)OCH₃ |
| A-2079 | 3,4-difluorophenyl | S(C═O)OCH₃ |
| A-2080 | 3,5-difluorophenyl | S(C═O)OCH₃ |
| A-2081 | 2,3-dimethylphenyl | S(C═O)OCH₃ |
| A-2082 | 2,4-dimethylphenyl | S(C═O)OCH₃ |
| A-2083 | 2,5-dimethylphenyl | S(C═O)OCH₃ |
| A-2084 | 2,6-dimethylphenyl | S(C═O)OCH₃ |
| A-2085 | 3,4-dimethylphenyl | S(C═O)OCH₃ |
| A-2086 | 3,5-dimethylphenyl | S(C═O)OCH₃ |
| A-2087 | 2,3-diethylphenyl | S(C═O)OCH₃ |
| A-2088 | 2,4-diethylphenyl | S(C═O)OCH₃ |
| A-2089 | 2,5-diethylphenyl | S(C═O)OCH₃ |
| A-2090 | 2,6-diethylphenyl | S(C═O)OCH₃ |
| A-2091 | 3,5-diethylphenyl | S(C═O)OCH₃ |
| A-2092 | 3,4-diethylphenyl | S(C═O)OCH₃ |
| A-2093 | 2,3-dimethoxyphenyl | S(C═O)OCH₃ |
| A-2094 | 2,4-dimethoxyphenyl | S(C═O)OCH₃ |
| A-2095 | 2,5-dimethoxyphenyl | S(C═O)OCH₃ |
| A-2096 | 2,6-dimethoxyphenyl | S(C═O)OCH₃ |
| A-2097 | 3,4-dimethoxyphenyl | S(C═O)OCH₃ |
| A-2098 | 3,5-dimethoxyphenyl | S(C═O)OCH₃ |
| A-2099 | 2,3-di(trifluoromethyl)phenyl | S(C═O)OCH₃ |
| A-2100 | 2,4-di(trifluoromethyl)phenyl | S(C═O)OCH₃ |
| A-2101 | 2,5-di(trifluoromethyl)phenyl | S(C═O)OCH₃ |
| A-2102 | 2,6-di(trifluoromethyl)phenyl | S(C═O)OCH₃ |
| A-2103 | 3,4-di(trifluoromethyl)phenyl | S(C═O)OCH₃ |
| A-2104 | 3,5-di(trifluoromethyl)phenyl | S(C═O)OCH₃ |
| A-2105 | 2,3-di(trifluoromethoxy)phenyl | S(C═O)OCH₃ |
| A-2106 | 2,4-di(trifluoromethoxy)phenyl | S(C═O)OCH₃ |
| A-2107 | 2,5-di(trifluoromethoxy)phenyl | S(C═O)OCH₃ |
| A-2108 | 2,6-di(trifluoromethoxy)phenyl | S(C═O)OCH₃ |
| A-2109 | 3,4-di(trifluoromethoxy)phenyl | S(C═O)OCH₃ |
| A-2110 | 3,5-di(trifluoromethoxy)phenyl | S(C═O)OCH₃ |
| A-2111 | 2,3-di(difluoromethoxy)phenyl | S(C═O)OCH₃ |
| A-2112 | 2,4-di(difluoromethoxy)phenyl | S(C═O)OCH₃ |
| A-2113 | 2,5-di(difluoromethoxy)phenyl | S(C═O)OCH₃ |
| A-2114 | 2,6-di(difluoromethoxy)phenyl | S(C═O)OCH₃ |
| A-2115 | 3,4-di(difluoromethoxy)phenyl | S(C═O)OCH₃ |
| A-2116 | 3,5-di(difluoromethoxy)phenyl | S(C═O)OCH₃ |
| A-2117 | 2,3-di(trifluoromethylthio)phenyl | S(C═O)OCH₃ |
| A-2118 | 2,4-di(trifluoromethylthio)phenyl | S(C═O)OCH₃ |
| A-2119 | 2,5-di(trifluoromethylthio)phenyl | S(C═O)OCH₃ |
| A-2120 | 2,6-di(trifluoromethylthio)phenyl | S(C═O)OCH₃ |
| A-2121 | 3,4-di(trifluoromethylthio)phenyl | S(C═O)OCH₃ |
| A-2122 | 3,5-di(trifluoromethylthio)phenyl | S(C═O)OCH₃ |
| A-2123 | 2-fluoro-3-chlorophenyl | S(C═O)OCH₃ |
| A-2124 | 2-fluoro-4-chlorophenyl | S(C═O)OCH₃ |
| A-2125 | 2-fluoro-5-chlorophenyl | S(C═O)OCH₃ |
| A-2126 | 2-fluoro-6-chlorophenyl | S(C═O)OCH₃ |
| A-2127 | 3-fluoro-4-chlorophenyl | S(C═O)OCH₃ |
| A-2128 | 3-fluoro-5-chlorophenyl | S(C═O)OCH₃ |
| A-2129 | 2-chloro-3-fluorophenyl | S(C═O)OCH₃ |
| A-2130 | 2-chloro-4-fluorophenyl | S(C═O)OCH₃ |
| A-2131 | 2-chloro-5-fluorophenyl | S(C═O)OCH₃ |
| A-2132 | 3-chloro-4-fluorophenyl | S(C═O)OCH₃ |
| A-2133 | 2-methyl-3-chlorophenyl | S(C═O)OCH₃ |
| A-2134 | 2-methyl-4-chlorophenyl | S(C═O)OCH₃ |
| A-2135 | 2-methyl-5-chlorophenyl | S(C═O)OCH₃ |
| A-2136 | 2-methyl-6-chlorophenyl | S(C═O)OCH₃ |
| A-2137 | 3-methyl-4-chlorophenyl | S(C═O)OCH₃ |
| A-2138 | 3-methyl-5-chlorophenyl | S(C═O)OCH₃ |
| A-2139 | 2-chloro-3-methylphenyl | S(C═O)OCH₃ |
| A-2140 | 2-chloro-4-methylphenyl | S(C═O)OCH₃ |
| A-2141 | 2-chloro-5-methylphenyl | S(C═O)OCH₃ |
| A-2142 | 3-chloro-4-methylphenyl | S(C═O)OCH₃ |
| A-2143 | 2-methyl-3-fluorophenyl | S(C═O)OCH₃ |
| A-2144 | 2-methyl-4-fluorophenyl | S(C═O)OCH₃ |
| A-2145 | 2-methyl-5-fluorophenyl | S(C═O)OCH₃ |
| A-2146 | 2-methyl-6-fluorophenyl | S(C═O)OCH₃ |
| A-2147 | 3-methyl-4-fluorophenyl | S(C═O)OCH₃ |
| A-2148 | 3-methyl-5-fluorophenyl | S(C═O)OCH₃ |
| A-2149 | 2-fluoro-3-methylphenyl | S(C═O)OCH₃ |
| A-2150 | 2-fluoro-4-methylphenyl | S(C═O)OCH₃ |
| A-2151 | 2-fluoro-5-methylphenyl | S(C═O)OCH₃ |
| A-2152 | 3-fluoro-4-methylphenyl | S(C═O)OCH₃ |
| A-2153 | 2-chloro-3-ethylphenyl | S(C═O)OCH₃ |
| A-2154 | 2-chloro-4-ethylphenyl | S(C═O)OCH₃ |
| A-2155 | 2-chloro-5-ethylphenyl | S(C═O)OCH₃ |
| A-2156 | 3-chloro-4-ethylphenyl | S(C═O)OCH₃ |
| A-2157 | 2-ethyl-3-chlorophenyl | S(C═O)OCH₃ |
| A-2158 | 2-ethyl-4-chlorophenyl | S(C═O)OCH₃ |
| A-2159 | 2-ethyl-5-chlorophenyl | S(C═O)OCH₃ |
| A-2160 | 2-ethyl-6-chlorophenyl | S(C═O)OCH₃ |
| A-2161 | 2-ethyl-3-fluorophenyl | S(C═O)OCH₃ |
| A-2162 | 2-ethyl-4-fluorophenyl | S(C═O)OCH₃ |
| A-2163 | 2-ethyl-5-fluorophenyl | S(C═O)OCH₃ |
| A-2164 | 2-ethyl-6-fluorophenyl | S(C═O)OCH₃ |
| A-2165 | 3-ethyl-4-fluorophenyl | S(C═O)OCH₃ |
| A-2166 | 3-ethyl-5-fluorophenyl | S(C═O)OCH₃ |
| A-2167 | 2-fluoro-3-ethylphenyl | S(C═O)OCH₃ |
| A-2168 | 2-fluoro-4-ethylphenyl | S(C═O)OCH₃ |
| A-2169 | 2-fluoro-5-ethylphenyl | S(C═O)OCH₃ |
| A-2170 | 3-fluoro-4-ethylphenyl | S(C═O)OCH₃ |
| A-2171 | 2-methoxy-3-chlorophenyl | S(C═O)OCH₃ |
| A-2172 | 2-methoxy-4-chlorophenyl | S(C═O)OCH₃ |
| A-2173 | 2-methoxy-5-chlorophenyl | S(C═O)OCH₃ |
| A-2174 | 2-methoxy-6-chlorophenyl | S(C═O)OCH₃ |
| A-2175 | 3-methoxy-4-chlorophenyl | S(C═O)OCH₃ |
| A-2176 | 3-methoxy-5-chlorophenyl | S(C═O)OCH₃ |
| A-2177 | 2-chloro-3-methoxyphenyl | S(C═O)OCH₃ |
| A-2178 | 2-chloro-4-methoxyphenyl | S(C═O)OCH₃ |
| A-2179 | 2-chloro-5-methoxyphenyl | S(C═O)OCH₃ |
| A-2180 | 3-chloro-4-methoxyphenyl | S(C═O)OCH₃ |
| A-2181 | 2-methoxy-3-fluorophenyl | S(C═O)OCH₃ |
| A-2182 | 2-methoxy-4-fluorophenyl | S(C═O)OCH₃ |
| A-2183 | 2-methoxy-5-fluorophenyl | S(C═O)OCH₃ |
| A-2184 | 2-methoxy-6-fluorophenyl | S(C═O)OCH₃ |
| A-2185 | 3-methoxy-4-fluorophenyl | S(C═O)OCH₃ |
| A-2186 | 3-methoxy-5-fluorophenyl | S(C═O)OCH₃ |
| A-2187 | 2-fluoro-3-methoxyphenyl | S(C═O)OCH₃ |
| A-2188 | 2-fluoro-4-methoxyphenyl | S(C═O)OCH₃ |
| A-2189 | 2-fluoro-5-methoxyphenyl | S(C═O)OCH₃ |
| A-2190 | 3-fluoro-4-methoxyphenyl | S(C═O)OCH₃ |
| A-2191 | 3-fluoro-5-methoxyphenyl | S(C═O)OCH₃ |
| A-2192 | 2-(trifluoromethyl)-3-chlorophenyl | S(C═O)OCH₃ |
| A-2193 | 2-(trifluoromethyl)-4-chlorophenyl | S(C═O)OCH₃ |
| A-2194 | 2-(trifluoromethyl)-5-chlorophenyl | S(C═O)OCH₃ |
| A-2195 | 2-(trifluoromethyl)-6-chlorophenyl | S(C═O)OCH₃ |
| A-2196 | 3-(trifluoromethyl)-4-chlorophenyl | S(C═O)OCH₃ |
| A-2197 | 3-(trifluoromethyl)-5-chlorophenyl | S(C═O)OCH₃ |
| A-2198 | 2-chloro-3-(trifluoromethyl)phenyl | S(C═O)OCH₃ |
| A-2199 | 2-chloro-4-(trifluoromethyl)phenyl | S(C═O)OCH₃ |
| A-2200 | 2-chloro-5-(trifluoromethyl)phenyl | S(C═O)OCH₃ |
| A-2201 | 3-chloro-4-(trifluoromethyl)phenyl | S(C═O)OCH₃ |
| A-2202 | 2-(trifluoromethyl)-3-fluorophenyl | S(C═O)OCH₃ |
| A-2203 | 2-(trifluoromethyl)-4-fluorophenyl | S(C═O)OCH₃ |
| A-2204 | 2-(trifluoromethyl)-5-fluorophenyl | S(C═O)OCH₃ |
| A-2205 | 2-(trifluoromethyl)-6-fluorophenyl | S(C═O)OCH₃ |
| A-2206 | 3-(trifluoromethyl)-4-fluorophenyl | S(C═O)OCH₃ |
| A-2207 | 3-(trifluoromethyl)-5-fluorophenyl | S(C═O)OCH₃ |
| A-2208 | 2-fluoro-3-(trifluoromethyl)phenyl | S(C═O)OCH₃ |
| A-2209 | 2-fluoro-4-(trifluoromethyl)phenyl | S(C═O)OCH₃ |
| A-2210 | 2-fluoro-5-(trifluoromethyl)phenyl | S(C═O)OCH₃ |
| A-2211 | 3-fluoro-4-(trifluoromethyl)phenyl | S(C═O)OCH₃ |
| A-2212 | 2-(trifluoromethoxy)-3-chlorophenyl | S(C═O)OCH₃ |
| A-2213 | 2-(trifluoromethoxy)-4-chlorophenyl | S(C═O)OCH₃ |
| A-2214 | 2-(trifluoromethoxy)-5-chlorophenyl | S(C═O)OCH₃ |
| A-2215 | 2-(trifluoromethoxy)-6-chlorophenyl | S(C═O)OCH₃ |
| A-2216 | 3-(trifluoromethoxy)-4-chlorophenyl | S(C═O)OCH₃ |
| A-2217 | 3-(trifluoromethoxy)-5-chlorophenyl | S(C═O)OCH₃ |
| A-2218 | 2-chloro-3-(trifluoromethoxy)phenyl | S(C═O)OCH₃ |
| A-2219 | 2-chloro-4-(trifluoromethoxy)phenyl | S(C═O)OCH₃ |
| A-2220 | 2-chloro-5-(trifluoromethoxy)phenyl | S(C═O)OCH₃ |
| A-2221 | 3-chloro-4-(trifluoromethoxy)phenyl | S(C═O)OCH₃ |
| A-2222 | 2-(trifluoromethoxy)-3-fluorophenyl | S(C═O)OCH₃ |
| A-2223 | 2-(trifluoromethoxy)-4-fluorophenyl | S(C═O)OCH₃ |
| A-2224 | 2-(trifluoromethoxy)-5-fluorophenyl | S(C═O)OCH₃ |
| A-2225 | 2-(trifluoromethoxy)-6-fluorophenyl | S(C═O)OCH₃ |
| A-2226 | 3-(trifluoromethoxy)-4-fluorophenyl | S(C═O)OCH₃ |
| A-2227 | 3-(trifluoromethoxy)-5-fluorophenyl | S(C═O)OCH₃ |
| A-2228 | 2-fluoro-3-(trifluoromethoxy)phenyl | S(C═O)OCH₃ |
| A-2229 | 2-fluoro-4-(trifluoromethoxy)phenyl | S(C═O)OCH₃ |
| A-2230 | 2-fluoro-5-(trifluoromethoxy)phenyl | S(C═O)OCH₃ |
| A-2231 | 3-fluoro-4-(trifluoromethoxy)phenyl | S(C═O)OCH₃ |
| A-2232 | 2-(difluoromethoxy)-3-chlorophenyl | S(C═O)OCH₃ |
| A-2233 | 2-(difluoromethoxy)-4-chlorophenyl | S(C═O)OCH₃ |
| A-2234 | 2-(difluoromethoxy)-5-chlorophenyl | S(C═O)OCH₃ |
| A-2235 | 2-(difluoromethoxy)-6-chlorophenyl | S(C═O)OCH₃ |
| A-2236 | 3-(difluoromethoxy)-4-chlorophenyl | S(C═O)OCH₃ |
| A-2237 | 3-(difluoromethoxy)-5-chlorophenyl | S(C═O)OCH₃ |
| A-2238 | 2-chloro-3-(difluoromethoxy)phenyl | S(C═O)OCH₃ |
| A-2239 | 2-chloro-4-(difluoromethoxy)phenyl | S(C═O)OCH₃ |
| A-2240 | 2-chloro-5-(difluoromethoxy)phenyl | S(C═O)OCH₃ |
| A-2241 | 3-chloro-4-(difluoromethoxy)phenyl | S(C═O)OCH₃ |
| A-2242 | 2-(difluoromethoxy)-3-fluorophenyl | S(C═O)OCH₃ |
| A-2243 | 2-(difluoromethoxy)-4-fluorophenyl | S(C═O)OCH₃ |
| A-2244 | 2-(difluoromethoxy)-5-fluorophenyl | S(C═O)OCH₃ |
| A-2245 | 2-(difluoromethoxy)-6-fluorophenyl | S(C═O)OCH₃ |
| A-2246 | 3-(difluoromethoxy)-4-fluorophenyl | S(C═O)OCH₃ |
| A-2247 | 3-(difluoromethoxy)-5-fluorophenyl | S(C═O)OCH₃ |
| A-2248 | 2-fluoro-3-(difluoromethoxy)phenyl | S(C═O)OCH₃ |
| A-2249 | 2-fluoro-4-(difluoromethoxy)phenyl | S(C═O)OCH₃ |
| A-2250 | 2-fluoro-5-(difluoromethoxy)phenyl | S(C═O)OCH₃ |
| A-2251 | 3-fluoro-4-(difluoromethoxy)phenyl | S(C═O)OCH₃ |
| A-2252 | 2-(trifluoromethylthio)-3-chlorophenyl | S(C═O)OCH₃ |
| A-2253 | 2-(trifluoromethylthio)-4-chlorophenyl | S(C═O)OCH₃ |
| A-2254 | 2-(trifluoromethylthio)-5-chlorophenyl | S(C═O)OCH₃ |
| A-2255 | 2-(trifluoromethylthio)-6-chlorophenyl | S(C═O)OCH₃ |
| A-2256 | 3-(trifluoromethylthio)-4-chlorophenyl | S(C═O)OCH₃ |
| A-2257 | 3-(trifluoromethylthio)-5-chlorophenyl | S(C═O)OCH₃ |
| A-2258 | 2-chloro-3-(trifluoromethylthio)phenyl | S(C═O)OCH₃ |
| A-2259 | 2-chloro-4-(trifluoromethylthio)phenyl | S(C═O)OCH₃ |
| A-2260 | 2-chloro-5-(trifluoromethylthio)phenyl | S(C═O)OCH₃ |
| A-2261 | 3-chloro-4-(trifluoromethylthio)phenyl | S(C═O)OCH₃ |
| A-2262 | 2-(trifluoromethylthio)-3-fluorophenyl | S(C═O)OCH₃ |
| A-2263 | 2-(trifluoromethylthio)-4-fluorophenyl | S(C═O)OCH₃ |
| A-2264 | 2-(trifluoromethylthio)-5-fluorophenyl | S(C═O)OCH₃ |
| A-2265 | 2-(trifluoromethylthio)-6-fluorophenyl | S(C═O)OCH₃ |
| A-2266 | 3-(trifluoromethylthio)-4-fluorophenyl | S(C═O)OCH₃ |
| A-2267 | 3-(trifluoromethylthio)-5-fluorophenyl | S(C═O)OCH₃ |
| A-2268 | 2-fluoro-3-(trifluoromethylthio)phenyl | S(C═O)OCH₃ |
| A-2269 | 2-fluoro-4-(trifluoromethylthio)phenyl | S(C═O)OCH₃ |
| A-2270 | 2-fluoro-5-(trifluoromethylthio)phenyl | S(C═O)OCH₃ |
| A-2271 | 3-fluoro-4-(trifluoromethylthio)phenyl | S(C═O)OCH₃ |
| A-2272 | 2,3,4-trichlorophenyl | S(C═O)OCH₃ |
| A-2273 | 2,3,5-trichlorophenyl | S(C═O)OCH₃ |
| A-2274 | 2,3,6-trichlorophenyl | S(C═O)OCH₃ |
| A-2275 | 2,4,5-trichlorophenyl | S(C═O)OCH₃ |
| A-2276 | 2,4,6-trichlorophenyl | S(C═O)OCH₃ |
| A-2277 | 3,4,5-trichlorophenyl | S(C═O)OCH₃ |
| A-2278 | 2,3,4-trifluorophenyl | S(C═O)OCH₃ |
| A-2279 | 2,3,5-trifluorophenyl | S(C═O)OCH₃ |
| A-2280 | 2,3,6-trifluorophenyl | S(C═O)OCH₃ |
| A-2281 | 2,4,5-trifluorophenyl | S(C═O)OCH₃ |
| A-2282 | 2,4,6-trifluorophenyl | S(C═O)OCH₃ |
| A-2283 | 3,4,5-trifluorophenyl | S(C═O)OCH₃ |
| A-2284 | 2,3,4-trimethylphenyl | S(C═O)OCH₃ |
| A-2285 | 2,3,5-trimethylphenyl | S(C═O)OCH₃ |
| A-2286 | 2,3,6-trimethylphenyl | S(C═O)OCH₃ |
| A-2287 | 2,4,5-trimethylphenyl | S(C═O)OCH₃ |
| A-2288 | 2,4,6-trimethylphenyl | S(C═O)OCH₃ |
| A-2289 | 3,4,5-trimethylphenyl | S(C═O)OCH₃ |
| A-2290 | 2,3,4-trimethoxyphenyl | S(C═O)OCH₃ |
| A-2291 | 2,3,5-trimethoxyphenyl | S(C═O)OCH₃ |
| A-2292 | 2,3,6-trimethoxyphenyl | S(C═O)OCH₃ |
| A-2293 | 2,4,5-trimethoxyphenyl | S(C═O)OCH₃ |
| A-2294 | 2,4,6-trimethoxyphenyl | S(C═O)OCH₃ |
| A-2295 | 3,4,5-trimethoxyphenyl | S(C═O)OCH₃ |

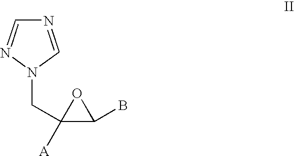

In particular with a view to their use, preference is given to the compounds II compiled in Tables 1c to 93c below. The groups mentioned in the tables for a substituent are furthermore per se, independently of the combination in which they are mentioned, a particularly preferred aspect of the substituent in question.

### Table 1c

Compounds II in which A is 2,3-difluorophenyl and B corresponds in each case to one row of Table C (compounds II.1cC-1 to II.1cC-234)

### Table 2c

Compounds II in which A is 2,4-difluorophenyl and B corresponds in each case to one row of Table C (compounds II.2cC-1 to II.2cC-234)

### Table 3c

Compounds II in which A is 2,5-difluorophenyl and B corresponds in each case to one row of Table C (compounds II.3cC-1 to II.3cC-234)

### Table 4c

Compounds II in which A is 2,6-difluorophenyl and B corresponds in each case to one row of Table C (compounds II.4cC-1 to II.4cC-234)

### Table 5c

Compounds II in which A is 3,4-difluorophenyl and B corresponds in each case to one row of Table C (compounds II.5cC-1 to II.5cC-255)

### Table 6c

Compounds II in which A is 3,5-difluorophenyl and B corresponds in each case to one row of Table C (compounds II.6cC-1 to II.6cC-234)

### Table 7c

Compounds II in which A is 2-fluoro-3-chlorophenyl and B corresponds in each case to one row of Table B (compounds II.7cB-1 to II.7cB-255)

### Table 8c

Compounds II in which A is 2-fluoro-4-chlorophenyl and B corresponds in each case to one row of Table C (compounds II.8cC-1 to II.8cC-234)

### Table 9c

Compounds II in which A is 2-fluoro-5-chlorophenyl and B corresponds in each case to one row of Table B (compounds II.9cB-1 to II.9cB-255)

### Table 10c

Compounds II in which A is 2-fluoro-6-chlorophenyl I and B corresponds in each case to one row of Table B (compounds II.10cB-1 to II.10cB-255)

### Table 11c

Compounds II in which A is 3-fluoro-4-chlorophenyl and B corresponds in each case to one row of Table B (compounds II.11cB-1 to II.11cB-255)

### Table 12c

Compounds II in which A is 3-fluoro-5-chlorophenyl and B corresponds in each case to one row of Table C (compounds II.12cC-1 to II.12cC-234)

### Table 13c

Compounds II in which A is 2-chloro-3-fluorophenyl and B corresponds in each case to one row of Table B (compounds II.13cB-1 to II.13cB-255)

### Table 14c

Compounds II in which A is 2-chloro-4-fluorophenyl and B corresponds in each case to one row of Table C (compounds II.14cC-1 to II.14cC-234)

### Table 15c

Compounds II in which A is 2-chloro-5-fluorophenyl and B corresponds in each case to one row of Table B (compounds II.15cB-1 to II.15cB-255)

### Table 16c

Compounds II in which A is 3-chloro-4-fluorophenyl and B corresponds in each case to one row of Table B (compounds II.16cB-1 to II.16cB-255)

### Table 17c

Compounds II in which A is 2-methyl-3-fluorophenyl and B corresponds in each case to one row of Table B (compounds II.17cB-1 to II.17cB-255)

### Table 18c

Compounds II in which A is 2-methyl-4-fluorophenyl and B corresponds in each case to one row of Table B (compounds II.18cB-1 to II.18cB-255)

### Table 19c

Compounds II in which A is 2-methyl-5-fluorophenyl and B corresponds in each case to one row of Table B (compounds II.19cB-1 to II.19cB-255)

### Table 20c

Compounds II in which A is 2-methyl-6-fluorophenyl and B corresponds in each case to one row of Table B (compounds II.20cB-1 to II.20cB-255)

### Table 21c

Compounds II in which A is 3-methyl-4-fluorophenyl and B corresponds in each case to one row of Table C (compounds II.21cC-1 to II.21cC-234)

### Table 22c

Compounds II in which A is 3-methyl-5-fluorophenyl and B corresponds in each case to one row of Table C (compounds II.22cC-1 to II.22cC-234)

### Table 23c

Compounds II in which A is 2-fluoro-3-methylphenyl and B corresponds in each case to one row of Table B (compounds II.23cB-1 to II.23cB-255)

### Table 24c

Compounds II in which A is 2-fluoro-4-methylphenyl and B corresponds in each case to one row of Table C (compounds II.24cC-1 to II.24cC-234)

### Table 25c

Compounds II in which A is 2-fluoro-5-methylphenyl and B corresponds in each case to one row of Table B (compounds II.25cB-1 to II.25cB-255)

### Table 26c

Compounds II in which A is 3-fluoro-4-methylphenyl and B corresponds in each case to one row of Table C (compounds II.26cC-1 to II.26cC-234)

### Table 27c

Compounds II in which A is 2-ethyl-3-fluorophenyl and B corresponds in each case to one row of Table B (compounds II.27cB-1 to II.27cB-255)

### Table 28c

Compounds II in which A is 2-ethyl-4-fluorophenyl and B corresponds in each case to one row of Table B (compounds II.28cB-1 to II.28cB-255)

### Table 29c

Compounds II in which A is 2-ethyl-5-fluorophenyl and B corresponds in each case to one row of Table B (compounds II.29cB-1 to II.29cB-255)

### Table 30c

Compounds II in which A is 2-ethyl-6-fluorophenyl and B corresponds in each case to one row of Table B (compounds II.30cB-1 to II.30cB-255)

### Table 31c

Compounds II in which A is 3-ethyl-4-fluorophenyl and B corresponds in each case to one row of Table B (compounds II.31cB-1 to II.31cB-255)

### Table 32c

Compounds II in which A is 3-ethyl-5-fluorophenyl and B corresponds in each case to one row of Table B (compounds II.32cB-1 to II.32cB-255)

### Table 33c

Compounds II in which A is 2-fluoro-3-ethylphenyl and B corresponds in each case to one row of Table B (compounds II.33cB-1 to II.33cB-255)

### Table 34c

Compounds II in which A is 2-fluoro-4-ethylphenyl and B corresponds in each case to one row of Table B (compounds II.34cB-1 to II.34cB-255)

### Table 35c

Compounds II in which A is 2-fluoro-5-ethylphenyl and B corresponds in each case to one row of Table B (compounds II.35cB-1 to II.35cB-255)

### Table 36c

Compounds II in which A is 3-fluoro-4-ethylphenyl and B corresponds in each case to one row of Table B (compounds II.36cB-1 to II.36cB-255)

### Table 37c

Compounds II in which A is 2-methoxy-3-fluorophenyl and B corresponds in each case to one row of Table B (compounds II.37cB-1 to II.37cB-255)

### Table 38c

Compounds II in which A is 2-methoxy-4-fluorophenyl and B corresponds in each case to one row of Table C (Compounds II.38cC-1 to II.38cC-234)

### Table 39c

Compounds II in which A is 2-methoxy-5-fluorophenyl and B corresponds in each case to one row of Table B (compounds II.39cB-1 to II.39cB-255)

### Table 40c

Compounds II in which A is 2-methoxy-6-fluorophenyl and B corresponds in each case to one row of Table B (compounds II.40cB-1 to II.40cB-255)

### Table 41c

Compounds II in which A is 3-methoxy-4-fluorophenyl and B corresponds in each case to one row of Table C (Compounds II.41cC-1 to II.41cC-234)

### Table 42c

Compounds II in which A is 3-methoxy-5-fluorophenyl and B corresponds in each case to one row of Table B (compounds II.42cB-1 to II.42cB-255)

### Table 43c

Compounds II in which A is 2-fluoro-3-methoxyphenyl and B corresponds in each case to one row of Table B (compounds II.43cB-1 to II.43cB-255)

### Table 44c

Compounds II in which A is 2-fluoro-4-methoxyphenyl and B corresponds in each case to one row of Table C (compounds II.44cC-1 to II.44cC-234)

### Table 45c

Compounds II in which A is 2-fluoro-5-methoxyphenyl and B corresponds in each case to one row of Table C (compounds II.45cC-1 to II.45cC-234)

### Table 46c

Compounds II in which A is 3-fluoro-4-methoxyphenyl and B corresponds in each case to one row of Table C (compounds II.46cC-1 to II.46cC-234)

### Table 47c

Compounds II in which A is 3-fluoro-5-methoxyphenyl and B corresponds in each case to one row of Table B (compounds II.47cB-1 to II.47cB-255)

### Table 48c

Compounds II in which A is 2-(trifluoromethyl)-3-fluorophenyl and B corresponds in each case to one row of Table B (compounds II.48cB-1 to II.48cB-255)

### Table 49c

Compounds II in which A is 2-(trifluoromethyl)-4-fluorophenyl and B corresponds in each case to one row of Table B (compounds II.49cB-1 to II.49cB-255)

### Table 50c

Compounds II in which A is 2-(trifluoromethyl)-5-fluorophenyl and B corresponds in each case to one row of Table B (compounds II.50cB-1 to II.50cB-255)

### Table 51c

Compounds II in which A is 2-(trifluoromethyl)-6-fluorophenyl and B corresponds in each case to one row of Table B (compounds II.51cB-1 to II.51cB-255)

### Table 52c

Compounds II in which A is 3-(trifluoromethyl)-4-fluorophenyl and B corresponds in each case to one row of Table B (compounds II.52cB-1 to II.52cB-255)

### Table 53c

Compounds II in which A is 3-(trifluoromethyl)-5-fluorophenyl and B corresponds in each case to one row of Table B (compounds II.53cB-1 to II.53cB-255)

### Table 54c

Compounds II in which A is 2-fluoro-3-(trifluoromethyl)phenyl and B corresponds in each case to one row of Table B (compounds II.54cB-1 to II.54cB-255)

### Table 55c

Compounds II in which A is 2-fluoro-4-(trifluoromethyl)phenyl and B corresponds in each case to one row of Table B (compounds II.55cB-1 to II.55cB-255)

### Table 56c

Compounds II in which A is 2-fluoro-5-(trifluoromethyl)phenyl and B corresponds in each case to one row of Table B (compounds II.56cB-1 to II.56cB-255)

### Table 57c

Compounds II in which A is 3-fluoro-4-(trifluoromethyl)phenyl and B corresponds in each case to one row of Table B (compounds II.57cB-1 to II.57cB-255)

### Table 58c

Compounds II in which A is 2-(trifluoromethoxy)-3-fluorophenyl and B corresponds in each case to one row of Table B (compounds II.58cB-1 to II.58cB-255)

### Table 59c

Compounds II in which A is 2-(trifluoromethoxy)-4-fluorophenyl and B corresponds in each case to one row of Table B (compounds II.59cB-1 to II.59cB-255)

### Table 60c

Compounds II in which A is 2-(trifluoromethoxy)-5-fluorophenyl and B corresponds in each case to one row of Table B (compounds II.60cB-1 to II.60cB-255)

### Table 61c

Compounds II in which A is 2-(trifluoromethoxy)-6-fluorophenyl and B corresponds in each case to one row of Table B (compounds II.61cB-1 to II.61cB-255)

### Table 62c

Compounds II in which A is 3-(trifluoromethoxy)-4-fluorophenyl and B corresponds in each case to one row of Table B (compounds II.62cB-1 to II.62cB-255)

### Table 63c

Compounds II in which A is 3-(trifluoromethoxy)-5-fluorophenyl and B corresponds in each case to one row of Table B (compounds II.63cB-1 to II.63cB-255)

### Table 64c

Compounds II in which A is 2-fluoro-3-(trifluoromethoxy)phenyl and B corresponds in each case to one row of Table B (compounds II.64cB-1 to II.64cB-255)

### Table 65c

Compounds II in which A is 2-fluoro-4-(trifluoromethoxy)phenyl and B corresponds in each case to one row of Table B (compounds II.65cB-1 to II.65cB-255)

### Table 66c

Compounds II in which A is 2-fluoro-5-(trifluoromethoxy)phenyl and B corresponds in each case to one row of Table B (compounds II.66cB-1 to II.66cB-255)

### Table 67c

Compounds II in which A is 3-fluoro-4-(trifluoromethoxy)phenyl and B corresponds in each case to one row of Table B (compounds II.67cB-1 to II.67cB-255)

### Table 68c

Compounds II in which A is 2-(difluoromethoxy)-3-fluorophenyl and B corresponds in each case to one row of Table B (compounds II.68cB-1 to II.68cB-255)

### Table 69c

Compounds II in which A is 2-(difluoromethoxy)-4-fluorophenyl and B corresponds in each case to one row of Table B (compounds II.69cB-1 to II.69cB-255)

### Table 70c

Compounds II in which A is 2-(difluoromethoxy)-5-fluorophenyl and B corresponds in each case to one row of Table B (compounds II.70cB-1 to II.70cB-255)

### Table 71c

Compounds II in which A is 2-(difluoromethoxy)-6-fluorophenyl and B corresponds in each case to one row of Table B (compounds II.71cB-1 to II.71cB-255)

### Table 72c

Compounds 11 in which A is 3-(difluoromethoxy)-4-fluorophenyl and B corresponds in each case to one row of Table B (compounds II.72cB-1 to II.72cB-255)

### Table 73c

Compounds II in which A is 3-(difluoromethoxy)-5-fluorophenyl and B corresponds in each case to one row of Table B (compounds II.73cB-1 to II.73cB-255)

### Table 74c

Compounds II in which A is 2-fluoro-3-(difluoromethoxy)phenyl and B corresponds in each case to one row of Table B (compounds II.74cB-1 to II.74cB-255)

### Table 75c

Compounds II in which A is 2-fluoro-4-(difluoromethoxy)phenyl and B corresponds in each case to one row of Table B (compounds II.75cB-1 to II.75cB-255)

### Table 76c

Compounds II in which A is 2-fluoro-5-(difluoromethoxy)phenyl and B corresponds in each case to one row of Table B (compounds II.76cB-1 to II.76cB-255)

### Table 77c

Compounds II in which A is 3-fluoro-4-(difluoromethoxy)phenyl and B corresponds in each case to one row of Table B (compounds II.77cB-1 to II.77cB-255)

### Table 78c

Compounds II in which A is 2-(trifluoromethylthio)-3-fluorophenyl and B corresponds in each case to one row of Table B (compounds II.78cB-1 to II.78cB-255)

### Table 79c

Compounds II in which A is 2-(trifluoromethylthio)-4-fluorophenyl and B corresponds in each case to one row of Table B (compounds II.79cB-1 to II.79cB-255)

### Table 80c

Compounds II in which A is 2-(trifluoromethylthio)-5-fluorophenyl and B corresponds in each case to one row of Table B (compounds II.80cB-1 to II.80cB-255)

### Table 81c

Compounds II in which A is 2-(trifluoromethylthio)-6-fluorophenyl and B corresponds in each case to one row of Table B (compounds II.81cB-1 to II.81cB-255)

### Table 82c

Compounds II in which A is 3-(trifluoromethylthio)-4-fluorophenyl and B corresponds in each case to one row of Table B (compounds II.82cB-1 to II.82cB-255)

### Table 83c

Compounds II in which A is 3-(trifluoromethylthio)-5-fluorophenyl and B corresponds in each case to one row of Table B (compounds II.83cB-1 to II.83cB-255)

### Table 84c

Compounds II in which A is 2-fluoro-3-(trifluoromethylthio)phenyl and B corresponds in each case to one row of Table B (compounds II.84cB-1 to II.84cB-255)

### Table 85c

Compounds II in which A is 2-fluoro-4-(trifluoromethylthio)phenyl and B corresponds in each case to one row of Table B (compounds II.85cB-1 to II.85cB-255)

### Table 86c

Compounds II in which A is 2-fluoro-5-(trifluoromethylthio)phenyl and B corresponds in each case to one row of Table B (compounds II.86cB-1 to II.86cB-255)

### Table 87c

Compounds II in which A is 3-fluoro-4-(trifluoromethylthio)phenyl and B corresponds in each case to one row of Table B (compounds II.87cB-1 to II.87cB-255)

### Table 88c

Compounds II in which A is 2,3,4-trifluorophenyl and B corresponds in each case to one row of Table C (compounds II.88cC-1 to II.88cC-234)

### Table 89c

Compounds II in which A is 2,3,5-Trifluorophenyl and B corresponds in each case to one row of Table C (compounds II.89cC-1 to II.89cC-234)

### Table 90c

Compounds II in which A is 2,3,6-Trifluorophenyl and B corresponds in each case to one row of Table C (compounds II.90cC-1 to II.90cC-234)

### Table 91c

Compounds II in which A is 2,4,5-trifluorophenyl and B corresponds in each case to one row of Table C (compounds II.91cC-1 to II.91cC-234)

### Table 92c

Compounds II in which A is 2,4,6-trifluorophenyl and B corresponds in each case to one row of Table C (compounds II.92cC-1 to II.92cC-234)

### Table 93c

Compounds II in which A is 3,4,5-trifluorophenyl and B corresponds in each case to one row of Table C (compounds II.93cC-1 to II.93cC-234)

TABLE B

| Row | B |
|---|---|
| B-1 | phenyl |
| B-2 | 2-chlorophenyl |
| B-3 | 3-chlorophenyl |
| B-4 | 4-chlorophenyl |
| B-5 | 2-fluorophenyl |
| B-6 | 3-fluorophenyl |
| B-7 | 4-fluorophenyl |
| B-8 | 2-methylphenyl |
| B-9 | 3-methylphenyl |
| B-10 | 4-methylphenyl |
| B-11 | 2-ethylphenyl |
| B-12 | 3-ethylphenyl |
| B-13 | 4-ethylphenyl |
| B-14 | 2-methoxyphenyl |
| B-15 | 3-methoxyphenyl |
| B-16 | 4-methoxyphenyl |
| B-17 | 2-trifluoromethylphenyl |
| B-18 | 3-trifluoromethylphenyl |
| B-19 | 4-trifluoromethylphenyl |
| B-20 | 2-trifluoromethoxyphenyl |
| B-21 | 3-trifluoromethoxyphenyl |
| B-22 | 4-trifluoromethoxyphenyl |
| B-23 | 2-difluoromethoxyphenyl |
| B-24 | 3-difluoromethoxyphenyl |
| B-25 | 4-difluoromethoxyphenyl |
| B-26 | 2-trifluoromethylthiophenyl |
| B-27 | 3-trifluoromethylthiophenyl |
| B-28 | 4-trifluoromethylthiophenyl |
| B-29 | 2,3-dichlorophenyl |
| B-30 | 2,4-dichlorophenyl |
| B-31 | 2,5-dichlorophenyl |
| B-32 | 2,6-dichlorophenyl |
| B-33 | 3,4-dichlorophenyl |
| B-34 | 3,5-dichlorophenyl |
| B-35 | 2,3-difluorophenyl |
| B-36 | 2,4-difluorophenyl |
| B-37 | 2,5-difluorophenyl |
| B-38 | 2,6-difluorophenyl |
| B-39 | 3,4-difluorophenyl |
| B-40 | 3,5-difluorophenyl |
| B-41 | 2,3-dimethylphenyl |
| B-42 | 2,4-dimethylphenyl |
| B-43 | 2,5-dimethylphenyl |
| B-44 | 2,6-dimethylphenyl |
| B-45 | 3,4-dimethylphenyl |
| B-46 | 3,5-dimethylphenyl |
| B-47 | 2,3-diethylphenyl |
| B-48 | 2,4-diethylphenyl |
| B-49 | 2,5-diethylphenyl |
| B-50 | 2,6-diethylphenyl |
| B-51 | 3,5-diethylphenyl |
| B-52 | 3,4-diethylphenyl |
| B-53 | 2,3-dimethoxyphenyl |
| B-54 | 2,4-dimethoxyphenyl |
| B-55 | 2,5-dimethoxyphenyl |
| B-56 | 2,6-dimethoxyphenyl |
| B-57 | 3,4-dimethoxyphenyl |
| B-58 | 3,5-dimethoxyphenyl |
| B-59 | 2,3-di(trifluoromethyl)phenyl |
| B-60 | 2,4-di(trifluoromethyl)phenyl |
| B-61 | 2,5-di(trifluoromethyl)phenyl |
| B-62 | 2,6-di(trifluoromethyl)phenyl |
| B-63 | 3,4-di(trifluoromethyl)phenyl |
| B-64 | 3,5-di(trifluoromethyl)phenyl |
| B-65 | 2,3-di(trifluoromethoxy)phenyl |
| B-66 | 2,4-di(trifluoromethoxy)phenyl |
| B-67 | 2,5-di(trifluoromethoxy)phenyl |
| B-68 | 2,6-di(trifluoromethoxy)phenyl |
| B-69 | 3,4-di(trifluoromethoxy)phenyl |
| B-70 | 3,5-di(trifluoromethoxy)phenyl |
| B-71 | 2,3-di(difluoromethoxy)phenyl |
| B-72 | 2,4-di(difluoromethoxy)phenyl |
| B-73 | 2,5-di(difluoromethoxy)phenyl |
| B-74 | 2,6-di(difluoromethoxy)phenyl |
| B-75 | 3,4-di(difluoromethoxy)phenyl |
| B-76 | 3,5-di(difluoromethoxy)phenyl |
| B-77 | 2,3-di(trifluoromethylthio)phenyl |
| B-78 | 2,4-di(trifluoromethylthio)phenyl |
| B-79 | 2,5-di(trifluoromethylthio)phenyl |
| B-80 | 2,6-di(trifluoromethylthio)phenyl |
| B-81 | 3,4-di(trifluoromethylthio)phenyl |
| B-82 | 3,5-di(trifluoromethylthio)phenyl |
| B-83 | 2-fluoro-3-chlorophenyl |
| B-84 | 2-fluoro-4-chlorophenyl |
| B-85 | 2-fluoro-5-chlorophenyl |
| B-86 | 2-fluoro-6-chlorophenyl |
| B-87 | 3-fluoro-4-chlorophenyl |
| B-88 | 3-fluoro-5-chlorophenyl |
| B-89 | 2-chloro-3-fluorophenyl |
| B-90 | 2-chloro-4-fluorophenyl |
| B-91 | 2-chloro-5-fluorophenyl |
| B-92 | 3-chloro-4-fluorophenyl |
| B-93 | 2-methyl-3-chlorophenyl |
| B-94 | 2-methyl-4-chlorophenyl |
| B-95 | 2-methyl-5-chlorophenyl |
| B-96 | 2-methyl-6-chlorophenyl |
| B-97 | 3-methyl-4-chlorophenyl |
| B-98 | 3-methyl-5-chlorophenyl |
| B-99 | 2-chloro-3-methylphenyl |
| B-100 | 2-chloro-4-methylphenyl |
| B-101 | 2-chloro-5-methylphenyl |
| B-102 | 3-chloro-4-methylphenyl |
| B-103 | 2-methyl-3-fluorophenyl |
| B-104 | 2-methyl-4-fluorophenyl |
| B-105 | 2-methyl-5-fluorophenyl |
| B-106 | 2-methyl-6-fluorophenyl |
| B-107 | 3-methyl-4-fluorophenyl |
| B-108 | 3-methyl-5-fluorophenyl |
| B-109 | 2-fluoro-3-methylphenyl |
| B-110 | 2-fluoro-4-methylphenyl |
| B-111 | 2-fluoro-5-methylphenyl |
| B-112 | 3-fluoro-4-methylphenyl |
| B-113 | 2-chloro-3-ethylphenyl |
| B-114 | 2-chloro-4-ethylphenyl |
| B-115 | 2-chloro-5-ethylphenyl |
| B-116 | 3-chloro-4-ethylphenyl |
| B-117 | 2-ethyl-3-chlorophenyl |
| B-118 | 2-ethyl-4-chlorophenyl |
| B-119 | 2-ethyl-5-chlorophenyl |
| B-120 | 2-ethyl-6-chlorophenyl |
| B-121 | 2-ethyl-3-fluorophenyl |
| B-122 | 2-ethyl-4-fluorophenyl |
| B-123 | 2-ethyl-5-fluorophenyl |
| B-124 | 2-ethyl-6-fluorophenyl |
| B-125 | 3-ethyl-4-fluorophenyl |
| B-126 | 3-ethyl-5-fluorophenyl |
| B-127 | 2-fluoro-3-ethylphenyl |
| B-128 | 2-fluoro-4-ethylphenyl |
| B-129 | 2-fluoro-5-ethylphenyl |
| B-130 | 3-fluoro-4-ethylphenyl |
| B-131 | 2-methoxy-3-chlorophenyl |
| B-132 | 2-methoxy-4-chlorophenyl |
| B-133 | 2-methoxy-5-chlorophenyl |
| B-134 | 2-methoxy-6-chlorophenyl |
| B-135 | 3-methoxy-4-chlorophenyl |
| B-136 | 3-methoxy-5-chlorophenyl |
| B-137 | 2-chloro-3-methoxyphenyl |
| B-138 | 2-chloro-4-methoxyphenyl |
| B-139 | 2-chloro-5-methoxyphenyl |
| B-140 | 3-chloro-4-methoxyphenyl |
| B-141 | 2-methoxy-3-fluorophenyl |
| B-142 | 2-methoxy-4-fluorophenyl |
| B-143 | 2-methoxy-5-fluorophenyl |
| B-144 | 2-methoxy-6-fluorophenyl |
| B-145 | 3-methoxy-4-fluorophenyl |
| B-146 | 3-methoxy-5-fluorophenyl |
| B-147 | 2-fluoro-3-methoxyphenyl |
| B-148 | 2-fluoro-4-methoxyphenyl |
| B-149 | 2-fluoro-5-methoxyphenyl |
| B-150 | 3-fluoro-4-methoxyphenyl |
| B-151 | 3-fluoro-5-methoxyphenyl |
| B-152 | 2-(trifluoromethyl)-3-chlorophenyl |
| B-153 | 2-(trifluoromethyl)-4-chlorophenyl |
| B-154 | 2-(trifluoromethyl)-5-chlorophenyl |
| B-155 | 2-(trifluoromethyl)-6-chlorophenyl |
| B-156 | 3-(trifluoromethyl)-4-chlorophenyl |
| B-157 | 3-(trifluoromethyl)-5-chlorophenyl |
| B-158 | 2-chloro-3-(trifluoromethyl)phenyl |
| B-159 | 2-chloro-4-(trifluoromethyl)phenyl |
| B-160 | 2-chloro-5-(trifluoromethyl)phenyl |
| B-161 | 3-chloro-4-(trifluoromethyl)phenyl |
| B-162 | 2-(trifluoromethyl)-3-fluorophenyl |
| B-163 | 2-(trifluoromethyl)-4-fluorophenyl |
| B-164 | 2-(trifluoromethyl)-5-fluorophenyl |
| B-165 | 2-(trifluoromethyl)-6-fluorophenyl |
| B-166 | 3-(trifluoromethyl)-4-fluorophenyl |
| B-167 | 3-(trifluoromethyl)-5-fluorophenyl |
| B-168 | 2-fluoro-3-(trifluoromethyl)phenyl |
| B-169 | 2-fluoro-4-(trifluoromethyl)phenyl |
| B-170 | 2-fluoro-5-(trifluoromethyl)phenyl |
| B-171 | 3-fluoro-4-(trifluoromethyl)phenyl |
| B-172 | 2-(trifluoromethoxy)-3-chlorophenyl |
| B-173 | 2-(trifluoromethoxy)-4-chlorophenyl |
| B-174 | 2-(trifluoromethoxy)-5-chlorophenyl |
| B-175 | 2-(trifluoromethoxy)-6-chlorophenyl |
| B-176 | 3-(trifluoromethoxy)-4-chlorophenyl |
| B-177 | 3-(trifluoromethoxy)-5-chlorophenyl |
| B-178 | 2-chloro-3-(trifluoromethoxy)phenyl |
| B-179 | 2-chloro-4-(trifluoromethoxy)phenyl |
| B-180 | 2-chloro-5-(trifluoromethoxy)phenyl |
| B-181 | 3-chloro-4-(trifluoromethoxy)phenyl |
| B-182 | 2-(trifluoromethoxy)-3-fluorophenyl |
| B-183 | 2-(trifluoromethoxy)-4-fluorophenyl |
| B-184 | 2-(trifluoromethoxy)-5-fluorophenyl |
| B-185 | 2-(trifluoromethoxy)-6-fluorophenyl |
| B-186 | 3-(trifluoromethoxy)-4-fluorophenyl |
| B-187 | 3-(trifluoromethoxy)-5-fluorophenyl |
| B-188 | 2-fluoro-3-(trifluoromethoxy)phenyl |
| B-189 | 2-fluoro-4-(trifluoromethoxy)phenyl |
| B-190 | 2-fluoro-5-(trifluoromethoxy)phenyl |
| B-191 | 3-fluoro-4-(trifluoromethoxy)phenyl |
| B-192 | 2-(difluoromethoxy)-3-chlorophenyl |
| B-193 | 2-(difluoromethoxy)-4-chlorophenyl |
| B-194 | 2-(difluoromethoxy)-5-chlorophenyl |
| B-195 | 2-(difluoromethoxy)-6-chlorophenyl |
| B-196 | 3-(difluoromethoxy)-4-chlorophenyl |
| B-197 | 3-(difluoromethoxy)-5-chlorophenyl |
| B-198 | 2-chloro-3-(difluoromethoxy)phenyl |
| B-199 | 2-chloro-4-(difluoromethoxy)phenyl |
| B-200 | 2-chloro-5-(difluoromethoxy)phenyl |
| B-201 | 3-chloro-4-(difluoromethoxy)phenyl |
| B-202 | 2-(difluoromethoxy)-3-fluorophenyl |
| B-203 | 2-(difluoromethoxy)-4-fluorophenyl |
| B-204 | 2-(difluoromethoxy)-5-fluorophenyl |
| B-205 | 2-(difluoromethoxy)-6-fluorophenyl |
| B-206 | 3-(difluoromethoxy)-4-fluorophenyl |
| B-207 | 3-(difluoromethoxy)-5-fluorophenyl |
| B-208 | 2-fluoro-3-(difluoromethoxy)phenyl |
| B-209 | 2-fluoro-4-(difluoromethoxy)phenyl |
| B-210 | 2-fluoro-5-(difluoromethoxy)phenyl |
| B-211 | 3-fluoro-4-(difluoromethoxy)phenyl |
| B-212 | 2-(trifluoromethylthio)-3-chlorophenyl |
| B-213 | 2-(trifluoromethylthio)-4-chlorophenyl |
| B-214 | 2-(trifluoromethylthio)-5-chlorophenyl |
| B-215 | 2-(trifluoromethylthio)-6-chlorophenyl |
| B-216 | 3-(trifluoromethylthio)-4-chlorophenyl |
| B-217 | 3-(trifluoromethylthio)-5-chlorophenyl |
| B-218 | 2-chloro-3-(trifluoromethylthio)phenyl |
| B-219 | 2-chloro-4-(trifluoromethylthio)phenyl |
| B-220 | 2-chloro-5-(trifluoromethylthio)phenyl |
| B-221 | 3-chloro-4-(trifluoromethylthio)phenyl |
| B-222 | 2-(trifluoromethylthio)-3-fluorophenyl |
| B-223 | 2-(trifluoromethylthio)-4-fluorophenyl |
| B-224 | 2-(trifluoromethylthio)-5-fluorophenyl |
| B-225 | 2-(trifluoromethylthio)-6-fluorophenyl |
| B-226 | 3-(trifluoromethylthio)-4-fluorophenyl |
| B-227 | 3-(trifluoromethylthio)-5-fluorophenyl |
| B-228 | 2-fluoro-3-(trifluoromethylthio)phenyl |
| B-229 | 2-fluoro-4-(trifluoromethylthio)phenyl |
| B-230 | 2-fluoro-5-(trifluoromethylthio)phenyl |
| B-231 | 3-fluoro-4-(trifluoromethylthio)phenyl |
| B-232 | 2,3,4-trichlorophenyl |
| B-233 | 2,3,5-trichlorophenyl |
| B-234 | 2,3,6-trichlorophenyl |
| B-235 | 2,4,5-trichlorophenyl |
| B-236 | 2,4,6-trichlorophenyl |
| B-237 | 3,4,5-trichlorophenyl |
| B-238 | 2,3,4-trifluorophenyl |
| B-239 | 2,3,5-trifluorophenyl |
| B-240 | 2,3,6-trifluorophenyl |
| B-241 | 2,4,5-trifluorophenyl |
| B-242 | 2,4,6-trifluorophenyl |
| B-243 | 3,4,5-trifluorophenyl |
| B-244 | 2,3,4-trimethylphenyl |
| B-245 | 2,3,5-trimethylphenyl |
| B-246 | 2,3,6-trimethylphenyl |
| B-247 | 2,4,5-trimethylphenyl |
| B-248 | 2,4,6-trimethylphenyl |
| B-249 | 3,4,5-trimethylphenyl |
| B-250 | 2,3,4-trimethoxyphenyl |
| B-251 | 2,3,5-trimethoxyphenyl |
| B-252 | 2,3,6-trimethoxyphenyl |
| B-253 | 2,4,5-trimethoxyphenyl |
| B-254 | 2,4,6-trimethoxyphenyl |
| B-255 | 3,4,5-trimethoxyphenyl |
| C-1 | phenyl |
| C-2 | 2-ethylphenyl |
| C-3 | 3-ethylphenyl |
| C-4 | 4-ethylphenyl |
| C-5 | 2-trifluoromethylphenyl |
| C-6 | 3-trifluoromethylphenyl |
| C-7 | 4-trifluoromethylphenyl |
| C-8 | 2-trifluoromethoxyphenyl |
| C-9 | 3-trifluoromethoxyphenyl |
| C-10 | 4-trifluoromethoxyphenyl |
| C-11 | 2-difluoromethoxyphenyl |
| C-12 | 3-difluoromethoxyphenyl |
| C-13 | 4-difluoromethoxyphenyl |
| C-14 | 2-trifluoromethylthiophenyl |
| C-15 | 3-trifluoromethylthiophenyl |
| C-16 | 4-trifluoromethylthiophenyl |
| C-17 | 2,5-dichlorophenyl |
| C-18 | 2,6-dichlorophenyl |
| C-19 | 3,5-dichlorophenyl |
| C-20 | 2,5-difluorophenyl |
| C-21 | 2,6-difluorophenyl |
| C-22 | 3,4-difluorophenyl |
| C-23 | 3,5-difluorophenyl |
| C-24 | 2,3-dimethylphenyl |
| C-25 | 2,4-dimethylphenyl |
| C-26 | 2,5-dimethylphenyl |
| C-27 | 2,6-dimethylphenyl |
| C-28 | 3,4-dimethylphenyl |
| C-29 | 3,5-dimethylphenyl |
| C-30 | 2,3-diethylphenyl |
| C-31 | 2,4-diethylphenyl |
| C-32 | 2,5-diethylphenyl |
| C-33 | 2,6-diethylphenyl |
| C-34 | 3,5-diethylphenyl |
| C-35 | 3,4-diethylphenyl |
| C-36 | 2,3-dimethoxyphenyl |
| C-37 | 2,4-dimethoxyphenyl |
| C-38 | 2,5-dimethoxyphenyl |
| C-39 | 2,6-dimethoxyphenyl |
| C-40 | 3,4-dimethoxyphenyl |
| C-41 | 3,5-dimethoxyphenyl |
| C-42 | 2,3-di(trifluoromethyl)phenyl |
| C-43 | 2,4-di(trifluoromethyl)phenyl |
| C-44 | 2,5-di(trifluoromethyl)phenyl |
| C-45 | 2,6-di(trifluoromethyl)phenyl |
| C-46 | 3,4-di(trifluoromethyl)phenyl |
| C-47 | 3,5-di(trifluoromethyl)phenyl |
| C-48 | 2,3-di(trifluoromethoxy)phenyl |
| C-49 | 2,4-di(trifluoromethoxy)phenyl |
| C-50 | 2,5-di(trifluoromethoxy)phenyl |
| C-51 | 2,6-di(trifluoromethoxy)phenyl |
| C-52 | 3,4-di(trifluoromethoxy)phenyl |
| C-53 | 3,5-di(trifluoromethoxy)phenyl |
| C-54 | 2,3-di(difluoromethoxy)phenyl |
| C-55 | 2,4-di(difluoromethoxy)phenyl |
| C-56 | 2,5-di(difluoromethoxy)phenyl |
| C-57 | 2,6-di(difluoromethoxy)phenyl |
| C-58 | 3,4-di(difluoromethoxy)phenyl |
| C-59 | 3,5-di(difluoromethoxy)phenyl |
| C-60 | 2,3-di(trifluoromethylthio)phenyl |
| C-61 | 2,4-di(trifluoromethylthio)phenyl |
| C-62 | 2,5-di(trifluoromethylthio)phenyl |
| C-63 | 2,6-di(trifluoromethylthio)phenyl |
| C-64 | 3,4-di(trifluoromethylthio)phenyl |
| C-65 | 3,5-di(trifluoromethylthio)phenyl |
| C-66 | 2-fluoro-3-chlorophenyl |
| C-67 | 2-fluoro-4-chlorophenyl |
| C-68 | 2-fluoro-5-chlorophenyl |
| C-69 | 2-fluoro-6-chlorophenyl |
| C-70 | 3-fluoro-4-chlorophenyl |
| C-71 | 3-fluoro-5-chlorophenyl |
| C-72 | 2-chloro-5-fluorophenyl |
| C-73 | 3-chloro-4-fluorophenyl |
| C-74 | 2-methyl-3-chlorophenyl |
| C-75 | 2-methyl-4-chlorophenyl |
| C-76 | 2-methyl-5-chlorophenyl |
| C-77 | 2-methyl-6-chlorophenyl |
| C-78 | 3-methyl-4-chlorophenyl |
| C-79 | 3-methyl-5-chlorophenyl |
| C-80 | 2-chloro-3-methylphenyl |
| C-81 | 2-chloro-4-methylphenyl |
| C-82 | 2-chloro-5-methylphenyl |
| C-83 | 3-chloro-4-methylphenyl |
| C-84 | 2-methyl-3-fluorophenyl |
| C-85 | 2-methyl-4-fluorophenyl |
| C-86 | 2-methyl-5-fluorophenyl |
| C-87 | 2-methyl-6-fluorophenyl |
| C-88 | 3-methyl-4-fluorophenyl |
| C-89 | 3-methyl-5-fluorophenyl |
| C-90 | 2-fluoro-3-methylphenyl |
| C-91 | 2-fluoro-4-methylphenyl |
| C-92 | 2-fluoro-5-methylphenyl |
| C-93 | 3-fluoro-4-methylphenyl |
| C-94 | 2-chloro-3-ethylphenyl |
| C-95 | 2-chloro-4-ethylphenyl |
| C-96 | 2-chloro-5-ethylphenyl |
| C-97 | 3-chloro-4-ethylphenyl |
| C-98 | 2-ethyl-3-chlorophenyl |
| C-99 | 2-ethyl-4-chlorophenyl |
| C-100 | 2-ethyl-5-chlorophenyl |
| C-101 | 2-ethyl-6-chlorophenyl |
| C-102 | 2-ethyl-3-fluorophenyl |
| C-103 | 2-ethyl-4-fluorophenyl |
| C-104 | 2-ethyl-5-fluorophenyl |
| C-105 | 2-ethyl-6-fluorophenyl |
| C-106 | 3-ethyl-4-fluorophenyl |
| C-107 | 3-ethyl-5-fluorophenyl |
| C-108 | 2-fluoro-3-ethylphenyl |
| C-109 | 2-fluoro-4-ethylphenyl |
| C-110 | 2-fluoro-5-ethylphenyl |
| C-111 | 3-fluoro-4-ethylphenyl |
| C-112 | 2-methoxy-3-chlorophenyl |
| C-113 | 2-methoxy-4-chlorophenyl |
| C-114 | 2-methoxy-5-chlorophenyl |
| C-115 | 2-methoxy-6-chlorophenyl |
| C-116 | 3-methoxy-4-chlorophenyl |
| C-117 | 3-methoxy-5-chlorophenyl |
| C-118 | 2-chloro-5-methoxyphenyl |
| C-119 | 3-chloro-4-methoxyphenyl |
| C-120 | 2-methoxy-3-fluorophenyl |
| C-121 | 2-methoxy-4-fluorophenyl |
| C-122 | 2-methoxy-5-fluorophenyl |
| C-123 | 2-methoxy-6-fluorophenyl |
| C-124 | 3-methoxy-4-fluorophenyl |
| C-125 | 3-methoxy-5-fluorophenyl |
| C-126 | 2-fluoro-3-methoxyphenyl |
| C-127 | 2-fluoro-4-methoxyphenyl |
| C-128 | 2-fluoro-5-methoxyphenyl |
| C-129 | 3-fluoro-4-methoxyphenyl |
| C-130 | 3-fluoro-5-methoxyphenyl |
| C-131 | 2-(trifluoromethyl)-3-chlorophenyl |
| C-132 | 2-(trifluoromethyl)-4-chlorophenyl |
| C-133 | 2-(trifluoromethyl)-5-chlorophenyl |
| C-134 | 2-(trifluoromethyl)-6-chlorophenyl |
| C-135 | 3-(trifluoromethyl)-4-chlorophenyl |
| C-136 | 3-(trifluoromethyl)-5-chlorophenyl |
| C-137 | 2-chloro-3-(trifluoromethyl)phenyl |
| C-138 | 2-chloro-4-(trifluoromethyl)phenyl |
| C-139 | 2-chloro-5-(trifluoromethyl)phenyl |
| C-140 | 3-chloro-4-(trifluoromethyl)phenyl |
| C-141 | 2-(trifluoromethyl)-3-fluorophenyl |
| C-142 | 2-(trifluoromethyl)-4-fluorophenyl |
| C-143 | 2-(trifluoromethyl)-5-fluorophenyl |
| C-144 | 2-(trifluoromethyl)-6-fluorophenyl |
| C-145 | 3-(trifluoromethyl)-4-fluorophenyl |
| C-146 | 3-(trifluoromethyl)-5-fluorophenyl |
| C-147 | 2-fluoro-3-(trifluoromethyl)phenyl |
| C-148 | 2-fluoro-4-(trifluoromethyl)phenyl |
| C-149 | 2-fluoro-5-(trifluoromethyl)phenyl |
| C-150 | 3-fluoro-4-(trifluoromethyl)phenyl |
| C-151 | 2-(trifluoromethoxy)-3-chlorophenyl |
| C-152 | 2-(trifluoromethoxy)-4-chlorophenyl |
| C-153 | 2-(trifluoromethoxy)-5-chlorophenyl |
| C-154 | 2-(trifluoromethoxy)-6-chlorophenyl |
| C-155 | 3-(trifluoromethoxy)-4-chlorophenyl |
| C-156 | 3-(trifluoromethoxy)-5-chlorophenyl |
| C-157 | 2-chloro-3-(trifluoromethoxy)phenyl |
| C-158 | 2-chloro-4-(trifluoromethoxy)phenyl |
| C-159 | 2-chloro-5-(trifluoromethoxy)phenyl |
| C-160 | 3-chloro-4-(trifluoromethoxy)phenyl |
| C-161 | 2-(trifluoromethoxy)-3-fluorophenyl |
| C-162 | 2-(trifluoromethoxy)-4-fluorophenyl |
| C-163 | 2-(trifluoromethoxy)-5-fluorophenyl |
| C-164 | 2-(trifluoromethoxy)-6-fluorophenyl |
| C-165 | 3-(trifluoromethoxy)-4-fluorophenyl |
| C-166 | 3-(trifluoromethoxy)-5-fluorophenyl |
| C-167 | 2-fluoro-3-(trifluoromethoxy)phenyl |
| C-168 | 2-fluoro-4-(trifluoromethoxy)phenyl |
| C-169 | 2-fluoro-5-(trifluoromethoxy)phenyl |
| C-170 | 3-fluoro-4-(trifluoromethoxy)phenyl |
| C-171 | 2-(difluoromethoxy)-3-chlorophenyl |
| C-172 | 2-(difluoromethoxy)-4-chlorophenyl |
| C-173 | 2-(difluoromethoxy)-5-chlorophenyl |
| C-174 | 2-(difluoromethoxy)-6-chlorophenyl |
| C-175 | 3-(difluoromethoxy)-4-chlorophenyl |
| C-176 | 3-(difluoromethoxy)-5-chlorophenyl |
| C-177 | 2-chloro-3-(difluoromethoxy)phenyl |
| C-178 | 2-chloro-4-(difluoromethoxy)phenyl |
| C-179 | 2-chloro-5-(difluoromethoxy)phenyl |
| C-180 | 3-chloro-4-(difluoromethoxy)phenyl |
| C-181 | 2-(difluoromethoxy)-3-fluorophenyl |
| C-182 | 2-(difluoromethoxy)-4-fluorophenyl |
| C-183 | 2-(difluoromethoxy)-5-fluorophenyl |
| C-184 | 2-(difluoromethoxy)-6-fluorophenyl |
| C-185 | 3-(difluoromethoxy)-4-fluorophenyl |
| C-186 | 3-(difluoromethoxy)-5-fluorophenyl |
| C-187 | 2-fluoro-3-(difluoromethoxy)phenyl |
| C-188 | 2-fluoro-4-(difluoromethoxy)phenyl |
| C-189 | 2-fluoro-5-(difluoromethoxy)phenyl |
| C-190 | 3-fluoro-4-(difluoromethoxy)phenyl |
| C-191 | 2-(trifluoromethylthio)-3-chlorophenyl |
| C-192 | 2-(trifluoromethylthio)-4-chlorophenyl |
| C-193 | 2-(trifluoromethylthio)-5-chlorophenyl |
| C-194 | 2-(trifluoromethylthio)-6-chlorophenyl |
| C-195 | 3-(trifluoromethylthio)-4-chlorophenyl |
| C-196 | 3-(trifluoromethylthio)-5-chlorophenyl |
| C-197 | 2-chloro-3-(trifluoromethylthio)phenyl |
| C-198 | 2-chloro-4-(trifluoromethylthio)phenyl |
| C-199 | 2-chloro-5-(trifluoromethylthio)phenyl |
| C-200 | 3-chloro-4-(trifluoromethylthio)phenyl |
| C-201 | 2-(trifluoromethylthio)-3-fluorophenyl |
| C-202 | 2-(trifluoromethylthio)-4-fluorophenyl |
| C-203 | 2-(trifluoromethylthio)-5-fluorophenyl |
| C-204 | 2-(trifluoromethylthio)-6-fluorophenyl |
| C-205 | 3-(trifluoromethylthio)-4-fluorophenyl |
| C-206 | 3-(trifluoromethylthio)-5-fluorophenyl |
| C-207 | 2-fluoro-3-(trifluoromethylthio)phenyl |
| C-208 | 2-fluoro-4-(trifluoromethylthio)phenyl |
| C-209 | 2-fluoro-5-(trifluoromethylthio)phenyl |
| C-210 | 3-fluoro-4-(trifluoromethylthio)phenyl |
| C-211 | 2,3,4-trichlorophenyl |
| C-212 | 2,3,5-trichlorophenyl |
| C-213 | 2,3,6-trichlorophenyl |
| C-214 | 2,4,5-trichlorophenyl |
| C-215 | 2,4,6-trichlorophenyl |
| C-216 | 3,4,5-trichlorophenyl |
| C-217 | 2,3,4-trifluorophenyl |
| C-218 | 2,3,5-trifluorophenyl |
| C-219 | 2,3,6-trifluorophenyl |
| C-220 | 2,4,5-trifluorophenyl |
| C-221 | 2,4,6-trifluorophenyl |
| C-222 | 3,4,5-trifluorophenyl |
| C-223 | 2,3,4-trimethylphenyl |
| C-224 | 2,3,5-trimethylphenyl |
| C-225 | 2,3,6-trimethylphenyl |
| C-226 | 2,4,5-trimethylphenyl |
| C-227 | 2,4,6-trimethylphenyl |
| C-228 | 3,4,5-trimethylphenyl |
| C-229 | 2,3,4-trimethoxyphenyl |
| C-230 | 2,3,5-trimethoxyphenyl |
| C-231 | 2,3,6-trimethoxyphenyl |
| C-232 | 2,4,5-trimethoxyphenyl |
| C-233 | 2,4,6-trimethoxyphenyl |
| C-234 | 3,4,5-trimethoxyphenyl |

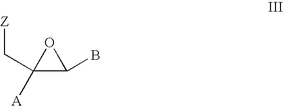

In particular with a view to their use, preference is given to the compounds III compiled in Tables 1d to 93d below. The groups mentioned in the tables for a substituent are furthermore per se, independently of the combination in which they are mentioned, a particularly preferred aspect of the substituent in question.

### Table 1d

Compounds III in which A is 2,3-difluorophenyl and the combination of B and Z corresponds in each case to one row of Table D (compounds III.1dD-1 to III.1dD-1275)

### Table 2d

Compounds III in which A is 2,4-difluorophenyl and the combination of B and Z corresponds in each case to one row of Table D (compounds III.2dD-1 to III.2dD-1275)

### Table 3d

Compounds III in which A is 2,5-difluorophenyl and the combination of B and Z corresponds in each case to one row of Table D (compounds III.3dD-1 to III.3dD-1275)

### Table 4d

Compounds III in which A is 2,6-difluorophenyl and the combination of B and Z corresponds in each case to one row of Table D (compounds III.4dD-1 to III.4dD-1275)

### Table 5d

Compounds III in which A is 3,4-difluorophenyl and the combination of B and Z corresponds in each case to one row of Table D (compounds III.5dD-1 to III.5dD-1275)

### Table 6d

Compounds III in which A is 3,5-difluorophenyl and the combination of B and Z corresponds in each case to one row of Table D (compounds III.6dD-1 to III.6dD-1275)

### Table 7d

Compounds III in which A is 2-fluoro-3-chlorophenyl and the combination of B and Z corresponds in each case to one row of Table D (compounds III.7dD-1 to III.7dD-1275)

### Table 8d

Compounds III in which A is 2-Fluoro-4-chlorophenyl and the combination of B and Z corresponds in each case to one row of Table D (compounds III.8dD-1 to III.8dD-1275)

### Table 9d

Compounds III in which A is 2-fluoro-5-chlorophenyl and the combination of B and Z corresponds in each case to one row of Table D (compounds III.9dD-1 to III.9dD-1275)

### Table 10d

Compounds III in which A is 2-fluoro-6-chlorophenyl I and the combination of B and Z corresponds in each case to one row of Table D (compounds III.10dD-1 to III.10dD-1275)

### Table 11d

Compounds III in which A is 3-fluoro-4-chlorophenyl and the combination of B and Z corresponds in each case to one row of Table D (compounds III.11dD-1 to III.11dD-1275)

### Table 12d

Compounds III in which A is 3-fluoro-5-chlorophenyl and the combination of B and Z corresponds in each case to one row of Table D (compounds III.12dD-1 to III.12dD-1275)

### Table 13d

Compounds III in which A is 2-chloro-3-fluorophenyl and the combination of B and Z corresponds in each case to one row of Table D (compounds III.13dD-1 to III.13dD-1275)

### Table 14d

Compounds III in which A is 2-chloro-4-fluorophenyl and the combination of B and Z corresponds in each case to one row of Table D (compounds III.14dD-1 to III.14dD-1275)

### Table 15d

Compounds III in which A is 2-chloro-5-fluorophenyl and the combination of B and Z corresponds in each case to one row of Table D (compounds III.15dD-1 to III.15dD-1275)

### Table 16d

Compounds III in which A is 3-chloro-4-fluorophenyl and the combination of B and Z corresponds in each case to one row of Table D (compounds III.16dD-1 to III.16dD-1275)

### Table 17d

Compounds III in which A is 2-methyl-3-fluorophenyl and the combination of B and Z corresponds in each case to one row of Table D (compounds III.17dD-1 to III.17dD-1275)

### Table 18d

Compounds III in which A is 2-methyl-4-fluorophenyl and the combination of B and Z corresponds in each case to one row of Table D (compounds III.18dD-1 to III.18dD-1275)

### Table 19d

Compounds III in which A is 2-methyl-5-fluorophenyl and the combination of B and Z corresponds in each case to one row of Table D (compounds III.19dD-1 to III.19dD-1275)

### Table 20d

Compounds III in which A is 2-methyl-6-fluorophenyl and the combination of B and Z corresponds in each case to one row of Table D (compounds III.20dD-1 to III.20dD-1275)

### Table 21d

Compounds III in which A is 3-methyl-4-fluorophenyl and the combination of B and Z corresponds in each case to one row of Table D (compounds III.21dD-1 to III.21dD-1275)

### Table 22d

Compounds III in which A is 3-methyl-5-fluorophenyl and the combination of B and Z corresponds in each case to one row of Table D (compounds III.22dD-1 to III.22dD-1275)

### Table 23d

Compounds III in which A is 2-fluoro-3-methylphenyl and the combination of B and Z corresponds in each case to one row of Table D (compounds III.23dD-1 to III.23dD-1275)

### Table 24d

Compounds III in which A is 2-fluoro-4-methylphenyl and the combination of B and Z corresponds in each case to one row of Table D (compounds III.24dD-1 to III.24dD-1275)

### Table 25d

Compounds III in which A is 2-fluoro-5-methylphenyl and the combination of B and Z corresponds in each case to one row of Table D (compounds III.25dD-1 to III.25dD-1275)

### Table 26d

Compounds III in which A is 3-fluoro-4-methylphenyl and the combination of B and Z corresponds in each case to one row of Table D (compounds III.26dD-1 to III.26dD-1275)

### Table 27d

Compounds III in which A is 2-ethyl-3-fluorophenyl and the combination of B and Z corresponds in each case to one row of Table D (compounds III.27dD-1 to III.27dD-1275)

### Table 28d

Compounds III in which A is 2-ethyl-4-fluorophenyl and the combination of B and Z corresponds in each case to one row of Table D (compounds III.28dD-1 to III.28dD-1275)

### Table 29d

Compounds III in which A is 2-ethyl-5-fluorophenyl and the combination of B and Z corresponds in each case to one row of Table D (compounds III.29dD-1 to III.29dD-1275)

### Table 30d

Compounds III in which A is 2-ethyl-6-fluorophenyl and the combination of B and Z corresponds in each case to one row of Table D (compounds III.30dD-1 to III.30dD-1275)

### Table 31d

Compounds III in which A is 3-ethyl-4-fluorophenyl and the combination of B and Z corresponds in each case to one row of Table D (compounds III.31dD-1 to III.31dD-1275)

### Table 32d

Compounds III in which A is 3-ethyl-5-fluorophenyl and the combination of B and Z corresponds in each case to one row of Table D (compounds III.32dD-1 to III.32dD-1275)

### Table 33d

Compounds III in which A is 2-fluoro-3-ethylphenyl and the combination of B and Z corresponds in each case to one row of Table D (compounds III.33dD-1 to III.33dD-1275)

### Table 34d

Compounds III in which A is 2-fluoro-4-ethylphenyl and the combination of B and Z corresponds in each case to one row of Table D (compounds III.34dD-1 to III.34dD-1275)

### Table 35d

Compounds III in which A is 2-fluoro-5-ethylphenyl and the combination of B and Z corresponds in each case to one row of Table D (compounds III.35dD-1 to III.35dD-1275)

### Table 36d

Compounds III in which A is 3-fluoro-4-ethylphenyl and the combination of B and Z corresponds in each case to one row of Table D (compounds III.36dD-1 to III.36dD-1275)

### Table 37d

Compounds III in which A is 2-methoxy-3-fluorophenyl and the combination of B and Z corresponds in each case to one row of Table D (compounds III.37dD-1 to III.37dD-1275)

### Table 38d

Compounds III in which A is 2-methoxy-4-fluorophenyl and the combination of B and Z corresponds in each case to one row of Table D (compounds III.38dD-1 to III.38dD-1275)

### Table 39d

Compounds III in which A is 2-methoxy-5-fluorophenyl and the combination of B and Z corresponds in each case to one row of Table D (compounds III.39dD-1 to III.39dD-1275)

### Table 40d

Compounds III in which A is 2-methoxy-6-fluorophenyl and the combination of B and Z corresponds in each case to one row of Table D (compounds III.40dD-1 to III.40dD-1275)

### Table 41d

Compounds III in which A is 3-methoxy-4-fluorophenyl and the combination of B and Z corresponds in each case to one row of Table D (compounds III.41dD-1 to III.41dD-1275)

### Table 42d

Compounds III in which A is 3-methoxy-5-fluorophenyl and the combination of B and Z corresponds in each case to one row of Table D (compounds III.42dD-1 to III.42dD-1275)

### Table 43d

Compounds III in which A is 2-fluoro-3-methoxyphenyl and the combination of B and Z corresponds in each case to one row of Table D (compounds III.43dD-1 to III.43dD-1275)

### Table 44d

Compounds III in which A is 2-fluoro-4-methoxyphenyl and the combination of B and Z corresponds in each case to one row of Table D (compounds III.44dD-1 to III.44dD-1275)

### Table 45d

Compounds III in which A is 2-fluoro-5-methoxyphenyl and the combination of B and Z corresponds in each case to one row of Table D (compounds III.45dD-1 to III.45dD-1275)

### Table 46d

Compounds III in which A is 3-fluoro-4-methoxyphenyl and the combination of B and Z corresponds in each case to one row of Table D (compounds III.46dD-1 to III.46dD-1275)

### Table 47d

Compounds III in which A is 3-fluoro-5-methoxyphenyl and the combination of B and Z corresponds in each case to one row of Table D (compounds III.47dD-1 to III.47dD-1275)

### Table 48d

Compounds III in which A is 2-(trifluoromethyl)-3-fluorophenyl and the combination of B and Z corresponds in each case to one row of Table D (compounds III.48dD-1 to III.48dD-1275)

### Table 49d

Compounds III in which A is 2-(trifluoromethyl)-4-fluorophenyl and the combination of B and Z corresponds in each case to one row of Table D (compounds III.49dD-1 to III.49dD-1275)

### Table 50d

Compounds III in which A is 2-(trifluoromethyl)-5-fluorophenyl and the combination of B and Z corresponds in each case to one row of Table D (compounds III.50dD-1 to III.50dD-1275)

### Table 51d

Compounds III in which A is 2-(trifluoromethyl)-6-fluorophenyl and the combination of B and Z corresponds in each case to one row of Table D (compounds III.51 dD-1 to III.51dD-1275)

### Table 52d

Compounds III in which A is 3-(trifluoromethyl)-4-fluorophenyl and the combination of B and Z corresponds in each case to one row of Table D (compounds III.52dD-1 to III.52dD-1275)

### Table 53d

Compounds III in which A is 3-(trifluoromethyl)-5-fluorophenyl and the combination of B and Z corresponds in each case to one row of Table D (compounds III.53dD-1 to III.53dD-1275)

### Table 54d

Compounds III in which A is 2-fluoro-3-(trifluoromethyl)phenyl and the combination of B and Z corresponds in each case to one row of Table D (compounds III.54dD-1 to III.54dD-1275)

### Table 55d

Compounds III in which A is 2-fluoro-4-(trifluoromethyl)phenyl and the combination of B and Z corresponds in each case to one row of Table D (compounds III.55dD-1 to III.55dD-1275)

### Table 56d

Compounds III in which A is 2-fluoro-5-(trifluoromethyl)phenyl and the combination of B and Z corresponds in each case to one row of Table D (compounds III.56dD-1 to III.56dD-1275)

### Table 57d

Compounds III in which A is 3-fluoro-4-(trifluoromethyl)phenyl and the combination of B and Z corresponds in each case to one row of Table D (compounds III.57dD-1 to III.57dD-1275)

### Table 58d

Compounds III in which A is 2-(trifluoromethoxy)-3-fluorophenyl and the combination of B and Z corresponds in each case to one row of Table D (compounds III.58dD-1 to III.58dD-1275)

### Table 59d

Compounds III in which A is 2-(trifluoromethoxy)-4-fluorophenyl and the combination of B and Z corresponds in each case to one row of Table D (compounds III.59dD-1 to III.59dD-1275)

### Table 60d

Compounds III in which A is 2-(trifluoromethoxy)-5-fluorophenyl and the combination of B and Z corresponds in each case to one row of Table D (compounds III.60dD-1 to III.60dD-1275)

### Table 61d

Compounds III in which A is 2-(trifluoromethoxy)-6-fluorophenyl and the combination of B and Z corresponds in each case to one row of Table D (compounds III.61dD-1 to III.61dD-1275)

### Table 62d

Compounds III in which A is 3-(trifluoromethoxy)-4-fluorophenyl and the combination of B and Z corresponds in each case to one row of Table D (compounds III.62dD-1 to III.62dD-1275)

### Table 63d

Compounds III in which A is 3-(trifluoromethoxy)-5-fluorophenyl and the combination of B and Z corresponds in each case to one row of Table D (compounds III.63dD-1 to III.63dD-1275)

### Table 64d

Compounds III in which A is 2-fluoro-3-(trifluoromethoxy)phenyl and the combination of B and Z corresponds in each case to one row of Table D (compounds III.64dD-1 to III.64dD-1275)

### Table 65d

Compounds III in which A is 2-fluoro-4-(trifluoromethoxy)phenyl and the combination of B and Z corresponds in each case to one row of Table D (compounds III.65dD-1 to III.65dD-1275)

### Table 66d

Compounds III in which A is 2-fluoro-5-(trifluoromethoxy)phenyl and the combination of B and Z corresponds in each case to one row of Table D (compounds III.66dD-1 to III.66dD-1275)

### Table 67d

Compounds III in which A is 3-fluoro-4-(trifluoromethoxy)phenyl and the combination of B and Z corresponds in each case to one row of Table D (compounds III.67dD-1 to III.67dD-1275)

### Table 68d

Compounds III in which A is 2-(difluoromethoxy)-3-fluorophenyl and the combination of B and Z corresponds in each case to one row of Table D (compounds III.68dD-1 to III.68dD-1275)

### Table 69d

Compounds III in which A is 2-(difluoromethoxy)-4-fluorophenyl and the combination of B and Z corresponds in each case to one row of Table D (compounds III.69dD-1 to III.69dD-1275)

### Table 70d

Compounds III in which A is 2-(difluoromethoxy)-5-fluorophenyl and the combination of B and Z corresponds in each case to one row of Table D (compounds III.70dD-1 to III.70dD-1275)

### Table 71d

Compounds III in which A is 2-(difluoromethoxy)-6-fluorophenyl and the combination of B and Z corresponds in each case to one row of Table D (compounds III.71dD-1 to III.71dD-1275)

### Table 72d

Compounds III in which A is 3-(difluoromethoxy)-4-fluorophenyl and the combination of B and Z corresponds in each case to one row of Table D (compounds III.72dD-1 to III.72dD-1275)

### Table 73d

Compounds III in which A is 3-(difluoromethoxy)-5-fluorophenyl and the combination of B and Z corresponds in each case to one row of Table D (compounds III.73dD-1 to III.73dD-1275)

### Table 74d

Compounds III in which A is 2-fluoro-3-(difluoromethoxy)phenyl and the combination of B and Z corresponds in each case to one row of Table D (compounds III.74dD-1 to III.74dD-1275)

### Table 75d

Compounds III in which A is 2-fluoro-4-(difluoromethoxy)phenyl and the combination of B and Z corresponds in each case to one row of Table D (compounds 111.75dD-1 to III.75dD-1275)

### Table 76d

Compounds III in which A is 2-fluoro-5-(difluoromethoxy)phenyl and the combination of B and Z corresponds in each case to one row of Table D (compounds III.76dD-1 to III.76dD-1275)

### Table 77d

Compounds III in which A is 3-fluoro-4-(difluoromethoxy)phenyl and the combination of B and Z corresponds in each case to one row of Table D (compounds III.77dD-1 to III.77dD-1275)

### Table 78d

Compounds III in which A is 2-(trifluoromethylthio)-3-fluorophenyl and the combination of B and Z corresponds in each case to one row of Table D (compounds III.78dD-1 to III.78dD-1275)

### Table 79d

Compounds III in which A is 2-(trifluoromethylthio)-4-fluorophenyl and the combination of B and Z corresponds in each case to one row of Table D (compounds III.79dD-1 to III.79dD-1275)

### Table 80d

Compounds III in which A is 2-(trifluoromethylthio)-5-fluorophenyl and the combination of B and Z corresponds in each case to one row of Table D (compounds III.80dD-1 to III.80dD-1275)

### Table 81d

Compounds III in which A is 2-(trifluoromethylthio)-6-fluorophenyl and the combination of B and Z corresponds in each case to one row of Table D (compounds III.81dD-1 to III.81dD-1275)

### Table 82d

Compounds III in which A is 3-(trifluoromethylthio)-4-fluorophenyl and the combination of B and Z corresponds in each case to one row of Table D (compounds III.82dD-1 to III.82dD-1275)

### Table 83d

Compounds III in which A is 3-(trifluoromethylthio)-5-fluorophenyl and the combination of B and Z corresponds in each case to one row of Table D (compounds III.83dD-1 to III.83dD-1275)

### Table 84d

Compounds III in which A is 2-fluoro-3-(trifluoromethylthio)phenyl and the combination of B and Z corresponds in each case to one row of Table D (compounds III.84dD-1 to III.84dD-1275)

### Table 85d

Compounds III in which A is 2-fluoro-4-(trifluoromethylthio)phenyl and the combination of B and Z corresponds in each case to one row of Table D (compounds III.85dD-1 to III.85dD-1275)

### Table 86d

Compounds III in which A is 2-fluoro-5-(trifluoromethylthio)phenyl and the combination of B and Z corresponds in each case to one row of Table D (compounds III.86dD-1 to III.86dD-1275)

### Table 87d

Compounds III in which A is 3-fluoro-4-(trifluoromethylthio)phenyl and the combination of B and Z corresponds in each case to one row of Table D (compounds III.87dD-1 to III.87dD-1275)

### Table 88d

Compounds III in which A is 2,3,4-trifluorophenyl and the combination of B and Z corresponds in each case to one row of Table D (compounds III.88dD-1 to III.88dD-1275)

### Table 89d

Compounds III in which A is 2,3,5-trifluorophenyl and the combination of B and Z corresponds in each case to one row of Table D (compounds III.89dD-1 to III.89dD-1275)

### Table 90d

Compounds III in which A is 2,3,6-trifluorophenyl and the combination of B and Z corresponds in each case to one row of Table D (compounds III.90dD-1 to III.90dD-1275)

### Table 91d

Compounds III in which A is 2,4,5-trifluorophenyl and the combination of B and Z corresponds in each case to one row of Table D (compounds III.91dD-1 to III.91dD-1275)

### Table 92d

Compounds III in which A is 2,4,6-trifluorophenyl and the combination of B and Z corresponds in each case to one row of Table D (compounds III.92dD-1 to III.92dD-1275)

### Table 93d

Compounds III in which A is 3,4,5-trifluorophenyl and the combination of B and Z corresponds in each case to one row of Table D (compounds III.93dD-1 to III.93dD-1275)

TABLE D

| Row | B | Z |
|---|---|---|
| D-1 | phenyl | Cl |
| D-2 | 2-chlorophenyl | Cl |
| D-3 | 3-chlorophenyl | Cl |
| D-4 | 4-chlorophenyl | Cl |
| D-5 | 2-fluorophenyl | Cl |
| D-6 | 3-fluorophenyl | Cl |
| D-7 | 4-fluorophenyl | Cl |
| D-8 | 2-methylphenyl | Cl |
| D-9 | 3-methylphenyl | Cl |
| D-10 | 4-methylphenyl | Cl |
| D-11 | 2-ethylphenyl | Cl |
| D-12 | 3-ethylphenyl | Cl |
| D-13 | 4-ethylphenyl | Cl |
| D-14 | 2-methoxyphenyl | Cl |
| D-15 | 3-methoxyphenyl | Cl |
| D-16 | 4-methoxyphenyl | Cl |
| D-17 | 2-trifluoromethylphenyl | Cl |
| D-18 | 3-trifluoromethylphenyl | Cl |
| D-19 | 4-trifluoromethylphenyl | Cl |
| D-20 | 2-trifluoromethoxyphenyl | Cl |
| D-21 | 3-trifluoromethoxyphenyl | Cl |
| D-22 | 4-trifluoromethoxyphenyl | Cl |
| D-23 | 2-difluoromethoxyphenyl | Cl |
| D-24 | 3-difluoromethoxyphenyl | Cl |
| D-25 | 4-difluoromethoxyphenyl | Cl |
| D-26 | 2-trifluoromethylthiophenyl | Cl |
| D-27 | 3-trifluoromethylthiophenyl | Cl |
| D-28 | 4-trifluoromethylthiophenyl | Cl |
| D-29 | 2,3-dichlorophenyl | Cl |
| D-30 | 2,4-dichlorophenyl | Cl |
| D-31 | 2,5-dichlorophenyl | Cl |
| D-32 | 2,6-dichlorophenyl | Cl |
| D-33 | 3,4-dichlorophenyl | Cl |
| D-34 | 3,5-dichlorophenyl | Cl |
| D-35 | 2,3-difluorophenyl | Cl |
| D-36 | 2,4-difluorophenyl | Cl |
| D-37 | 2,5-difluorophenyl | Cl |
| D-38 | 2,6-difluorophenyl | Cl |
| D-39 | 3,4-difluorophenyl | Cl |
| D-40 | 3,5-difluorophenyl | Cl |
| D-41 | 2,3-dimethylphenyl | Cl |
| D-42 | 2,4-dimethylphenyl | Cl |
| D-43 | 2,5-dimethylphenyl | Cl |
| D-44 | 2,6-dimethylphenyl | Cl |
| D-45 | 3,4-dimethylphenyl | Cl |
| D-46 | 3,5-dimethylphenyl | Cl |
| D-47 | 2,3-diethylphenyl | Cl |
| D-48 | 2,4-diethylphenyl | Cl |
| D-49 | 2,5-diethylphenyl | Cl |
| D-50 | 2,6-diethylphenyl | Cl |
| D-51 | 3,5-diethylphenyl | Cl |
| D-52 | 3,4-diethylphenyl | Cl |
| D-53 | 2,3-dimethoxyphenyl | Cl |
| D-54 | 2,4-dimethoxyphenyl | Cl |
| D-55 | 2,5-dimethoxyphenyl | Cl |
| D-56 | 2,6-dimethoxyphenyl | Cl |
| D-57 | 3,4-dimethoxyphenyl | Cl |
| D-58 | 3,5-dimethoxyphenyl | Cl |
| D-59 | 2,3-di(trifluoromethyl)phenyl | Cl |
| D-60 | 2,4-di(trifluoromethyl)phenyl | Cl |
| D-61 | 2,5-di(trifluoromethyl)phenyl | Cl |
| D-62 | 2,6-di(trifluoromethyl)phenyl | Cl |
| D-63 | 3,4-di(trifluoromethyl)phenyl | Cl |
| D-64 | 3,5-di(trifluoromethyl)phenyl | Cl |
| D-65 | 2,3-di(trifluoromethoxy)phenyl | Cl |
| D-66 | 2,4-di(trifluoromethoxy)phenyl | Cl |
| D-67 | 2,5-di(trifluoromethoxy)phenyl | Cl |
| D-68 | 2,6-di(trifluoromethoxy)phenyl | Cl |
| D-69 | 3,4-di(trifluoromethoxy)phenyl | Cl |
| D-70 | 3,5-di(trifluoromethoxy)phenyl | Cl |
| D-71 | 2,3-di(difluoromethoxy)phenyl | Cl |
| D-72 | 2,4-di(difluoromethoxy)phenyl | Cl |
| D-73 | 2,5-di(difluoromethoxy)phenyl | Cl |
| D-74 | 2,6-di(difluoromethoxy)phenyl | Cl |
| D-75 | 3,4-di(difluoromethoxy)phenyl | Cl |
| D-76 | 3,5-di(difluoromethoxy)phenyl | Cl |
| D-77 | 2,3-di(trifluoromethylthio)phenyl | Cl |
| D-78 | 2,4-di(trifluoromethylthio)phenyl | Cl |
| D-79 | 2,5-di(trifluoromethylthio)phenyl | Cl |
| D-80 | 2,6-di(trifluoromethylthio)phenyl | Cl |
| D-81 | 3,4-di(trifluoromethylthio)phenyl | Cl |
| D-82 | 3,5-di(trifluoromethylthio)phenyl | Cl |
| D-83 | 2-fluoro-3-chlorophenyl | Cl |
| D-84 | 2-fluoro-4-chlorophenyl | Cl |
| D-85 | 2-fluoro-5-chlorophenyl | Cl |
| D-86 | 2-fluoro-6-chlorophenyl | Cl |
| D-87 | 3-fluoro-4-chlorophenyl | Cl |
| D-88 | 3-fluoro-5-chlorophenyl | Cl |
| D-89 | 2-chloro-3-fluorophenyl | Cl |
| D-90 | 2-chloro-4-fluorophenyl | Cl |
| D-91 | 2-chloro-5-fluorophenyl | Cl |
| D-92 | 3-chloro-4-fluorophenyl | Cl |
| D-93 | 2-methyl-3-chlorophenyl | Cl |
| D-94 | 2-methyl-4-chlorophenyl | Cl |
| D-95 | 2-methyl-5-chlorophenyl | Cl |
| D-96 | 2-methyl-6-chlorophenyl | Cl |
| D-97 | 3-methyl-4-chlorophenyl | Cl |
| D-98 | 3-methyl-5-chlorophenyl | Cl |
| D-99 | 2-chloro-3-methylphenyl | Cl |
| D-100 | 2-chloro-4-methylphenyl | Cl |
| D-101 | 2-chloro-5-methylphenyl | Cl |
| D-102 | 3-chloro-4-methylphenyl | Cl |
| D-103 | 2-methyl-3-fluorophenyl | Cl |
| D-104 | 2-methyl-4-fluorophenyl | Cl |
| D-105 | 2-methyl-5-fluorophenyl | Cl |
| D-106 | 2-methyl-6-fluorophenyl | Cl |
| D-107 | 3-methyl-4-fluorophenyl | Cl |
| D-108 | 3-methyl-5-fluorophenyl | Cl |
| D-109 | 2-fluoro-3-methylphenyl | Cl |
| D-110 | 2-fluoro-4-methylphenyl | Cl |
| D-111 | 2-fluoro-5-methylphenyl | Cl |
| D-112 | 3-fluoro-4-methylphenyl | Cl |
| D-113 | 2-chloro-3-ethylphenyl | Cl |
| D-114 | 2-chloro-4-ethylphenyl | Cl |
| D-115 | 2-chloro-5-ethylphenyl | Cl |
| D-116 | 3-chloro-4-ethylphenyl | Cl |
| D-117 | 2-ethyl-3-chlorophenyl | Cl |
| D-118 | 2-ethyl-4-chlorophenyl | Cl |
| D-119 | 2-ethyl-5-chlorophenyl | Cl |
| D-120 | 2-ethyl-6-chlorophenyl | Cl |
| D-121 | 2-ethyl-3-fluorophenyl | Cl |
| D-122 | 2-ethyl-4-fluorophenyl | Cl |
| D-123 | 2-ethyl-5-fluorophenyl | Cl |
| D-124 | 2-ethyl-6-fluorophenyl | Cl |
| D-125 | 3-ethyl-4-fluorophenyl | Cl |
| D-126 | 3-ethyl-5-fluorophenyl | Cl |
| D-127 | 2-fluoro-3-ethylphenyl | Cl |
| D-128 | 2-fluoro-4-ethylphenyl | Cl |
| D-129 | 2-fluoro-5-ethylphenyl | Cl |
| D-130 | 3-fluoro-4-ethylphenyl | Cl |
| D-131 | 2-methoxy-3-chlorophenyl | Cl |
| D-132 | 2-methoxy-4-chlorophenyl | Cl |
| D-133 | 2-methoxy-5-chlorophenyl | Cl |
| D-134 | 2-methoxy-6-chlorophenyl | Cl |
| D-135 | 3-methoxy-4-chlorophenyl | Cl |
| D-136 | 3-methoxy-5-chlorophenyl | Cl |
| D-137 | 2-chloro-3-methoxyphenyl | Cl |
| D-138 | 2-chloro-4-methoxyphenyl | Cl |
| D-139 | 2-chloro-5-methoxyphenyl | Cl |
| D-140 | 3-chloro-4-methoxyphenyl | Cl |
| D-141 | 2-methoxy-3-fluorophenyl | Cl |
| D-142 | 2-methoxy-4-fluorophenyl | Cl |
| D-143 | 2-methoxy-5-fluorophenyl | Cl |
| D-144 | 2-methoxy-6-fluorophenyl | Cl |
| D-145 | 3-methoxy-4-fluorophenyl | Cl |
| D-146 | 3-methoxy-5-fluorophenyl | Cl |
| D-147 | 2-fluoro-3-methoxyphenyl | Cl |
| D-148 | 2-fluoro-4-methoxyphenyl | Cl |
| D-149 | 2-fluoro-5-methoxyphenyl | Cl |
| D-150 | 3-fluoro-4-methoxyphenyl | Cl |
| D-151 | 3-fluoro-5-methoxyphenyl | Cl |
| D-152 | 2-(trifluoromethyl)-3-chlorophenyl | Cl |
| D-153 | 2-(trifluoromethyl)-4-chlorophenyl | Cl |
| D-154 | 2-(trifluoromethyl)-5-chlorophenyl | Cl |
| D-155 | 2-(trifluoromethyl)-6-chlorophenyl | Cl |
| D-156 | 3-(trifluoromethyl)-4-chlorophenyl | Cl |
| D-157 | 3-(trifluoromethyl)-5-chlorophenyl | Cl |
| D-158 | 2-chloro-3-(trifluoromethyl)phenyl | Cl |
| D-159 | 2-chloro-4-(trifluoromethyl)phenyl | Cl |
| D-160 | 2-chloro-5-(trifluoromethyl)phenyl | Cl |
| D-161 | 3-chloro-4-(trifluoromethyl)phenyl | Cl |
| D-162 | 2-(trifluoromethyl)-3-fluorophenyl | Cl |
| D-163 | 2-(trifluoromethyl)-4-fluorophenyl | Cl |
| D-164 | 2-(trifluoromethyl)-5-fluorophenyl | Cl |
| D-165 | 2-(trifluoromethyl)-6-fluorophenyl | Cl |
| D-166 | 3-(trifluoromethyl)-4-fluorophenyl | Cl |
| D-167 | 3-(trifluoromethyl)-5-fluorophenyl | Cl |
| D-168 | 2-fluoro-3-(trifluoromethyl)phenyl | Cl |
| D-169 | 2-fluoro-4-(trifluoromethyl)phenyl | Cl |
| D-170 | 2-fluoro-5-(trifluoromethyl)phenyl | Cl |
| D-171 | 3-fluoro-4-(trifluoromethyl)phenyl | Cl |
| D-172 | 2-(trifluoromethoxy)-3-chlorophenyl | Cl |
| D-173 | 2-(trifluoromethoxy)-4-chlorophenyl | Cl |
| D-174 | 2-(trifluoromethoxy)-5-chlorophenyl | Cl |
| D-175 | 2-(trifluoromethoxy)-6-chlorophenyl | Cl |
| D-176 | 3-(trifluoromethoxy)-4-chlorophenyl | Cl |
| D-177 | 3-(trifluoromethoxy)-5-chlorophenyl | Cl |
| D-178 | 2-chloro-3-(trifluoromethoxy)phenyl | Cl |
| D-179 | 2-chloro-4-(trifluoromethoxy)phenyl | Cl |
| D-180 | 2-chloro-5-(trifluoromethoxy)phenyl | Cl |
| D-181 | 3-chloro-4-(trifluoromethoxy)phenyl | Cl |
| D-182 | 2-(trifluoromethoxy)-3-fluorophenyl | Cl |
| D-183 | 2-(trifluoromethoxy)-4-fluorophenyl | Cl |
| D-184 | 2-(trifluoromethoxy)-5-fluorophenyl | Cl |
| D-185 | 2-(trifluoromethoxy)-6-fluorophenyl | Cl |
| D-186 | 3-(trifluoromethoxy)-4-fluorophenyl | Cl |
| D-187 | 3-(trifluoromethoxy)-5-fluorophenyl | Cl |
| D-188 | 2-fluoro-3-(trifluoromethoxy)phenyl | Cl |
| D-189 | 2-fluoro-4-(trifluoromethoxy)phenyl | Cl |
| D-190 | 2-fluoro-5-(trifluoromethoxy)phenyl | Cl |
| D-191 | 3-fluoro-4-(trifluoromethoxy)phenyl | Cl |
| D-192 | 2-(difluoromethoxy)-3-chlorophenyl | Cl |
| D-193 | 2-(difluoromethoxy)-4-chlorophenyl | Cl |
| D-194 | 2-(difluoromethoxy)-5-chlorophenyl | Cl |
| D-195 | 2-(difluoromethoxy)-6-chlorophenyl | Cl |
| D-196 | 3-(difluoromethoxy)-4-chlorophenyl | Cl |
| D-197 | 3-(difluoromethoxy)-5-chlorophenyl | Cl |
| D-198 | 2-chloro-3-(difluoromethoxy)phenyl | Cl |
| D-199 | 2-chloro-4-(difluoromethoxy)phenyl | Cl |
| D-200 | 2-chloro-5-(difluoromethoxy)phenyl | Cl |
| D-201 | 3-chloro-4-(difluoromethoxy)phenyl | Cl |
| D-202 | 2-(difluoromethoxy)-3-fluorophenyl | Cl |
| D-203 | 2-(difluoromethoxy)-4-fluorophenyl | Cl |
| D-204 | 2-(difluoromethoxy)-5-fluorophenyl | Cl |
| D-205 | 2-(difluoromethoxy)-6-fluorophenyl | Cl |
| D-206 | 3-(difluoromethoxy)-4-fluorophenyl | Cl |
| D-207 | 3-(difluoromethoxy)-5-fluorophenyl | Cl |
| D-208 | 2-fluoro-3-(difluoromethoxy)phenyl | Cl |
| D-209 | 2-fluoro-4-(difluoromethoxy)phenyl | Cl |
| D-210 | 2-fluoro-5-(difluoromethoxy)phenyl | Cl |
| D-211 | 3-fluoro-4-(difluoromethoxy)phenyl | Cl |
| D-212 | 2-(trifluoromethylthio)-3-chlorophenyl | Cl |
| D-213 | 2-(trifluoromethylthio)-4-chlorophenyl | Cl |
| D-214 | 2-(trifluoromethylthio)-5-chlorophenyl | Cl |
| D-215 | 2-(trifluoromethylthio)-6-chlorophenyl | Cl |
| D-216 | 3-(trifluoromethylthio)-4-chlorophenyl | Cl |
| D-217 | 3-(trifluoromethylthio)-5-chlorophenyl | Cl |
| D-218 | 2-chloro-3-(trifluoromethylthio)phenyl | Cl |
| D-219 | 2-chloro-4-(trifluoromethylthio)phenyl | Cl |
| D-220 | 2-chloro-5-(trifluoromethylthio)phenyl | Cl |
| D-221 | 3-chloro-4-(trifluoromethylthio)phenyl | Cl |
| D-222 | 2-(trifluoromethylthio)-3-fluorophenyl | Cl |
| D-223 | 2-(trifluoromethylthio)-4-fluorophenyl | Cl |
| D-224 | 2-(trifluoromethylthio)-5-fluorophenyl | Cl |
| D-225 | 2-(trifluoromethylthio)-6-fluorophenyl | Cl |
| D-226 | 3-(trifluoromethylthio)-4-fluorophenyl | Cl |
| D-227 | 3-(trifluoromethylthio)-5-fluorophenyl | Cl |
| D-228 | 2-fluoro-3-(trifluoromethylthio)phenyl | Cl |
| D-229 | 2-fluoro-4-(trifluoromethylthio)phenyl | Cl |
| D-230 | 2-fluoro-5-(trifluoromethylthio)phenyl | Cl |
| D-231 | 3-fluoro-4-(trifluoromethylthio)phenyl | Cl |
| D-232 | 2,3,4-trichlorophenyl | Cl |
| D-233 | 2,3,5-trichlorophenyl | Cl |
| D-234 | 2,3,6-trichlorophenyl | Cl |
| D-235 | 2,4,5-trichlorophenyl | Cl |
| D-236 | 2,4,6-trichlorophenyl | Cl |
| D-237 | 3,4,5-trichlorophenyl | Cl |
| D-238 | 2,3,4-trifluorophenyl | Cl |
| D-239 | 2,3,5-trifluorophenyl | Cl |
| D-240 | 2,3,6-trifluorophenyl | Cl |
| D-241 | 2,4,5-trifluorophenyl | Cl |
| D-242 | 2,4,6-trifluorophenyl | Cl |
| D-243 | 3,4,5-trifluorophenyl | Cl |
| D-244 | 2,3,4-trimethylphenyl | Cl |
| D-245 | 2,3,5-trimethylphenyl | Cl |
| D-246 | 2,3,6-trimethylphenyl | Cl |
| D-247 | 2,4,5-trimethylphenyl | Cl |
| D-248 | 2,4,6-trimethylphenyl | Cl |
| D-249 | 3,4,5-trimethylphenyl | Cl |
| D-250 | 2,3,4-trimethoxyphenyl | Cl |
| D-251 | 2,3,5-trimethoxyphenyl | Cl |
| D-252 | 2,3,6-trimethoxyphenyl | Cl |
| D-253 | 2,4,5-trimethoxyphenyl | Cl |
| D-254 | 2,4,6-trimethoxyphenyl | Cl |
| D-255 | 3,4,5-trimethoxyphenyl | Cl |
| D-256 | phenyl | Br |
| D-257 | 2-chlorophenyl | Br |
| D-258 | 3-chlorophenyl | Br |
| D-259 | 4-chlorophenyl | Br |
| D-260 | 2-fluorophenyl | Br |
| D-261 | 3-fluorophenyl | Br |
| D-262 | 4-fluorophenyl | Br |
| D-263 | 2-methylphenyl | Br |
| D-264 | 3-methylphenyl | Br |
| D-265 | 4-methylphenyl | Br |
| D-266 | 2-ethylphenyl | Br |
| D-267 | 3-ethylphenyl | Br |
| D-268 | 4-ethylphenyl | Br |
| D-269 | 2-methoxyphenyl | Br |
| D-270 | 3-methoxyphenyl | Br |
| D-271 | 4-methoxyphenyl | Br |
| D-272 | 2-trifluoromethylphenyl | Br |
| D-273 | 3-trifluoromethylphenyl | Br |
| D-274 | 4-trifluoromethylphenyl | Br |
| D-275 | 2-trifluoromethoxyphenyl | Br |
| D-276 | 3-trifluoromethoxyphenyl | Br |
| D-277 | 4-trifluoromethoxyphenyl | Br |
| D-278 | 2-difluoromethoxyphenyl | Br |
| D-279 | 3-difluoromethoxyphenyl | Br |
| D-280 | 4-difluoromethoxyphenyl | Br |
| D-281 | 2-trifluoromethylthiophenyl | Br |
| D-282 | 3-trifluoromethylthiophenyl | Br |
| D-283 | 4-trifluoromethylthiophenyl | Br |
| D-284 | 2,3-dichlorophenyl | Br |
| D-285 | 2,4-dichlorophenyl | Br |
| D-286 | 2,5-dichlorophenyl | Br |
| D-287 | 2,6-dichlorophenyl | Br |
| D-288 | 3,4-dichlorophenyl | Br |
| D-289 | 3,5-dichlorophenyl | Br |
| D-290 | 2,3-difluorophenyl | Br |
| D-291 | 2,4-difluorophenyl | Br |
| D-292 | 2,5-difluorophenyl | Br |
| D-293 | 2,6-difluorophenyl | Br |
| D-294 | 3,4-difluorophenyl | Br |
| D-295 | 3,5-difluorophenyl | Br |
| D-296 | 2,3-dimethylphenyl | Br |
| D-297 | 2,4-dimethylphenyl | Br |
| D-298 | 2,5-dimethylphenyl | Br |
| D-299 | 2,6-dimethylphenyl | Br |
| D-300 | 3,4-dimethylphenyl | Br |
| D-301 | 3,5-dimethylphenyl | Br |
| D-302 | 2,3-diethylphenyl | Br |
| D-303 | 2,4-diethylphenyl | Br |
| D-304 | 2,5-diethylphenyl | Br |
| D-305 | 2,6-diethylphenyl | Br |
| D-306 | 3,5-diethylphenyl | Br |
| D-307 | 3,4-diethylphenyl | Br |
| D-308 | 2,3-dimethoxyphenyl | Br |
| D-309 | 2,4-dimethoxyphenyl | Br |
| D-310 | 2,5-dimethoxyphenyl | Br |
| D-311 | 2,6-dimethoxyphenyl | Br |
| D-312 | 3,4-dimethoxyphenyl | Br |
| D-313 | 3,5-dimethoxyphenyl | Br |
| D-314 | 2,3-di(trifluoromethyl)phenyl | Br |
| D-315 | 2,4-di(trifluoromethyl)phenyl | Br |
| D-316 | 2,5-di(trifluoromethyl)phenyl | Br |
| D-317 | 2,6-di(trifluoromethyl)phenyl | Br |
| D-318 | 3,4-di(trifluoromethyl)phenyl | Br |
| D-319 | 3,5-di(trifluoromethyl)phenyl | Br |
| D-320 | 2,3-di(trifluoromethoxy)phenyl | Br |
| D-321 | 2,4-di(trifluoromethoxy)phenyl | Br |
| D-322 | 2,5-di(trifluoromethoxy)phenyl | Br |
| D-323 | 2,6-di(trifluoromethoxy)phenyl | Br |
| D-324 | 3,4-di(trifluoromethoxy)phenyl | Br |
| D-325 | 3,5-di(trifluoromethoxy)phenyl | Br |
| D-326 | 2,3-di(difluoromethoxy)phenyl | Br |
| D-327 | 2,4-di(difluoromethoxy)phenyl | Br |
| D-328 | 2,5-di(difluoromethoxy)phenyl | Br |
| D-329 | 2,6-di(difluoromethoxy)phenyl | Br |
| D-330 | 3,4-di(difluoromethoxy)phenyl | Br |
| D-331 | 3,5-di(difluoromethoxy)phenyl | Br |
| D-332 | 2,3-di(trifluoromethylthio)phenyl | Br |
| D-333 | 2,4-di(trifluoromethylthio)phenyl | Br |
| D-334 | 2,5-di(trifluoromethylthio)phenyl | Br |
| D-335 | 2,6-di(trifluoromethylthio)phenyl | Br |
| D-336 | 3,4-di(trifluoromethylthio)phenyl | Br |
| D-337 | 3,5-di(trifluoromethylthio)phenyl | Br |
| D-338 | 2-fluoro-3-chlorophenyl | Br |
| D-339 | 2-fluoro-4-chlorophenyl | Br |
| D-340 | 2-fluoro-5-chlorophenyl | Br |
| D-341 | 2-fluoro-6-chlorophenyl | Br |
| D-342 | 3-fluoro-4-chlorophenyl | Br |
| D-343 | 3-fluoro-5-chlorophenyl | Br |
| D-344 | 2-chloro-3-fluorophenyl | Br |
| D-345 | 2-chloro-4-fluorophenyl | Br |
| D-346 | 2-chloro-5-fluorophenyl | Br |
| D-347 | 3-chloro-4-fluorophenyl | Br |
| D-348 | 2-methyl-3-chlorophenyl | Br |
| D-349 | 2-methyl-4-chlorophenyl | Br |
| D-350 | 2-methyl-5-chlorophenyl | Br |
| D-351 | 2-methyl-6-chlorophenyl | Br |
| D-352 | 3-methyl-4-chlorophenyl | Br |
| D-353 | 3-methyl-5-chlorophenyl | Br |
| D-354 | 2-chloro-3-methylphenyl | Br |
| D-355 | 2-chloro-4-methylphenyl | Br |
| D-356 | 2-chloro-5-methylphenyl | Br |
| D-357 | 3-chloro-4-methylphenyl | Br |
| D-358 | 2-methyl-3-fluorophenyl | Br |
| D-359 | 2-methyl-4-fluorophenyl | Br |
| D-360 | 2-methyl-5-fluorophenyl | Br |
| D-361 | 2-methyl-6-fluorophenyl | Br |
| D-362 | 3-methyl-4-fluorophenyl | Br |
| D-363 | 3-methyl-5-fluorophenyl | Br |
| D-364 | 2-fluoro-3-methylphenyl | Br |
| D-365 | 2-fluoro-4-methylphenyl | Br |
| D-366 | 2-fluoro-5-methylphenyl | Br |
| D-367 | 3-fluoro-4-methylphenyl | Br |
| D-368 | 2-chloro-3-ethylphenyl | Br |
| D-369 | 2-chloro-4-ethylphenyl | Br |
| D-370 | 2-chloro-5-ethylphenyl | Br |
| D-371 | 3-chloro-4-ethylphenyl | Br |
| D-372 | 2-ethyl-3-chlorophenyl | Br |
| D-373 | 2-ethyl-4-chlorophenyl | Br |
| D-374 | 2-ethyl-5-chlorophenyl | Br |
| D-375 | 2-ethyl-6-chlorophenyl | Br |
| D-376 | 2-ethyl-3-fluorophenyl | Br |
| D-377 | 2-ethyl-4-fluorophenyl | Br |
| D-378 | 2-ethyl-5-fluorophenyl | Br |
| D-379 | 2-ethyl-6-fluorophenyl | Br |
| D-380 | 3-ethyl-4-fluorophenyl | Br |
| D-381 | 3-ethyl-5-fluorophenyl | Br |
| D-382 | 2-fluoro-3-ethylphenyl | Br |
| D-383 | 2-fluoro-4-ethylphenyl | Br |
| D-384 | 2-fluoro-5-ethylphenyl | Br |
| D-385 | 3-fluoro-4-ethylphenyl | Br |
| D-386 | 2-methoxy-3-chlorophenyl | Br |
| D-387 | 2-methoxy-4-chlorophenyl | Br |
| D-388 | 2-methoxy-5-chlorophenyl | Br |
| D-389 | 2-methoxy-6-chlorophenyl | Br |
| D-390 | 3-methoxy-4-chlorophenyl | Br |
| D-391 | 3-methoxy-5-chlorophenyl | Br |
| D-392 | 2-chloro-3-methoxyphenyl | Br |
| D-393 | 2-chloro-4-methoxyphenyl | Br |
| D-394 | 2-chloro-5-methoxyphenyl | Br |
| D-395 | 3-chloro-4-methoxyphenyl | Br |
| D-396 | 2-methoxy-3-fluorophenyl | Br |
| D-397 | 2-methoxy-4-fluorophenyl | Br |
| D-398 | 2-methoxy-5-fluorophenyl | Br |
| D-399 | 2-methoxy-6-fluorophenyl | Br |
| D-400 | 3-methoxy-4-fluorophenyl | Br |
| D-401 | 3-methoxy-5-fluorophenyl | Br |
| D-402 | 2-fluoro-3-methoxyphenyl | Br |
| D-403 | 2-fluoro-4-methoxyphenyl | Br |
| D-404 | 2-fluoro-5-methoxyphenyl | Br |
| D-405 | 3-fluoro-4-methoxyphenyl | Br |
| D-406 | 3-fluoro-5-methoxyphenyl | Br |
| D-407 | 2-(trifluoromethyl)-3-chlorophenyl | Br |
| D-408 | 2-(trifluoromethyl)-4-chlorophenyl | Br |
| D-409 | 2-(trifluoromethyl)-5-chlorophenyl | Br |
| D-410 | 2-(trifluoromethyl)-6-chlorophenyl | Br |
| D-411 | 3-(trifluoromethyl)-4-chlorophenyl | Br |
| D-412 | 3-(trifluoromethyl)-5-chlorophenyl | Br |
| D-413 | 2-chloro-3-(trifluoromethyl)phenyl | Br |
| D-414 | 2-chloro-4-(trifluoromethyl)phenyl | Br |
| D-415 | 2-chloro-5-(trifluoromethyl)phenyl | Br |
| D-416 | 3-chloro-4-(trifluoromethyl)phenyl | Br |
| D-417 | 2-(trifluoromethyl)-3-fluorophenyl | Br |
| D-418 | 2-(trifluoromethyl)-4-fluorophenyl | Br |
| D-419 | 2-(trifluoromethyl)-5-fluorophenyl | Br |
| D-420 | 2-(trifluoromethyl)-6-fluorophenyl | Br |
| D-421 | 3-(trifluoromethyl)-4-fluorophenyl | Br |
| D-422 | 3-(trifluoromethyl)-5-fluorophenyl | Br |
| D-423 | 2-fluoro-3-(trifluoromethyl)phenyl | Br |
| D-424 | 2-fluoro-4-(trifluoromethyl)phenyl | Br |
| D-425 | 2-fluoro-5-(trifluoromethyl)phenyl | Br |
| D-426 | 3-fluoro-4-(trifluoromethyl)phenyl | Br |
| D-427 | 2-(trifluoromethoxy)-3-chlorophenyl | Br |
| D-428 | 2-(trifluoromethoxy)-4-chlorophenyl | Br |
| D-429 | 2-(trifluoromethoxy)-5-chlorophenyl | Br |
| D-430 | 2-(trifluoromethoxy)-6-chlorophenyl | Br |
| D-431 | 3-(trifluoromethoxy)-4-chlorophenyl | Br |
| D-432 | 3-(trifluoromethoxy)-5-chlorophenyl | Br |
| D-433 | 2-chloro-3-(trifluoromethoxy)phenyl | Br |
| D-434 | 2-chloro-4-(trifluoromethoxy)phenyl | Br |
| D-435 | 2-chloro-5-(trifluoromethoxy)phenyl | Br |
| D-436 | 3-chloro-4-(trifluoromethoxy)phenyl | Br |
| D-437 | 2-(trifluoromethoxy)-3-fluorophenyl | Br |
| D-438 | 2-(trifluoromethoxy)-4-fluorophenyl | Br |
| D-439 | 2-(trifluoromethoxy)-5-fluorophenyl | Br |
| D-440 | 2-(trifluoromethoxy)-6-fluorophenyl | Br |
| D-441 | 3-(trifluoromethoxy)-4-fluorophenyl | Br |
| D-442 | 3-(trifluoromethoxy)-5-fluorophenyl | Br |
| D-443 | 2-fluoro-3-(trifluoromethoxy)phenyl | Br |
| D-444 | 2-fluoro-4-(trifluoromethoxy)phenyl | Br |
| D-445 | 2-fluoro-5-(trifluoromethoxy)phenyl | Br |
| D-446 | 3-fluoro-4-(trifluoromethoxy)phenyl | Br |
| D-447 | 2-(difluoromethoxy)-3-chlorophenyl | Br |
| D-448 | 2-(difluoromethoxy)-4-chlorophenyl | Br |
| D-449 | 2-(difluoromethoxy)-5-chlorophenyl | Br |
| D-450 | 2-(difluoromethoxy)-6-chlorophenyl | Br |
| D-451 | 3-(difluoromethoxy)-4-chlorophenyl | Br |
| D-452 | 3-(difluoromethoxy)-5-chlorophenyl | Br |
| D-453 | 2-chloro-3-(difluoromethoxy)phenyl | Br |
| D-454 | 2-chloro-4-(difluoromethoxy)phenyl | Br |
| D-455 | 2-chloro-5-(difluoromethoxy)phenyl | Br |
| D-456 | 3-chloro-4-(difluoromethoxy)phenyl | Br |
| D-457 | 2-(difluoromethoxy)-3-fluorophenyl | Br |
| D-458 | 2-(difluoromethoxy)-4-fluorophenyl | Br |
| D-459 | 2-(difluoromethoxy)-5-fluorophenyl | Br |
| D-460 | 2-(difluoromethoxy)-6-fluorophenyl | Br |
| D-461 | 3-(difluoromethoxy)-4-fluorophenyl | Br |
| D-462 | 3-(difluoromethoxy)-5-fluorophenyl | Br |
| D-463 | 2-fluoro-3-(difluoromethoxy)phenyl | Br |
| D-464 | 2-fluoro-4-(difluoromethoxy)phenyl | Br |
| D-465 | 2-fluoro-5-(difluoromethoxy)phenyl | Br |
| D-466 | 3-fluoro-4-(difluoromethoxy)phenyl | Br |
| D-467 | 2-(trifluoromethylthio)-3-chlorophenyl | Br |
| D-468 | 2-(trifluoromethylthio)-4-chlorophenyl | Br |
| D-469 | 2-(trifluoromethylthio)-5-chlorophenyl | Br |
| D-470 | 2-(trifluoromethylthio)-6-chlorophenyl | Br |
| D-471 | 3-(trifluoromethylthio)-4-chlorophenyl | Br |
| D-472 | 3-(trifluoromethylthio)-5-chlorophenyl | Br |
| D-473 | 2-chloro-3-(trifluoromethylthio)phenyl | Br |
| D-474 | 2-chloro-4-(trifluoromethylthio)phenyl | Br |
| D-475 | 2-chloro-5-(trifluoromethylthio)phenyl | Br |
| D-476 | 3-chloro-4-(trifluoromethylthio)phenyl | Br |
| D-477 | 2-(trifluoromethylthio)-3-fluorophenyl | Br |
| D-478 | 2-(trifluoromethylthio)-4-fluorophenyl | Br |
| D-479 | 2-(trifluoromethylthio)-5-fluorophenyl | Br |
| D-480 | 2-(trifluoromethylthio)-6-fluorophenyl | Br |
| D-481 | 3-(trifluoromethylthio)-4-fluorophenyl | Br |
| D-482 | 3-(trifluoromethylthio)-5-fluorophenyl | Br |
| D-483 | 2-fluoro-3-(trifluoromethylthio)phenyl | Br |
| D-484 | 2-fluoro-4-(trifluoromethylthio)phenyl | Br |
| D-485 | 2-fluoro-5-(trifluoromethylthio)phenyl | Br |
| D-486 | 3-fluoro-4-(trifluoromethylthio)phenyl | Br |
| D-487 | 2,3,4-trichlorophenyl | Br |
| D-488 | 2,3,5-trichlorophenyl | Br |
| D-489 | 2,3,6-trichlorophenyl | Br |
| D-490 | 2,4,5-trichlorophenyl | Br |
| D-491 | 2,4,6-trichlorophenyl | Br |
| D-492 | 3,4,5-trichlorophenyl | Br |
| D-493 | 2,3,4-trifluorophenyl | Br |
| D-494 | 2,3,5-trifluorophenyl | Br |
| D-495 | 2,3,6-trifluorophenyl | Br |
| D-496 | 2,4,5-trifluorophenyl | Br |
| D-497 | 2,4,6-trifluorophenyl | Br |
| D-498 | 3,4,5-trifluorophenyl | Br |
| D-499 | 2,3,4-trimethylphenyl | Br |
| D-500 | 2,3,5-trimethylphenyl | Br |
| D-501 | 2,3,6-trimethylphenyl | Br |
| D-502 | 2,4,5-trimethylphenyl | Br |
| D-503 | 2,4,6-trimethylphenyl | Br |
| D-504 | 3,4,5-trimethylphenyl | Br |
| D-505 | 2,3,4-trimethoxyphenyl | Br |
| D-506 | 2,3,5-trimethoxyphenyl | Br |
| D-507 | 2,3,6-trimethoxyphenyl | Br |
| D-508 | 2,4,5-trimethoxyphenyl | Br |
| D-509 | 2,4,6-trimethoxyphenyl | Br |
| D-510 | 3,4,5-trimethoxyphenyl | Br |
| D-511 | phenyl | OTs |
| D-512 | 2-chlorophenyl | OTs |
| D-513 | 3-chlorophenyl | OTs |
| D-514 | 4-chlorophenyl | OTs |
| D-515 | 2-fluorophenyl | OTs |
| D-516 | 3-fluorophenyl | OTs |
| D-517 | 4-fluorophenyl | OTs |
| D-518 | 2-methylphenyl | OTs |
| D-519 | 3-methylphenyl | OTs |
| D-520 | 4-methylphenyl | OTs |
| D-521 | 2-ethylphenyl | OTs |
| D-522 | 3-ethylphenyl | OTs |
| D-523 | 4-ethylphenyl | OTs |
| D-524 | 2-methoxyphenyl | OTs |
| D-525 | 3-methoxyphenyl | OTs |
| D-526 | 4-methoxyphenyl | OTs |
| D-527 | 2-trifluoromethylphenyl | OTs |
| D-528 | 3-trifluoromethylphenyl | OTs |
| D-529 | 4-trifluoromethylphenyl | OTs |
| D-530 | 2-trifluoromethoxyphenyl | OTs |
| D-531 | 3-trifluoromethoxyphenyl | OTs |
| D-532 | 4-trifluoromethoxyphenyl | OTs |
| D-533 | 2-difluoromethoxyphenyl | OTs |
| D-534 | 3-difluoromethoxyphenyl | OTs |
| D-535 | 4-difluoromethoxyphenyl | OTs |
| D-536 | 2-trifluoromethylthiophenyl | OTs |
| D-537 | 3-trifluoromethylthiophenyl | OTs |
| D-538 | 4-trifluoromethylthiophenyl | OTs |
| D-539 | 2,3-dichlorophenyl | OTs |
| D-540 | 2,4-dichlorophenyl | OTs |
| D-541 | 2,5-dichlorophenyl | OTs |
| D-542 | 2,6-dichlorophenyl | OTs |
| D-543 | 3,4-dichlorophenyl | OTs |
| D-544 | 3,5-dichlorophenyl | OTs |
| D-545 | 2,3-difluorophenyl | OTs |
| D-546 | 2,4-difluorophenyl | OTs |
| D-547 | 2,5-difluorophenyl | OTs |
| D-548 | 2,6-difluorophenyl | OTs |
| D-549 | 3,4-difluorophenyl | OTs |
| D-550 | 3,5-difluorophenyl | OTs |
| D-551 | 2,3-dimethylphenyl | OTs |
| D-552 | 2,4-dimethylphenyl | OTs |
| D-553 | 2,5-dimethylphenyl | OTs |
| D-554 | 2,6-dimethylphenyl | OTs |
| D-555 | 3,4-dimethylphenyl | OTs |
| D-556 | 3,5-dimethylphenyl | OTs |
| D-557 | 2,3-diethylphenyl | OTs |
| D-558 | 2,4-diethylphenyl | OTs |
| D-559 | 2,5-diethylphenyl | OTs |
| D-560 | 2,6-diethylphenyl | OTs |
| D-561 | 3,5-diethylphenyl | OTs |
| D-562 | 3,4-diethylphenyl | OTs |
| D-563 | 2,3-dimethoxyphenyl | OTs |
| D-564 | 2,4-dimethoxyphenyl | OTs |
| D-565 | 2,5-dimethoxyphenyl | OTs |
| D-566 | 2,6-dimethoxyphenyl | OTs |
| D-567 | 3,4-dimethoxyphenyl | OTs |
| D-568 | 3,5-dimethoxyphenyl | OTs |
| D-569 | 2,3-di(trifluoromethyl)phenyl | OTs |
| D-570 | 2,4-di(trifluoromethyl)phenyl | OTs |
| D-571 | 2,5-di(trifluoromethyl)phenyl | OTs |
| D-572 | 2,6-di(trifluoromethyl)phenyl | OTs |
| D-573 | 3,4-di(trifluoromethyl)phenyl | OTs |
| D-574 | 3,5-di(trifluoromethyl)phenyl | OTs |
| D-575 | 2,3-di(trifluoromethoxy)phenyl | OTs |
| D-576 | 2,4-di(trifluoromethoxy)phenyl | OTs |
| D-577 | 2,5-di(trifluoromethoxy)phenyl | OTs |
| D-578 | 2,6-di(trifluoromethoxy)phenyl | OTs |
| D-579 | 3,4-di(trifluoromethoxy)phenyl | OTs |
| D-580 | 3,5-di(trifluoromethoxy)phenyl | OTs |
| D-581 | 2,3-di(difluoromethoxy)phenyl | OTs |
| D-582 | 2,4-di(difluoromethoxy)phenyl | OTs |
| D-583 | 2,5-di(difluoromethoxy)phenyl | OTs |
| D-584 | 2,6-di(difluoromethoxy)phenyl | OTs |
| D-585 | 3,4-di(difluoromethoxy)phenyl | OTs |
| D-586 | 3,5-di(difluoromethoxy)phenyl | OTs |
| D-587 | 2,3-di(trifluoromethylthio)phenyl | OTs |
| D-588 | 2,4-di(trifluoromethylthio)phenyl | OTs |
| D-589 | 2,5-di(trifluoromethylthio)phenyl | OTs |
| D-590 | 2,6-di(trifluoromethylthio)phenyl | OTs |
| D-591 | 3,4-di(trifluoromethylthio)phenyl | OTs |
| D-592 | 3,5-di(trifluoromethylthio)phenyl | OTs |
| D-593 | 2-fluoro-3-chlorophenyl | OTs |
| D-594 | 2-fluoro-4-chlorophenyl | OTs |
| D-595 | 2-fluoro-5-chlorophenyl | OTs |
| D-596 | 2-fluoro-6-chlorophenyl | OTs |
| D-597 | 3-fluoro-4-chlorophenyl | OTs |
| D-598 | 3-fluoro-5-chlorophenyl | OTs |
| D-599 | 2-chloro-3-fluorophenyl | OTs |
| D-600 | 2-chloro-4-fluorophenyl | OTs |
| D-601 | 2-chloro-5-fluorophenyl | OTs |
| D-602 | 3-chloro-4-fluorophenyl | OTs |
| D-603 | 2-methyl-3-chlorophenyl | OTs |
| D-604 | 2-methyl-4-chlorophenyl | OTs |
| D-605 | 2-methyl-5-chlorophenyl | OTs |
| D-606 | 2-methyl-6-chlorophenyl | OTs |
| D-607 | 3-methyl-4-chlorophenyl | OTs |
| D-608 | 3-methyl-5-chlorophenyl | OTs |
| D-609 | 2-chloro-3-methylphenyl | OTs |
| D-610 | 2-chloro-4-methylphenyl | OTs |
| D-611 | 2-chloro-5-methylphenyl | OTs |
| D-612 | 3-chloro-4-methylphenyl | OTs |
| D-613 | 2-methyl-3-fluorophenyl | OTs |
| D-614 | 2-methyl-4-fluorophenyl | OTs |
| D-615 | 2-methyl-5-fluorophenyl | OTs |
| D-616 | 2-methyl-6-fluorophenyl | OTs |
| D-617 | 3-methyl-4-fluorophenyl | OTs |
| D-618 | 3-methyl-5-fluorophenyl | OTs |
| D-619 | 2-fluoro-3-methylphenyl | OTs |
| D-620 | 2-fluoro-4-methylphenyl | OTs |
| D-621 | 2-fluoro-5-methylphenyl | OTs |
| D-622 | 3-fluoro-4-methylphenyl | OTs |
| D-623 | 2-chloro-3-ethylphenyl | OTs |
| D-624 | 2-chloro-4-ethylphenyl | OTs |
| D-625 | 2-chloro-5-ethylphenyl | OTs |
| D-626 | 3-chloro-4-ethylphenyl | OTs |
| D-627 | 2-ethyl-3-chlorophenyl | OTs |
| D-628 | 2-ethyl-4-chlorophenyl | OTs |
| D-629 | 2-ethyl-5-chlorophenyl | OTs |
| D-630 | 2-ethyl-6-chlorophenyl | OTs |
| D-631 | 2-ethyl-3-fluorophenyl | OTs |
| D-632 | 2-ethyl-4-fluorophenyl | OTs |
| D-633 | 2-ethyl-5-fluorophenyl | OTs |
| D-634 | 2-ethyl-6-fluorophenyl | OTs |
| D-635 | 3-ethyl-4-fluorophenyl | OTs |
| D-636 | 3-ethyl-5-fluorophenyl | OTs |
| D-637 | 2-fluoro-3-ethylphenyl | OTs |
| D-638 | 2-fluoro-4-ethylphenyl | OTs |
| D-639 | 2-fluoro-5-ethylphenyl | OTs |
| D-640 | 3-fluoro-4-ethylphenyl | OTs |
| D-641 | 2-methoxy-3-chlorophenyl | OTs |
| D-642 | 2-methoxy-4-chlorophenyl | OTs |
| D-643 | 2-methoxy-5-chlorophenyl | OTs |
| D-644 | 2-methoxy-6-chlorophenyl | OTs |
| D-645 | 3-methoxy-4-chlorophenyl | OTs |
| D-646 | 3-methoxy-5-chlorophenyl | OTs |
| D-647 | 2-chloro-3-methoxyphenyl | OTs |
| D-648 | 2-chloro-4-methoxyphenyl | OTs |
| D-649 | 2-chloro-5-methoxyphenyl | OTs |
| D-650 | 3-chloro-4-methoxyphenyl | OTs |
| D-651 | 2-methoxy-3-fluorophenyl | OTs |
| D-652 | 2-methoxy-4-fluorophenyl | OTs |
| D-653 | 2-methoxy-5-fluorophenyl | OTs |
| D-654 | 2-methoxy-6-fluorophenyl | OTs |
| D-655 | 3-methoxy-4-fluorophenyl | OTs |
| D-656 | 3-methoxy-5-fluorophenyl | OTs |
| D-657 | 2-fluoro-3-methoxyphenyl | OTs |
| D-658 | 2-fluoro-4-methoxyphenyl | OTs |
| D-659 | 2-fluoro-5-methoxyphenyl | OTs |
| D-660 | 3-fluoro-4-methoxyphenyl | OTs |
| D-661 | 3-fluoro-5-methoxyphenyl | OTs |
| D-662 | 2-(trifluoromethyl)-3-chlorophenyl | OTs |
| D-663 | 2-(trifluoromethyl)-4-chlorophenyl | OTs |
| D-664 | 2-(trifluoromethyl)-5-chlorophenyl | OTs |
| D-665 | 2-(trifluoromethyl)-6-chlorophenyl | OTs |
| D-666 | 3-(trifluoromethyl)-4-chlorophenyl | OTs |
| D-667 | 3-(trifluoromethyl)-5-chlorophenyl | OTs |
| D-668 | 2-chloro-3-(trifluoromethyl)phenyl | OTs |
| D-669 | 2-chloro-4-(trifluoromethyl)phenyl | OTs |
| D-670 | 2-chloro-5-(trifluoromethyl)phenyl | OTs |
| D-671 | 3-chloro-4-(trifluoromethyl)phenyl | OTs |
| D-672 | 2-(trifluoromethyl)-3-fluorophenyl | OTs |
| D-673 | 2-(trifluoromethyl)-4-fluorophenyl | OTs |
| D-674 | 2-(trifluoromethyl)-5-fluorophenyl | OTs |
| D-675 | 2-(trifluoromethyl)-6-fluorophenyl | OTs |
| D-676 | 3-(trifluoromethyl)-4-fluorophenyl | OTs |
| D-677 | 3-(trifluoromethyl)-5-fluorophenyl | OTs |
| D-678 | 2-fluoro-3-(trifluoromethyl)phenyl | OTs |
| D-679 | 2-fluoro-4-(trifluoromethyl)phenyl | OTs |
| D-680 | 2-fluoro-5-(trifluoromethyl)phenyl | OTs |
| D-681 | 3-fluoro-4-(trifluoromethyl)phenyl | OTs |
| D-682 | 2-(trifluoromethoxy)-3-chlorophenyl | OTs |
| D-683 | 2-(trifluoromethoxy)-4-chlorophenyl | OTs |
| D-684 | 2-(trifluoromethoxy)-5-chlorophenyl | OTs |
| D-685 | 2-(trifluoromethoxy)-6-chlorophenyl | OTs |
| D-686 | 3-(trifluoromethoxy)-4-chlorophenyl | OTs |
| D-687 | 3-(trifluoromethoxy)-5-chlorophenyl | OTs |
| D-688 | 2-chloro-3-(trifluoromethoxy)phenyl | OTs |
| D-689 | 2-chloro-4-(trifluoromethoxy)phenyl | OTs |
| D-690 | 2-chloro-5-(trifluoromethoxy)phenyl | OTs |
| D-691 | 3-chloro-4-(trifluoromethoxy)phenyl | OTs |
| D-692 | 2-(trifluoromethoxy)-3-fluorophenyl | OTs |
| D-693 | 2-(trifluoromethoxy)-4-fluorophenyl | OTs |
| D-694 | 2-(trifluoromethoxy)-5-fluorophenyl | OTs |
| D-695 | 2-(trifluoromethoxy)-6-fluorophenyl | OTs |
| D-696 | 3-(trifluoromethoxy)-4-fluorophenyl | OTs |
| D-697 | 3-(trifluoromethoxy)-5-fluorophenyl | OTs |
| D-698 | 2-fluoro-3-(trifluoromethoxy)phenyl | OTs |
| D-699 | 2-fluoro-4-(trifluoromethoxy)phenyl | OTs |
| D-700 | 2-fluoro-5-(trifluoromethoxy)phenyl | OTs |
| D-701 | 3-fluoro-4-(trifluoromethoxy)phenyl | OTs |
| D-702 | 2-(difluoromethoxy)-3-chlorophenyl | OTs |
| D-703 | 2-(difluoromethoxy)-4-chlorophenyl | OTs |
| D-704 | 2-(difluoromethoxy)-5-chlorophenyl | OTs |
| D-705 | 2-(difluoromethoxy)-6-chlorophenyl | OTs |
| D-706 | 3-(difluoromethoxy)-4-chlorophenyl | OTs |
| D-707 | 3-(difluoromethoxy)-5-chlorophenyl | OTs |
| D-708 | 2-chloro-3-(difluoromethoxy)phenyl | OTs |
| D-709 | 2-chloro-4-(difluoromethoxy)phenyl | OTs |
| D-710 | 2-chloro-5-(difluoromethoxy)phenyl | OTs |
| D-711 | 3-chloro-4-(difluoromethoxy)phenyl | OTs |
| D-712 | 2-(difluoromethoxy)-3-fluorophenyl | OTs |
| D-713 | 2-(difluoromethoxy)-4-fluorophenyl | OTs |
| D-714 | 2-(difluoromethoxy)-5-fluorophenyl | OTs |
| D-715 | 2-(difluoromethoxy)-6-fluorophenyl | OTs |
| D-716 | 3-(difluoromethoxy)-4-fluorophenyl | OTs |
| D-717 | 3-(difluoromethoxy)-5-fluorophenyl | OTs |
| D-718 | 2-fluoro-3-(difluoromethoxy)phenyl | OTs |
| D-719 | 2-fluoro-4-(difluoromethoxy)phenyl | OTs |
| D-720 | 2-fluoro-5-(difluoromethoxy)phenyl | OTs |
| D-721 | 3-fluoro-4-(difluoromethoxy)phenyl | OTs |
| D-722 | 2-(trifluoromethylthio)-3-chlorophenyl | OTs |
| D-723 | 2-(trifluoromethylthio)-4-chlorophenyl | OTs |
| D-724 | 2-(trifluoromethylthio)-5-chlorophenyl | OTs |
| D-725 | 2-(trifluoromethylthio)-6-chlorophenyl | OTs |
| D-726 | 3-(trifluoromethylthio)-4-chlorophenyl | OTs |
| D-727 | 3-(trifluoromethylthio)-5-chlorophenyl | OTs |
| D-728 | 2-chloro-3-(trifluoromethylthio)phenyl | OTs |
| D-729 | 2-chloro-4-(trifluoromethylthio)phenyl | OTs |
| D-730 | 2-chloro-5-(trifluoromethylthio)phenyl | OTs |
| D-731 | 3-chloro-4-(trifluoromethylthio)phenyl | OTs |
| D-732 | 2-(trifluoromethylthio)-3-fluorophenyl | OTs |
| D-733 | 2-(trifluoromethylthio)-4-fluorophenyl | OTs |
| D-734 | 2-(trifluoromethylthio)-5-fluorophenyl | OTs |
| D-735 | 2-(trifluoromethylthio)-6-fluorophenyl | OTs |
| D-736 | 3-(trifluoromethylthio)-4-fluorophenyl | OTs |
| D-737 | 3-(trifluoromethylthio)-5-fluorophenyl | OTs |
| D-738 | 2-fluoro-3-(trifluoromethylthio)phenyl | OTs |
| D-739 | 2-fluoro-4-(trifluoromethylthio)phenyl | OTs |
| D-740 | 2-fluoro-5-(trifluoromethylthio)phenyl | OTs |
| D-741 | 3-fluoro-4-(trifluoromethylthio)phenyl | OTs |
| D-742 | 2,3,4-trichlorophenyl | OTs |
| D-743 | 2,3,5-trichlorophenyl | OTs |
| D-744 | 2,3,6-trichlorophenyl | OTs |
| D-745 | 2,4,5-trichlorophenyl | OTs |
| D-746 | 2,4,6-trichlorophenyl | OTs |
| D-747 | 3,4,5-trichlorophenyl | OTs |
| D-748 | 2,3,4-trifluorophenyl | OTs |
| D-749 | 2,3,5-trifluorophenyl | OTs |
| D-750 | 2,3,6-trifluorophenyl | OTs |
| D-751 | 2,4,5-trifluorophenyl | OTs |
| D-752 | 2,4,6-trifluorophenyl | OTs |
| D-753 | 3,4,5-trifluorophenyl | OTs |
| D-754 | 2,3,4-trimethylphenyl | OTs |
| D-755 | 2,3,5-trimethylphenyl | OTs |
| D-756 | 2,3,6-trimethylphenyl | OTs |
| D-757 | 2,4,5-trimethylphenyl | OTs |
| D-758 | 2,4,6-trimethylphenyl | OTs |
| D-759 | 3,4,5-trimethylphenyl | OTs |
| D-760 | 2,3,4-trimethoxyphenyl | OTs |
| D-761 | 2,3,5-trimethoxyphenyl | OTs |
| D-762 | 2,3,6-trimethoxyphenyl | OTs |
| D-763 | 2,4,5-trimethoxyphenyl | OTs |
| D-764 | 2,4,6-trimethoxyphenyl | OTs |
| D-765 | 3,4,5-trimethoxyphenyl | OTs |
| D-766 | phenyl | OMs |
| D-767 | 2-chlorophenyl | OMs |
| D-768 | 3-chlorophenyl | OMs |
| D-769 | 4-chlorophenyl | OMs |
| D-770 | 2-fluorophenyl | OMs |
| D-771 | 3-fluorophenyl | OMs |
| D-772 | 4-fluorophenyl | OMs |
| D-773 | 2-methylphenyl | OMs |
| D-774 | 3-methylphenyl | OMs |
| D-775 | 4-methylphenyl | OMs |
| D-776 | 2-ethylphenyl | OMs |
| D-777 | 3-ethylphenyl | OMs |
| D-778 | 4-ethylphenyl | OMs |
| D-779 | 2-methoxyphenyl | OMs |
| D-780 | 3-methoxyphenyl | OMs |
| D-781 | 4-methoxyphenyl | OMs |
| D-782 | 2-trifluoromethylphenyl | OMs |
| D-783 | 3-trifluoromethylphenyl | OMs |
| D-784 | 4-trifluoromethylphenyl | OMs |
| D-785 | 2-trifluoromethoxyphenyl | OMs |
| D-786 | 3-trifluoromethoxyphenyl | OMs |
| D-787 | 4-trifluoromethoxyphenyl | OMs |
| D-788 | 2-difluoromethoxyphenyl | OMs |
| D-789 | 3-difluoromethoxyphenyl | OMs |
| D-790 | 4-difluoromethoxyphenyl | OMs |
| D-791 | 2-trifluoromethylthiophenyl | OMs |
| D-792 | 3-trifluoromethylthiophenyl | OMs |
| D-793 | 4-trifluoromethylthiophenyl | OMs |
| D-794 | 2,3-dichlorophenyl | OMs |
| D-795 | 2,4-dichlorophenyl | OMs |
| D-796 | 2,5-dichlorophenyl | OMs |
| D-797 | 2,6-dichlorophenyl | OMs |
| D-798 | 3,4-dichlorophenyl | OMs |
| D-799 | 3,5-dichlorophenyl | OMs |
| D-800 | 2,3-difluorophenyl | OMs |
| D-801 | 2,4-difluorophenyl | OMs |
| D-802 | 2,5-difluorophenyl | OMs |
| D-803 | 2,6-difluorophenyl | OMs |
| D-804 | 3,4-difluorophenyl | OMs |
| D-805 | 3,5-difluorophenyl | OMs |
| D-806 | 2,3-dimethylphenyl | OMs |
| D-807 | 2,4-dimethylphenyl | OMs |
| D-808 | 2,5-dimethylphenyl | OMs |
| D-809 | 2,6-dimethylphenyl | OMs |
| D-810 | 3,4-dimethylphenyl | OMs |
| D-811 | 3,5-dimethylphenyl | OMs |
| D-812 | 2,3-diethylphenyl | OMs |
| D-813 | 2,4-diethylphenyl | OMs |
| D-814 | 2,5-diethylphenyl | OMs |
| D-815 | 2,6-diethylphenyl | OMs |
| D-816 | 3,5-diethylphenyl | OMs |
| D-817 | 3,4-diethylphenyl | OMs |
| D-818 | 2,3-dimethoxyphenyl | OMs |
| D-819 | 2,4-dimethoxyphenyl | OMs |
| D-820 | 2,5-dimethoxyphenyl | OMs |
| D-821 | 2,6-dimethoxyphenyl | OMs |
| D-822 | 3,4-dimethoxyphenyl | OMs |
| D-823 | 3,5-dimethoxyphenyl | OMs |
| D-824 | 2,3-di(trifluoromethyl)phenyl | OMs |
| D-825 | 2,4-di(trifluoromethyl)phenyl | OMs |
| D-826 | 2,5-di(trifluoromethyl)phenyl | OMs |
| D-827 | 2,6-di(trifluoromethyl)phenyl | OMs |
| D-828 | 3,4-di(trifluoromethyl)phenyl | OMs |
| D-829 | 3,5-di(trifluoromethyl)phenyl | OMs |
| D-830 | 2,3-di(trifluoromethoxy)phenyl | OMs |
| D-831 | 2,4-di(trifluoromethoxy)phenyl | OMs |
| D-832 | 2,5-di(trifluoromethoxy)phenyl | OMs |
| D-833 | 2,6-di(trifluoromethoxy)phenyl | OMs |
| D-834 | 3,4-di(trifluoromethoxy)phenyl | OMs |
| D-835 | 3,5-di(trifluoromethoxy)phenyl | OMs |
| D-836 | 2,3-di(difluoromethoxy)phenyl | OMs |
| D-837 | 2,4-di(difluoromethoxy)phenyl | OMs |
| D-838 | 2,5-di(difluoromethoxy)phenyl | OMs |
| D-839 | 2,6-di(difluoromethoxy)phenyl | OMs |
| D-840 | 3,4-di(difluoromethoxy)phenyl | OMs |
| D-841 | 3,5-di(difluoromethoxy)phenyl | OMs |
| D-842 | 2,3-di(trifluoromethylthio)phenyl | OMs |
| D-843 | 2,4-di(trifluoromethylthio)phenyl | OMs |
| D-844 | 2,5-di(trifluoromethylthio)phenyl | OMs |
| D-845 | 2,6-di(trifluoromethylthio)phenyl | OMs |
| D-846 | 3,4-di(trifluoromethylthio)phenyl | OMs |
| D-847 | 3,5-di(trifluoromethylthio)phenyl | OMs |
| D-848 | 2-fluoro-3-chlorophenyl | OMs |
| D-849 | 2-fluoro-4-chlorophenyl | OMs |
| D-850 | 2-fluoro-5-chlorophenyl | OMs |
| D-851 | 2-fluoro-6-chlorophenyl | OMs |
| D-852 | 3-fluoro-4-chlorophenyl | OMs |
| D-853 | 3-fluoro-5-chlorophenyl | OMs |
| D-854 | 2-chloro-3-fluorophenyl | OMs |
| D-855 | 2-chloro-4-fluorophenyl | OMs |
| D-856 | 2-chloro-5-fluorophenyl | OMs |
| D-857 | 3-chloro-4-fluorophenyl | OMs |
| D-858 | 2-methyl-3-chlorophenyl | OMs |
| D-859 | 2-methyl-4-chlorophenyl | OMs |
| D-860 | 2-methyl-5-chlorophenyl | OMs |
| D-861 | 2-methyl-6-chlorophenyl | OMs |
| D-862 | 3-methyl-4-chlorophenyl | OMs |
| D-863 | 3-methyl-5-chlorophenyl | OMs |
| D-864 | 2-chloro-3-methylphenyl | OMs |
| D-865 | 2-chloro-4-methylphenyl | OMs |
| D-866 | 2-chloro-5-methylphenyl | OMs |
| D-867 | 3-chloro-4-methylphenyl | OMs |
| D-868 | 2-methyl-3-fluorophenyl | OMs |
| D-869 | 2-methyl-4-fluorophenyl | OMs |
| D-870 | 2-methyl-5-fluorophenyl | OMs |
| D-871 | 2-methyl-6-fluorophenyl | OMs |
| D-872 | 3-methyl-4-fluorophenyl | OMs |
| D-873 | 3-methyl-5-fluorophenyl | OMs |
| D-874 | 2-fluoro-3-methylphenyl | OMs |
| D-875 | 2-fluoro-4-methylphenyl | OMs |
| D-876 | 2-fluoro-5-methylphenyl | OMs |
| D-877 | 3-fluoro-4-methylphenyl | OMs |
| D-878 | 2-chloro-3-ethylphenyl | OMs |
| D-879 | 2-chloro-4-ethylphenyl | OMs |
| D-880 | 2-chloro-5-ethylphenyl | OMs |
| D-881 | 3-chloro-4-ethylphenyl | OMs |
| D-882 | 2-ethyl-3-chlorophenyl | OMs |
| D-883 | 2-ethyl-4-chlorophenyl | OMs |
| D-884 | 2-ethyl-5-chlorophenyl | OMs |
| D-885 | 2-ethyl-6-chlorophenyl | OMs |
| D-886 | 2-ethyl-3-fluorophenyl | OMs |
| D-887 | 2-ethyl-4-fluorophenyl | OMs |
| D-888 | 2-ethyl-5-fluorophenyl | OMs |
| D-889 | 2-ethyl-6-fluorophenyl | OMs |
| D-890 | 3-ethyl-4-fluorophenyl | OMs |
| D-891 | 3-ethyl-5-fluorophenyl | OMs |
| D-892 | 2-fluoro-3-ethylphenyl | OMs |
| D-893 | 2-fluoro-4-ethylphenyl | OMs |
| D-894 | 2-fluoro-5-ethylphenyl | OMs |
| D-895 | 3-fluoro-4-ethylphenyl | OMs |
| D-896 | 2-methoxy-3-chlorophenyl | OMs |
| D-897 | 2-methoxy-4-chlorophenyl | OMs |
| D-898 | 2-methoxy-5-chlorophenyl | OMs |
| D-899 | 2-methoxy-6-chlorophenyl | OMs |
| D-900 | 3-methoxy-4-chlorophenyl | OMs |
| D-901 | 3-methoxy-5-chlorophenyl | OMs |
| D-902 | 2-chloro-3-methoxyphenyl | OMs |
| D-903 | 2-chloro-4-methoxyphenyl | OMs |
| D-904 | 2-chloro-5-methoxyphenyl | OMs |
| D-905 | 3-chloro-4-methoxyphenyl | OMs |
| D-906 | 2-methoxy-3-fluorophenyl | OMs |
| D-907 | 2-methoxy-4-fluorophenyl | OMs |
| D-908 | 2-methoxy-5-fluorophenyl | OMs |
| D-909 | 2-methoxy-6-fluorophenyl | OMs |
| D-910 | 3-methoxy-4-fluorophenyl | OMs |
| D-911 | 3-methoxy-5-fluorophenyl | OMs |
| D-912 | 2-fluoro-3-methoxyphenyl | OMs |
| D-913 | 2-fluoro-4-methoxyphenyl | OMs |
| D-914 | 2-fluoro-5-methoxyphenyl | OMs |
| D-915 | 3-fluoro-4-methoxyphenyl | OMs |
| D-916 | 3-fluoro-5-methoxyphenyl | OMs |
| D-917 | 2-(trifluoromethyl)-3-chlorophenyl | OMs |
| D-918 | 2-(trifluoromethyl)-4-chlorophenyl | OMs |
| D-919 | 2-(trifluoromethyl)-5-chlorophenyl | OMs |
| D-920 | 2-(trifluoromethyl)-6-chlorophenyl | OMs |
| D-921 | 3-(trifluoromethyl)-4-chlorophenyl | OMs |
| D-922 | 3-(trifluoromethyl)-5-chlorophenyl | OMs |
| D-923 | 2-chloro-3-(trifluoromethyl)phenyl | OMs |
| D-924 | 2-chloro-4-(trifluoromethyl)phenyl | OMs |
| D-925 | 2-chloro-5-(trifluoromethyl)phenyl | OMs |
| D-926 | 3-chloro-4-(trifluoromethyl)phenyl | OMs |
| D-927 | 2-(trifluoromethyl)-3-fluorophenyl | OMs |
| D-928 | 2-(trifluoromethyl)-4-fluorophenyl | OMs |
| D-929 | 2-(trifluoromethyl)-5-fluorophenyl | OMs |
| D-930 | 2-(trifluoromethyl)-6-fluorophenyl | OMs |
| D-931 | 3-(trifluoromethyl)-4-fluorophenyl | OMs |
| D-932 | 3-(trifluoromethyl)-5-fluorophenyl | OMs |
| D-933 | 2-fluoro-3-(trifluoromethyl)phenyl | OMs |
| D-934 | 2-fluoro-4-(trifluoromethyl)phenyl | OMs |
| D-935 | 2-fluoro-5-(trifluoromethyl)phenyl | OMs |
| D-936 | 3-fluoro-4-(trifluoromethyl)phenyl | OMs |
| D-937 | 2-(trifluoromethoxy)-3-chlorophenyl | OMs |
| D-938 | 2-(trifluoromethoxy)-4-chlorophenyl | OMs |
| D-939 | 2-(trifluoromethoxy)-5-chlorophenyl | OMs |
| D-940 | 2-(trifluoromethoxy)-6-chlorophenyl | OMs |
| D-941 | 3-(trifluoromethoxy)-4-chlorophenyl | OMs |
| D-942 | 3-(trifluoromethoxy)-5-chlorophenyl | OMs |
| D-943 | 2-chloro-3-(trifluoromethoxy)phenyl | OMs |
| D-944 | 2-chloro-4-(trifluoromethoxy)phenyl | OMs |
| D-945 | 2-chloro-5-(trifluoromethoxy)phenyl | OMs |
| D-946 | 3-chloro-4-(trifluoromethoxy)phenyl | OMs |
| D-947 | 2-(trifluoromethoxy)-3-fluorophenyl | OMs |
| D-948 | 2-(trifluoromethoxy)-4-fluorophenyl | OMs |
| D-949 | 2-(trifluoromethoxy)-5-fluorophenyl | OMs |
| D-950 | 2-(trifluoromethoxy)-6-fluorophenyl | OMs |
| D-951 | 3-(trifluoromethoxy)-4-fluorophenyl | OMs |
| D-952 | 3-(trifluoromethoxy)-5-fluorophenyl | OMs |
| D-953 | 2-fluoro-3-(trifluoromethoxy)phenyl | OMs |
| D-954 | 2-fluoro-4-(trifluoromethoxy)phenyl | OMs |
| D-955 | 2-fluoro-5-(trifluoromethoxy)phenyl | OMs |
| D-956 | 3-fluoro-4-(trifluoromethoxy)phenyl | OMs |
| D-957 | 2-(difluoromethoxy)-3-chlorophenyl | OMs |
| D-958 | 2-(difluoromethoxy)-4-chlorophenyl | OMs |
| D-959 | 2-(difluoromethoxy)-5-chlorophenyl | OMs |
| D-960 | 2-(difluoromethoxy)-6-chlorophenyl | OMs |
| D-961 | 3-(difluoromethoxy)-4-chlorophenyl | OMs |
| D-962 | 3-(difluoromethoxy)-5-chlorophenyl | OMs |
| D-963 | 2-chloro-3-(difluoromethoxy)phenyl | OMs |
| D-964 | 2-chloro-4-(difluoromethoxy)phenyl | OMs |
| D-965 | 2-chloro-5-(difluoromethoxy)phenyl | OMs |
| D-966 | 3-chloro-4-(difluoromethoxy)phenyl | OMs |
| D-967 | 2-(difluoromethoxy)-3-fluorophenyl | OMs |
| D-968 | 2-(difluoromethoxy)-4-fluorophenyl | OMs |
| D-969 | 2-(difluoromethoxy)-5-fluorophenyl | OMs |
| D-970 | 2-(difluoromethoxy)-6-fluorophenyl | OMs |
| D-971 | 3-(difluoromethoxy)-4-fluorophenyl | OMs |
| D-972 | 3-(difluoromethoxy)-5-fluorophenyl | OMs |
| D-973 | 2-fluoro-3-(difluoromethoxy)phenyl | OMs |
| D-974 | 2-fluoro-4-(difluoromethoxy)phenyl | OMs |
| D-975 | 2-fluoro-5-(difluoromethoxy)phenyl | OMs |
| D-976 | 3-fluoro-4-(difluoromethoxy)phenyl | OMs |
| D-977 | 2-(trifluoromethylthio)-3-chlorophenyl | OMs |
| D-978 | 2-(trifluoromethylthio)-4-chlorophenyl | OMs |
| D-979 | 2-(trifluoromethylthio)-5-chlorophenyl | OMs |
| D-980 | 2-(trifluoromethylthio)-6-chlorophenyl | OMs |
| D-981 | 3-(trifluoromethylthio)-4-chlorophenyl | OMs |
| D-982 | 3-(trifluoromethylthio)-5-chlorophenyl | OMs |
| D-983 | 2-chloro-3-(trifluoromethylthio)phenyl | OMs |
| D-984 | 2-chloro-4-(trifluoromethylthio)phenyl | OMs |
| D-985 | 2-chloro-5-(trifluoromethylthio)phenyl | OMs |
| D-986 | 3-chloro-4-(trifluoromethylthio)phenyl | OMs |
| D-987 | 2-(trifluoromethylthio)-3-fluorophenyl | OMs |
| D-988 | 2-(trifluoromethylthio)-4-fluorophenyl | OMs |
| D-989 | 2-(trifluoromethylthio)-5-fluorophenyl | OMs |
| D-990 | 2-(trifluoromethylthio)-6-fluorophenyl | OMs |
| D-991 | 3-(trifluoromethylthio)-4-fluorophenyl | OMs |
| D-992 | 3-(trifluoromethylthio)-5-fluorophenyl | OMs |
| D-993 | 2-fluoro-3-(trifluoromethylthio)phenyl | OMs |
| D-994 | 2-fluoro-4-(trifluoromethylthio)phenyl | OMs |
| D-995 | 2-fluoro-5-(trifluoromethylthio)phenyl | OMs |
| D-996 | 3-fluoro-4-(trifluoromethylthio)phenyl | OMs |
| D-997 | 2,3,4-trichlorophenyl | OMs |
| D-998 | 2,3,5-trichlorophenyl | OMs |
| D-999 | 2,3,6-trichlorophenyl | OMs |
| D-1000 | 2,4,5-trichlorophenyl | OMs |
| D-1001 | 2,4,6-trichlorophenyl | OMs |
| D-1002 | 3,4,5-trichlorophenyl | OMs |
| D-1003 | 2,3,4-trifluorophenyl | OMs |
| D-1004 | 2,3,5-trifluorophenyl | OMs |
| D-1005 | 2,3,6-trifluorophenyl | OMs |
| D-1006 | 2,4,5-trifluorophenyl | OMs |
| D-1007 | 2,4,6-trifluorophenyl | OMs |
| D-1008 | 3,4,5-trifluorophenyl | OMs |
| D-1009 | 2,3,4-trimethylphenyl | OMs |
| D-1010 | 2,3,5-trimethylphenyl | OMs |
| D-1011 | 2,3,6-trimethylphenyl | OMs |
| D-1012 | 2,4,5-trimethylphenyl | OMs |
| D-1013 | 2,4,6-trimethylphenyl | OMs |
| D-1014 | 3,4,5-trimethylphenyl | OMs |
| D-1015 | 2,3,4-trimethoxyphenyl | OMs |
| D-1016 | 2,3,5-trimethoxyphenyl | OMs |
| D-1017 | 2,3,6-trimethoxyphenyl | OMs |
| D-1018 | 2,4,5-trimethoxyphenyl | OMs |
| D-1019 | 2,4,6-trimethoxyphenyl | OMs |
| D-1020 | 3,4,5-trimethoxyphenyl | OMs |
| D-1021 | phenyl | OH |
| D-1022 | 2-chlorophenyl | OH |
| D-1023 | 3-chlorophenyl | OH |
| D-1024 | 4-chlorophenyl | OH |
| D-1025 | 2-fluorophenyl | OH |
| D-1026 | 3-fluorophenyl | OH |
| D-1027 | 4-fluorophenyl | OH |
| D-1028 | 2-methylphenyl | OH |
| D-1029 | 3-methylphenyl | OH |
| D-1030 | 4-methylphenyl | OH |
| D-1031 | 2-ethylphenyl | OH |
| D-1032 | 3-ethylphenyl | OH |
| D-1033 | 4-ethylphenyl | OH |
| D-1034 | 2-methoxyphenyl | OH |
| D-1035 | 3-methoxyphenyl | OH |
| D-1036 | 4-methoxyphenyl | OH |
| D-1037 | 2-trifluoromethylphenyl | OH |
| D-1038 | 3-trifluoromethylphenyl | OH |
| D-1039 | 4-trifluoromethylphenyl | OH |
| D-1040 | 2-trifluoromethoxyphenyl | OH |
| D-1041 | 3-trifluoromethoxyphenyl | OH |
| D-1042 | 4-trifluoromethoxyphenyl | OH |
| D-1043 | 2-difluoromethoxyphenyl | OH |
| D-1044 | 3-difluoromethoxyphenyl | OH |
| D-1045 | 4-difluoromethoxyphenyl | OH |
| D-1046 | 2-trifluoromethylthiophenyl | OH |
| D-1047 | 3-trifluoromethylthiophenyl | OH |
| D-1048 | 4-trifluoromethylthiophenyl | OH |
| D-1049 | 2,3-dichlorophenyl | OH |
| D-1050 | 2,4-dichlorophenyl | OH |
| D-1051 | 2,5-dichlorophenyl | OH |
| D-1052 | 2,6-dichlorophenyl | OH |
| D-1053 | 3,4-dichlorophenyl | OH |
| D-1054 | 3,5-dichlorophenyl | OH |
| D-1055 | 2,3-difluorophenyl | OH |
| D-1056 | 2,4-difluorophenyl | OH |
| D-1057 | 2,5-difluorophenyl | OH |
| D-1058 | 2,6-difluorophenyl | OH |
| D-1059 | 3,4-difluorophenyl | OH |
| D-1060 | 3,5-difluorophenyl | OH |
| D-1061 | 2,3-dimethylphenyl | OH |
| D-1062 | 2,4-dimethylphenyl | OH |
| D-1063 | 2,5-dimethylphenyl | OH |
| D-1064 | 2,6-dimethylphenyl | OH |
| D-1065 | 3,4-dimethylphenyl | OH |
| D-1066 | 3,5-dimethylphenyl | OH |
| D-1067 | 2,3-diethylphenyl | OH |
| D-1068 | 2,4-diethylphenyl | OH |
| D-1069 | 2,5-diethylphenyl | OH |
| D-1070 | 2,6-diethylphenyl | OH |
| D-1071 | 3,5-diethylphenyl | OH |
| D-1072 | 3,4-diethylphenyl | OH |
| D-1073 | 2,3-dimethoxyphenyl | OH |
| D-1074 | 2,4-dimethoxyphenyl | OH |
| D-1075 | 2,5-dimethoxyphenyl | OH |
| D-1076 | 2,6-dimethoxyphenyl | OH |
| D-1077 | 3,4-dimethoxyphenyl | OH |
| D-1078 | 3,5-dimethoxyphenyl | OH |
| D-1079 | 2,3-di(trifluoromethyl)phenyl | OH |
| D-1080 | 2,4-di(trifluoromethyl)phenyl | OH |
| D-1081 | 2,5-di(trifluoromethyl)phenyl | OH |
| D-1082 | 2,6-di(trifluoromethyl)phenyl | OH |
| D-1083 | 3,4-di(trifluoromethyl)phenyl | OH |
| D-1084 | 3,5-di(trifluoromethyl)phenyl | OH |
| D-1085 | 2,3-di(trifluoromethoxy)phenyl | OH |
| D-1086 | 2,4-di(trifluoromethoxy)phenyl | OH |
| D-1087 | 2,5-di(trifluoromethoxy)phenyl | OH |
| D-1088 | 2,6-di(trifluoromethoxy)phenyl | OH |
| D-1089 | 3,4-di(trifluoromethoxy)phenyl | OH |
| D-1090 | 3,5-di(trifluoromethoxy)phenyl | OH |
| D-1091 | 2,3-di(difluoromethoxy)phenyl | OH |
| D-1092 | 2,4-di(difluoromethoxy)phenyl | OH |
| D-1093 | 2,5-di(difluoromethoxy)phenyl | OH |
| D-1094 | 2,6-di(difluoromethoxy)phenyl | OH |
| D-1095 | 3,4-di(difluoromethoxy)phenyl | OH |
| D-1096 | 3,5-di(difluoromethoxy)phenyl | OH |
| D-1097 | 2,3-di(trifluoromethylthio)phenyl | OH |
| D-1098 | 2,4-di(trifluoromethylthio)phenyl | OH |
| D-1099 | 2,5-di(trifluoromethylthio)phenyl | OH |
| D-1100 | 2,6-di(trifluoromethylthio)phenyl | OH |
| D-1101 | 3,4-di(trifluoromethylthio)phenyl | OH |
| D-1102 | 3,5-di(trifluoromethylthio)phenyl | OH |
| D-1103 | 2-fluoro-3-chlorophenyl | OH |
| D-1104 | 2-fluoro-4-chlorophenyl | OH |
| D-1105 | 2-fluoro-5-chlorophenyl | OH |
| D-1106 | 2-fluoro-6-chlorophenyl | OH |
| D-1107 | 3-fluoro-4-chlorophenyl | OH |
| D-1108 | 3-fluoro-5-chlorophenyl | OH |
| D-1109 | 2-chloro-3-fluorophenyl | OH |
| D-1110 | 2-chloro-4-fluorophenyl | OH |
| D-1111 | 2-chloro-5-fluorophenyl | OH |
| D-1112 | 3-chloro-4-fluorophenyl | OH |
| D-1113 | 2-methyl-3-chlorophenyl | OH |
| D-1114 | 2-methyl-4-chlorophenyl | OH |
| D-1115 | 2-methyl-5-chlorophenyl | OH |
| D-1116 | 2-methyl-6-chlorophenyl | OH |
| D-1117 | 3-methyl-4-chlorophenyl | OH |
| D-1118 | 3-methyl-5-chlorophenyl | OH |
| D-1119 | 2-chloro-3-methylphenyl | OH |
| D-1120 | 2-chloro-4-methylphenyl | OH |
| D-1121 | 2-chloro-5-methylphenyl | OH |
| D-1122 | 3-chloro-4-methylphenyl | OH |
| D-1123 | 2-methyl-3-fluorophenyl | OH |
| D-1124 | 2-methyl-4-fluorophenyl | OH |
| D-1125 | 2-methyl-5-fluorophenyl | OH |
| D-1126 | 2-methyl-6-fluorophenyl | OH |
| D-1127 | 3-methyl-4-fluorophenyl | OH |
| D-1128 | 3-methyl-5-fluorophenyl | OH |
| D-1129 | 2-fluoro-3-methylphenyl | OH |
| D-1130 | 2-fluoro-4-methylphenyl | OH |
| D-1131 | 2-fluoro-5-methylphenyl | OH |
| D-1132 | 3-fluoro-4-methylphenyl | OH |
| D-1133 | 2-chloro-3-ethylphenyl | OH |
| D-1134 | 2-chloro-4-ethylphenyl | OH |
| D-1135 | 2-chloro-5-ethylphenyl | OH |
| D-1136 | 3-chloro-4-ethylphenyl | OH |
| D-1137 | 2-ethyl-3-chlorophenyl | OH |
| D-1138 | 2-ethyl-4-chlorophenyl | OH |
| D-1139 | 2-ethyl-5-chlorophenyl | OH |
| D-1140 | 2-ethyl-6-chlorophenyl | OH |
| D-1141 | 2-ethyl-3-fluorophenyl | OH |
| D-1142 | 2-ethyl-4-fluorophenyl | OH |
| D-1143 | 2-ethyl-5-fluorophenyl | OH |
| D-1144 | 2-ethyl-6-fluorophenyl | OH |
| D-1145 | 3-ethyl-4-fluorophenyl | OH |
| D-1146 | 3-ethyl-5-fluorophenyl | OH |
| D-1147 | 2-fluoro-3-ethylphenyl | OH |
| D-1148 | 2-fluoro-4-ethylphenyl | OH |
| D-1149 | 2-fluoro-5-ethylphenyl | OH |
| D-1150 | 3-fluoro-4-ethylphenyl | OH |
| D-1151 | 2-methoxy-3-chlorophenyl | OH |
| D-1152 | 2-methoxy-4-chlorophenyl | OH |
| D-1153 | 2-methoxy-5-chlorophenyl | OH |
| D-1154 | 2-methoxy-6-chlorophenyl | OH |
| D-1155 | 3-methoxy-4-chlorophenyl | OH |
| D-1156 | 3-methoxy-5-chlorophenyl | OH |
| D-1157 | 2-chloro-3-methoxyphenyl | OH |
| D-1158 | 2-chloro-4-methoxyphenyl | OH |
| D-1159 | 2-chloro-5-methoxyphenyl | OH |
| D-1160 | 3-chloro-4-methoxyphenyl | OH |
| D-1161 | 2-methoxy-3-fluorophenyl | OH |
| D-1162 | 2-methoxy-4-fluorophenyl | OH |
| D-1163 | 2-methoxy-5-fluorophenyl | OH |
| D-1164 | 2-methoxy-6-fluorophenyl | OH |
| D-1165 | 3-methoxy-4-fluorophenyl | OH |
| D-1166 | 3-methoxy-5-fluorophenyl | OH |
| D-1167 | 2-fluoro-3-methoxyphenyl | OH |
| D-1168 | 2-fluoro-4-methoxyphenyl | OH |
| D-1169 | 2-fluoro-5-methoxyphenyl | OH |
| D-1170 | 3-fluoro-4-methoxyphenyl | OH |
| D-1171 | 3-fluoro-5-methoxyphenyl | OH |
| D-1172 | 2-(trifluoromethyl)-3-chlorophenyl | OH |
| D-1173 | 2-(trifluoromethyl)-4-chlorophenyl | OH |
| D-1174 | 2-(trifluoromethyl)-5-chlorophenyl | OH |
| D-1175 | 2-(trifluoromethyl)-6-chlorophenyl | OH |
| D-1176 | 3-(trifluoromethyl)-4-chlorophenyl | OH |
| D-1177 | 3-(trifluoromethyl)-5-chlorophenyl | OH |
| D-1178 | 2-chloro-3-(trifluoromethyl)phenyl | OH |
| D-1179 | 2-chloro-4-(trifluoromethyl)phenyl | OH |
| D-1180 | 2-chloro-5-(trifluoromethyl)phenyl | OH |
| D-1181 | 3-chloro-4-(trifluoromethyl)phenyl | OH |
| D-1182 | 2-(trifluoromethyl)-3-fluorophenyl | OH |
| D-1183 | 2-(trifluoromethyl)-4-fluorophenyl | OH |
| D-1184 | 2-(trifluoromethyl)-5-fluorophenyl | OH |
| D-1185 | 2-(trifluoromethyl)-6-fluorophenyl | OH |
| D-1186 | 3-(trifluoromethyl)-4-fluorophenyl | OH |
| D-1187 | 3-(trifluoromethyl)-5-fluorophenyl | OH |
| D-1188 | 2-fluoro-3-(trifluoromethyl)phenyl | OH |
| D-1189 | 2-fluoro-4-(trifluoromethyl)phenyl | OH |
| D-1190 | 2-fluoro-5-(trifluoromethyl)phenyl | OH |
| D-1191 | 3-fluoro-4-(trifluoromethyl)phenyl | OH |
| D-1192 | 2-(trifluoromethoxy)-3-chlorophenyl | OH |
| D-1193 | 2-(trifluoromethoxy)-4-chlorophenyl | OH |
| D-1194 | 2-(trifluoromethoxy)-5-chlorophenyl | OH |
| D-1195 | 2-(trifluoromethoxy)-6-chlorophenyl | OH |
| D-1196 | 3-(trifluoromethoxy)-4-chlorophenyl | OH |
| D-1197 | 3-(trifluoromethoxy)-5-chlorophenyl | OH |
| D-1198 | 2-chloro-3-(trifluoromethoxy)phenyl | OH |
| D-1199 | 2-chloro-4-(trifluoromethoxy)phenyl | OH |
| D-1200 | 2-chloro-5-(trifluoromethoxy)phenyl | OH |
| D-1201 | 3-chloro-4-(trifluoromethoxy)phenyl | OH |
| D-1202 | 2-(trifluoromethoxy)-3-fluorophenyl | OH |
| D-1203 | 2-(trifluoromethoxy)-4-fluorophenyl | OH |
| D-1204 | 2-(trifluoromethoxy)-5-fluorophenyl | OH |
| D-1205 | 2-(trifluoromethoxy)-6-fluorophenyl | OH |
| D-1206 | 3-(trifluoromethoxy)-4-fluorophenyl | OH |
| D-1207 | 3-(trifluoromethoxy)-5-fluorophenyl | OH |
| D-1208 | 2-fluoro-3-(trifluoromethoxy)phenyl | OH |
| D-1209 | 2-fluoro-4-(trifluoromethoxy)phenyl | OH |
| D-1210 | 2-fluoro-5-(trifluoromethoxy)phenyl | OH |
| D-1211 | 3-fluoro-4-(trifluoromethoxy)phenyl | OH |
| D-1212 | 2-(difluoromethoxy)-3-chlorophenyl | OH |
| D-1213 | 2-(difluoromethoxy)-4-chlorophenyl | OH |
| D-1214 | 2-(difluoromethoxy)-5-chlorophenyl | OH |
| D-1215 | 2-(difluoromethoxy)-6-chlorophenyl | OH |
| D-1216 | 3-(difluoromethoxy)-4-chlorophenyl | OH |
| D-1217 | 3-(difluoromethoxy)-5-chlorophenyl | OH |
| D-1218 | 2-chloro-3-(difluoromethoxy)phenyl | OH |
| D-1219 | 2-chloro-4-(difluoromethoxy)phenyl | OH |
| D-1220 | 2-chloro-5-(difluoromethoxy)phenyl | OH |
| D-1221 | 3-chloro-4-(difluoromethoxy)phenyl | OH |
| D-1222 | 2-(difluoromethoxy)-3-fluorophenyl | OH |
| D-1223 | 2-(difluoromethoxy)-4-fluorophenyl | OH |
| D-1224 | 2-(difluoromethoxy)-5-fluorophenyl | OH |
| D-1225 | 2-(difluoromethoxy)-6-fluorophenyl | OH |
| D-1226 | 3-(difluoromethoxy)-4-fluorophenyl | OH |
| D-1227 | 3-(difluoromethoxy)-5-fluorophenyl | OH |
| D-1228 | 2-fluoro-3-(difluoromethoxy)phenyl | OH |
| D-1229 | 2-fluoro-4-(difluoromethoxy)phenyl | OH |
| D-1230 | 2-fluoro-5-(difluoromethoxy)phenyl | OH |
| D-1231 | 3-fluoro-4-(difluoromethoxy)phenyl | OH |
| D-1232 | 2-(trifluoromethylthio)-3-chlorophenyl | OH |
| D-1233 | 2-(trifluoromethylthio)-4-chlorophenyl | OH |
| D-1234 | 2-(trifluoromethylthio)-5-chlorophenyl | OH |
| D-1235 | 2-(trifluoromethylthio)-6-chlorophenyl | OH |
| D-1236 | 3-(trifluoromethylthio)-4-chlorophenyl | OH |
| D-1237 | 3-(trifluoromethylthio)-5-chlorophenyl | OH |
| D-1238 | 2-chloro-3-(trifluoromethylthio)phenyl | OH |
| D-1239 | 2-chloro-4-(trifluoromethylthio)phenyl | OH |
| D-1240 | 2-chloro-5-(trifluoromethylthio)phenyl | OH |
| D-1241 | 3-chloro-4-(trifluoromethylthio)phenyl | OH |
| D-1242 | 2-(trifluoromethylthio)-3-fluorophenyl | OH |
| D-1243 | 2-(trifluoromethylthio)-4-fluorophenyl | OH |
| D-1244 | 2-(trifluoromethylthio)-5-fluorophenyl | OH |
| D-1245 | 2-(trifluoromethylthio)-6-fluorophenyl | OH |
| D-1246 | 3-(trifluoromethylthio)-4-fluorophenyl | OH |
| D-1247 | 3-(trifluoromethylthio)-5-fluorophenyl | OH |
| D-1248 | 2-fluoro-3-(trifluoromethylthio)phenyl | OH |
| D-1249 | 2-fluoro-4-(trifluoromethylthio)phenyl | OH |
| D-1250 | 2-fluoro-5-(trifluoromethylthio)phenyl | OH |
| D-1251 | 3-fluoro-4-(trifluoromethylthio)phenyl | OH |
| D-1252 | 2,3,4-trichlorophenyl | OH |
| D-1253 | 2,3,5-trichlorophenyl | OH |
| D-1254 | 2,3,6-trichlorophenyl | OH |
| D-1255 | 2,4,5-trichlorophenyl | OH |
| D-1256 | 2,4,6-trichlorophenyl | OH |
| D-1257 | 3,4,5-trichlorophenyl | OH |
| D-1258 | 2,3,4-trifluorophenyl | OH |
| D-1259 | 2,3,5-trifluorophenyl | OH |
| D-1260 | 2,3,6-trifluorophenyl | OH |
| D-1261 | 2,4,5-trifluorophenyl | OH |
| D-1262 | 2,4,6-trifluorophenyl | OH |
| D-1263 | 3,4,5-trifluorophenyl | OH |
| D-1264 | 2,3,4-trimethylphenyl | OH |
| D-1265 | 2,3,5-trimethylphenyl | OH |
| D-1266 | 2,3,6-trimethylphenyl | OH |
| D-1267 | 2,4,5-trimethylphenyl | OH |
| D-1268 | 2,4,6-trimethylphenyl | OH |
| D-1269 | 3,4,5-trimethylphenyl | OH |
| D-1270 | 2,3,4-trimethoxyphenyl | OH |
| D-1271 | 2,3,5-trimethoxyphenyl | OH |
| D-1272 | 2,3,6-trimethoxyphenyl | OH |
| D-1273 | 2,4,5-trimethoxyphenyl | OH |
| D-1274 | 2,4,6-trimethoxyphenyl | OH |
| D-1275 | 3,4,5-trimethoxyphenyl | OH |

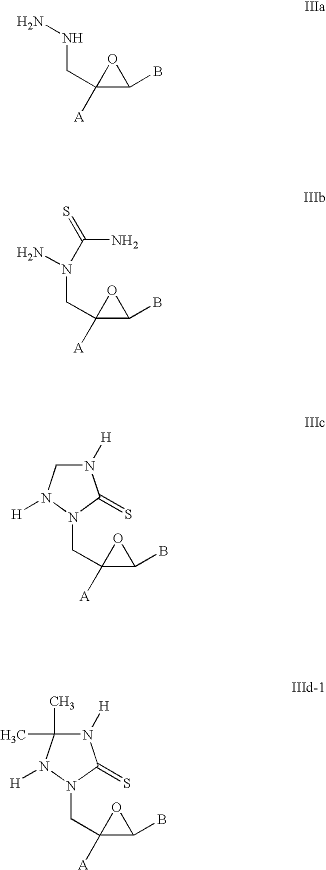

In particular with a view to their use, preference is given to the compounds IIIa, IIIb, IIIc and IIId-1 compiled in Tables 1e to 93e, Tables 1f to 93f, Tables 1 g to 93 g and Tables 1h to 93h, respectively, below. The groups mentioned in the tables for a substituent are furthermore per se, independently of the combination in which they are mentioned, a particularly preferred aspect of the substituent in question.

### Table 1e

Compounds IIIa, in which A is 2,3-difluorophenyl and B corresponds in each case to one row of Table B (compounds IIIa.1eB-1 to IIIa.1eB-255)

### Table 2e

Compounds IIIa, in which A is 2,4-difluorophenyl and B corresponds in each case to one row of Table B (compounds IIIa.2eB-1 to IIIa.2eB-255)

### Table 3e

Compounds IIIa, in which A is 2,5-difluorophenyl and B corresponds in each case to one row of Table B (compounds IIIa.3eB-1 to IIIa.3eB-255)

### Table 4e

Compounds IIIa, in which A is 2,6-difluorophenyl and B corresponds in each case to one row of Table B (compounds IIIa.4eB-1 to IIIa.4eB-255)

### Table 5e

Compounds IIIa, in which A is 3,4-difluorophenyl and B corresponds in each case to one row of Table B (compounds IIIa.5eB-1 to IIIa.5eB-255)

### Table 6e

Compounds IIIa, in which A is 3,5-difluorophenyl and B corresponds in each case to one row of Table B (compounds IIIa.6eB-1 to IIIa.6eB-255)

### Table 7e

Compounds IIIa, in which A is 2-fluoro-3-chlorophenyl and B corresponds in each case to one row of Table B (compounds IIIa.7eB-1 to IIIa.7eB-255)

### Table 8e

Compounds IIIa, in which A is 2-fluoro-4-chlorophenyl and B corresponds in each case to one row of Table B (compounds IIIa.8eB-1 to IIIa.8eB-255)

### Table 9e

Compounds IIIa, in which A is 2-fluoro-5-chlorophenyl and B corresponds in each case to one row of Table B (compounds IIIa.9eB-1 to IIIa.9eB-255)

### Table 10e

Compounds IIIa, in which A is 2-fluoro-6-chlorophenyl I and B corresponds in each case to one row of Table B (compounds IIIa.10eB-1 to IIIa.10eB-255)
   Table 11e Compounds IIIa, in which A is 3-fluoro-4-chlorophenyl and B corresponds in each case to one row of Table B (compounds IIIa.11eB-1 to IIIa.11eB-255)

### Table 12e

Compounds IIIa, in which A is 3-fluoro-5-chlorophenyl and B corresponds in each case to one row of Table B (compounds IIIa.12eB-1 to IIIa.12eB-255)

### Table 13e

Compounds IIIa, in which A is 2-chloro-3-fluorophenyl and B corresponds in each case to one row of Table B (compounds IIIa.13eB-1 to IIIa.13eB-255)

### Table 14e

Compounds IIIa, in which A is 2-chloro-4-fluorophenyl and B corresponds in each case to one row of Table B (compounds IIIa.14eB-1 to IIIa.14eB-255)

### Table 15e

Compounds IIIa, in which A is 2-chloro-5-fluorophenyl and B corresponds in each case to one row of Table B (compounds IIIa.15eB-1 to IIIa.15eB-255)

### Table 16e

Compounds IIIa, in which A is 3-chloro-4-fluorophenyl and B corresponds in each case to one row of Table B (compounds IIIa.16eB-1 to IIIa.16eB-255)

### Table 17e

Compounds IIIa, in which A is 2-methyl-3-fluorophenyl and B corresponds in each case to one row of Table B (compounds IIIa.17eB-1 to IIIa.17eB-255)

### Table 18e

Compounds IIIa, in which A is 2-methyl-4-fluorophenyl and B corresponds in each case to one row of Table B (compounds IIIa.18eB-1 to IIIa.18eB-255)

### Table 19e

Compounds IIIa, in which A is 2-methyl-5-fluorophenyl and B corresponds in each case to one row of Table B (compounds IIIa.19eB-1 to IIIa.19eB-255)

### Table 20e

Compounds IIIa, in which A is 2-methyl-6-fluorophenyl and B corresponds in each case to one row of Table B (compounds IIIa.20eB-1 to IIIa.20eB-255)

### Table 21e

Compounds IIIa, in which A is 3-methyl-4-fluorophenyl and B corresponds in each case to one row of Table B (compounds IIIa.21eB-1 to IIIa.21eB-255)

### Table 22e

Compounds IIIa, in which A is 3-methyl-5-fluorophenyl and B corresponds in each case to one row of Table B (compounds IIIa.22eB-1 to IIIa.22eB-255)

### Table 23e

Compounds IIIa, in which A is 2-fluoro-3-methylphenyl and B corresponds in each case to one row of Table B (compounds IIIa.23eB-1 to IIIa.23eB-255)

### Table 24e

Compounds IIIa, in which A is 2-fluoro-4-methylphenyl and B corresponds in each case to one row of Table B (compounds IIIa.24eB-1 to IIIa.24eB-255)

### Table 25e

Compounds IIIa, in which A is 2-fluoro-5-methylphenyl and B corresponds in each case to one row of Table B (compounds IIIa.25eB-1 to IIIa.25eB-255)

### Table 26e

Compounds IIIa, in which A is 3-fluoro-4-methylphenyl and B corresponds in each case to one row of Table B (compounds IIIa.26eB-1 to IIIa.26eB-255)

### Table 27e

Compounds IIIa, in which A is 2-ethyl-3-fluorophenyl and B corresponds in each case to one row of Table B (compounds IIIa.27eB-1 to IIIa.27eB-255)

### Table 28e

Compounds IIIa, in which A is 2-ethyl-4-fluorophenyl and B corresponds in each case to one row of Table B (compounds IIIa.28eB-1 to IIIa.28eB-255)

### Table 29e

Compounds IIIa, in which A is 2-ethyl-5-fluorophenyl and B corresponds in each case to one row of Table B (compounds IIIa.29eB-1 to IIIa.29eB-255)

### Table 30e

Compounds IIIa, in which A is 2-ethyl-6-fluorophenyl and B corresponds in each case to one row of Table B (compounds IIIa.30eB-1 to IIIa.30eB-255)

### Table 31e

Compounds IIIa, in which A is 3-ethyl-4-fluorophenyl and B corresponds in each case to one row of Table B (compounds IIIa.31eB-1 to IIIa.31eB-255)

### Table 32e

Compounds IIIa, in which A is 3-ethyl-5-fluorophenyl and B corresponds in each case to one row of Table B (compounds IIIa.32eB-1 to IIIa.32eB-255)

### Table 33e

Compounds IIIa, in which A is 2-fluoro-3-ethylphenyl and B corresponds in each case to one row of Table B (compounds IIIa.33eB-1 to IIIa.33eB-255)

### Table 34e

Compounds IIIa, in which A is 2-fluoro-4-ethylphenyl and B corresponds in each case to one row of Table B (compounds IIIa.34eB-1 to IIIa.34eB-255)

### Table 35e

Compounds IIIa, in which A is 2-fluoro-5-ethylphenyl and B corresponds in each case to one row of Table B (compounds IIIa.35eB-1 to IIIa.35eB-255)

### Table 36e

Compounds IIIa, in which A is 3-fluoro-4-ethylphenyl and B corresponds in each case to one row of Table B (compounds IIIa.36eB-1 to IIIa.36eB-255)

### Table 37e

Compounds IIIa, in which A is 2-methoxy-3-fluorophenyl and B corresponds in each case to one row of Table B (compounds IIIa.37eB-1 to IIIa.37eB-255)

### Table 38e

Compounds IIIa, in which A is 2-methoxy-4-fluorophenyl and B corresponds in each case to one row of Table B (compounds IIIa.38eB-1 to IIIa.38eB-255)

### Table 39e

Compounds IIIa, in which A is 2-methoxy-5-fluorophenyl and B corresponds in each case to one row of Table B (compounds IIIa.39eB-1 to IIIa.39eB-255)

### Table 40e

Compounds IIIa, in which A is 2-methoxy-6-fluorophenyl and B corresponds in each case to one row of Table B (compounds IIIa.40eB-1 to IIIa.40eB-255)

### Table 41e

Compounds IIIa, in which A is 3-methoxy-4-fluorophenyl and B corresponds in each case to one row of Table B (compounds IIIa.41eB-1 to IIIa.41eB-255)

### Table 42e

Compounds IIIa, in which A is 3-methoxy-5-fluorophenyl and B corresponds in each case to one row of Table B (compounds IIIa.42eB-1 to IIIa.42eB-255)

### Table 43e

Compounds IIIa, in which A is 2-fluoro-3-methoxyphenyl and B corresponds in each case to one row of Table B (compounds IIIa.43eB-1 to IIIa.43eB-255)

### Table 44e

Compounds IIIa, in which A is 2-fluoro-4-methoxyphenyl and B corresponds in each case to one row of Table B (compounds IIIa.44eB-1 to IIIa.44eB-255)

### Table 45e

Compounds IIIa, in which A is 2-fluoro-5-methoxyphenyl and B corresponds in each case to one row of Table B (compounds IIIa.45eB-1 to IIIa.45eB-255)

### Table 46e

Compounds IIIa, in which A is 3-fluoro-4-methoxyphenyl and B corresponds in each case to one row of Table B (compounds IIIa.46eB-1 to IIIa.46eB-255)

### Table 47e

Compounds IIIa, in which A is 3-fluoro-5-methoxyphenyl and B corresponds in each case to one row of Table B (compounds IIIa.47eB-1 to IIIa.47eB-255)

### Table 48e

Compounds IIIa, in which A is 2-(trifluoromethyl)-3-fluorophenyl and B corresponds in each case to one row of Table B (compounds IIIa.48eB-1 to IIIa.48eB-255)

### Table 49e

Compounds IIIa, in which A is 2-(trifluoromethyl)-4-fluorophenyl and B corresponds in each case to one row of Table B (compounds IIIa.49eB-1 to IIIa.49eB-255)

### Table 50e

Compounds IIIa, in which A is 2-(trifluoromethyl)-5-fluorophenyl and B corresponds in each case to one row of Table B (compounds IIIa.50eB-1 to IIIa.50eB-255)

### Table 51e

Compounds IIIa, in which A is 2-(trifluoromethyl)-6-fluorophenyl and B corresponds in each case to one row of Table B (compounds IIIa.51eB-1 to IIIa.51eB-255)

### Table 52e

Compounds IIIa, in which A is 3-(trifluoromethyl)-4-fluorophenyl and B corresponds in each case to one row of Table B (compounds IIIa.52eB-1 to IIIa.52eB-255)

### Table 53e

Compounds IIIa, in which A is 3-(trifluoromethyl)-5-fluorophenyl and B corresponds in each case to one row of Table B (compounds IIIa.53eB-1 to IIIa.53eB-255)

### Table 54e

Compounds IIIa, in which A is 2-fluoro-3-(trifluoromethyl)phenyl and B corresponds in each case to one row of Table B (compounds IIIa.54eB-1 to IIIa.54eB-255)

### Table 55e

Compounds IIIa, in which A is 2-fluoro-4-(trifluoromethyl)phenyl and B corresponds in each case to one row of Table B (compounds IIIa.55eB-1 to IIIa.55eB-255)

### Table 56e

Compounds IIIa, in which A is 2-fluoro-5-(trifluoromethyl)phenyl and B corresponds in each case to one row of Table B (compounds IIIa.56eB-1 to IIIa.56eB-255)

### Table 57e

Compounds IIIa, in which A is 3-fluoro-4-(trifluoromethyl)phenyl and B corresponds in each case to one row of Table B (compounds IIIa.57eB-1 to IIIa.57eB-255)

### Table 58e

Compounds IIIa, in which A is 2-(trifluoromethoxy)-3-fluorophenyl and B corresponds in each case to one row of Table B (compounds IIIa.58eB-1 to IIIa.58eB-255)

### Table 59e

Compounds IIIa, in which A is 2-(trifluoromethoxy)-4-fluorophenyl and B corresponds in each case to one row of Table B (compounds IIIa.59eB-1 to IIIa.59eB-255)

### Table 60e

Compounds IIIa, in which A is 2-(trifluoromethoxy)-5-fluorophenyl and B corresponds in each case to one row of Table B (compounds IIIa.60eB-1 to IIIa.60eB-255)

### Table 61e

Compounds IIIa, in which A is 2-(trifluoromethoxy)-6-fluorophenyl and B corresponds in each case to one row of Table B (compounds IIIa.61eB-1 to IIIa.61eB-255)

### Table 62e

Compounds IIIa, in which A is 3-(trifluoromethoxy)-4-fluorophenyl and B corresponds in each case to one row of Table B (compounds IIIa.62eB-1 to IIIa.62eB-255)

### Table 63e

Compounds IIIa, in which A is 3-(trifluoromethoxy)-5-fluorophenyl and B corresponds in each case to one row of Table B (compounds IIIa.63eB-1 to IIIa.63eB-255)

### Table 64e

Compounds IIIa, in which A is 2-fluoro-3-(trifluoromethoxy)phenyl and B corresponds in each case to one row of Table B (compounds IIIa.64eB-1 to IIIa.64eB-255)

### Table 65e

Compounds IIIa, in which A is 2-fluoro-4-(trifluoromethoxy)phenyl and B corresponds in each case to one row of Table B (compounds IIIa.65eB-1 to IIIa.65eB-255)

### Table 66e

Compounds IIIa, in which A is 2-fluoro-5-(trifluoromethoxy)phenyl and B corresponds in each case to one row of Table B (compounds IIIa.66eB-1 to IIIa.66eB-255)

### Table 67e

Compounds IIIa, in which A is 3-fluoro-4-(trifluoromethoxy)phenyl and B corresponds in each case to one row of Table B (compounds IIIa.67eB-1 to IIIa.67eB-255)

### Table 68e

Compounds IIIa, in which A is 2-(difluoromethoxy)-3-fluorophenyl and B corresponds in each case to one row of Table B (compounds IIIa.68eB-1 to IIIa.68eB-255)

### Table 69e

Compounds IIIa, in which A is 2-(difluoromethoxy)-4-fluorophenyl and B corresponds in each case to one row of Table B (compounds IIIa.69eB-1 to IIIa.69eB-255)

### Table 70e

Compounds IIIa, in which A is 2-(difluoromethoxy)-5-fluorophenyl and B corresponds in each case to one row of Table B (compounds IIIa.70eB-1 to IIIa.70eB-255)

### Table 71e

Compounds IIIa, in which A is 2-(difluoromethoxy)-6-fluorophenyl and B corresponds in each case to one row of Table B (compounds IIIa.71eB-1 to IIIa.71eB-255)

### Table 72e

Compounds IIIa, in which A is 3-(difluoromethoxy)-4-fluorophenyl and B corresponds in each case to one row of Table B (compounds IIIa.72eB-1 to IIIa.72eB-255)

### Table 73e

Compounds IIIa, in which A is 3-(difluoromethoxy)-5-fluorophenyl and B corresponds in each case to one row of Table B (compounds IIIa.73eB-1 to IIIa.73eB-255)

### Table 74e

Compounds IIIa, in which A is 2-fluoro-3-(difluoromethoxy)phenyl and B corresponds in each case to one row of Table B (compounds IIIa.74eB-1 to IIIa.74eB-255)

### Table 75e

Compounds IIIa, in which A is 2-fluoro-4-(difluoromethoxy)phenyl and B corresponds in each case to one row of Table B (compounds IIIa.75eB-1 to IIIa.75eB-255)

### Table 76e

Compounds IIIa, in which A is 2-fluoro-5-(difluoromethoxy)phenyl and B corresponds in each case to one row of Table B (compounds IIIa.76eB-1 to IIIa.76eB-255)

### Table 77e

Compounds IIIa, in which A is 3-fluoro-4-(difluoromethoxy)phenyl and B corresponds in each case to one row of Table B (compounds IIIa.77eB-1 to IIIa.77eB-255)

### Table 78e

Compounds IIIa, in which A is 2-(trifluoromethylthio)-3-fluorophenyl and B corresponds in each case to one row of Table B (compounds IIIa.78eB-1 to IIIa.78eB-255)

### Table 79e

Compounds IIIa, in which A is 2-(trifluoromethylthio)-4-fluorophenyl and B corresponds in each case to one row of Table B (compounds IIIa.79eB-1 to IIIa.79eB-255)

### Table 80e

Compounds IIIa, in which A is 2-(trifluoromethylthio)-5-fluorophenyl and B corresponds in each case to one row of Table B (compounds IIIa.80eB-1 to IIIa.80eB-255)

### Table 81e

Compounds IIIa, in which A is 2-(trifluoromethylthio)-6-fluorophenyl and B corresponds in each case to one row of Table B (compounds IIIa.81eB-1 to IIIa.81eB-255)

### Table 82e

Compounds IIIa, in which A is 3-(trifluoromethylthio)-4-fluorophenyl and B corresponds in each case to one row of Table B (compounds IIIa.82eB-1 to IIIa.82eB-255)

### Table 83e

Compounds IIIa, in which A is 3-(trifluoromethylthio)-5-fluorophenyl and B corresponds in each case to one row of Table B (compounds IIIa.83eB-1 to IIIa.83eB-255)

### Table 84e

Compounds IIIa, in which A is 2-fluoro-3-(trifluoromethylthio)phenyl and B corresponds in each case to one row of Table B (compounds IIIa.84eB-1 to IIIa.84eB-255)

### Table 85e

Compounds IIIa, in which A is 2-fluoro-4-(trifluoromethylthio)phenyl and B corresponds in each case to one row of Table B (compounds IIIa.85eB-1 to IIIa.85eB-255)

### Table 86e

Compounds IIIa, in which A is 2-fluoro-5-(trifluoromethylthio)phenyl and B corresponds in each case to one row of Table B (compounds IIIa.86eB-1 to IIIa.86eB-255)

### Table 87e

Compounds IIIa, in which A is 3-fluoro-4-(trifluoromethylthio)phenyl and B corresponds in each case to one row of Table B (compounds IIIa.87eB-1 to IIIa.87eB-255)

### Table 88e

Compounds IIIa, in which A is 2,3,4-trifluorophenyl and B corresponds in each case to one row of Table B (compounds IIIa.88eB-1 to IIIa.88eB-255)

### Table 89e

Compounds IIIa, in which A is 2,3,5-trifluorophenyl and B corresponds in each case to one row of Table B (compounds IIIa.89eB-1 to IIIa.89eB-255)

### Table 90e

Compounds IIIa, in which A is 2,3,6-trifluorophenyl and B corresponds in each case to one row of Table B (compounds IIIa.90eB-1 to IIIa.90eB-255)

### Table 91e

Compounds IIIa, in which A is 2,4,5-trifluorophenyl and B corresponds in each case to one row of Table B (compounds IIIa.91eB-1 to IIIa.91eB-255)

### Table 92e

Compounds IIIa, in which A is 2,4,6-trifluorophenyl and B corresponds in each case to one row of Table B (compounds IIIa.92eB-1 to IIIa.92eB-255)

### Table 93e

Compounds IIIa, in which A is 3,4,5-trifluorophenyl and B corresponds in each case to one row of Table B (compounds IIIa.93eB-1 to IIIa.93eB-255)

### Table 1f

Compounds IIIb, in which A is 2,3-difluorophenyl and B corresponds in each case to one row of Table B (compounds IIIb.1fB-1 to IIIb.1fB-255)

### Table 2f

Compounds IIIb, in which A is 2,4-difluorophenyl and B corresponds in each case to one row of Table B (compounds IIIb.2fB-1 to IIIb.2fB-255)

### Table 3f

Compounds IIIb, in which A is 2,5-difluorophenyl and B corresponds in each case to one row of Table B (compounds IIIb.3fB-1 to IIIb.3fB-255)

### Table 4f

Compounds IIIb, in which A is 2,6-difluorophenyl and B corresponds in each case to one row of Table B (compounds IIIb.4fB-1 to IIIb.4fB-255)

### Table 5f

Compounds IIIb, in which A is 3,4-difluorophenyl and B corresponds in each case to one row of Table B (compounds IIIb.5fB-1 to IIIb.5fB-255)

### Table 6f

Compounds IIIb, in which A is 3,5-difluorophenyl and B corresponds in each case to one row of Table B (compounds IIIb.6fB-1 to IIIb.6fB-255)

### Table 7f

Compounds IIIb, in which A is 2-fluoro-3-chlorophenyl and B corresponds in each case to one row of Table B (compounds IIIb.7fB-1 to IIIb.7fB-255)

### Table 8f

Compounds IIIb, in which A is 2-fluoro-4-chlorophenyl and B corresponds in each case to one row of Table B (compounds IIIb.8fB-1 to IIIb.8fB-255)

### Table 9f

Compounds IIIb, in which A is 2-fluoro-5-chlorophenyl and B corresponds in each case to one row of Table B (compounds IIIb.9fB-1 to IIIb.9fB-255)

### Table 10f

Compounds IIIb, in which A is 2-fluoro-6-chlorophenyl I and B corresponds in each case to one row of Table B (compounds IIIb.10fB-1 to IIIb.10fB-255)

### Table 11f

Compounds IIIb, in which A is 3-fluoro-4-chlorophenyl and B corresponds in each case to one row of Table B (compounds IIIb.11fB-1 to IIIb.11fB-255)

### Table 12f

Compounds IIIb, in which A is 3-fluoro-5-chlorophenyl and B corresponds in each case to one row of Table B (compounds IIIb.12fB-1 to IIIb.12fB-255)

### Table 13f

Compounds IIIb, in which A is 2-chloro-3-fluorophenyl and B corresponds in each case to one row of Table B (compounds IIIb.13fB-1 to IIIb.13fB-255)

### Table 14f

Compounds IIIb, in which A is 2-chloro-4-fluorophenyl and B corresponds in each case to one row of Table B (compounds IIIb.14fB-1 to IIIb.14fB-255)

### Table 15f

Compounds IIIb, in which A is 2-chloro-5-fluorophenyl and B corresponds in each case to one row of Table B (compounds IIIb.15fB-1 to IIIb.15fB-255)

### Table 16f

Compounds IIIb, in which A is 3-chloro-4-fluorophenyl and B corresponds in each case to one row of Table B (compounds IIIb.16fB-1 to IIIb.16fB-255)

### Table 17f

Compounds IIIb, in which A is 2-methyl-3-fluorophenyl and B corresponds in each case to one row of Table B (compounds IIIb.17fB-1 to IIIb.17fB-255)

### Table 18f

Compounds IIIb, in which A is 2-methyl-4-fluorophenyl and B corresponds in each case to one row of Table B (compounds IIIb.18fB-1 to IIIb.18fB-255)

### Table 19f

Compounds IIIb, in which A is 2-methyl-5-fluorophenyl and B corresponds in each case to one row of Table B (compounds IIIb.19fB-1 to IIIb.19fB-255)

### Table 20f

Compounds IIIb, in which A is 2-methyl-6-fluorophenyl and B corresponds in each case to one row of Table B (compounds IIIb.20fB-1 to IIIb.20fB-255)

### Table 21f

Compounds IIIb, in which A is 3-methyl-4-fluorophenyl and B corresponds in each case to one row of Table B (compounds IIIb.21fB-1 to IIIb.21fB-255)

### Table 22f

Compounds IIIb, in which A is 3-methyl-5-fluorophenyl and B corresponds in each case to one row of Table B (compounds IIIb.22fB-1 to IIIb.22fB-255)

### Table 23f

Compounds IIIb, in which A is 2-fluoro-3-methylphenyl and B corresponds in each case to one row of Table B (compounds IIIb.23fB-1 to IIIb.23fB-255)

### Table 24f

Compounds IIIb, in which A is 2-fluoro-4-methylphenyl and B corresponds in each case to one row of Table B (compounds IIIb.24fB-1 to IIIb.24fB-255)

### Table 25f

Compounds IIIb, in which A is 2-fluoro-5-methylphenyl and B corresponds in each case to one row of Table B (compounds IIIb.25fB-1 to IIIb.25fB-255)

### Table 26f

Compounds IIIb, in which A is 3-fluoro-4-methylphenyl and B corresponds in each case to one row of Table B (compounds IIIb.26fB-1 to IIIb.26fB-255)

### Table 27f

Compounds IIIb, in which A is 2-ethyl-3-fluorophenyl and B corresponds in each case to one row of Table B (compounds IIIb.27fB-1 to IIIb.27fB-255)

### Table 28f

Compounds IIIb, in which A is 2-ethyl-4-fluorophenyl and B corresponds in each case to one row of Table B (compounds IIIb.28fB-1 to IIIb.28fB-255)

### Table 29f

Compounds IIIb, in which A is 2-ethyl-5-fluorophenyl and B corresponds in each case to one row of Table B (compounds IIIb.29fB-1 to IIIb.29fB-255)

### Table 30f

Compounds IIIb, in which A is 2-ethyl-6-fluorophenyl and B corresponds in each case to one row of Table B (compounds IIIb.30fB-1 to IIIb.30fB-255)

### Table 31f

Compounds IIIb, in which A is 3-ethyl-4-fluorophenyl and B corresponds in each case to one row of Table B (compounds IIIb.31fB-1 to IIIb.31fB-255)

### Table 32f

Compounds IIIb, in which A is 3-ethyl-5-fluorophenyl and B corresponds in each case to one row of Table B (compounds IIIb.32fB-1 to IIIb.32fB-255)

### Table 33f

Compounds IIIb, in which A is 2-fluoro-3-ethylphenyl and B corresponds in each case to one row of Table B (compounds IIIb.33fB-1 to IIIb.33fB-255)

### Table 34f

Compounds IIIb, in which A is 2-fluoro-4-ethylphenyl and B corresponds in each case to one row of Table B (compounds IIIb.34fB-1 to IIIb.34fB-255)

### Table 35f

Compounds IIIb, in which A is 2-fluoro-5-ethylphenyl and B corresponds in each case to one row of Table B (compounds IIIb.35fB-1 to IIIb.35fB-255)

### Table 36f

Compounds IIIb, in which A is 3-fluoro-4-ethylphenyl and B corresponds in each case to one row of Table B (compounds IIIb.36fB-1 to IIIb.36fB-255)

### Table 37f

Compounds IIIb, in which A is 2-methoxy-3-fluorophenyl and B corresponds in each case to one row of Table B (compounds IIIb.37fB-1 to IIIb.37fB-255)

### Table 38f

Compounds IIIb, in which A is 2-methoxy-4-fluorophenyl and B corresponds in each case to one row of Table B (compounds IIIb.38fB-1 to IIIb.38fB-255)

### Table 39f

Compounds IIIb, in which A is 2-methoxy-5-fluorophenyl and B corresponds in each case to one row of Table B (compounds IIIb.39fB-1 to IIIb.39fB-255)

### Table 40f

Compounds IIIb, in which A is 2-methoxy-6-fluorophenyl and B corresponds in each case to one row of Table B (compounds IIIb.40fB-1 to IIIb.40fB-255)

### Table 41f

Compounds IIIb, in which A is 3-methoxy-4-fluorophenyl and B corresponds in each case to one row of Table B (compounds IIIb.41fB-1 to IIIb.41fB-255)

### Table 42f

Compounds IIIb, in which A is 3-methoxy-5-fluorophenyl and B corresponds in each case to one row of Table B (compounds IIIb.42fB-1 to IIIb.42fB-255)

### Table 43f

Compounds IIIb, in which A is 2-fluoro-3-methoxyphenyl and B corresponds in each case to one row of Table B (compounds IIIb.43fB-1 to IIIb.43fB-255)

### Table 44f

Compounds IIIb, in which A is 2-fluoro-4-methoxyphenyl and B corresponds in each case to one row of Table B (compounds IIIb.44fB-1 to IIIb.44fB-255)

### Table 45f

Compounds IIIb, in which A is 2-fluoro-5-methoxyphenyl and B corresponds in each case to one row of Table B (compounds IIIb.45fB-1 to IIIb.45fB-255)

### Table 46f

Compounds IIIb, in which A is 3-fluoro-4-methoxyphenyl and B corresponds in each case to one row of Table B (compounds IIIb.46fB-1 to IIIb.46fB-255)

### Table 47f

Compounds IIIb, in which A is 3-fluoro-5-methoxyphenyl and B corresponds in each case to one row of Table B (compounds IIIb.47fB-1 to IIIb.47fB-255)

### Table 48f

Compounds IIIb, in which A is 2-(trifluoromethyl)-3-fluorophenyl and B corresponds in each case to one row of Table B (compounds IIIb.48fB-1 to IIIb.48fB-255)

### Table 49f

Compounds IIIb, in which A is 2-(trifluoromethyl)-4-fluorophenyl and B corresponds in each case to one row of Table B (compounds IIIb.49fB-1 to IIIb.49fB-255)

### Table 50f

Compounds IIIb, in which A is 2-(trifluoromethyl)-5-fluorophenyl and B corresponds in each case to one row of Table B (compounds IIIb.50fB-1 to IIIb.50fB-255)

### Table 51f

Compounds IIIb, in which A is 2-(trifluoromethyl)-6-fluorophenyl and B corresponds in each case to one row of Table B (compounds IIIb.51fB-1 to IIIb.51fB-255)

### Table 52f

Compounds IIIb, in which A is 3-(trifluoromethyl)-4-fluorophenyl and B corresponds in each case to one row of Table B (compounds IIIb.52fB-1 to IIIb.52fB-255)

### Table 53f

Compounds IIIb, in which A is 3-(trifluoromethyl)-5-fluorophenyl and B corresponds in each case to one row of Table B (compounds IIIb.53fB-1 to IIIb.53fB-255)

### Table 54f

Compounds IIIb, in which A is 2-fluoro-3-(trifluoromethyl)phenyl and B corresponds in each case to one row of Table B (compounds IIIb.54fB-1 to IIIb.54fB-255)

### Table 55f

Compounds IIIb, in which A is 2-fluoro-4-(trifluoromethyl)phenyl and B corresponds in each case to one row of Table B (compounds IIIb.55fB-1 to IIIb.55fB-255)

### Table 56f

Compounds IIIb, in which A is 2-fluoro-5-(trifluoromethyl)phenyl and B corresponds in each case to one row of Table B (compounds IIIb.56fB-1 to IIIb.56fB-255)

### Table 57f

Compounds IIIb, in which A is 3-fluoro-4-(trifluoromethyl)phenyl and B corresponds in each case to one row of Table B (compounds IIIb.57fB-1 to IIIb.57fB-255)

### Table 58f

Compounds IIIb, in which A is 2-(trifluoromethoxy)-3-fluorophenyl and B corresponds in each case to one row of Table B (compounds IIIb.58fB-1 to IIIb.58fB-255)

### Table 59f

Compounds IIIb, in which A is 2-(trifluoromethoxy)-4-fluorophenyl and B corresponds in each case to one row of Table B (compounds IIIb.59fB-1 to IIIb.59fB-255)

### Table 60f

Compounds IIIb, in which A is 2-(trifluoromethoxy)-5-fluorophenyl and B corresponds in each case to one row of Table B (compounds IIIb.60fB-1 to IIIb.60fB-255)

### Table 61f

Compounds IIIb, in which A is 2-(trifluoromethoxy)-6-fluorophenyl and B corresponds in each case to one row of Table B (compounds IIIb.61fB-1 to IIIb.61fB-255)

### Table 62f

Compounds IIIb, in which A is 3-(trifluoromethoxy)-4-fluorophenyl and B corresponds in each case to one row of Table B (compounds IIIb.62fB-1 to IIIb.62fB-255)

### Table 63f

Compounds IIIb, in which A is 3-(trifluoromethoxy)-5-fluorophenyl and B corresponds in each case to one row of Table B (compounds IIIb.63fB-1 to IIIb.63fB-255)

### Table 64f

Compounds IIIb, in which A is 2-fluoro-3-(trifluoromethoxy)phenyl and B corresponds in each case to one row of Table B (compounds IIIb.64fB-1 to IIIb.64fB-255)

### Table 65f

Compounds IIIb, in which A is 2-fluoro-4-(trifluoromethoxy)phenyl and B corresponds in each case to one row of Table B (compounds IIIb.65fB-1 to IIIb.65fB-255)

### Table 66f

Compounds IIIb, in which A is 2-fluoro-5-(trifluoromethoxy)phenyl and B corresponds in each case to one row of Table B (compounds IIIb.66fB-1 to IIIb.66fB-255)

### Table 67f

Compounds IIIb, in which A is 3-fluoro-4-(trifluoromethoxy)phenyl and B corresponds in each case to one row of Table B (compounds IIIb.67fB-1 to IIIb.67fB-255)

### Table 68f

Compounds IIIb, in which A is 2-(difluoromethoxy)-3-fluorophenyl and B corresponds in each case to one row of Table B (compounds IIIb.68fB-1 to IIIb.68fB-255)

### Table 69f

Compounds IIIb, in which A is 2-(difluoromethoxy)-4-fluorophenyl and B corresponds in each case to one row of Table B (compounds IIIb.69fB-1 to IIIb.69fB-255)

### Table 70f

Compounds IIIb, in which A is 2-(difluoromethoxy)-5-fluorophenyl and B corresponds in each case to one row of Table B (compounds IIIb.70fB-1 to IIIb.70fB-255)

### Table 71f

Compounds IIIb, in which A is 2-(difluoromethoxy)-6-fluorophenyl and B corresponds in each case to one row of Table B (compounds IIIb.71fB-1 to IIIb.71fB-255)

### Table 72f

Compounds IIIb, in which A is 3-(difluoromethoxy)-4-fluorophenyl and B corresponds in each case to one row of Table B (compounds IIIb.72fB-1 to IIIb.72fB-255)

### Table 73f

Compounds IIIb, in which A is 3-(difluoromethoxy)-5-fluorophenyl and B corresponds in each case to one row of Table B (compounds IIIb.73fB-1 to IIIb.73fB-255)

### Table 74f

Compounds IIIb, in which A is 2-fluoro-3-(difluoromethoxy)phenyl and B corresponds in each case to one row of Table B (compounds IIIb.74fB-1 to IIIb.74fB-255)

### Table 75f

Compounds IIIb, in which A is 2-fluoro-4-(difluoromethoxy)phenyl and B corresponds in each case to one row of Table B (compounds IIIb.75fB-1 to IIIb.75fB-255)

### Table 76f

Compounds IIIb, in which A is 2-fluoro-5-(difluoromethoxy)phenyl and B corresponds in each case to one row of Table B (compounds IIIb.76fB-1 to IIIb.76fB-255)

### Table 77f

Compounds IIIb, in which A is 3-fluoro-4-(difluoromethoxy)phenyl and B corresponds in each case to one row of Table B (compounds IIIb.77fB-1 to IIIb.77fB-255)

### Table 78f

Compounds IIIb, in which A is 2-(trifluoromethylthio)-3-fluorophenyl and B corresponds in each case to one row of Table B (compounds IIIb.78fB-1 to IIIb.78fB-255)

### Table 79f

Compounds IIIb, in which A is 2-(trifluoromethylthio)-4-fluorophenyl and B corresponds in each case to one row of Table B (compounds IIIb.79fB-1 to IIIb.79fB-255)

### Table 80f

Compounds IIIb, in which A is 2-(trifluoromethylthio)-5-fluorophenyl and B corresponds in each case to one row of Table B (compounds IIIb.80fB-1 to IIIb.80f8-255)

### Table 81f

Compounds IIIb, in which A is 2-(trifluoromethylthio)-6-fluorophenyl and B corresponds in each case to one row of Table B (compounds IIIb.81fB-1 to IIIb.81fB-255)

### Table 82f

Compounds IIIb, in which A is 3-(trifluoromethylthio)-4-fluorophenyl and B corresponds in each case to one row of Table B (compounds IIIb.82fB-1 to IIIb.82fB-255)

### Table 83f

Compounds IIIb, in which A is 3-(trifluoromethylthio)-5-fluorophenyl and B corresponds in each case to one row of Table B (compounds IIIb.83fB-1 to IIIb.83fB-255)

### Table 84f

Compounds IIIb, in which A is 2-fluoro-3-(trifluoromethylthio)phenyl and B corresponds in each case to one row of Table B (compounds IIIb.84fB-1 to IIIb.84fB-255)

### Table 85f

Compounds IIIb, in which A is 2-fluoro-4-(trifluoromethylthio)phenyl and B corresponds in each case to one row of Table B (compounds IIIb.85fB-1 to IIIb.85fB-255)

### Table 86f

Compounds IIIb, in which A is 2-fluoro-5-(trifluoromethylthio)phenyl and B corresponds in each case to one row of Table B (compounds IIIb.86fB-1 to IIIb.86fB-255)

### Table 87f

Compounds IIIb, in which A is 3-fluoro-4-(trifluoromethylthio)phenyl and B corresponds in each case to one row of Table B (compounds IIIb.87fB-1 to IIIb.87fB-255)

### Table 88f

Compounds IIIb, in which A is 2,3,4-trifluorophenyl and B corresponds in each case to one row of Table B (compounds IIIb.88fB-1 to IIIb.88fB-255)

### Table 89f

Compounds IIIb, in which A is 2,3,5-trifluorophenyl and B corresponds in each case to one row of Table B (compounds IIIb.89fB-1 to IIIb.89fB-255)

### Table 90f

Compounds IIIb, in which A is 2,3,6-trifluorophenyl and B corresponds in each case to one row of Table B (compounds IIIb.90fB-1 to IIIb.90fB-255)

### Table 91f

Compounds IIIb, in which A is 2,4,5-trifluorophenyl and B corresponds in each case to one row of Table B (compounds IIIb.91fB-1 to IIIb.91fB-255)

### Table 92f

Compounds IIIb, in which A is 2,4,6-trifluorophenyl and B corresponds in each case to one row of Table B (compounds IIIb.92fB-1 to IIIb.92fB-255)

### Table 93f

Compounds IIIb, in which A is 3,4,5-trifluorophenyl and B corresponds in each case to one row of Table B (compounds IIIb.93fB-1 to IIIb.93fB-255)

### Table 1g

Compounds IIIc, in which A is 2,3-difluorophenyl and B corresponds in each case to one row of Table B (compounds IIIc.1gB-1 to IIIc.1gB-255)

### Table 2g

Compounds IIIc, in which A is 2,4-difluorophenyl and B corresponds in each case to one row of Table B (compounds IIIc.2gB-1 to IIIc.2gB-255)

### Table 3g

Compounds IIIc, in which A is 2,5-difluorophenyl and B corresponds in each case to one row of Table B (compounds IIIc.3gB-1 to IIIc.3gB-255)

### Table 4g

Compounds IIIc, in which A is 2,6-difluorophenyl and B corresponds in each case to one row of Table B (compounds IIIc.4gB-1 to IIIc.4gB-255)

### Table 5g

Compounds IIIc, in which A is 3,4-difluorophenyl and B corresponds in each case to one row of Table B (compounds IIIc.5gB-1 to IIIc.5gB-255)

### Table 6g

Compounds IIIc, in which A is 3,5-difluorophenyl and B corresponds in each case to one row of Table B (compounds IIIc.6gB-1 to IIIc.6gB-255)

### Table 7g

Compounds IIIc, in which A is 2-fluoro-3-chlorophenyl and B corresponds in each case to one row of Table B (compounds IIIc.7gB-1 to IIIc.7gB-255)

### Table 8g

Compounds IIIc, in which A is 2-fluoro-4-chlorophenyl and B corresponds in each case to one row of Table B (compounds IIIc.8gB-1 to IIIc.8gB-255)

### Table 9g

Compounds IIIc, in which A is 2-fluoro-5-chlorophenyl and B corresponds in each case to one row of Table B (compounds IIIc.9gB-1 to IIIc.9gB-255)

### Table 10g

Compounds IIIc, in which A is 2-fluoro-6-chlorophenyl I and B corresponds in each case to one row of Table B (compounds IIIc.10gB-1 to IIIc.10gB-255)

### Table 11g

Compounds IIIc, in which A is 3-fluoro-4-chlorophenyl and B corresponds in each case to one row of Table B (compounds IIIc.11gB-1 to IIIc.11gB-255)

### Table 12g

Compounds IIIc, in which A is 3-fluoro-5-chlorophenyl and B corresponds in each case to one row of Table B (compounds IIIc.12gB-1 to IIIc.12gB-255)

### Table 13g

Compounds IIIc, in which A is 2-chloro-3-fluorophenyl and B corresponds in each case to one row of Table B (compounds IIIc.13gB-1 to IlIc.13gB-255)

### Table 14g

Compounds IIIc, in which A is 2-chloro-4-fluorophenyl and B corresponds in each case to one row of Table B (compounds IIIc.14gB-1 to IIIc.14gB-255)

### Table 15g

Compounds IIIc, in which A is 2-chloro-5-fluorophenyl and B corresponds in each case to one row of Table B (compounds IIIc.15gB-1 to IIIc.15gB-255)

### Table 16g

Compounds IIIc, in which A is 3-chloro-4-fluorophenyl and B corresponds in each case to one row of Table B (compounds IIIc.16gB-1 to IIIc.16gB-255)

### Table 17g

Compounds IIIc, in which A is 2-methyl-3-fluorophenyl and B corresponds in each case to one row of Table B (compounds IIIc.17gB-1 to IIIc.17gB-255)

### Table 18g

Compounds IIIc, in which A is 2-methyl-4-fluorophenyl and B corresponds in each case to one row of Table B (compounds IIIc.18gB-1 to IIIc.18gB-255)

### Table 19g

Compounds IIIc, in which A is 2-methyl-5-fluorophenyl and B corresponds in each case to one row of Table B (compounds IIIc.19gB-1 to IIIc.19gB-255)

### Table 20g

Compounds IIIc, in which A is 2-methyl-6-fluorophenyl and B corresponds in each case to one row of Table B (compounds IIIc.20gB-1 to IIIc.20gB-255)

### Table 21g

Compounds IIIc, in which A is 3-methyl-4-fluorophenyl and B corresponds in each case to one row of Table B (compounds IIIc.21gB-1 to IIIc.21gB-255)

### Table 22g

Compounds IIIc, in which A is 3-methyl-5-fluorophenyl and B corresponds in each case to one row of Table B (compounds IIIc.22gB-1 to IIIc.22gB-255)

### Table 23g

Compounds IIIc, in which A is 2-fluoro-3-methylphenyl and B corresponds in each case to one row of Table B (compounds IIIc.23gB-1 to IIIc.23gB-255)

### Table 24g

Compounds IIIc, in which A is 2-fluoro-4-methylphenyl and B corresponds in each case to one row of Table B (compounds IIIc.24gB-1 to IIIc.24gB-255)

### Table 25g

Compounds IIIc, in which A is 2-fluoro-5-methylphenyl and B corresponds in each case to one row of Table B (compounds IIIc.25gB-1 to IIIc.25gB-255)

### Table 26g

Compounds IIIc, in which A is 3-fluoro-4-methylphenyl and B corresponds in each case to one row of Table B (compounds IIIc.26gB-1 to IIIc.26gB-255)

### Table 27g

Compounds IIIc, in which A is 2-ethyl-3-fluorophenyl and B corresponds in each case to one row of Table B (compounds IIIc.27gB-1 to IIIc.27gB-255)

### Table 28g

Compounds IIIc, in which A is 2-ethyl-4-fluorophenyl and B corresponds in each case to one row of Table B (compounds IIIc.28gB-1 to IIIc.28gB-255)

### Table 29g

Compounds IIIc, in which A is 2-ethyl-5-fluorophenyl and B corresponds in each case to one row of Table B (compounds IIIc.29gB-1 to IIIc.29gB-255)

### Table 30g

Compounds IIIc, in which A is 2-ethyl-6-fluorophenyl and B corresponds in each case to one row of Table B (compounds IIIc.30gB-1 to IIIc.30gB-255)

### Table 31g

Compounds IIIc, in which A is 3-ethyl-4-fluorophenyl and B corresponds in each case to one row of Table B (compounds IIIc.31gB-1 to IIIc.31gB-255)

### Table 32g

Compounds IIIc, in which A is 3-ethyl-5-fluorophenyl and B corresponds in each case to one row of Table B (compounds IIIc.32gB-1 to IIIc.32gB-255)

### Table 33g

Compounds IIIc, in which A is 2-fluoro-3-ethylphenyl and B corresponds in each case to one row of Table B (compounds IIIc.33gB-1 to IIIc.33gB-255)

### Table 34g

Compounds IIIc, in which A is 2-fluoro-4-ethylphenyl and B corresponds in each case to one row of Table B (compounds IIIc.34gB-1 to IIIc.34gB-255)

### Table 35g

Compounds IIIc, in which A is 2-fluoro-5-ethylphenyl and B corresponds in each case to one row of Table B (compounds IIIc.35gB-1 to IIIc.35gB-255)

### Table 36g

Compounds IIIc, in which A is 3-fluoro-4-ethylphenyl and B corresponds in each case to one row of Table B (compounds IIIc.36gB-1 to IIIc.36gB-255)

### Table 37g

Compounds IIIc, in which A is 2-methoxy-3-fluorophenyl and B corresponds in each case to one row of Table B (compounds IIIc.37gB-1 to IIIc.37gB-255)

### Table 38g

Compounds IIIc, in which A is 2-methoxy-4-fluorophenyl and B corresponds in each case to one row of Table B (compounds IIIc.38gB-1 to IIIc.38gB-255)

### Table 39g

Compounds IIIc, in which A is 2-methoxy-5-fluorophenyl and B corresponds in each case to one row of Table B (compounds IIIc.39gB-1 to IIIc.39gB-255)

### Table 40g

Compounds IIIc, in which A is 2-methoxy-6-fluorophenyl and B corresponds in each case to one row of Table B (compounds IIIc.40gB-1 to IIIc.40gB-255)

### Table 41g

Compounds IIIc, in which A is 3-methoxy-4-fluorophenyl and B corresponds in each case to one row of Table B (compounds IIIc.41gB-1 to IIIc.41gB-255)

### Table 42g

Compounds IIIc, in which A is 3-methoxy-5-fluorophenyl and B corresponds in each case to one row of Table B (compounds IIIc.42gB-1 to IIIc.42gB-255)

### Table 43g

Compounds IIIc, in which A is 2-fluoro-3-methoxyphenyl and B corresponds in each case to one row of Table B (compounds IIIc.43gB-1 to IIIc.43gB-255)

### Table 44g

Compounds IIIc, in which A is 2-fluoro-4-methoxyphenyl and B corresponds in each case to one row of Table B (compounds IIIc.44gB-1 to IIIc.44gB-255)

### Table 45g

Compounds IIIc, in which A is 2-fluoro-5-methoxyphenyl and B corresponds in each case to one row of Table B (compounds IIIc.45gB-1 to IIIc.45gB-255)

### Table 46g

Compounds IIIc, in which A is 3-fluoro-4-methoxyphenyl and B corresponds in each case to one row of Table B (compounds IIIc.46gB-1 to IIIc.46gB-255)

### Table 47g

Compounds IIIc, in which A is 3-fluoro-5-methoxyphenyl and B corresponds in each case to one row of Table B (compounds IIIc.47gB-1 to IIIc.47gB-255)

### Table 48g

Compounds IIIc, in which A is 2-(trifluoromethyl)-3-fluorophenyl and B corresponds in each case to one row of Table B (compounds IIIc.48gB-1 to IIIc.48gB-255)

### Table 49g

Compounds IIIc, in which A is 2-(trifluoromethyl)-4-fluorophenyl and B corresponds in each case to one row of Table B (compounds IIIc.49gB-1 to IIIc.49gB-255)

### Table 50g

Compounds IIIc, in which A is 2-(trifluoromethyl)-5-fluorophenyl and B corresponds in each case to one row of Table B (compounds IIIc.50gB-1 to IIIc.50gB-255)

### Table 51g

Compounds IIIc, in which A is 2-(trifluoromethyl)-6-fluorophenyl and B corresponds in each case to one row of Table B (compounds IIIc.51gB-1 to IIIc.51gB-255)

### Table 52g

Compounds IIIc, in which A is 3-(trifluoromethyl)-4-fluorophenyl and B corresponds in each case to one row of Table B (compounds IIIc.52gB-1 to IIIc.52gB-255)

### Table 53g

Compounds IIIc, in which A is 3-(trifluoromethyl)-5-fluorophenyl and B corresponds in each case to one row of Table B (compounds IIIc.53gB-1 to IIIc.53gB-255)

### Table 54g

Compounds IIIc, in which A is 2-fluoro-3-(trifluoromethyl)phenyl and B corresponds in each case to one row of Table B (compounds IIIc.54gB-1 to IIIc.54gB-255)

### Table 55g

Compounds IIIc, in which A is 2-fluoro-4-(trifluoromethyl)phenyl and B corresponds in each case to one row of Table B (compounds IIIc.55gB-1 to IIIc.55gB-255)

### Table 56g

Compounds IIIc, in which A is 2-fluoro-5-(trifluoromethyl)phenyl and B corresponds in each case to one row of Table B (compounds IIIc.56gB-1 to IIIc.56gB-255)

### Table 57g

Compounds IIIc, in which A is 3-fluoro-4-(trifluoromethyl)phenyl and B corresponds in each case to one row of Table B (compounds IIIc.57gB-1 to IIIc.57gB-255)

### Table 58g

Compounds IIIc, in which A is 2-(trifluoromethoxy)-3-fluorophenyl and B corresponds in each case to one row of Table B (compounds IIIc.58gB-1 to IIIc.58gB-255)

### Table 59g

Compounds IIIc, in which A is 2-(trifluoromethoxy)-4-fluorophenyl and B corresponds in each case to one row of Table B (compounds IIIc.59gB-1 to IIIc.59gB-255)

### Table 60g

Compounds IIIc, in which A is 2-(trifluoromethoxy)-5-fluorophenyl and B corresponds in each case to one row of Table B (compounds IIIc.60gB-1 to IIIc.60gB-255)

### Table 61g

Compounds IIIc, in which A is 2-(trifluoromethoxy)-6-fluorophenyl and B corresponds in each case to one row of Table B (compounds IIIc.61gB-1 to IIIc.61gB-255)

### Table 62g

Compounds IIIc, in which A is 3-(trifluoromethoxy)-4-fluorophenyl and B corresponds in each case to one row of Table B (compounds IIIc.62gB-1 to IIIc.62gB-255)

### Table 63g

Compounds IIIc, in which A is 3-(trifluoromethoxy)-5-fluorophenyl and B corresponds in each case to one row of Table B (compounds IIIc.63gB-1 to IIIc.63gB-255)

### Table 64g

Compounds IIIc, in which A is 2-fluoro-3-(trifluoromethoxy)phenyl and B corresponds in each case to one row of Table B (compounds IIIc.64gB-1 to IIIc.64gB-255)

### Table 65g

Compounds IIIc, in which A is 2-fluoro-4-(trifluoromethoxy)phenyl and B corresponds in each case to one row of Table B (compounds IIIc.65gB-1 to IIIc.65gB-255)

### Table 66g

Compounds IIIc, in which A is 2-fluoro-5-(trifluoromethoxy)phenyl and B corresponds in each case to one row of Table B (compounds IIIc.66gB-1 to IIIc.66gB-255)

### Table 67g

Compounds IIIc, in which A is 3-fluoro-4-(trifluoromethoxy)phenyl and B corresponds in each case to one row of Table B (compounds IIIc.67gB-1 to IIIc.67gB-255)

### Table 68g

Compounds IIIc, in which A is 2-(difluoromethoxy)-3-fluorophenyl and B corresponds in each case to one row of Table B (compounds IIIc.68gB-1 to IIIc.68gB-255)

### Table 69g

Compounds IIIc, in which A is 2-(difluoromethoxy)-4-fluorophenyl and B corresponds in each case to one row of Table B (compounds IIIc.69gB-1 to IIIc.69gB-255)

### Table 70g

Compounds IIIc, in which A is 2-(difluoromethoxy)-5-fluorophenyl and B corresponds in each case to one row of Table B (compounds IIIc.70gB-1 to IIIc.70gB-255)

### Table 71g

Compounds IIIc, in which A is 2-(difluoromethoxy)-6-fluorophenyl and B corresponds in each case to one row of Table B (compounds IIIc.71gB-1 to IIIc.71gB-255)

### Table 72g

Compounds IIIc, in which A is 3-(difluoromethoxy)-4-fluorophenyl and B corresponds in each case to one row of Table B (compounds IIIc.72gB-1 to IIIc.72gB-255)

### Table 73g

Compounds IIIc, in which A is 3-(difluoromethoxy)-5-fluorophenyl and B corresponds in each case to one row of Table B (compounds IIIc.73gB-1 to IIIc.73gB-255)

### Table 74g

Compounds IIIc, in which A is 2-fluoro-3-(difluoromethoxy)phenyl and B corresponds in each case to one row of Table B (compounds IIIc.74gB-1 to IIIc.74gB-255)

### Table 75g

Compounds IIIc, in which A is 2-fluoro-4-(difluoromethoxy)phenyl and B corresponds in each case to one row of Table B (compounds IIIc.75gB-1 to IIIc.75gB-255)

### Table 76g

Compounds IIIc, in which A is 2-fluoro-5-(difluoromethoxy)phenyl and B corresponds in each case to one row of Table B (compounds IIIc.76gB-1 to IIIc.76gB-255)

### Table 77g

Compounds IIIc, in which A is 3-fluoro-4-(difluoromethoxy)phenyl and B corresponds in each case to one row of Table B (compounds IIIc.77gB-1 to IIIc.77gB-255)

### Table 78g

Compounds IIIc, in which A is 2-(trifluoromethylthio)-3-fluorophenyl and B corresponds in each case to one row of Table B (compounds IIIc.78gB-1 to IIIc.78gB-255)

### Table 79g

Compounds IIIc, in which A is 2-(trifluoromethylthio)-4-fluorophenyl and B corresponds in each case to one row of Table B (compounds IIIc.79gB-1 to IIIc.79gB-255)

### Table 80g

Compounds IIIc, in which A is 2-(trifluoromethylthio)-5-fluorophenyl and B corresponds in each case to one row of Table B (compounds IIIc.80gB-1 to IIIc.80gB-255)

### Table 81g

Compounds IIIc, in which A is 2-(trifluoromethylthio)-6-fluorophenyl and B corresponds in each case to one row of Table B (compounds IIIc.81gB-1 to IIIc.81gB-255)

### Table 82g

Compounds IIIc, in which A is 3-(trifluoromethylthio)-4-fluorophenyl and B corresponds in each case to one row of Table B (compounds IIIc.82gB-1 to IIIc.82gB-255)

### Table 83g

Compounds IIIc, in which A is 3-(trifluoromethylthio)-5-fluorophenyl and B corresponds in each case to one row of Table B (compounds IIIc.83gB-1 to IIIc.83gB-255)

### Table 84g

Compounds IIIc, in which A is 2-fluoro-3-(trifluoromethylthio)phenyl and B corresponds in each case to one row of Table B (compounds IIIc.84gB-1 to IIIc.84gB-255)

### Table 85g

Compounds IIIc, in which A is 2-fluoro-4-(trifluoromethylthio)phenyl and B corresponds in each case to one row of Table B (compounds IIIc.85gB-1 to IIIc.85gB-255)

### Table 86g

Compounds IIIc, in which A is 2-fluoro-5-(trifluoromethylthio)phenyl and B corresponds in each case to one row of Table B (compounds IIIc.86gB-1 to IIIc.86gB-255)

### Table 87g

Compounds IIIc, in which A is 3-fluoro-4-(trifluoromethylthio)phenyl and B corresponds in each case to one row of Table B (compounds IIIc.87gB-1 to IIIc.87gB-255)

### Table 88g

Compounds IIIc, in which A is 2,3,4-trifluorophenyl and B corresponds in each case to one row of Table B (compounds IIIc.88gB-1 to IIIc.88gB-255)

### Table 89g

Compounds IIIc, in which A is 2,3,5-trifluorophenyl and B corresponds in each case to one row of Table B (compounds IIIc.89gB-1 to IIIc.89gB-255)

### Table 90g

Compounds IIIc, in which A is 2,3,6-trifluorophenyl and B corresponds in each case to one row of Table B (compounds IIIc.90gB-1 to IIIc.90gB-255)

### Table 91g

Compounds IIIc, in which A is 2,4,5-trifluorophenyl and B corresponds in each case to one row of Table B (compounds IIIc.91gB-1 to IIIc.91gB-255)

### Table 92g

Compounds IIIc, in which A is 2,4,6-trifluorophenyl and B corresponds in each case to one row of Table B (compounds IIIc.92gB-1 to IIIc.92gB-255)

### Table 93g

Compounds IIIc, in which A is 3,4,5-trifluorophenyl and B corresponds in each case to one row of Table B (compounds IIIc.93gB-1 to IIIc.93gB-255)

### Table 1h

Compounds IIId-1, in which A is 2,3-difluorophenyl and B corresponds in each case to one row of Table B (compounds IIId-1.1hB-1 to IIId-1.1hB-255)

### Table 2h

Compounds IIId-1, in which A is 2,4-difluorophenyl and B corresponds in each case to one row of Table B (compounds IIId-1.2hB-1 to IIId-1.2hB-255)

### Table 3h

Compounds IIId-1, in which A is 2,5-difluorophenyl and B corresponds in each case to one row of Table B (compounds IIId-1.3hB-1 to IIId-1.3hB-255)

### Table 4h

Compounds IIId-1, in which A is 2,6-difluorophenyl and B corresponds in each case to one row of Table B (compounds IIId-1.4hB-1 to IIId-1.4hB-255)

### Table 5h

Compounds IIId-1, in which A is 3,4-difluorophenyl and B corresponds in each case to one row of Table B (compounds IIId-1.5hB-1 to IIId-1.5hB-255)

### Table 6h

Compounds IIId-1, in which A is 3,5-difluorophenyl and B corresponds in each case to one row of Table B (compounds IIId-1.6hB-1 to IIId-1.6hB-255)

### Table 7h

Compounds IIId-1, in which A is 2-fluoro-3-chlorophenyl and B corresponds in each case to one row of Table B (compounds IIId-1.7hB-1 to IIId-1.7hB-255)

### Table 8h

Compounds IIId-1, in which A is 2-fluoro-4-chlorophenyl and B corresponds in each case to one row of Table B (compounds IIId-1.8hB-1 to IIId-1.8hB-255)

### Table 9h

Compounds IIId-1, in which A is 2-fluoro-5-chlorophenyl and B corresponds in each case to one row of Table B (compounds IIId-1.9hB-1 to IIId-1.9hB-255)

### Table 10h

Compounds IIId-1, in which A is 2-fluoro-6-chlorophenyl I and B corresponds in each case to one row of Table B (compounds IIId-1.10hB-1 to IIId-1.10hB-255)

### Table 11h

Compounds IIId-1, in which A is 3-fluoro-4-chlorophenyl and B corresponds in each case to one row of Table B (compounds IIId-1.11hB-1 to IIId-1.11hB-255)

### Table 12h

Compounds IIId-1, in which A is 3-fluoro-5-chlorophenyl and B corresponds in each case to one row of Table B (compounds IIId-1.12hB-1 to IIId-1.12hB-255)

### Table 13h

Compounds IIId-1, in which A is 2-chloro-3-fluorophenyl and B corresponds in each case to one row of Table B (compounds IIId-1.13hB-1 to IIId-1.13hB-255)

### Table 14h

Compounds IIId-1, in which A is 2-chloro-4-fluorophenyl and B corresponds in each case to one row of Table B (compounds IIId-1.14hB-1 to IIId-1.14hB-255)

### Table 15h

Compounds IIId-1, in which A is 2-chloro-5-fluorophenyl and B corresponds in each case to one row of Table B (compounds IIId-1.15hB-1 to IIId-1.15hB-255)

### Table 16h

Compounds IIId-1, in which A is 3-chloro-4-fluorophenyl and B corresponds in each case to one row of Table B (compounds IIId-1.16hB-1 to IIId-1.16hB-255)

### Table 17h

Compounds IIId-1, in which A is 2-methyl-3-fluorophenyl and B corresponds in each case to one row of Table B (compounds IIId-1.17hB-1 to IIId-1.17hB-255)

### Table 18h

Compounds IIId-1, in which A is 2-methyl-4-fluorophenyl and B corresponds in each case to one row of Table B (compounds IIId-1.18hB-1 to IIId-1.18hB-255)

### Table 19h

Compounds IIId-1, in which A is 2-methyl-5-fluorophenyl and B corresponds in each case to one row of Table B (compounds IIId-1.19hB-1 to IIId-1.19hB-255)

### Table 20h

Compounds IIId-1, in which A is 2-methyl-6-fluorophenyl and B corresponds in each case to one row of Table B (compounds IIId-1.20hB-1 to IIId-1.20hB-255)

### Table 21h

Compounds IIId-1, in which A is 3-methyl-4-fluorophenyl and B corresponds in each case to one row of Table B (compounds IIId-1.21hB-1 to IIId-1.21hB-255)

### Table 22h

Compounds IIId-1, in which A is 3-methyl-5-fluorophenyl and B corresponds in each case to one row of Table B (compounds IIId-1.22hB-1 to IIId-1.22hB-255)

### Table 23h

Compounds IIId-1, in which A is 2-fluoro-3-methylphenyl and B corresponds in each case to one row of Table B (compounds IIId-1.23hB-1 to IIId-1.23hB-255)

### Table 24h

Compounds IIId-1, in which A is 2-fluoro-4-methylphenyl and B corresponds in each case to one row of Table B (compounds IIId-1.24hB-1 to IIId-1.24hB-255)

### Table 25h

Compounds IIId-1, in which A is 2-fluoro-5-methylphenyl and B corresponds in each case to one row of Table B (compounds IIId-1.25hB-1 to IIId-1.25hB-255)

### Table 26h

Compounds IIId-1, in which A is 3-fluoro-4-methylphenyl and B corresponds in each case to one row of Table B (compounds IIId-1.26hB-1 to IIId-1.26hB-255)

### Table 27h

Compounds IIId-1, in which A is 2-ethyl-3-fluorophenyl and B corresponds in each case to one row of Table B (compounds IIId-1.27hB-1 to IIId-1.27hB-255)

### Table 28h

Compounds IIId-1, in which A is 2-ethyl-4-fluorophenyl and B corresponds in each case to one row of Table B (compounds IIId-1.28hB-1 to IIId-1.28hB-255)

### Table 29h

Compounds IIId-1, in which A is 2-ethyl-5-fluorophenyl and B corresponds in each case to one row of Table B (compounds IIId-1.29hB-1 to IIId-1.29hB-255)

### Table 30h

Compounds IIId-1, in which A is 2-ethyl-6-fluorophenyl and B corresponds in each case to one row of Table B (compounds IIId-1.30hB-1 to IIId-1.30hB-255)

### Table 31h

Compounds IIId-1, in which A is 3-ethyl-4-fluorophenyl and B corresponds in each case to one row of Table B (compounds IIId-1.31hB-1 to IIId-1.31hB-255)

### Table 32h

Compounds IIId-1, in which A is 3-ethyl-5-fluorophenyl and B corresponds in each case to one row of Table B (compounds IIId-1.32hB-1 to IIId-1.32hB-255)

### Table 33h

Compounds IIId-1, in which A is 2-fluoro-3-ethylphenyl and B corresponds in each case to one row of Table B (compounds IIId-1.33hB-1 to IIId-1.33hB-255)

### Table 34h

Compounds IIId-1, in which A is 2-fluoro-4-ethylphenyl and B corresponds in each case to one row of Table B (compounds IIId-1.34hB-1 to IIId-1.34hB-255)

### Table 35h

Compounds IIId-1, in which A is 2-fluoro-5-ethylphenyl and B corresponds in each case to one row of Table B (compounds IIId-1.35hB-1 to IIId-1.35hB-255)

### Table 36h

Compounds IIId-1, in which A is 3-fluoro-4-ethylphenyl and B corresponds in each case to one row of Table B (compounds IIId-1.36hB-1 to IIId-1.36hB-255)

### Table 37h

Compounds IIId-1, in which A is 2-methoxy-3-fluorophenyl and B corresponds in each case to one row of Table B (compounds IIId-1.37hB-1 to IIId-1.37hB-255)

### Table 38h

Compounds IIId-1, in which A is 2-methoxy-4-fluorophenyl and B corresponds in each case to one row of Table B (compounds IIId-1.38hB-1 to IIId-1.38hB-255)

### Table 39h

Compounds IIId-1, in which A is 2-methoxy-5-fluorophenyl and B corresponds in each case to one row of Table B (compounds IIId-1.39hB-1 to IIId-1.39hB-255)

### Table 40h

Compounds IIId-1, in which A is 2-methoxy-6-fluorophenyl and B corresponds in each case to one row of Table B (compounds IIId-1.40hB-1 to IIId-1.40hB-255)

### Table 41h

Compounds IIId-1, in which A is 3-methoxy-4-fluorophenyl and B corresponds in each case to one row of Table B (compounds IIId-1.41hB-1 to IIId-1.41hB-255)

### Table 42h

Compounds IIId-1, in which A is 3-methoxy-5-fluorophenyl and B corresponds in each case to one row of Table B (compounds IIId-1.42hB-1 to IIId-1.42hB-255)

### Table 43h

Compounds IIId-1, in which A is 2-fluoro-3-methoxyphenyl and B corresponds in each case to one row of Table B (compounds IIId-1.43hB-1 to IIId-1.43hB-255)

### Table 44h

Compounds IIId-1, in which A is 2-fluoro-4-methoxyphenyl and B corresponds in each case to one row of Table B (compounds IIId-1.44hB-1 to IIId-1.44hB-255)

### Table 45h

Compounds IIId-1, in which A is 2-fluoro-5-methoxyphenyl and B corresponds in each case to one row of Table B (compounds IIId-1.45hB-1 to IIId-1.45hB-255)

### Table 46h

Compounds IIId-1, in which A is 3-fluoro-4-methoxyphenyl and B corresponds in each case to one row of Table B (compounds IIId-1.46hB-1 to IIId-1.46hB-255)

### Table 47h

Compounds IIId-1, in which A is 3-fluoro-5-methoxyphenyl and B corresponds in each case to one row of Table B (compounds IIId-1.47hB-1 to IIId-1.47hB-255)

### Table 48h

Compounds IIId-1, in which A is 2-(trifluoromethyl)-3-fluorophenyl and B corresponds in each case to one row of Table B (compounds IIId-1.48hB-1 to IIId-1.48hB-255)

### Table 49h

Compounds IIId-1, in which A is 2-(trifluoromethyl)-4-fluorophenyl and B corresponds in each case to one row of Table B (compounds IIId-1.49hB-1 to IIId-1.49hB-255)

### Table 50h

Compounds IIId-1, in which A is 2-(trifluoromethyl)-5-fluorophenyl and B corresponds in each case to one row of Table B (compounds IIId-1.50hB-1 to IIId-1.50hB-255)

### Table 51h

Compounds IIId-1, in which A is 2-(trifluoromethyl)-6-fluorophenyl and B corresponds in each case to one row of Table B (compounds IIId-1.51hB-1 to IIId-1.51hB-255)

### Table 52h

Compounds IIId-1, in which A is 3-(trifluoromethyl)-4-fluorophenyl and B corresponds in each case to one row of Table B (compounds IIId-1.52hB-1 to IIId-1.52hB-255)

### Table 53h

Compounds IIId-1, in which A is 3-(trifluoromethyl)-5-fluorophenyl and B corresponds in each case to one row of Table B (compounds IIId-1.53hB-1 to IIId-1.53hB-255)

### Table 54h

Compounds IIId-1, in which A is 2-fluoro-3-(trifluoromethyl)phenyl and B corresponds in each case to one row of Table B (compounds IIId-1.54hB-1 to IIId-1.54hB-255)

### Table 55h

Compounds IIId-1, in which A is 2-fluoro-4-(trifluoromethyl)phenyl and B corresponds in each case to one row of Table B (compounds IIId-1.55hB-1 to IIId-1.55hB-255)

### Table 56h

Compounds IIId-1, in which A is 2-fluoro-5-(trifluoromethyl)phenyl and B corresponds in each case to one row of Table B (compounds IIId-1.56hB-1 to IIId-1.56hB-255)

### Table 57h

Compounds IIId-1, in which A is 3-fluoro-4-(trifluoromethyl)phenyl and B corresponds in each case to one row of Table B (compounds IIId-1.57hB-1 to IIId-1.57hB-255)

### Table 58h

Compounds IIId-1, in which A is 2-(trifluoromethoxy)-3-fluorophenyl and B corresponds in each case to one row of Table B (compounds IIId-1.58hB-1 to IIId-1.58hB-255)

### Table 59h

Compounds IIId-1, in which A is 2-(trifluoromethoxy)-4-fluorophenyl and B corresponds in each case to one row of Table B (compounds IIId-1.59hB-1 to IIId-1.59hB-255)

### Table 60h

Compounds IIId-1, in which A is 2-(trifluoromethoxy)-5-fluorophenyl and B corresponds in each case to one row of Table B (compounds IIId-1.60hB-1 to IIId-1.60hB-255)

### Table 61h

Compounds IIId-1, in which A is 2-(trifluoromethoxy)-6-fluorophenyl and B corresponds in each case to one row of Table B (compounds IIId-1.61hB-1 to IIId-1.61hB-255)

### Table 62h

Compounds IIId-1, in which A is 3-(trifluoromethoxy)-4-fluorophenyl and B corresponds in each case to one row of Table B (compounds IIId-1.62hB-1 to IIId-1.62hB-255)

### Table 63h

Compounds IIId-1, in which A is 3-(trifluoromethoxy)-5-fluorophenyl and B corresponds in each case to one row of Table B (compounds IIId-1.63hB-1 to IIId-1.63hB-255)

### Table 64h

Compounds IIId-1, in which A is 2-fluoro-3-(trifluoromethoxy)phenyl and B corresponds in each case to one row of Table B (compounds IIId-1.64hB-1 to IIId-1.64hB-255)

### Table 65h

Compounds IIId-1, in which A is 2-fluoro-4-(trifluoromethoxy)phenyl and B corresponds in each case to one row of Table B (compounds IIId-1.65hB-1 to IIId-1.65hB-255)

### Table 66h

Compounds IIId-1, in which A is 2-fluoro-5-(trifluoromethoxy)phenyl and B corresponds in each case to one row of Table B (compounds IIId-1.66hB-1 to IIId-1.66hB-255)

### Table 67h

Compounds IIId-1, in which A is 3-fluoro-4-(trifluoromethoxy)phenyl and B corresponds in each case to one row of Table B (compounds IIId-1.67hB-1 to IIId-1.67hB-255)

### Table 68h

Compounds IIId-1, in which A is 2-(difluoromethoxy)-3-fluorophenyl and B corresponds in each case to one row of Table B (compounds IIId-1.68hB-1 to IIId-1.68hB-255)

### Table 69h

Compounds IIId-1, in which A is 2-(difluoromethoxy)-4-fluorophenyl and B corresponds in each case to one row of Table B (compounds IIId-1.69hB-1 to IIId-1.69hB-255)

### Table 70h

Compounds IIId-1, in which A is 2-(difluoromethoxy)-5-fluorophenyl and B corresponds in each case to one row of Table B (compounds IIId-1.70hB-1 to IIId-1.70hB-255)

### Table 71h

Compounds IIId-1, in which A is 2-(difluoromethoxy)-6-fluorophenyl and B corresponds in each case to one row of Table B (compounds IIId-1.71hB-1 to IIId-1.71hB-255)

### Table 72h

Compounds IIId-1, in which A is 3-(difluoromethoxy)-4-fluorophenyl and B corresponds in each case to one row of Table B (compounds IIId-1.72hB-1 to IIId-1.72hB-255)

### Table 73h

Compounds IIId-1, in which A is 3-(difluoromethoxy)-5-fluorophenyl and B corresponds in each case to one row of Table B (compounds IIId-1.73hB-1 to IIId-1.73hB-255)

### Table 74h

Compounds IIId-1, in which A is 2-fluoro-3-(difluoromethoxy)phenyl and B corresponds in each case to one row of Table B (compounds IIId-1.74hB-1 to IIId-1.74hB-255)

### Table 75h

Compounds IIId-1, in which A is 2-fluoro-4-(difluoromethoxy)phenyl and B corresponds in each case to one row of Table B (compounds IIId-1.75hB-1 to IIId-1.75hB-255)

### Table 76h

Compounds IIId-1, in which A is 2-fluoro-5-(difluoromethoxy)phenyl and B corresponds in each case to one row of Table B (compounds IIId-1.76hB-1 to IIId-1.76hB-255)

### Table 77h

Compounds IIId-1, in which A is 3-fluoro-4-(difluoromethoxy)phenyl and B corresponds in each case to one row of Table B (compounds IIId-1.77hB-1 to IIId-1.77hB-255)

### Table 78h

Compounds IIId-1, in which A is 2-(trifluoromethylthio)-3-fluorophenyl and B corresponds in each case to one row of Table B (compounds IIId-1.78hB-1 to IIId-1.78hB-255)

### Table 79h

Compounds IIId-1, in which A is 2-(trifluoromethylthio)-4-fluorophenyl and B corresponds in each case to one row of Table B (compounds IIId-1.79hB-1 to IIId-1.79hB-255)

### Table 80h

Compounds IIId-1, in which A is 2-(trifluoromethylthio)-5-fluorophenyl and B corresponds in each case to one row of Table B (compounds IIId-1.80hB-1 to IIId-1.80hB-255)

### Table 81h

Compounds IIId-1, in which A is 2-(trifluoromethylthio)-6-fluorophenyl and B corresponds in each case to one row of Table B (compounds IIId-1.81hB-1 to IIId-1.81hB-255)

### Table 82h

Compounds IIId-1, in which A is 3-(trifluoromethylthio)-4-fluorophenyl and B corresponds in each case to one row of Table B (compounds IIId-1.82hB-1 to IIId-1.82hB-255)

### Table 83h

Compounds IIId-1, in which A is 3-(trifluoromethylthio)-5-fluorophenyl and B corresponds in each case to one row of Table B (compounds IIId-1.83hB-1 to IIId-1.83hB-255)

### Table 84h

Compounds IIId-1, in which A is 2-fluoro-3-(trifluoromethylthio)phenyl and B corresponds in each case to one row of Table B (compounds IIId-1.84hB-1 to IIId-1.84hB-255)

### Table 85h

Compounds IIId-1, in which A is 2-fluoro-4-(trifluoromethylthio)phenyl and B corresponds in each case to one row of Table B (compounds IIId-1.85hB-1 to IIId-1.85hB-255)

### Table 86h

Compounds IIId-1, in which A is 2-fluoro-5-(trifluoromethylthio)phenyl and B corresponds in each case to one row of Table B (compounds IIId-1.86hB-1 to IIId-1.86hB-255)

### Table 87h

Compounds IIId-1, in which A is 3-fluoro-4-(trifluoromethylthio)phenyl and B corresponds in each case to one row of Table B (compounds IIId-1.87hB-1 to IIId-1.87hB-255)

### Table 88h

Compounds IIId-1, in which A is 2,3,4-trifluorophenyl and B corresponds in each case to one row of Table B (compounds IIId-1.88hB-1 to IIId-1.88hB-255)

### Table 89h

Compounds IIId-1, in which A is 2,3,5-trifluorophenyl and B corresponds in each case to one row of Table B (compounds IIId-1.89hB-1 to IIId-1.89hB-255)

### Table 90h

Compounds IIId-1, in which A is 2,3,6-trifluorophenyl and B corresponds in each case to one row of Table B (compounds IIId-1.90hB-1 to IIId-1.90hB-255)

### Table 91h

Compounds IIId-1, in which A is 2,4,5-trifluorophenyl and B corresponds in each case to one row of Table B (compounds IIId-1.91hB-1 to IIId-1.91hB-255)

### Table 92h

Compounds IIId-1, in which A is 2,4,6-trifluorophenyl and B corresponds in each case to one row of Table B (compounds IIId-1.92hB-1 to IIId-1.92hB-255)

### Table 93h

Compounds IIId-1, in which A is 3,4,5-trifluorophenyl and B corresponds in each case to one row of Table B (compounds IIId-1.93hB-1 to IIId-1.93hB-255)

From the tables above, the compound names for the individual compounds can be derived as follows: the “compound 1.3aA-10” (emphases added), for example, is the compound of the formula I according to the invention in which A is 2,5-difluorophenyl (as stated in Table 3a), B is 4-methylphenyl and D is SH (as stated in row 10 of Table A).

The compounds according to the invention, in particular the compounds of the formulae I and II, and their compositions according to the invention are suitable as fungicides for controlling harmful fungi. They are distinguished by excellent activity against a broad spectrum of phytopathogenic fungi including soilborne pathogens which originate in particular from the classes of the Plasmodiophoramycetes, Peronosporomycetes (syn. Oomycetes), Chytridiomycetes, Zygomycetes, Ascomycetes, Basidiomycetes and Deuteromycetes (syn. Fungi imperfecti). Some of them are systemically active and can be used in crop protection as foliar fungicides, fungicides for seed dressing and soil fungicides. In addition, they are suitable for controlling fungi which, inter alia, attack the wood or the roots of plants.

The compounds according to the invention are of particular importance for the control of a large number of pathogenic fungi on various crop plants such as cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currents or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw material plants, for example corn, soybeans, wheat, oilseed rape, sugar cane or oil palms; corn; tobacco; nuts; coffee; tea; bananas; grape vines (grapes for eating and grapes for wine making); hops; grass, for example lawns; rubber plants; ornamental and forest plants, for example flowers, shrubs, deciduous trees and coniferous trees, and also on the propagation material, for example seeds, and on the harvested material of these plants.

Preferably, the compounds I according to the invention and the compositions according to the invention are used for controlling a large number of fungal pathogens in agricultural crops, for example potatoes, sugar beets, tobacco, wheat, rye, barley, oats, rice, corn, cotton, soybeans, oilseed rape, leguminous plants, sunflowers, coffee or sugarcane; fruit plants, grapevines and ornamental plants and vegetables, for example cucumbers, tomatoes, beans and cucurbits and also on the propagation material, for example seeds, and the harvested products of these plants.

The term “plant propagation materials” includes all generative parts of the plant, for example seeds, and vegetative plant parts, such as seedlings and tubers (for example potatoes) which can be utilized for propagating a plant. These include seeds, roots, fruits, tubers, bulbs, rhizomes, shoots and other plant parts including seedlings and young plants which are transplanted after germination or after emergence. The young plants can be protected by partial or complete treatment, for example by immersion or watering, against harmful fungi.

The treatment of plant propagation materials with compounds I or the compositions according to the invention is used for controlling a large number of fungal pathogens in cereal crops, for example wheat, rye, barley or oats; rice, corn, cotton and soybeans.

The term crop plants also includes those plants which have been modified by breeding, mutagenesis or genetic engineering methods including the biotechnological agricultural products which are on the market or under development (see, for example, http://www.bio.org/speeches/pubs/er/agri_products.asp). Genetically modified plants are plants whose genetic material has been modified in a manner which does not occur under natural conditions by crossing, mutations or by natural recombination (that is a recombination of the genetic information). In general, one or more genes are integrated into the genetic material of the plant in order to improve the properties of the plant. Such modifications by genetic engineering include post-translational modifications of proteins, oligopeptides or polypeptides, for example by glycosylation or attachment of polymers such as, for example, prenylated, acetylated or farnesylated radicals or PEG radicals.

By way of example, mention may be made of plants which, by breeding and genetic engineering, are tolerant to certain classes of herbicides, such as hydroxyphenylpyruvate dioxygenase (HPPD) inhibitors, acetolactate synthase (ALS) inhibitors, such as, for example, sulfonylureas (EP-A 257 993, U.S. Pat. No. 5,013,659) or imidazolinones (for example U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, gluphosinate (see, for example, EP-A 242 236, EP-A 242 246) or oxynil herbicides (see, for example, U.S. Pat. No. 5,559,024). Clearfield® oilseed rape (BASF SE, Germany), for example, which is tolerant to imidazolinones, for example imazamox, was generated by breeding and mutagenesis. With the aid of genetic engineering methods, crop plants such as soybeans, cotton, corn, beets and oilseed rape were generated which are resistant to glyphosate or glufosinate, and which are obtainable under the trade names RoundupReady® (glyphosate-resistant, Monsanto, U.S.A.) and Liberty Link® (glufosinate-resistant, Bayer CropScience, Germany).

Also included are plants which, owing to interventions by genetic engineering, produce one or more toxins, for example those of the bacterial strain Bacillus. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; protease inhibitors, for example trypsin inhibitors, serine protease inhibitors, patatin, cystatin or papain inhibitors; ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdyson inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/07278, WO 95/34656, EP-A 427 529, EP-A 451 878, WO 03/18810 and WO 03/52073. The methods for producing these genetically modified plants are known to the person skilled in the art and disclosed, for example, in the publications mentioned above. Many of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda). Genetically modified plants which produce one or more genes coding for insecticidal toxins are described, for example, in the publications mentioned above, and some of them are commercially available, such as, for example, YieldGard® (corn varieties producing the toxin CrylAb), YieldGard® Plus (corn varieties which produce the toxins CrylAb and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin CrylAc), Bollgard® I (cotton varieties which produce the toxin CrylAc), Bollgard® II (cotton varieties which produce the toxins CrylAc and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin CrylAb and the PAT enyzme), MIR604 from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin CrylAc) and 1507 from Pioneer Overseas Corporation, Belgium (corn varieties which produce the toxin CrylF and the PAT enzyme).

Also included are plants which, with the aid of genetic engineering, produce one or more proteins which are more robust or have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum bulbocastanum) or T4 lysozyme (for example potato varieties which, by producing this protein, are resistant to bacteria such as Erwinia amylvora).

Also included are plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

Also included are plants whose ingredients have been modified with the aid of genetic engineering methods in particular for improving human or animal diet, for example oil plants producing health-promoting long-chain omega 3 fatty acids or monounsaturated omega 9 fatty acids (for example Nexera® oilseed rape, DOW Agro Sciences, Canada).

Also included are plants which have been modified with the aid of genetic engineering methods for improving the production of raw materials, for example by increasing the amylopectin content of potatoes (Amflora® potato, BASF SE, Germany).

Accordingly, the present invention also embraces the use of compounds according to the invention or their compositions for treating transgenic plants, in particular transgenic soybean plants or transgenic corn plants. Transgenic plants are plants as described above which have been changed using genetic engineering methods, in particular plants whose properties have been improved using genetic engineering methods. The invention embraces in particular the use of compounds according to the invention or their compositions for treating transgenic plants resistant to glyphosate, glufosinate or glufosinate-ammonium. In a further embodiment, the invention also embraces the use of compounds according to the invention or their compositions for treating herbicide-resistant plants. In a further embodiment, the invention also embraces the use of compounds according to the invention or their compositions for treating herbicide-sensitive plants.

Specifically, the compounds according to the invention and their compositions according to the invention are suitable for controlling the following plant diseases:

Albugo spp. (white rust) on ornamental plants, vegetable crops (for example A. candida) and sunflowers (for example A. tragopogonis); Alternaria spp. (black spot disease, black blotch) on vegetables, oilseed rape (for example A. brassicola or A. brassicae), sugar beet (for example A. tenuis), fruit, rice, soybeans and also on potatoes (for example A. solani or A. alternata) and tomatoes (for example A. solani or A. alternata) and Alternaria spp. (black head) on wheat; Aphanomyces spp. on sugar beet and vegetables; Ascochyta spp. on cereals and vegetables, for example A. tritici (Ascochyta leaf blight) on wheat and A. hordei on barley; Bipolaris and Drechslera spp. (teleomorph: Cochliobolus spp.) for example leaf spot diseases (D. maydis and B. zeicola) on corn, for example glume blotch (B. sorokiniana) on cereals and B. oryzae on rice and on lawn; Blumeria (old name: Erysiphe) graminis (powdery mildew) on cereals (for example wheat or barley); Botryosphaeria spp. (Black Dead Arm Disease') on grapevines (for example B. obtusa); Botrytis cinerea (teleomorph: Botryotinia fuckeliana: gray mold, gray rot) on soft fruit and pome fruit (inter alia strawberries), vegetables (inter alia lettuce, carrots, celeriac and cabbage), oilseed rape, flowers, grapevines, forest crops and wheat (ear mold); Bremia lactucae (downy mildew) on lettuce; Ceratocystis (syn. Ophiostoma) spp. (blue stain fungus) on deciduous trees and coniferous trees, for example C. ulmi (Dutch elm disease) on elms; Cercospora spp. (Cercospora leaf spot) on corn (for example C.zeae-maydis), rice, sugar beet (for example C. beticola), sugar cane, vegetables, coffee, soybeans (for example C. sojina or C. kikuchii) and rice; Cladosporium spp. on tomato (for example C. fulvum: tomato leaf mold) and cereals, for example C. herbarum (ear rot) on wheat; Claviceps purpurea (ergot) on cereals; Cochliobolus (anamorph: Helminthosporium or Bipolaris) spp. (leaf spot) on corn (for example C. carbonum), cereals (for example C. sativus, anamorph: B. sorokiniana: glume blotch) and rice (for example C. miyabeanus, anamorph: H. oryzae); Colletotrichum (teleomorph: Glomerella) spp. (anthracnosis) on cotton (for example C. gossypii), corn (for example C. graminicola: stem rot and anthracnosis), soft fruit, potatoes (for example C. coccodes: wilt disease), beans (for example C. findemuthianum) and soybeans (for example C. truncatum); Corticium spp., for example C. sasakii (sheath blight) on rice; Corynespora cassiicola (leaf spot) on soybeans and ornamental plants; Cycloconium spp., for example C. oleaginum on olive; Cylindrocarpon spp. (for example fruit tree cancer or black foot disease of grapevine, teleomorph: Nectria or Neonectria spp.) on fruit trees, grapevines (for example C. liriodendri, teleomorph: Neonectria liriodendri, black foot disease) and many ornamental trees; Dematophora (teleomorph: Rosellinia) necatrix (root/stem rot) on soybeans; Diaporthe spp. for example D. phaseolorum (stem disease) on soybeans; Drechslera (syn. Helminthosporium, teleomorph: Pyrenophora) spp. on corn, cereals, such as barley (for example D. teres, net blotch) and on wheat (for example D. triticirepentis: DTR leaf spot), rice and lawn; esca disease (dieback of grapevine, apoplexia) on grapevines, caused by Formitiporia (syn. Phellinus) punctata, F. mediterranea, Phaeomoniella chlamydospora (old name Phaeoacremonium chlamydosporum), Phaeoacremonium aleophilum and/or Botryosphaeria obtuse: Elsinoe spp. on pome fruit (E. pyri) and soft fruit (E. veneta: anthracnosis) and also grapevines (E. ampelina: anthracnosis); Entyloma oryzae (leaf smut) on rice; Epicoccum spp. (black head) on wheat; Erysiphe spp. (powdery mildew) on sugar beet (E. betae), vegetables (for example E. pisi) such as cucumber species (for example E. cichoracearum) and cabbage species, such as oilseed rape (for example E. cruciferarum); Eutypa lata (Eutypa cancer or dieback, anamorph: Cytosporina lata, syn. Libertella blepharis) on fruit trees, grapevines and many ornamental trees; Exserohilum (syn. Helminthosporium) spp. on corn (for example E. turcicum); Fusarium (teleomorph: Gibberella) spp. (wilt disease, root and stem rot) on various plants, such as for example F. graminearum or F. culmorum (root rot and silver-top) on cereals (for example wheat or barley), F. oxysporum on tomatoes, F. solani on soybeans and F. verticillioides on corn; Gaeumannomyces graminis (take-all) on cereals (for example wheat or barley) and corn; Gibberella spp. on cereals (for example G. zeae) and rice (for example G. fujikuroi: bakanae disease); Glomerella cingulata on grapevines, pome fruit and other plants and G. gossypii on cotton; Grainstaining complex on rice; Guignardia (black rot) on grapevines; Gymnosporangium spp. on Rosaceae and juniper, for example G. sabinae (pear rust) on pears; Helminthosporium spp. (syn. Drechslera, teleomorph: Cochlibbolus) on corn, cereals and rice; Hemilela spp., for example H. vastatrix (coffee leaf rust) on coffee; Isariopsis clavispora (syn. Cladosporium vitis) on grapevines; Macrophomina phaseolina (syn. phaseoll) (root/stem rot) on soybeans and cotton; Microdochium (syn. Fusarium) nivale (pink snow mold) on cereals (for example wheat or barley); Microsphaera diffusa (powdery mildew) on soybeans; Monilinia spp., for example M. laxa, M. fructicola and M. fructigena (blossom and twig blight) on stone fruit and other Rosaceae; Mycosphaerella spp. on cereals, bananas, soft fruit and peanuts, such as for example M. graminicola (anamorph: Septoria tritici, Septoria leaf blotch) on wheat or M. fijiensis (sigatoka disease) on bananas; Peronospora spp. (downy mildew) on cabbage (for example P. brassicae), oilseed rape (for example P. parasitica), bulbous plants (for example P. destructor), tobacco (P. tabacina) and soybeans (for example P. manshurica); Phakopsora pachyrhizi and P. meibomiae (soybean rust) on soybeans; Phialophora spp. for example on grapevines (for example P. tracheiphila and P. tetraspora) and soybeans (for example P. gregata: stem disease); Phoma lingam (root and stem rot) on oilseed rape and cabbage and P. betae (leaf spot) on sugar beet; Phomopsis spp. on sunflowers, grapevines (for example P. viticola: dead-arm disease) and soybeans (for example stem canker/stem blight: P. phaseoli, teleomorph: Diaporthe phaseolorum); Physoderma maydis (brown spot) on corn; Phytophthora spp. (wilt disease, root, leaf, stem and fruit rot) on various plants, such as on bell peppers and cucumber species (for example P. capsid), soybeans (for example P. megasperma, syn. P. sojae), potatoes and tomatoes (for example P. infestans: late blight and brown rot) and deciduous trees (for example P. ramorum: sudden oak death); Plasmodiophora brassicae (club-root) on cabbage, oilseed rape, radish and other plants; Plasmopara spp., for example P. viticola (peronospora of grapevines, downy mildew) on grapevines and P. halstedii on sunflowers; Podosphaera spp. (powdery mildew) on Rosaceae, hops, pome fruit and soft fruit, for example P. leucotricha on apple; Polymyxa spp., for example on cereals, such as barley and wheat (P. graminis) and sugar beet (P. betae) and the viral diseases transmitted thereby; Pseudocercosporella herpotrichoides (eyespot/stem break, teleomorph: Tapesia yallundae) on cereals, for example wheat or barley; Pseudoperonospora (downy mildew) on various plants, for example P. cubensis on cucumber species or P. humili on hops; Pseudopezicula tracheiphila (angular leaf scorch, anamorph: Phialophora) on grapevines; Puccinia spp. (rust disease) on various plants, for example P. triticina (also brown rust of wheat), P. striiformis (yellow rust), P. hordei (dwarf leaf rust of barley), P. graminis (black rust) or P. recondita (brown rust of rye) on cereals, such as for example wheat, barley or rye, and on asparagus (for example P. asparagi); Pyrenophora (anamorph: Drechslera) tritici-repentis (speckled leaf blotch) on wheat and P. teres (net blotch) on barley; Pyricularia spp., for example P. oryzae (teleomorph: Magnaporthe grisea, rice blast) on rice and P. grisea on lawn and cereals; Pythium spp. (damping-off disease) on lawn, rice, corn, wheat, cotton, oilseed rape, sunflowers, sugar beet, vegetables and other plants (for example P. ultimum or P. aphanidermatum); Ramularia spp., for example R. collo-cygni (Ramularia leaf and awn spot/physiological leaf spot) on barley and R. beticola on sugar beet; Rhizoctonia spp. on cotton, rice, potatoes, lawn, corn, oilseed rape, potatoes, sugar beet, vegetables and on various other plants, for example R. solani (root and stem rot) on soybeans, R. solani (sheath blight) on rice or R. cerealis (sharp eyespot) on wheat or barley; Rhizopus stolonifer (soft rot) on strawberries, carrots, cabbage, grapevines and tomato; Rhynchosporium secalis (leaf spot) on barley, rye and triticale; Sarocladium oryzae and S. attenuatum (sheath rot) on rice; Sclerotinia spp. (stem or white rot) on vegetable and field crops, such as oilseed rape, sunflowers (for example Sclerotinia sclerotiorum) and soybeans (for example S. rolfsii); Septoria spp. on various plants, for example S. glycines (leaf spot) on soybeans, S. tritici (Septoria leaf blotch) on wheat and S. (syn. Stagonospora) nodorum (leaf blotch and glume blotch) on cereals; Uncinula (syn. Erysiphe) necator (powdery mildew, anamorph: Oidium tuckeri) on grapevines; Setosphaeria spp. (leaf spot) on corn (for example S. turcicum, syn. Helminthosporium turcicum) and lawn; Sphacelotheca spp. (head smut) on corn, (for example S. reiliana: kernel smut), millet and sugar cane; Sphaerotheca fuliginea (powdery mildew) on cucumber species; Spongospora subterranea (powdery scab) on potatoes and the viral diseases transmitted thereby; Stagonospora spp. on cereals, for example S. nodorum (leaf blotch and glume blotch, teleomorph: Leptosphaeria [syn. Phaeosphaeria] nodorum) on wheat; Synchytrium endobioticum on potatoes (potato wart disease); Taphrina spp., for example T. deformans (curly-leaf disease) on peach and T. pruni (plum-pocket disease) on plums; Thielaviopsis spp. (black root rot) on tobacco, pome fruit, vegetable crops, soybeans and cotton, for example T. basicola (syn. Cha/ara elegans); Tilletia spp. (bunt or stinking smut) on cereals, such as for example T. tritici (syn. T. caries, wheat bunt) and T. controversa (dwarf bunt) on wheat; Typhula incarnata (gray snow mold) on barley or wheat; Urocystis spp., for example U. occulta (flag smut) on rye; Uromyces spp. (rust) on vegetable plants, such as beans (for example U. appendiculatus, syn. U. phaseoli) and sugar beet (for example U. betae); Ustilago spp. (loose smut) on cereals (for example U. nuda and U. avaenae), corn (for example U. maydis corn smut) and sugar cane; Venturia spp. (scab) on apples (for example V. inaequalis) and pears and Verticillium spp. (leaf and shoot wilt) on various plants, such as fruit trees and ornamental trees, grapevines, soft fruit, vegetable and field crops, such as for example V. dahliae on strawberries, oilseed rape, potatoes and tomatoes.

Moreover, the compounds according to the invention and their compositions according to the invention are suitable for controlling harmful fungi in the protection of materials and buildings (for example wood, paper, paint dispersions, fibers or tissues) and in the protection of stored products. In the protection of wood and buildings, particular attention is paid to the following harmful fungi: Ascomycetes, such as Ophiostoma spp., Ceratocystis spp., Aureobasidium pullulans, Scierophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of materials to the following yeast fungi: Candida spp. and Saccharomyces cerevisae.

The compounds according to the invention and their compositions according to the invention are suitable for improving plant health. Moreover, the invention relates to a method for improving plant health by treating the plants, the plant propagation material and/or the site at which the plants grow or are intended to grow with an effective amount of the compounds according to the invention or their compositions according to the invention.

The term “plant health” comprises states of a plant and/or its harvested material which are determined by various indicators individually or in combination, such as, for example, yield (for example increased biomass and/or increased content of utilizable ingredients), plant vitality (for example increased plant growth and/or greener leaves (“greening effect”)), quality (for example increased content or composition of certain ingredients) and tolerance to biotic and/or abiotic stress. The indicators mentioned here for a state of plant health may occur independently of one another or may influence each other.

Accordingly, the invention also provides the use of compounds according to the invention and/or their agriculturally acceptable salts for controlling phytopathogenic fungi.

The invention furthermore provides a method for controlling phytopathogenic fungi which comprises treating the fungi or the materials, plants, the soil or seeds to be protected against fungal attack with an effective amount of a compound according to the invention and/or an agriculturally acceptable salt thereof.

The compounds according to the invention are employed as such or in the form of a composition by treating the harmful fungi, their habitat or the plants or plant propagation materials, for example seed materials to be protected against fungal attack, the soil, areas, materials or spaces with a fungicidally effective amount of the compounds according to the invention. The application can be carried out both before and after the infection of the plants, plant propagation materials, for example seed materials, the soil, the areas, materials or spaces by the fungi.

Plant propagation materials can be treated prophylactically during or even before sowing or during or even before transplanting with compounds according to the invention or their compositions according to the invention.

Moreover, the invention relates to preparations and agrochemical compositions comprising a solvent or solid carrier and at least one compound according to the invention, and to the use of these compositions for controlling harmful fungi. The present invention also provides a preparation or an agrochemical composition comprising at least one compound according to the invention and/or an agriculturally acceptable salt thereof for use in crop protection. Such a preparation usually comprises at least one liquid or solid carrier.

Accordingly, the invention also embraces preparations and agrochemical compositions comprising a solid or liquid carrier and a fungicidal compound according to the invention. In the present context, the term “liquid carrier” is synonymous with solvent.

An agrochemical composition comprises a fungicidally effective amount of a compound according to the invention. The term “effective amount” refers to an amount of the agrochemical composition or of the compound according to the invention which is sufficient for controlling harmful fungi on crop plants or in the protection of materials and buildings and does not cause any significant damage to the treated crop plants.

Such an amount may vary within a wide range and is influenced by numerous factors, such as, for example, the harmful fungus to be controlled, the respective crop plant or materials treated, the climatic conditions and compounds.

The compounds according to the invention, their N-oxides and their salts can be converted into the types customary for agrochemical compositions, for example solutions, emulsions, suspensions, dusts, powders, pastes and granules. The type of composition depends on the respective intended purpose; in each case, it should ensure a fine and even distribution of the compound according to the invention.

In the present context, the term “preparation” is synonymous with the term “composition”, in particular “agrochemical composition”, and “formulation”.

Here, examples of types of compositions are suspensions (SC, OD, FS), pastes, pastilles, wettable powders or dusts (WP, SP, SS, WS, DP, DS) or granules (GR, FG, GG, MG) which may either be water-soluble or dispersible (wettable), and also gels for treating plant propagation materials such as seed (GF).

In general, the composition types (for example SC, OD, FS, WG, SG, WP, SP, SS, WS, GF) are used in diluted form. Composition types such as DP, DS, GR, FG, GG and MG are generally employed in undiluted form.

The agrochemical compositions are prepared in a known manner (see, for example, U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th edition, McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation Technology (Wiley VCH Verlag, Weinheim, 2001).

The agrochemical compositions may furthermore also comprise auxiliaries customary for crop protection compositions, the selection of the auxiliaries depending on the use form or the active compound in question.

Examples of suitable auxiliaries are solvents, solid carriers, surfactants (such as further solubilizers, protective colloids, wetting agents and tackifiers), organic and inorganic thickeners, bactericides, antifreeze agents, antifoams, if appropriate colorants and adhesives (for example for the treatment of seed).

Suitable solvents are water, organic solvents, such as mineral oil fractions having a medium to high boiling point, such as kerosene and diesel oil, furthermore coal tar oils, and also oils of vegetable or animal origin, aliphatic, cyclic and aromatic hydrocarbons, for example paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl fatty amides, fatty acids and fatty acid esters and strongly polar solvents, for example amines, such as N-methylpyrrolidone. In principle, it is also possible to use solvent mixtures, and also mixtures of the solvents mentioned above and water.

Solid carriers are mineral earths, such as silicic acids, silica gels, silicates, talc, kaolin, limestone, lime, chalk, bole, loess, clay, dolomite, diatomaceous earth, calcium sulfate and magnesium sulfate, magnesium oxide, ground synthetic substances, fertilizers, such as ammonium sulfate, ammonium phosphate, ammonium nitrate, ureas and vegetable products, such as cereal meal, tree bark meal, sawdust and nutshell meal, cellulose powder or other solid carriers.

Suitable surfactants (adjuvants, wetting agents, tackifiers, dispersants or emulsifiers) are the alkali metal, alkaline earth metal and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and also of fatty acids, alkyl- and alkylarylsulfonates, alkyl, lauryl ether and fatty alcohol sulfates, and also salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octyl phenol ether, ethoxylated isooctylphenol, octylphenol or nonylphenol, alkylphenyl polyglycol ether, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfide waste liquors, and also proteins, denatured proteins, polysaccharides (for example methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart modified flow properties to the composition, i.e. high viscosity in the state of rest and low viscosity in motion) are polysaccharides and also organic and inorganic sheet minerals, such as xanthan gum (Kelzan®, CP Kelco, USA), Rhodopol® 23 (Rhodia, France) or Veegum® (R. T. Vanderbilt, USA) or Attaclay® (Engelhard Corp., NJ, USA).

Bactericides can be added for stabilizing the composition. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivatives, such as alkylisothiazolinones and benzisothiazolinones (Acticide® MBS from Thor Chemie).

Examples of suitable antifreeze agents are ethylene glycol, propylene glycol, urea and glycerol.

Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and mixtures thereof.

Examples of colorants are both sparingly water-soluble pigments and water-soluble dyes. Examples which may be mentioned are the dyes and pigments known under the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of adhesives are polyvinylpyrrolidone, polyvinyl acetate, polyvinyl alcohol and cellulose ether (Tylose®, Shin-Etsu, Japan).

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the compounds I and, if present further active compounds with at least one solid carrier.

Granules, for example coated granules, impregnated granules and homogeneous granules, can be prepared by binding the active compounds to at least one solid carrier. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

The following are examples of types of composition:

1. Types of composition for dilution with water

i) Water-soluble concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a composition having an active compound content of 10% by weight.

ii) Dispersible concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

iii) Emulsifiable concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The composition has an active compound content of 15% by weight.

iv) Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The composition has an active compound content of 25% by weight.

v) Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the composition is 20% by weight.

vi) Water-dispersible granules and water-soluble granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The composition has an active compound content of 50% by weight.

vii) Water-dispersible powders and water-soluble powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the composition is 75% by weight.

viii) Gels (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

2. Types of composition to be applied undiluted

ix) Dusts (DP, DS)

5 parts by weight of the active compounds are ground finely and mixed intimately with 95 parts by weight of finely divided kaolin. This gives a dustable product with an active compound content of 5% by weight.

x) Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

xi) ULV solutions (UL)

10 parts by weight of the active compounds are dissolved in 90 parts by weight of an organic solvent, for example xylene. This gives a composition with an active compound content of 10% by weight to be applied undiluted.

In general, the compositions of the compounds according to the invention comprise from 0.1 to 95% by weight, preferably from 0.1 to 90% by weight, preferably from 0.5 to 90% by weight, of active compound (compound according to the invention). The compounds I and II are employed in a purity of from 90% to 100%, preferably 95% to 100% (according to the NMR spectrum).

Water-soluble concentrates (LS), suspensions (FS), dusts (DS), water-dispersible and water-soluble powders (WS, SS), emulsions (ES), emulsifiable concentrates (EC) and gels (GF) are usually used for the treatment of plant propagation materials, in particular seed. These compositions can be applied to the propagation materials, in particular seed, in undiluted or, preferably, diluted form. In this case, the corresponding composition can be diluted 2 to 10 times so that in the compositions used for the seed dressing from 0.01 to 60% by weight, preferably from 0.1 to 40% by weight of active compound are present. The application can be carried out before or during sowing. The treatment of plant propagation material, in particular the treatment of seed, is known to the person skilled in the art and is carried out by dusting, coating, pelleting, dipping or drenching the plant propagation material, the treatment preferably being carried out by pelleting, coating and dusting or by furrow treatment, such that, for example, premature germination of the seed is prevented.

For seed treatment, preference is given to using suspensions. Such compositions usually comprise from 1 to 800g of active compound/l, from 1 to 200g of surfactants/l, from 0 to 200g of antifreeze agent/l, from 0 to 400g of binders/l, from 0 to 200g of colorants/l and solvents, preferably water.

The compounds can be used as such or in the form of their compositions, for example in the form of directly sprayable solutions, powders, suspensions, dispersions, emulsions, oil dispersions, pastes, dustable products, materials for spreading or granules, by means of spraying, atomizing, dusting, spreading, raking in, immersing or pouring. The types of composition depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply compositions comprising over 95% by weight of active compound, or even to apply the active compound without additives.

When used in crop protection, the application rates are from 0.001 to 2.0 kg of active compound per ha, preferably from 0.005 to 2 kg per ha, particularly preferably from 0.05 to 0.9 kg per ha, especially from 0.1 to 0.75 kg per ha, depending on the nature of the desired effect.

In the treatment of plant propagation materials, for example seed, the amounts of active compound used are generally from 0.1 to 1000g/100 kg of propagation material or seed, preferably from 1 to 1000g/100 kg, particularly preferably from 1 to 100g/100 kg, especially from 5 to 100g/100 kg. Accordingly, the invention furthermore provides seed comprising at least one compound according to the invention and/or an agriculturally acceptable salt thereof in an amount of from 1 to 1000g per 100 kg.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

Various types of oils, wetters, adjuvants, herbicides, bactericides, other fungicides and/or pesticides may be added to the compounds according to the invention (active compounds) or the compositions comprising them, if appropriate not until immediately prior to use (tank mix). These compositions can be admixed with the compositions according to the invention in a weight ratio of from 1:100 to 100:1, preferably from 1:10 to 10:1

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compounds according to the invention and their compositions in the application form as fungicides can also be present together with other active compounds, for example with herbicides, insecticides, growth regulators, fungicides or else with fertilizers, as premix or if appropriate also only immediately prior to use (tank mix). When mixing the compounds I or the compositions comprising them with one or more further active compounds, in particular fungicides, it is in many cases possible, for example, to widen the activity spectrum or to prevent the development of resistance. In many cases, synergistic effects are obtained.

Accordingly, the invention also provides a composition for crop protection which comprises a compound according to the invention, in particular a compound I or a compound II, and at least one further fungicidally, insecticidally and/or herbicidally active compound. According to one embodiment, the further active compound is a fungicidally active compound, in particular one selected from the list below. Here, the active compounds are preferably present in synergistic amounts.

A further subject matter of the invention relates to a composition comprising a compound according to the invention and/or an acid addition salt or metal salt thereof. As crop protection composition, this composition furthermore comprises at least one solid or liquid carrier. According to a further embodiment, the compositions mentioned may furthermore comprise at least one further fungicidally, insecticidally and/or herbicidally active compound. According to a further embodiment, the compositions comprise at least two further fungicidally active compounds, in particular two active compounds selected from the fungicides mentioned below.

The fungicides are preferably selected from the following groups: Strobilurins, carboxamides, such as carboxanilides, carboxylic acid morpholides, benzamides, other carboxamides, azoles, such as triazoles, imidazoles, benzimidazoles, others, nitrogenous heterocyclyl compounds, such as pyridines, pyrimidines, pyrroles, morpholines, dicarboximides, other nitrogenous heterocyclyl compounds, thio- and dithiocarbamates, carbamates, guanidines, antibiotics, nitrophenyl derivatives, organometal compounds, sulfur-containing heterocyclyl compounds, organophosphorus compounds, organochlorine compounds, inorganic active compounds, other fungicides.

The following list of fungicides with which the compounds according to the invention can be applied together is meant to illustrate the possible combinations, but not to limit them:

A) strobilurins
azoxystrobin, dimoxystrobin, enestroburin, fluoxastrobin, kresoxim-methyl, methominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)cyclopropanecarboximidoylsulfanylmethyl)phenyl)acrylate, 2-(2-(3-(2,6-dichlorophenyl)-1-methylallylideneaminooxymethyl)phenyl-2-methoxyimino-N-methyl acetamide;
   B) carboxamides
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethylindan-4-yl)-nicotinamide, N-(3′,4′-dichloro-5-fluorobiphenyl-2-yl)-3-difluoromethyl-1-methyl-1H-pyrazole-4-carboxamide, [2-(1,3-dimethylbutyl)phenyl]-5-fluoro-1,3-dimethyl-1H-pyrazole-4-carboxamide, N-(4′-chloro-3′,5-difluorobiphenyl-2-yl)-3-difluoromethyl-1-methyl-1H-pyrazole-4-carboxamide, N-(4′-chloro-3′,5-difluorobiphenyl-2-yl)-3-trifluoromethyl-1-methyl-1H-pyrazole-4-carboxamide, N-(3′,4′-dichloro-4-fluorobiphenyl-2-yl)-3-trifluoromethyl-1-methyl-1H-pyrazole-4-carboxamide, N-(3′,5-difluoro-4′-methylbiphenyl-2-yl)-3-difluoromethyl-1-methyl-1H-pyrazole-4-carboxamide, N-(3′,5-difluoro-4′-methylbiphenyl-2-yl)-3-trifluoromethyl-1-methyl-1H-pyrazole-4-carboxamide, N-(2-bicyclopropyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, 3,4-dichloro-N-(2-cyanophenyl)isothiazole-5-carboxamide, N-(2′,4′-difluorobiphenyl-2-yl)-1-methyl-3-trifluoromethyl-1H-pyrazole-4-carboxamide, N-(2′,4′-dichlorobiphenyl-2-yl)-1-methyl-3-trifluoromethyl-1H-pyrazole-4-carboxamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-1-methyl-3-trifluoromethyl-1H-pyrazole-4-carboxamide, N-(2′,5′-dichlorobiphenyl-2-yl)-1-methyl-3-trifluoromethyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-1-methyl-3-trifluoromethyl-1H-pyrazole-4-carboxamide, N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-1-methyl-3-trifluoromethyl-1H-pyrazole-4-carboxamide, N-(3′-chlorobiphenyl-2-yl)-1-methyl-3-trifluoromethyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-1-methyl-3-trifluoromethyl-1H-pyrazole-4-carboxamide, N-(2′-chlorobiphenyl-2-yl)-1-methyl-3-trifluoromethyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(2′-fluoro-4′-chloro-5′-methylbiphenyl-2-yl)-1-methyl-3-trifluoromethyl-1H-pyrazole-4-carboxamide, N-(3′,4′,5′-trifluorobiphenyl-2-yl)-1-methyl-3-trifluoromethyl-1H-pyrazole-4-carboxamide, N-(3′,4′,5′-trifluorobiphenyl-2-yl)-1-methyl-3-difluoromethyl-1H-pyrazole-4-carboxamide, N-(2′,4′,5′-trifluorobiphenyl-2-yl)-1-methyl-3-difluoromethyl-1H-pyrazole-4-carboxamide, N-(3′,4′,5′-trifluorobiphenyl-2-yl)-3-chlorofluoromethyl-1-methyl-1H-pyrazole-4-carboxamide, N-[2-(1,1,2,3,3,3-hexafluoropropoxy)phenyl]-1-methyl-3-trifluoromethyl-1H-pyrazole-4-carboxamide, N-[2-(1,1,2,3,3,3-hexafluoropropoxy)phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide, N-[2-(2-chloro-1,1,2-trifluoroethoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-[2-(1,1,2,2-tetrafluoroethoxy)phenyl]-1-methyl-3-trifluoromethyl-1H-pyrazole-4-carboxamide, N-(4′-(trifluoromethylthio)biphenyl-2-yl)-3-difluoromethyl-1-methyl-1H-pyrazole-4-carboxamide, N-(4′-(trifluoromethylthio)biphenyl-2-yl)-1-methyl-3-trifluoromethyl-1H-pyrazole-4-carboxamide, [2-(1,2-dimethylpropyl)phenyl]-5-fluoro-1,3-dimethyl-1H-pyrazole-4-carboxamide, N-(2-{4-[3-(4-chlorophenyl)prop-2-ynyloxy]-3-methoxyphenyl}ethyl)-2-methanesulfonylamino-3-methylbutyramide, N-(2-{4-[3-(4-chlorophenyl)prop-2-ynyloxy]-3-methoxyphenyl}ethyl)-2-ethanesulfonylamino-3-methylbutyramide; N-(3′,4′-dichloro-5-fluorobiphenyl-2-yl)-3-trifluoromethyl-1-methyl-1H-pyrazol-4-carboxamide, N-(2-(1,3,3-trimethylbutyl)phenyl)-1,3-dimethyl-5-fluoro-1H-pyrazol-4-carboxamide, N-[1,2,3,4-tetrahydro-9-(1-methylethyl)-1,4-methanonaphthalene-5-yl]-3-(difluoromethyl)-1-methyl-1H-pyrazol-4-carboxamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine and N′-(5-difluoromethyl-2-methyl-4-(3-trimethylsilanylpropoxy)phenyl)-N-ethyl-N-methylformamidine;
carboxylic acid morpholides: dimethomorph, flumorph, pyrimorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;
   C) azoles
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)cycloheptanol;
imidazoles: cyazofamid, imazalil, imazalil-sulfate, pefurazoate, prochloraz, triflumizole;
benzimidazoles: benomyl, carbendazim, fuberidazole, thiabendazole;
others: ethaboxam, etridiazole, hymexazole, 1-(4-chlorophenyl)-1-(propyn-2-yloxy)-3-(4-(3,4-dimethoxyphenyl)isoxazol-5-yl)propan-2-one, 2-(4-chlorophenyl)-N-[4-(3,4-dimethoxyphenyl)isoxazol-5-yl]-2-prop-2-inyloxy acetamide;
   D) nitrogenous heterocyclyl compounds
pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]-pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph-acetate, fenpropimorph, tridemorph;
piperidines: fenpropidine;
dicarboximides: fluoroimide, iprodione, procymidone, vinclozolin;
non-aromatic 5-membered heterocycles: famoxadone, fenamidone, flutianil, octhilinon, probenazol, S-ally 5-amino-2-isopropyl-3-oxo-4-orthotolyl-2,3-dihydropyrazol-1-thiocarboxylate;
others: acibenzolar-5-methyl, amisulbrom, anilazine, blasticidin-S, captafol, captan, quinomethionate, dazomet, debacarb, diclomezine, difenzoquat, difenzoquat-methyl sulfate, fenoxanil, folpet, oxolinic acid, piperalin, proquinazid, pyroquilon, quinoxyfen, triazoxide, tricyclazole, 5-chloro-7-(4-methylpiperidin-1-yl)-6-(2,4,6-trifluorophenyl)-[1,2,4]triazolo[1,5-a]pyrimidine, 6-(4-tert-butylphenyl)-5-methyl-[1,2,4]triazolo[1,5-a]pyrimidin-7-ylamine, 5-methyl-6-(3,5,5-trimethylhexyl)-[1,2,4]triazolo[1,5-a]pyrimidin-7-ylamine, 5-methyl-6-octyl-[1,2,4]triazolo[1,5-a]pyrimidin-7-ylamine, 6-methyl-5-octyl[1,2,4]triazolo[1,5-a]pyrimidin-7-ylamine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl[1,2,4]triazolo[1,5-a]pyrimidin-7-ylamine, 5-trifluoromethyl-6-(3,5,5-trimethylhexyl)-[1,2,4]triazolo[1,5-a]pyrimidin-7-ylamine, 2-butoxy-6-iodo-3-propylchromen-4-one, 5-chloro-1-(4,6-dimethoxypyrimidin-2-yl)-2-methyl-1H-benzoimidazole, 6-(3,4-dichlorophenyl)-5-methyl-[1,2,4]triazolo[1,5-a]pyrimidin-7-ylamine;
   E) carbamates and dithiocarbamates
thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, (4-fluorophenyl) N-(1-(1-(4-cyanophenyl)ethanesulfonyl)but-2-yl)carbamate;
   F) other fungicides
guanidines: dodine, dodine-free base, guazatine, guazatine-acetate, iminoctadine, iminoctadine-triacetate, iminoctadine-tris(albesilate);
antibiotics: kasugamycin, kasugamycin-hydrochloride hydrate, polyoxins, streptomycin, validamycin A;
nitrophenyl derivatives:
binapacryl, dicloran, dinobuton, dinocap, nitrothal-isopropyl, tecnazen;
organometallic compounds: fentin salts such as, for example, fentin-acetate, fentinchloride, fentin-hydroxide;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl;
organochlorine compounds: chlorothalonil, dichlofluanid, dichlorophen, flusulfamide, hexachlorobenzene, pencycuron, pentachlorophenol and salts thereof, phthalide, quintozene, thiophanate-methyl, tolylfluanid, N-(4-chloro-2-nitrophenyl)-N-ethyl-4-methylbenzenesulfonamide;
inorganic active compounds: phosphorous acid and salts thereof, sulfur, Bordeaux mixture, copper salts such as, for example, copper acetate, copper hydroxide, copper oxychloride, basic copper sulfate;
others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenyl acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methylformamidine, methyl N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, methyl (R)—N-(1,2,3,4-tetrahydronaphthalen-1-yl)-2-{1-[2-(5-methyl-3-trifluoromethylpyrazol-1-yl)acetyl]-piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-yl methoxyacetate;
   G) growth regulators
   abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapac-ethyl and uniconazole;
   H) herbicides
acetamides: acetochlor, alachlor, butachior, dimethachior, dimethenamid, flufenacet, mefenacet, metolachlor, metazachlor, napropamide, naproanilide, pethoxamid, pretilachlor, propachlor, thenylchlor;
amino acid analogues: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-p-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichiobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazone, flufenpyr-ethyl, fluthiacet, norflurazone, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chlorimuron-ethyl, chiorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propylimidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrion, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-ene-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;
   I) insecticides
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
inhibitors of insect growth: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitors (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorylation: cyhexatin, diafenthiuron, fenbutatin oxide, propargite;
insect molting inhibitors: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamid, chlorantraniliprol, cyazypyr (HGW86); cyenopyrafen, flupyrazofos, cyflumetofen, amidoflumet, imicyafos, bistrifluoron, and pyrifluquinazon.

The present invention relates in particular also to fungicidal compositions which comprise at least one compound of the general formula I and at least one further crop protection agent, selected, for example, from the active compounds of groups A) to I) mentioned above, in particular at least one fungicidally active compound, in particular selected from the active compounds of groups A) to F) mentioned above, and, if appropriate, one or more agriculturally suitable carriers. With a view to reducing the application rates, these mixtures are of interest, since many show, at a reduced total amount of active compounds applied, an improved activity against harmful fungi, in particular for certain indications. By simultaneous joint or separate application of compound(s) I with at least one active compound of groups A) to I), the fungicidal activity can be increased in a superadditive manner.

In the sense of the present application, joint application means that the at least one compound of the formula I and the at least one further active compound are present simultaneously at the site of action (i.e. the plant-damaging fungi to be controlled and their habitat, such as infected plants, plant propagation materials, in particular seed, soils, materials or spaces and also plants, plant propagation materials, in particular seed, soils, materials or spaces to be protected against fungal attack) in an amount sufficient for an effective control of fungal growth. This can be achieved by applying the compounds I and at least one further active compound jointly in a joint active compound preparation or in at least two separate active compound preparations simultaneously, or by applying the active compounds successively to the site of action, the interval between the individual active compound applications being chosen such that the active compound applied first is, at the time of application of the further active compound(s), present at the site of action in a sufficient amount. The order in which the active compounds are applied is of minor importance.

In binary mixtures, i.e. compositions according to the invention comprising a compound I and a further active compound, for example an active compound of groups A) to I), the weight ratio of compound I to further active compound depends on the properties of the active compounds in question; usually, it is in the range of from 1:100 to 100:1, frequently in the range of from 1:50 to 50:1, preferably in the range of from 1:20 to 20:1, particularly preferably in the range of from 1:10 to 10:1, especially in the range of from 1:3 to 3:1.

The components of the composition according to the invention can be packaged and used individually or as a ready-mix or as a kit of parts.

In one embodiment of the invention, the kits may comprise one or more, and even all, components used for preparing an agrochemical composition according to the invention. For example, these kits may comprise one or more fungicide components and/or an adjuvant component and/or an insecticide component and/or a growth regulator component and/or a herbicide. One or more components may be present combined or preformulated with one another. In the embodiments where more than two components are provided in a kit, the components can be combined with one another and be packaged in a single container, such as a vessel, a bottle, a tin, a bag, a sack or canister. In other embodiments, two or more components of a kit may be packaged separately, i.e. not preformulated or mixed. Kits may comprise one or more separate containers, such as vessels, bottles, tins, bags, sacks or canisters, each container comprising a separate component of the agrochemical composition. The components of the composition according to the invention may be packaged and used individually or as a ready-mix or as a kit of parts. In both forms, a component may be used separately or together with the other components or as a part of a kit of parts according to the invention for preparing the mixture according to the invention.

The user uses the composition according to the invention usually for use in a predosage device, a knapsack sprayer, a spray tank or a spray plane. Here, the agrochemical composition is diluted with water and/or buffer to the desired application concentration, with further auxiliaries being added, if appropriate, thus giving the ready-to-use spray liquor or the agrochemical composition according to the invention. Usually, from 50 to 500 liters of the ready-to-use spray liquor are applied per hectare of agricultural utilized area, preferably from 100 to 400 liters.

According to one embodiment, the user may himself mix individual components, such as, for example, parts of a kit or a two- or three-component mixture of the composition according to the invention in a spray tank and, if appropriate, add further auxiliaries (tank mix).

In a further embodiment, the user may mix both individual components of the composition according to the invention and partially pre-mixed components, for example components comprising compounds I and/or active compounds from groups A) to I), in a spray tank and, if appropriate, add further auxiliaries (tank mix).

In a further embodiment, the user may use both individual components of the composition according to the invention and partially pre-mixed components, for example components comprising compounds I and/or active compounds from groups A) to I), jointly (for example as a tank mix) or in succession.

Preference is given to compositions of a compound I (component 1) with at least one active compound from group A) (component 2) of the strobilurins and in particular selected from the group consisting of azoxystrobin, dimoxystrobin, fluoxastrobin, kresoxim-methyl, orysastrobin, picoxystrobin, pyraclostrobin and trifloxystrobin.

Preference is also given to compositions of a compound I (component I) with at least one active compound selected from group B) (component 2) of the carboxamides and in particular selected from the group consisting of fenhexamid, metalaxyl, mefenoxam, ofurace, dimethomorph, flumorph, fluopicolid (picobenzamid), zoxamide, carpropamid and mandipropamid.

Preference is also given to compositions of a compound I (component I) with at least one active compound selected from group C) (component 2) of the azoles and in particular selected from the group consisting of cyproconazole, difenoconazole, epoxiconazole, fluquinconazole, flusilazole, flutriafole, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to compositions of a compound I (component I) with at least one active compound selected from group D) (component 2) of the nitrogenous heterocyclyl compounds and in particular selected from the group consisting of fluazinam, cyprodinil, fenarimol, mepanipyrim, pyrimethanil, triforin, fludioxonil, fodemorph, fenpropimorph, tridemorph, fenpropidin, iprodion, vinclozolin, famoxadone, fenamidone, probenazole, proquinazid, acibenzolar-s-methyl, captafol, folpet, fenoxanil and quinoxyfen.

Preference is also given to compositions of a compound I (component I) with at least one active compound selected from group E) (component 2) of the carbamates and in particular selected from the group consisting of mancozeb, metiram, propineb, thiram, iprovalicarb, flubenthiavalicarb and propamocarb.

Preference is also given to compositions of a compound I (component I) with at least one active compound selected from the fungicides of group F) (component 2) and in particular selected from the group consisting of dithianon, fentin salts, such as fentin acetate, fosetyl, fosetyl-aluminum, H₃PO₃ and salts thereof, chlorothalonil, dichlofluanid, thiophanate-methyl, copper acetate, copper hydroxide, copper oxychloride, copper sulfate, sulfur, cymoxanil, metrafenone, spiroxamine and 5-chloro-7-(4-methylpiperidin-1-yl)-6-(2,4,6-trifluorophenyl)-[1,2,4]triazolo[1,5-a]pyrimidine.

Accordingly, the present invention furthermore relates to compositions of a compound I (component 1) with a further active compound (component 2), the letter being selected from rows E-1 to E-416 in the column “component 2” of Table E. A further embodiment of the invention relates to the compositions E-1 to E-416 listed in Table E, each line of Table E corresponding to an agrochemical composition comprising a compound of the formula I (component 1), which is preferably one of the compounds described herein as being preferred, and comprising the further active compound from groups A to I indicated in each case in the line in question (component 2). According to one embodiment of the invention, component 1 in each line of Table E is in each case one of the compounds of the formula I that are specifically individualized in Tables 1a to 93a. The active compounds in the compositions described are in each case preferably present in synergistically effective amounts.

TABLE E
Active compound composition comprising an individualized compound I and a
further active compound from the groups A) to I).

| Row | Component 1 | Component 2 |
|---|---|---|
| E-1 | a compound of the formula I | azoxystrobin |
| E-2 | a compound of the formula I | dimoxystrobin |
| E-3 | a compound of the formula I | enestroburin |
| E-4 | a compound of the formula I | fluoxastrobin |
| E-5 | a compound of the formula I | kresoxim-methyl |
| E-6 | a compound of the formula I | metominostrobin |
| E-7 | a compound of the formula I | orysastrobin |
| E-8 | a compound of the formula I | picoxystrobin |
| E-9 | a compound of the formula I | pyraclostrobin |
| E-10 | a compound of the formula I | pyribencarb |
| E-11 | a compound of the formula I | trifloxystrobin |
| E-12 | a compound of the formula I | 2-(2-(6-(3-chloro-2-methylphenoxy)- |
| | | 5-fluoropyrimidin-4-yloxy)phenyl)- |
| | | 2-methoxyimino-N-methylacetamide |
| E-13 | a compound of the formula I | 2-(ortho-((2,5-dimethylphenyloxy- |
| | | methylene)phenyl)-3-methoxyacrylic acid |
| | | methyl ester |
| E-14 | a compound of the formula I | 3-methoxy-2-(2-(N-(4-methoxyphenyl)- |
| | | cyclopropanecarboximidoylsulfanyl- |
| | | methyl)phenyl)acrylic acid methyl ester |
| E-15 | a compound of the formula I | benalaxyl |
| E-16 | a compound of the formula I | benalaxyl-M |
| E-17 | a compound of the formula I | benodanil |
| E-18 | a compound of the formula I | bixafen |
| E-19 | a compound of the formula I | boscalid |
| E-20 | a compound of the formula I | carboxin |
| E-21 | a compound of the formula I | fenfuram |
| E-22 | a compound of the formula I | fenhexamid |
| E-23 | a compound of the formula I | flutolanil |
| E-24 | a compound of the formula I | furametpyr |
| E-25 | a compound of the formula I | isotianil |
| E-26 | a compound of the formula I | kiralaxyl |
| E-27 | a compound of the formula I | mepronil |
| E-28 | a compound of the formula I | metalaxyl |
| E-29 | a compound of the formula I | ofurace |
| E-30 | a compound of the formula I | oxadixyl |
| E-31 | a compound of the formula I | oxycarboxin |
| E-32 | a compound of the formula I | penthiopyrad |
| E-33 | a compound of the formula I | sedaxane |
| E-34 | a compound of the formula I | thifluzamide |
| E-35 | a compound of the formula I | tecloftalam |
| E-36 | a compound of the formula I | tiadinil |
| E-37 | a compound of the formula I | 2-amino-4-methylthiazole-5-carboxanilide |
| E-38 | a compound of the formula I | 2-chloro-N-(1,1,3-trimethylindan-4-yl)- |
| | | nicotinamide |
| E-39 | a compound of the formula I | N-(3′,4′-dichloro-5-fluorobiphenyl-2-yl)- |
| | | 3-difluoromethyl-1-methyl-1H-pyrazole- |
| | | 4-carboxamide |
| E-40 | a compound of the formula I | 5-fluoro-1,3-dimethyl-1H-pyrazole- |
| | | 4-carboxylic acid [2-(1,3-dimethylbutyl)-phenyl]amide |
| E-41 | a compound of the formula I | N-(4′-chloro-3′,5-difluorobiphenyl-2-yl)- |
| | | 3-difluoromethyl-1-methyl-1H-pyrazole- |
| | | 4-carboxamide |
| E-42 | a compound of the formula I | N-(4′-chloro-3′,5-difluorobiphenyl-2-yl)- |
| | | 3-trifluoromethyl-1-methyl-1H-pyrazole- |
| | | 4-carboxamide |
| E-43 | a compound of the formula I | N-(3′,4′-dichloro-4-fluorobiphenyl-2-yl)- |
| | | 3-trifluoromethyl-1-methyl-1H-pyrazole- |
| | | 4-carboxamide |
| E-44 | a compound of the formula I | N-(3′,5-difluoro-4′-methylbiphenyl-2-yl)- |
| | | 3-difluoromethyl-1-methyl-1H-pyrazole- |
| | | 4-carboxamide |
| E-45 | a compound of the formula I | N-(3′,5-difluoro-4′-methylbiphenyl-2-yl)- |
| | | 3-trifluoromethyl-1-methyl-1H-pyrazole- |
| | | 4-carboxamide |
| E-46 | a compound of the formula I | N-(2-bicyclopropyl-2-ylphenyl)-3-difluoro- |
| | | methyl-1-methyl-1H-pyrazole- |
| | | 4-carboxamide |
| E-47 | a compound of the formula I | N-(cis-2-bicyclopropyl-2-ylphenyl)- |
| | | 3-difluoromethyl-1-methyl-1H-pyrazole- |
| | | 4-carboxamide |
| E-48 | a compound of the formula I | N-(trans-2-bicyclopropyl-2-ylphenyl)-3- |
| | | difluoromethyl-1-methyl-1H-pyrazole- |
| | | 4-carboxamide |
| E-49 | a compound of the formula I | N-(4′-bromobiphenyl-2-yl)-4- |
| | | difluoromethyl-2-methylthiazole-5- |
| | | carboxamide |
| E-50 | a compound of the formula I | N-(4′-trifluoromethylbiphenyl-2-yl)-4- |
| | | difluoromethyl-2-methylthiazole-5- |
| | | carboxamide |
| E-51 | a compound of the formula I | N-(4′-chloro-3′-fluorobiphenyl-2-yl)-4- |
| | | difluoromethyl-2-methylthiazole-5- |
| | | carboxamide |
| E-52 | a compound of the formula I | 3,4-dichloro-N-(2-cyanophenyl)isothiazole- |
| | | 5-carboxamide |
| E-53 | a compound of the formula I | N-(2′,4′-difluorobiphenyl-2-yl)-1-methyl-3- |
| | | trifluoromethyl-1H-pyrazole-4-carboxamide |
| E-54 | a compound of the formula I | N-(2′,4′-dichlorobiphenyl-2-yl)-1-methyl-3- |
| | | trifluoromethyl-1H-pyrazole-4-carboxamide |
| E-55 | a compound of the formula I | N-(2′,4′-difluorobiphenyl-2-yl)-3- |
| | | difluoromethyl-1-methyl-1H-pyrazole-4- |
| | | carboxamide |
| E-56 | a compound of the formula I | N-(2′,4′-dichlorobiphenyl-2-yl)-3- |
| | | difluoromethyl-1-methyl-1H-pyrazole-4- |
| | | carboxamide |
| E-57 | a compound of the formula I | N-(2′,5′-difluorobiphenyl-2-yl)-1-methyl-3- |
| | | trifluoromethyl-1H-pyrazole-4-carboxamide |
| E-58 | a compound of the formula I | N-(2′,5′-dichlorobiphenyl-2-yl)-1-methyl-3- |
| | | trifluoromethyl-1H-pyrazole-4-carboxamide |
| E-59 | a compound of the formula I | N-(2′,5′-difluorobiphenyl-2-yl)-3- |
| | | difluoromethyl-1-methyl-1H-pyrazole-4- |
| | | carboxamide |
| E-60 | a compound of the formula I | N-(2′,5′-dichlorobiphenyl-2-yl)-3- |
| | | difluoromethyl-1-methyl-1H-pyrazole-4- |
| | | carboxamide |
| E-61 | a compound of the formula I | N-(3′,5′-difluorobiphenyl-2-yl)-1-methyl-3- |
| | | trifluoromethyl-1H-pyrazole-4-carboxamide |
| E-62 | a compound of the formula I | N-(3′,5′-dichlorobiphenyl-2-yl)-1-methyl-3- |
| | | trifluoromethyl-1H-pyrazole-4-carboxamide |
| E-63 | a compound of the formula I | N-(3′,5′-difluorobiphenyl-2-yl)-3- |
| | | difluoromethyl-1-methyl-1H-pyrazole-4- |
| | | carboxamide |
| E-64 | a compound of the formula I | N-(3′,5′-dichlorobiphenyl-2-yl)-3- |
| | | difluoromethyl-1-methyl-1H-pyrazole-4- |
| | | carboxamide |
| E-65 | a compound of the formula I | N-(3′-fluorobiphenyl-2-yl)-1-methyl-3- |
| | | trifluoromethyl-1H-pyrazole-4-carboxamide |
| E-66 | a compound of the formula I | N-(3′-chlorobiphenyl-2-yl)-1-methyl-3- |
| | | trifluoromethyl-1H-pyrazole-4-carboxamide |
| E-67 | a compound of the formula I | N-(3′-fluorobiphenyl-2-yl)-3-difluoromethyl- |
| | | 1-methyl-1H-pyrazole-4-carboxamide |
| E-68 | a compound of the formula I | N-(3′-chlorobiphenyl-2-yl)-3-difluoromethyl- |
| | | 1-methyl-1H-pyrazole-4-carboxamide |
| E-69 | a compound of the formula I | N-(2′-fluorobiphenyl-2-yl)-1-methyl-3- |
| | | trifluoromethyl-1H-pyrazole-4-carboxamide |
| E-70 | a compound of the formula I | N-(2′-chlorobiphenyl-2-yl)-1-methyl-3- |
| | | trifluoromethyl-1H-pyrazole-4-carboxamide |
| E-71 | a compound of the formula I | N-(2′-fluorobiphenyl-2-yl)-3-difluoromethyl- |
| | | 1-methyl-1H-pyrazole-4-carboxamide |
| E-72 | a compound of the formula I | N-(2′-chlorobiphenyl-2-yl)-3-difluoromethyl- |
| | | 1-methyl-1H-pyrazole-4-carboxamide |
| E-73 | a compound of the formula I | N-(2′-fluoro-4′-chloro-5′-methylbiphenyl-2- |
| | | yl)-1-methyl-3-trifluoromethyl-1H-pyrazole- |
| | | 4-carboxamide |
| E-74 | a compound of the formula I | N-(3′,4′,5′-trifluorobiphenyl-2-yl)-1-methyl- |
| | | 3-trifluoromethyl-1H-pyrazole-4- |
| | | carboxamide |
| E-75 | a compound of the formula I | N-(3′,4′,5′-trifluorobiphenyl-2-yl)-1-methyl- |
| | | 3-difluoromethyl-1H-pyrazole-4- |
| | | carboxamide |
| E-76 | a compound of the formula I | N-(2′,4′,5′-trifluorobiphenyl-2-yl)-1-methyl- |
| | | 3-difluoromethyl-1H-pyrazole-4- |
| | | carboxamide |
| E-77 | a compound of the formula I | N-(3′,4′,5′-trifluorobiphenyl-2-yl)-3- |
| | | chlorofluoromethyl-1-methyl-1H-pyrazole- |
| | | 4-carboxamide |
| E-78 | a compound of the formula I | N-[2-(1,1,2,3,3,3- |
| | | hexafluoropropoxy)phenyl]-1-methyl-3- |
| | | trifluoromethyl-1H-pyrazole-4-carboxamide |
| E-79 | a compound of the formula I | N-[2-(1,1,2,3,3,3-hexafluoropropoxy)- |
| | | phenyl]-3-difluoromethyl-1-methyl-1H- |
| | | pyrazole-4-carboxamide |
| E-80 | a compound of the formula I | N-[2-(2-chloro-1,1,2-trifluoroethoxy)- |
| | | phenyl]-1-methyl-3-trifluoromethyl-1H- |
| | | pyrazole-4-carboxamide |
| E-81 | a compound of the formula I | N-[2-(2-chloro-1,1,2- |
| | | trifluoroethoxy)phenyl]-3-difluoromethyl-1- |
| | | methyl-1H-pyrazole-4-carboxamide |
| E-82 | a compound of the formula I | N-[2-(1,1,2,2-tetrafluoroethoxy)phenyl]-3- |
| | | difluoromethyl-1-methyl-1H-pyrazole-4- |
| | | carboxamide |
| E-83 | a compound of the formula I | N-[2-(1,1,2,2-tetrafluorethoxy)phenyl]-1- |
| | | methyl-3-trifluoromethyl-1H-pyrazole-4- |
| | | carboxamide |
| E-84 | a compound of the formula I | N-(4′-(trifluoromethylthio)biphenyl-2-yl)-3- |
| | | difluoromethyl-1-methyl-1H-pyrazole-4- |
| | | carboxamide |
| E-85 | a compound of the formula I | N-(4′-(trifluoromethylthio)biphenyl-2-yl)-1- |
| | | methyl-3-trifluoromethyl-1H-pyrazole-4- |
| | | carboxamide |
| E-86 | a compound of the formula I | [2-(1,2-dimethylpropyl)phenyl]-5-fluoro- |
| | | 1,3-dimethyl-1H-pyrazole-4-carboxamide |
| E-87 | a compound of the formula I | N-(2-{4-[3-(4-chlorophenyl)prop-2- |
| | | ynyloxy]-3-methoxyphenyl}ethyl)-2- |
| | | methanesulfonylamino-3- |
| | | methylbutyramide |
| E-88 | a compound of the formula I | N-(2-{4-[3-(4-chlorophenyl)prop-2- |
| | | ynyloxy]-3-methoxyphenyl}ethyl)-2- |
| | | ethanesulfonylamino-3-methylbutyramide |
| E-89 | a compound of the formula I | dimethomorph |
| E-90 | a compound of the formula I | flumorph |
| E-91 | a compound of the formula I | flumetover |
| E-92 | a compound of the formula I | fluopicolide (picobenzamid) |
| E-93 | a compound of the formula I | fluopyram |
| E-94 | a compound of the formula I | zoxamide |
| E-95 | a compound of the formula I | N-(3-ethyl-3,5,5-trimethylcyclohexyl)- |
| | | 3-formylamino-2-hydroxybenzamide |
| E-96 | a compound of the formula I | carpropamid |
| E-97 | a compound of the formula I | diclocymet |
| E-98 | a compound of the formula I | mandipropamid |
| E-99 | a compound of the formula I | oxytetracyclin |
| E-100 | a compound of the formula I | silthiofam |
| E-101 | a compound of the formula I | N-(6-methoxypyridin-3-yl)cyclopropane- |
| | | carboxamide |
| E-102 | a compound of the formula I | azaconazole |
| E-103 | a compound of the formula I | bitertanol |
| E-104 | a compound of the formula I | bromuconazole |
| E-105 | a compound of the formula I | cyproconazole |
| E-106 | a compound of the formula I | difenoconazole |
| E-107 | a compound of the formula I | diniconazole |
| E-108 | a compound of the formula I | diniconazole-M |
| E-109 | a compound of the formula I | enilconazole |
| E-110 | a compound of the formula I | epoxiconazole |
| E-111 | a compound of the formula I | fenbuconazole |
| E-112 | a compound of the formula I | flusilazole |
| E-113 | a compound of the formula I | fluquinconazole |
| E-114 | a compound of the formula I | flutriafol |
| E-115 | a compound of the formula I | hexaconazole |
| E-116 | a compound of the formula I | imibenconazole |
| E-117 | a compound of the formula I | ipconazole |
| E-118 | a compound of the formula I | metconazole |
| E-119 | a compound of the formula I | myclobutanil |
| E-120 | a compound of the formula I | oxpoconazole |
| E-121 | a compound of the formula I | paclobutrazole |
| E-122 | a compound of the formula I | penconazole |
| E-123 | a compound of the formula I | propiconazole |
| E-124 | a compound of the formula I | prothioconazole |
| E-125 | a compound of the formula I | simeconazole |
| E-126 | a compound of the formula I | tebuconazole |
| E-127 | a compound of the formula I | tetraconazole |
| E-128 | a compound of the formula I | triadimenol |
| E-129 | a compound of the formula I | triadimefon |
| E-130 | a compound of the formula I | triticonazole |
| E-131 | a compound of the formula I | uniconazole |
| E-132 | a compound of the formula I | 1-(4-chlorophenyl)-2-([1,2,4]triazol-1-yl)- |
| | | cycloheptanol |
| E-133 | a compound of the formula I | cyazofamid |
| E-134 | a compound of the formula I | imazalil |
| E-135 | a compound of the formula I | imazalil-sulfate |
| E-136 | a compound of the formula I | pefurazoate |
| E-137 | a compound of the formula I | prochloraz |
| E-138 | a compound of the formula I | triflumizole |
| E-139 | a compound of the formula I | benomyl |
| E-140 | a compound of the formula I | carbendazim |
| E-141 | a compound of the formula I | fuberidazole |
| E-142 | a compound of the formula I | thiabendazole |
| E-143 | a compound of the formula I | ethaboxam |
| E-144 | a compound of the formula I | etridiazole |
| E-145 | a compound of the formula I | hymexazole |
| E-146 | a compound of the formula I | fluazinam |
| E-147 | a compound of the formula I | pyrifenox |
| E-148 | a compound of the formula I | 1-(4-chlorophenyl)-1-(propyn-2-yloxy)- |
| | | 3-(4-(3,4-dimethoxyphenyl)isoxazol-5-yl)- |
| | | propan-2-one |
| E-149 | a compound of the formula I | 3-[5-(4-chlorophenyl)-2,3-dimethyl- |
| | | isoxazolidin-3-yl]pyridine |
| E-150 | a compound of the formula I | 2,3,5,6-tetrachloro-4-methanesulfonyl- |
| | | pyridine |
| E-151 | a compound of the formula I | 3,4,5-trichloropyridine-2,6-dicarbonitrile |
| E-152 | a compound of the formula I | N-(1-(5-bromo-3-chloropyridin-2-yl)ethyl)- |
| | | 2,4-dichloronicotinamide |
| E-153 | a compound of the formula I | N-((5-bromo-3-chloropyridin-2-yl)methyl)- |
| | | 2,4-dichloronicotinamide |
| E-154 | a compound of the formula I | bupirimate |
| E-155 | a compound of the formula I | cyprodinil |
| E-156 | a compound of the formula I | diflumetorim |
| E-157 | a compound of the formula I | ferimzone |
| E-158 | a compound of the formula I | fenarimol |
| E-159 | a compound of the formula I | mepanipyrim |
| E-160 | a compound of the formula I | nitrapyrin |
| E-161 | a compound of the formula I | nuarimol |
| E-162 | a compound of the formula I | pyrimethanil |
| E-163 | a compound of the formula I | fludioxonil |
| E-164 | a compound of the formula I | fenpiclonil |
| E-165 | a compound of the formula I | aldimorph |
| E-166 | a compound of the formula I | dodemorph |
| E-167 | a compound of the formula I | dodemorph acetate |
| E-168 | a compound of the formula I | fenpropimorph |
| E-169 | a compound of the formula I | tridemorph |
| E-170 | a compound of the formula I | fluoroimid |
| E-171 | a compound of the formula I | iprodione |
| E-172 | a compound of the formula I | procymidone |
| E-173 | a compound of the formula I | vinclozolin |
| E-174 | a compound of the formula I | acibenzolar-S-methyl |
| E-175 | a compound of the formula I | amisulbrom |
| E-176 | a compound of the formula I | anilazin |
| E-177 | a compound of the formula I | blasticidin-S |
| E-178 | a compound of the formula I | captan |
| E-179 | a compound of the formula I | captafol |
| E-180 | a compound of the formula I | quinomethionate |
| E-181 | a compound of the formula I | dazomet |
| E-182 | a compound of the formula I | debacarb |
| E-183 | a compound of the formula I | diclomezine |
| E-184 | a compound of the formula I | difenzoquat |
| E-185 | a compound of the formula I | difenzoquat methylsulfate |
| E-186 | a compound of the formula I | famoxadone |
| E-187 | a compound of the formula I | fenamidone |
| E-188 | a compound of the formula I | fenoxanil |
| E-189 | a compound of the formula I | fenpropidin |
| E-190 | a compound of the formula I | folpet |
| E-191 | a compound of the formula I | octhilinone |
| E-192 | a compound of the formula I | oxolinic acid |
| E-193 | a compound of the formula I | piperalin |
| E-194 | a compound of the formula I | probenazole |
| E-195 | a compound of the formula I | proquinazid |
| E-196 | a compound of the formula I | pyroquilon |
| E-197 | a compound of the formula I | quinoxyfen |
| E-198 | a compound of the formula I | triazoxid |
| E-199 | a compound of the formula I | tricyclazole |
| E-200 | a compound of the formula I | triforine |
| E-201 | a compound of the formula I | 5-chloro-7-(4-methylpiperidin-1-yl)- |
| | | 6-(2,4,6-trifluorophenyl)-[1,2,4]triazolo- |
| | | [1,5-a]pyrimidine |
| E-202 | a compound of the formula I | 6-(4-tert-butylphenyl)-5-methyl- |
| | | [1,2,4]triazolo[1,5-a]pyrimidin-7-ylamine |
| E-203 | a compound of the formula I | 5-methyl-6-(3,5,5-trimethylhexyl)- |
| | | [1,2,4]triazolo[1,5-a]pyrimidin-7-ylamine |
| E-204 | a compound of the formula I | 5-methyl-6-octyl-[1,2,4]triazolo[1,5-a]- |
| | | pyrimidin-7-ylamine |
| E-205 | a compound of the formula I | 6-methyl-5-octyl-[1,2,4]triazolo[1,5-a]- |
| | | pyrimidin-7-ylamine |
| E-206 | a compound of the formula I | 6-ethyl-5-octyl-[1,2,4]triazolo[1,5-a]- |
| | | pyrimidin-7-ylamine |
| E-207 | a compound of the formula I | 5-ethyl-6-octyl-[1,2,4]triazolo[1,5- |
| | | a]pyrimidin-7-ylamine |
| E-208 | a compound of the formula I | 5-ethyl-6-(3,5,5-trimethylhexyl)- |
| | | [1,2,4]triazolo[1,5-a]pyrimidin-7-ylamine |
| E-209 | a compound of the formula I | 6-octyl-5-propyl-[1,2,4]triazolo[1,5-a]- |
| | | pyrimidin-7-ylamine |
| E-210 | a compound of the formula I | 5-methoxymethyl-6-octyl- |
| | | [1,2,4]triazolo[1,5-a]pyrimidin-7-ylamine |
| E-211 | a compound of the formula I | 6-octyl-5-trifluoromethyl[1,2,4]triazolo[1,5- |
| | | a]pyrimidin-7-ylamine |
| E-212 | a compound of the formula I | 5-trifluoromethyl-6-(3,5,5-trimethylhexyl)- |
| | | [1,2,4]triazolo[1,5-a]pyrimidin-7-ylamine |
| E-213 | a compound of the formula I | 2-butoxy-6-iodo-3-propylchromen-4-one |
| E-214 | a compound of the formula I | ferbam |
| E-215 | a compound of the formula I | mancozeb |
| E-216 | a compound of the formula I | maneb |
| E-217 | a compound of the formula I | metiram |
| E-218 | a compound of the formula I | metam |
| E-219 | a compound of the formula I | methasulphocarb |
| E-220 | a compound of the formula I | propineb |
| E-221 | a compound of the formula I | thiram |
| E-222 | a compound of the formula I | zineb |
| E-223 | a compound of the formula I | ziram |
| E-224 | a compound of the formula I | diethofencarb |
| E-225 | a compound of the formula I | benthiavalicarb |
| E-226 | a compound of the formula I | iprovalicarb |
| E-227 | a compound of the formula I | propamocarb |
| E-228 | a compound of the formula I | propamocarb hydrochloride |
| E-229 | a compound of the formula I | 3-(4-chlorophenyl)-3-(2-isopropoxy- |
| | | carbonylamino-3-methylbutyrylamino)- |
| | | propionic acid methyl ester |
| E-230 | a compound of the formula I | valiphenal |
| E-231 | a compound of the formula I | 4-fluorophenyl N-(1-(1-(4-cyanophenyl)- |
| | | ethanesulfonyl)but-2-yl)carbamate |
| E-232 | a compound of the formula I | dodine |
| E-233 | a compound of the formula I | dodine free base |
| E-234 | a compound of the formula I | iminoctadine |
| E-235 | a compound of the formula I | iminoctadine triacetate |
| E-236 | a compound of the formula I | iminoctadine tris(albesilate) |
| E-237 | a compound of the formula I | guazatine |
| E-238 | a compound of the formula I | guazatine acetate |
| E-239 | a compound of the formula I | kasugamycin |
| E-240 | a compound of the formula I | kasugamycin hydrochloride hydrate |
| E-241 | a compound of the formula I | polyoxine |
| E-242 | a compound of the formula I | streptomycin |
| E-243 | a compound of the formula I | validamycin A |
| E-244 | a compound of the formula I | binapacryl |
| E-245 | a compound of the formula I | dicloran |
| E-246 | a compound of the formula I | dinobuton |
| E-247 | a compound of the formula I | dinocap |
| E-248 | a compound of the formula I | nitrothal-isopropyl |
| E-249 | a compound of the formula I | tecnazen |
| E-250 | a compound of the formula I | fentin acetate |
| E-251 | a compound of the formula I | fentin chloride |
| E-252 | a compound of the formula I | fentin hydroxide |
| E-253 | a compound of the formula I | isoprothiolane |
| E-254 | a compound of the formula I | dithianon |
| E-255 | a compound of the formula I | edifenphos |
| E-256 | a compound of the formula I | fosetyl |
| E-257 | a compound of the formula I | fosetyl aluminum |
| E-258 | a compound of the formula I | iprobenfos |
| E-259 | a compound of the formula I | pyrazophos |
| E-260 | a compound of the formula I | tolclofos-methyl |
| E-261 | a compound of the formula I | chlorothalonil |
| E-262 | a compound of the formula I | dichlofluanid |
| E-263 | a compound of the formula I | dichlorophen |
| E-264 | a compound of the formula I | flusulfamide |
| E-265 | a compound of the formula I | hexachlorobenzene |
| E-266 | a compound of the formula I | pencycuron |
| E-267 | a compound of the formula I | pentachlorophenol and salts thereof |
| E-268 | a compound of the formula I | phthalide |
| E-269 | a compound of the formula I | quintozene |
| E-270 | a compound of the formula I | thiophanate methyl |
| E-271 | a compound of the formula I | tolylfluanid |
| E-272 | a compound of the formula I | N-(4-chloro-2-nitrophenyl)-N-ethyl- |
| | | 4-methylbenzenesulfonamide |
| E-273 | a compound of the formula I | phosphorous acid and its salts |
| E-274 | a compound of the formula I | sulfur |
| E-275 | a compound of the formula I | Bordeaux mixture |
| E-276 | a compound of the formula I | copper acetate |
| E-277 | a compound of the formula I | copper hydroxide |
| E-278 | a compound of the formula I | copper oxychloride |
| E-279 | a compound of the formula I | basic copper sulfate |
| E-280 | a compound of the formula I | biphenyl |
| E-281 | a compound of the formula I | bronopol |
| E-282 | a compound of the formula I | cyflufenamid |
| E-283 | a compound of the formula I | cymoxanil |
| E-284 | a compound of the formula I | diphenylamine |
| E-285 | a compound of the formula I | metrafenon |
| E-286 | a compound of the formula I | mildiomycin |
| E-287 | a compound of the formula I | oxine-copper |
| E-288 | a compound of the formula I | prohexadione-calcium |
| E-289 | a compound of the formula I | spiroxamine |
| E-290 | a compound of the formula I | tolylfluanid |
| E-291 | a compound of the formula I | N-(cyclopropylmethoxyimino-(6-difluoro- |
| | | methoxy-2,3-difluorophenyl)methyl)- |
| | | 2-phenylacetamide |
| E-292 | a compound of the formula I | N′-(4-(4-chloro-3-trifluoromethylphenoxy)- |
| | | 2,5-dimethylphenyl)-N-ethyl-N-methyl- |
| | | formamidine |
| E-293 | a compound of the formula I | N′-(4-(4-fluoro-3-trifluoromethylphenoxy)- |
| | | 2,5-dimethylphenyl)-N-ethyl-N-methyl- |
| | | formamidine |
| E-294 | a compound of the formula I | N′-(2-methyl-5-trifluoromethyl-4-(3-tri- |
| | | methylsilanylpropoxy)phenyl)-N-ethyl- |
| | | N-methylformamidine |
| E-295 | a compound of the formula I | N′-(5-difluoromethyl-2-methyl-4-(3-tri- |
| | | methylsilanylpropoxy)phenyl)-N-ethyl- |
| | | N-methylformamidine |
| E-296 | a compound of the formula I | 2-(2-(3-(2,6-dichlorophenyl)-1-methyl- |
| | | allylideneaminooxymethyl)phenyl)-2- |
| | | methoxyimino-N-methylacetamide |
| E-297 | a compound of the formula I | isopyrazam |
| E-298 | a compound of the formula I | metalaxyl-M (mefenoxam) |
| E-299 | a compound of the formula I | N-(3′,4′-dichloro-5-fluorobiphenyl-2-yl)-3- |
| | | trifluoromethyl-1-methyl-1H-pyrazol-4- |
| | | carboxamide |
| E-300 | a compound of the formula I | N-(2-(1,3,3-trimethylbutyl)phenyl)-1,3- |
| | | dimethyl-5-fluoro-1H-pyrazole-4- |
| | | carboxamide |
| E-301 | a compound of the formula I | N-[1,2,3,4-tetrahydro-9-(1-methylethyl)- |
| | | 1,4-methanonaphthalene-5-yl]-3- |
| | | (difluoromethyl)-1-methyl-1H-pyrazole-4- |
| | | carboxamide |
| E-302 | a compound of the formula I | N′-(4-(4-chloro-3-trifluoromethylphenoxy)- |
| | | 2,5-dimethylphenyl)-N-ethyl-N- |
| | | methylformamidine |
| E-303 | a compound of the formula I | N′-(4-(4-fluoro-3-trifluoromethylphenoxy)- |
| | | 2,5-dimethylphenyl)-N-ethyl-N- |
| | | methylformamidine |
| E-304 | a compound of the formula I | N′-(2-methyl-5-trifluoromethyl-4-(3- |
| | | trimethylsilanylpropoxy)phenyl)-N-ethyl-N- |
| | | methylformamidine |
| E-305 | a compound of the formula I | N′-(5-difluoromethyl-2-methyl-4-(3- |
| | | trimethylsilanylpropoxy)phenyl)-N-ethyl-N- |
| | | methylformamidine |
| E-306 | a compound of the formula I | 2-(4-chlorophenyl)-N-[4-(3,4- |
| | | dimethoxyphenyl)isoxazol-5-yl]-2-prop-2- |
| | | inyloxyacetamide |
| E-307 | a compound of the formula I | 3-[5-(4-methylphenyl)-2,3- |
| | | dimethylisoxazolidin-3-yl]-pyridine |
| E-308 | a compound of the formula I | flutianil |
| E-309 | a compound of the formula I | S-allyl 5-amino-2-isopropyl-3-oxo-4-ortho- |
| | | tolyl-2,3-dihydropyrazol-1-thiocarboxylate |
| E-310 | a compound of the formula I | 5-chloro-1-(4,6-dimethoxypyrimidin-2-yl)-2- |
| | | methyl-1H-benzoimidazol |
| E-311 | a compound of the formula I | 6-(3,4-dichlorophenyl)-5-methyl- |
| | | [1,2,4]triazolo[1,5-a]pyrimidin-7-ylamine |
| E-312 | a compound of the formula I | methyl N-(1,2,3,4-tetrahydronaphthalene- |
| | | 1-yl)-2-{1-[2-(5-methyl-3- |
| | | trifluoromethylpyrazol-1-yl)acetyl]piperidin- |
| | | 4-yl}thiazole-4-carboxamide |
| E-313 | a compound of the formula I | methyl (R)—N-(1,2,3,4- |
| | | tetrahydronaphthalene-1-yl)-2-{1-[2-(5- |
| | | methyl-3-trifluoromethylpyrazol-1- |
| | | yl)acetyl]piperidin-4-yl}thiazole-4- |
| | | carboxamide |
| E-314 | a compound of the formula I | 6-tert-butyl-8-fluoro-2,3-dimethylquinolin-4- |
| | | yl acetate |
| E-315 | a compound of the formula I | 6-tert-butyl-8-fluoro-2,3-dimethylquinolin-4- |
| | | yl methoxy acetate |
| E-316 | a compound of the formula I | carbaryl |
| E-317 | a compound of the formula I | carbofuran |
| E-318 | a compound of the formula I | carbosulfan |
| E-319 | a compound of the formula I | methomylthiodicarb |
| E-320 | a compound of the formula I | bifenthrin |
| E-321 | a compound of the formula I | cyfluthrin |
| E-322 | a compound of the formula I | cypermethrin |
| E-323 | a compound of the formula I | alpha-cypermethrin |
| E-324 | a compound of the formula I | zeta-cypermethrin |
| E-325 | a compound of the formula I | deltamethrin |
| E-326 | a compound of the formula I | esfenvalerate |
| E-327 | a compound of the formula I | lambda-cyhalothrin |
| E-328 | a compound of the formula I | permethrin |
| E-329 | a compound of the formula I | tefluthrin |
| E-330 | a compound of the formula I | diflubenzuron |
| E-331 | a compound of the formula I | flufenoxuron |
| E-332 | a compound of the formula I | lufenuron |
| E-333 | a compound of the formula I | teflubenzuron |
| E-334 | a compound of the formula I | spirotetramate |
| E-335 | a compound of the formula I | clothianidin |
| E-336 | a compound of the formula I | dinotefuran |
| E-337 | a compound of the formula I | imidacloprid |
| E-338 | a compound of the formula I | thiamethoxam |
| E-339 | a compound of the formula I | acetamiprid |
| E-340 | a compound of the formula I | thiacloprid |
| E-341 | a compound of the formula I | endosulfan |
| E-342 | a compound of the formula I | fipronil |
| E-343 | a compound of the formula I | abamectin |
| E-344 | a compound of the formula I | emamectin |
| E-345 | a compound of the formula I | spinosad |
| E-346 | a compound of the formula I | spinetoram |
| E-347 | a compound of the formula I | hydramethylnon |
| E-348 | a compound of the formula I | chlorfenapyr |
| E-349 | a compound of the formula I | fenbutatin oxide |
| E-350 | a compound of the formula I | indoxacarb |
| E-351 | a compound of the formula I | metaflumizone |
| E-352 | a compound of the formula I | flonicamid |
| E-353 | a compound of the formula I | lubendiamid |
| E-354 | a compound of the formula I | chlorantraniliprol |
| E-355 | a compound of the formula I | cyazypyr (HGW86) |
| E-356 | a compound of the formula I | cyflumetofen |
| E-357 | a compound of the formula I | acetochlor |
| E-358 | a compound of the formula I | dimethenamid |
| E-359 | a compound of the formula I | metolachlor |
| E-360 | a compound of the formula I | metazachlor |
| E-361 | a compound of the formula I | glyphosate |
| E-362 | a compound of the formula I | glufosinate |
| E-363 | a compound of the formula I | sulfosate |
| E-364 | a compound of the formula I | clodinafop |
| E-365 | a compound of the formula I | fenoxaprop |
| E-366 | a compound of the formula I | fluazifop |
| E-367 | a compound of the formula I | haloxyfop |
| E-368 | a compound of the formula I | paraquat |
| E-369 | a compound of the formula I | phenmedipham |
| E-370 | a compound of the formula I | clethodim |
| E-371 | a compound of the formula I | cycloxydim |
| E-372 | a compound of the formula I | profoxydim |
| E-373 | a compound of the formula I | sethoxydim |
| E-374 | a compound of the formula I | tepraloxydim |
| E-375 | a compound of the formula I | pendimethalin |
| E-376 | a compound of the formula I | prodiamine |
| E-377 | a compound of the formula I | trifluralin |
| E-378 | a compound of the formula I | acifluorfen |
| E-379 | a compound of the formula I | bromoxynil |
| E-380 | a compound of the formula I | imazamethabenz |
| E-381 | a compound of the formula I | imazamox |
| E-382 | a compound of the formula I | imazapic |
| E-383 | a compound of the formula I | imazapyr |
| E-384 | a compound of the formula I | imazaquin |
| E-385 | a compound of the formula I | imazethapyr |
| E-386 | a compound of the formula I | 2,4-dichlorophenoxy acetic acid (2,4-D) |
| E-387 | a compound of the formula I | chloridazon |
| E-388 | a compound of the formula I | clopyralid |
| E-389 | a compound of the formula I | fluroxypyr |
| E-390 | a compound of the formula I | picloram |
| E-391 | a compound of the formula I | picolinafen |
| E-392 | a compound of the formula I | bensulfuron |
| E-393 | a compound of the formula I | chlorimuron-ethyl |
| E-394 | a compound of the formula I | cyclosulfamuron |
| E-395 | a compound of the formula I | iodosulfuron |
| E-396 | a compound of the formula I | mesosulfuron |
| E-397 | a compound of the formula I | metsulfuron-methyl |
| E-398 | a compound of the formula I | nicosulfuron |
| E-399 | a compound of the formula I | rimsulfuron |
| E-400 | a compound of the formula I | triflusulfuron |
| E-401 | a compound of the formula I | atrazine |
| E-402 | a compound of the formula I | hexazinone |
| E-403 | a compound of the formula I | diuron |
| E-404 | a compound of the formula I | florasulam |
| E-405 | a compound of the formula I | pyroxasulfon |
| E-406 | a compound of the formula I | bentazone |
| E-407 | a compound of the formula I | cinidon-ethlyl |
| E-408 | a compound of the formula I | cinmethylin |
| E-409 | a compound of the formula I | dicamba |
| E-410 | a compound of the formula I | diflufenzopyr |
| E-411 | a compound of the formula I | quinclorac |
| E-412 | a compound of the formula I | quinmerac |
| E-413 | a compound of the formula I | mesotrione |
| E-414 | a compound of the formula I | saflufenacil |
| E-415 | a compound of the formula I | topramezone |
| E-416 | a compound of the formula I | pyrimorph |

The active compounds specified above as component 2, their preparation, and their effect against fungal pathogens are known (cf.: http://www.hclrss.demon.co.uk/index.html; http://www.alanwood.net/pesticides/); they are available commercially. The compounds with IUPAC nomenclature, their preparation, and their fungicidal activity are likewise known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The preparation of the compositions for mixtures of active compounds is carried out in a manner known per se in the form of compositions comprising, in addition to the active compounds, a solvent or solid carrier, for example as stated for compositions of the compounds I.

With respect to the customary ingredients of such compositions, reference is made to what was said about the compositions comprising the compounds I. The compositions for mixtures of active compounds are suitable as fungicides for controlling harmful fungi. They are distinguished by excellent efficacy against a broad spectrum of phytopathogenic fungi including soil-borne pathogens originating, in particular, from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Reference is furthermore made to what was said about the efficacy of the compounds I and the compositions comprising the compounds I.

The present invention also provides the use of compounds I and their pharmaceutically acceptable salts for treating diseases, in particular to the use of the compounds I as antimycotic. Thus, one embodiment of the invention relates to a medicament comprising at least one compound of the formula I and/or a pharmaceutically acceptable salt thereof. A further embodiment relates to the use of a compound I and/or a pharmaceutically effective salt thereof for preparing an antimycotic.

The present invention furthermore provides the use of compounds II and their pharmaceutically acceptable salts for treating diseases, in particular to the use of the compounds II as antimycotic. Thus, one embodiment of the invention relates to a medicament comprising at least one compound of the formula II and/or a pharmaceutically acceptable salt thereof. A further embodiment relates to the use of a compound II and/or a pharmaceutically effective salt thereof for preparing an antimycotic.

The present invention furthermore provides the use of compounds I and their pharmaceutically acceptable salts for treating tumors in mammals such as, for example, in humans. Thus, one embodiment of the invention relates to the use of a compound I and/or a pharmaceutically active salt thereof for preparing a composition which inhibits the growth of tumors and cancer in mammals. “Cancer” refers in particular to malignant tumors, for example breast cancer, prostate cancer, lung cancer, cancer of the CNS, melanocarcinomas, ovarial carcinomas or renal cancer, in particular in humans.

The present invention furthermore provides the use of compounds I and their pharmaceutically acceptable salts for treating virus infections, in particular virus infections causing dieses in warm-blooded animals. Thus, one embodiment of the invention relates to the use of a compound I and/or a pharmaceutically active salt thereof for preparing a composition for treating virus infections. The virus dieses to be treated include retrovirus diseases, such as, for example: HIV and HTLV, influenza virus, rhino virus diseases, herpes and the like.

### SYNTHESIS EXAMPLES

With appropriate modification of the starting materials, the procedures given in the synthesis examples below were used to obtain further compounds of the formula I or precursors thereof:

Melting points were determined on a MeI-Temp II instrument and are uncorrected.

¹H-NMR spectra were measured on a Bruker AC 300 spectrometer at 300 MHz and are based on tetramethylsilane as internal standard (from Aldrich or Cambridge Isotope Laboratories).

ESI mass spectra were measured on a Shimadzu LCMS-2010 EV mass spectrometer.

APCI mass spectra were measured on a Shimadzu LCMS-2010 EV mass spectrometer.

HPLC analyses were carried out using an Alltech Alltima C18 Rocket column with PDA detection at 254 nm on a Shimadzu Prominence HPLC System, unless stated otherwise. The time program below was used at a flow rate of 2.5 ml per minute:

| | Percent by volume of A | Percent by volume of B |
|---|---|---|
| Time | (H₂O with | (CH₃CN with |
| [min] | 0.05% trifluoroacetic acid TFA) | 0.05% trifluoroacetic acid TFA) |
| 1.00 | 90 | 10 |
| 4.50 | 0 | 100 |
| 10.00 | 0 | 100 |
| 11.50 | 90 | 10 |

### EXAMPLE 1

### Synthesis of 1-[rel-(2R,3S)-3-(2-chlorophenyl)-2-(2,4-difluorophenyl)oxiranylmethyl]-1H-[1,2,4]triazole-3-thiol: (=1-[rel-(2S,3R)-3-(2-chlorophenyl)-2-(2,4-difluorophenyl)oxiranylmethyl]-1H-[1,2,4]triazole-5(4H-thione)

At −78° C., 60.6 ml of a 1.7M solution of n-butyllithium in hexane were added dropwise to solution of 30g of 1-[rel-(2R,3S)-3-(2-chlorophenyl)-2-(2,4-difluorophenyl)oxiranylmethyl]-1H-[1,2,4]triazole in 800 ml of anhydrous THF. 5.5 g of sulfur were then added, and the mixture was stirred at −78° C. for another 90 minutes. At −78° C., saturated ammonium chloride solution was added to the solution, and, after thawing to room temperature, the solution was extracted three times with ethyl acetate. The combined organic phases were washed with saturated sodium chloride solution, dried and concentrated. The residue was repeatedly taken up in a methyl tert-butyl ether/ethyl acetate mixture until the excess sulfur had been removed. Finally, the product was crystallized from ethyl acetate/diisopropyl ether/cyclohexane. This gave 25 g of 1-[rel-(2R,3S)-3-(2-chlorophenyl)-2-(2,4-difluorophenyl)-oxiranylmethyl]-1H-[1,2,4]triazole-3-thiol in the form of a white powder (melting point 159-160° C.). ¹H-NMR (DMSO-d₆): 13.3 (s, 1H), 8.2 (s, 1H), 7.6 (m, 2H), 7.5 (m, 2H), 7.4 (m, 1H), 7.3 (m, 1H), 7.0 (m, 1H), 4.5 (d, 1H), 4.4 (s, 1H), 4.1 (d, 1H)].

### EXAMPLE 2

### Synthesis of 1-[(2S,3R)-2-(3,4-difluorophenyl)-3-o-tolyloxiran-2-ylmethyl]-1H-1,2,4-triazole-5(4H)-thione

At −78° C., lithium diisopropylamide (LDA, 11.8 ml, 23.9 mmol, 2.0 M in THF) was added dropwise to a solution of 1-[(2S,3R)-2-(3,4-difluorophenyl)-3-o-tolyloxiran-2-ylmethyl]-1H-1,2,4-triazole (6.0 g, 18.3 mmol) in dry tetrahydrofuran (THF, 80 ml). After 30 minutes, sulfur (1.2 g, 36.6 mmol) was added. The reaction mixture was stirred for 24 hours and allowed to warm from −78° C. to 20° C. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by column chromatography (silica gel, 3:2 hexane/ethyl acetate) and recrystallized from hexane/methylene chloride. This gave the target compound (3.6 g, 55%) as a beige solid.

HPLC-MS: 3.481 min (360)

¹H NMR (300 MHz, CDCl₃) δ 7.93 (s, 1H), 7.48-7.17 (m, 7H), 4.88 (d, 1H), 4.08 (s, 1H), 3.81 (d, 1H), 2.41 (s, 3H).

### EXAMPLE 3

### Synthesis of 1-[rel-(2S,3R)-3-(2-chlorophenyl)-2-(3,4-difluorophenyl)oxiran-2-ylmethyl]-1H-1,2,4-triazole-5(4-H)-thione

At −78° C., lithium diisopropylamide (LDA, 10.5 ml, 21.0 mmol, 2.0 M in THF) was added dropwise to a solution of 1-[rel-(2S,3R)-3-(2-chlorophenyl)-2-(3,4-difluorophenyl)oxiran-2-ylmethyl]-1H-1,2,4-triazole (6.0 g, 17.5 mmol) in dry tetrahydrofuran (THF, 60 ml). After 25 minutes, sulfur (1.2 g, 38.0 mmol) was added. The reaction mixture was stirred at −78° C. for 5 hours. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by recrystallization from hexane/methylene chloride. This gave the target compound (1.24 g, 19%) as a white solid (melting point 165° C.).

### EXAMPLE 4

### Synthesis of 1-[rel-(2S,3R)-2-(2,4-difluorophenyl)-3-(2-fluorophenyl)oxiran-2-ylmethyl]-1H-1,2,4-triazole-5(4H)-thione

At −78° C., lithium diisopropylamide (4.8 ml, 9.6 mmol, 2.0 M in THF) was added dropwise to a solution of 1-[rel-(2S,3R)-2-(2,4-difluorophenyl)-3-(2-fluorophenyl)oxiran-2-ylmethyl]-1H-1,2,4-triazole (2.65 g, 8.0 mmol) in dry tetrahydrofuran (THF, 55 ml). After 20 minutes, sulfur (510 mg, 16.0 mmol) was added. The reaction mixture was stirred at −78° C. for 5 hours. Methanol (10 ml) and saturated ammonium chloride solution (30 ml) were added, and the reaction mixture was then extracted with ethyl acetate (50 ml). The organic phase was washed with saturated sodium chloride solution (three times in each case 40 ml), dried with sodium sulfate and concentrated. The residue was purified by column chromatography (silica gel, 22:3 to 83:17 methylene chloride/ethyl acetate). This gave the target compound (1.4 g, 48%) as a beige solid (melting point 144-148° C.).

### EXAMPLE 5

### Synthesis of 1-[rel-(2S,3R)-2-(3,4-difluorophenyl)-3-(2-(trifluoromethyl)phenyl)oxiran-2-ylmethyl]-1H-1,2,4-triazole-5(4H)-thione

At −78° C., lithium diisopropylamide (5.5 ml, 10.9 mmol, 2.0 M in THF) was added dropwise to a solution of 1-[rel-(2S,3R)-2-(3,4-difluorophenyl)-3-(2-(trifluoromethyl)phenypoxiran-2-ylmethyl]-1H-1,2,4-triazole (3.5 g, 9.1 mmol) in dry tetrahydrofuran (THF, 100 ml). After 20 minutes, sulfur (582 mg, 18.2 mmol) was added. Starting at −70° C., the reaction mixture was stirred for 4 hours and slowly brought to room temperature overnight. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by recrystallization from hexane/methylene chloride. This gave the target compound (1.7 g, 45%) as a beige solid (melting point 143-146° C.).

### EXAMPLE 6

### Synthesis of 1-[rel-(2S,3R)-2-(3,4-difluorophenyl)-3-o-tolyloxiran-2-ylmethyl]-5-(methylthio)-1H-1,2,4-triazole

At −78° C., LDA (0.65 ml, 1.3 mmol, 2.0 M in THF) was added dropwise to a solution of 1-[rel-(2S,3R)-2-(3,4-difluorophenyl)-3-o-tolyloxiran-2-ylmethyl]-1H-1,2,4-triazole (327 mg, 1.0 mmol) in dry tetrahydrofuran (THF, 5 ml). After 30 minutes, dimethyl disulfide (134 μl, 1.5 mmol) was added, and the solution was stirred at −78° C. for 4 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 3:1 to 1:1 hexane/ethyl acetate). This gave the target compound (250 mg, 67%) as a yellow oil.

HPLC-MS: 3.776 min (374)

### EXAMPLE 7

### Synthesis of 1-[rel-(2S,3R)-3-(2-chlorophenyl)-2-(3,4-difluorophenyl)oxiran-2-ylmethyl]-5-(methylthio)-1H-1,2,4-triazole

At −78° C., n-butyllithium (0.61 ml, 0.95 mmol, 1.6 M in hexane) was added dropwise to a solution of 1-[rel-(2S,3R)-3-(2-chlorophenyl)-2-(3,4-difluorophenyl)oxiran-2-ylmethyl]-1H-1,2,4-triazole (300 mg, 0.86 mmol) in dry tetrahydrofuran (THF, 5 ml). After 15 minutes, dimethyl disulfide (79 μl, 0.87 mmol) was added, and the solution was stirred at −78° C. for 1.5 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 9:1 to 2:1 hexane/ethyl acetate). This gave the target compound (59 mg, 17%) as a white solid (melting point 79-81° C.).

### EXAMPLE 8

### Synthesis of 1-[rel-(2S,3R)-2-(2,4-difluorophenyl)-3-(2-fluorophenyl)oxiran-2-ylmethyl]-5-(methylthio)-1H-1,2,4-triazole

At −78° C., n-butyllithium (0.67 ml, 1.09 mmol, 1.6 M in hexane) was added dropwise to a solution of 1-[rel-(2S,3R)-2-(2,4-difluorophenyl)-3-(2-fluorophenyl)oxiran-2-ylmethyl]-1H-2,4-triazole (300 mg, 0.91 mmol) in dry tetrahydrofuran (THF, 10 ml). After 20 minutes, dimethyl disulfide (81 μl, 0.91 mmol) was added, and the solution was stirred at −78° C. for 30 minutes. Methanol (10 ml) and saturated ammonium chloride solution (15 ml) were added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 43:7 methylene chloride/ethyl acetate). This gave the target compound (135 mg, 40%) as a white solid (melting point 65-68° C.).

### EXAMPLE 9

### Synthesis of 1-[rel-(2S,3R)-2-(3,4-difluorophenyl)-3-(2-(trifluoromethyl)phenyl)oxiran-2-ylmethyl]-5-(methylthio)-1H-1,2,4-triazole

At −78° C., LDA (0.32 ml, 0.64 mmol, 2.0 M in THF) was added dropwise to a solution of 1-[rel-(2S,3R)-2-(3,4-difluorophenyl)-3-(2-(trifluoromethyl)phenyl)oxiran-2-ylmethyl]-1H-1,2,4-triazole (200 mg, 0.53 mmol) in dry tetrahydrofuran (THF, 8 ml). After 15 minutes, dimethyl disulfide (56.7 μl, 0.63 mmol) was added, and the solution was stirred at −78° C. for 4 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 3:1 to 1:1 hexane/ethyl acetate). This gave the target compound (90 mg, 40%) as a beige solid (melting point 83-85° C.).

### EXAMPLE 10

### Synthesis of 1-[rel-(2S,3R)-3-(2-chlorophenyl)-2-(2,4-difluorophenyl)oxiran-2-ylmethyl]-5-(methylthio)-1H-1,2,4-triazole

300 mg of 1-[rel-(2S,3R)-3-(2-chlorophenyl)-2-(2,4-difluorophenyl)oxiranylmethyl]-1H-[1,2,4]triazole-5(4H)-thione were dissolved in 6 ml of THF, and 21 mg of sodium hydride were added at room temperature. The mixture was stirred briefly, and 112 mg of methyl iodide were then added. The mixture was stirred at room temperature overnight, water was added and the mixture was extracted repeatedly with ethyl acetate. The organic phase was washed with water, dried and concentrated. The crude product was purified by column chromatography using cyclohexane/ethyl acetate. This gave 270 mg of the desired product (87%, melting point 116-117° C.).

### EXAMPLE 11

### Synthesis of 1-[rel-(2S,3R)-3-(2-chlorophenyl)-2-(2,4-difluorophenyl)oxiran-2-ylmethyl]-5-(methylsulfonyl)-1H-1,2,4-triazole

At room temperature, a mixture of 1-[rel-(2S,3R)-3-(2-chlorophenyl)-2-(2,4-difluorophenyl)oxiran-2-ylmethyl]-5-(methylthio)-1H-1,2,4-triazole (200 mg, 0.52 mmol) and m-CPBA (270 mg, 1.56 mmol) in CH₂Cl₂ (5 ml) was stirred for 24 hours. The reaction mixture was washed with 1N sodium hydroxide solution (two times in each case 20 ml) and saturated sodium chloride solution (two times in each case 20 ml), dried with sodium sulfate and freed from the solvent. The residue was purified by column chromatography (silica gel, 5:1 hexane/ethyl acetate). This gave the target compound (150 mg, 69%) as a white solid.

HPLC-MS: 3.648 min (426)

### EXAMPLE 12

### Synthesis of 1-[rel-(2S,3R)-3-(2-chlorophenyl)-2-(2,4-difluorophenyl)oxiran-2-ylmethyl]-5-thiocyanato-1H-1,2,4-triazole

At room temperature, a mixture of 1-[rel-(2S,3R)-3-(2-chlorophenyl)-2-(2,4-difluorophenyl)oxiranylmethyl]-1H-[1,2,4]triazole-5(4H)thione (150 mg, 0.4 mmol), triethylamine (110 m, 0.8 mmol) and cyanogen bromide BrCN (63 mg, 0.59 mmol) in dry tetrahydrofuran (THF, 5 ml) was stirred for 3 hours. Ethyl acetate (20 ml) was added, and the mixture was washed with saturated sodium chloride solution (three times in each case 20 ml), dried with sodium sulfate and freed from the solvent. The residue was purified by column chromatography (silica gel, 10:1 methylene chloride/ethyl acetate). This gave the target compound (110 mg, 69%) as a white solid.

HPLC-MS: 3.756 min (405)

### EXAMPLE 13

### Synthesis of S-1-[rel-(2S,3R)-3-(2-chlorophenyl)-2-(2,4-difluorophenyl)oxiran-2-ylmethyl]-1H-1,2,4-triazol-5-yl O,O-diethyl phosphorodithioate

At room temperature, P(S)(OEt)₂Cl (105 mg, 87.5 μl, 0.56 mmol) and triethylamine (108 μl, 0.79 mmol), dropwise, and a catalytic amount of DMAP were added to a solution of 1-[rel-(2S,3R)-3-(2-chlorophenyl)-2-(2,4-difluorophenyl)oxiranylmethyl]-1H-[1,2,4]triazole-5(4H)thione (200 mg, 0.53 mmol) in dry tetrahydrofuran (THF, 8 ml). The reaction mixture was stirred at room temperature for 3 hours, saturated sodium chloride solution (30 ml) was then added and the mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 9:1 to 3:1 hexane/ethyl acetate). This gave the target compound (150 mg, 54%) as a light-yellow oil.

1H NMR (300 MHz, CDCl₃) δ 8.00 (s, 1H), 7.62-7.55 (m, 1H), 7.49-7.28 (m, 4H), 6.83-6.71 (m, 2H), 4.77 (d, 1H), 4.30-4.06 (m, 5H), 4.01 (d, 1H), 1.39-1.20 (m, 6H).

### EXAMPLE 14

### Synthesis of S-{2-[3-(2-chlorophenyl)-2-(2,4-difluorophenyl)oxiranylmethyl]-2H-[1,2,4]triazole-3-yl} rel-(2S,3R)thio acetate

300 mg of 2-[rel-(2S,3R)-3-(2-chlorophenyl)-2-(2,4-difluorophenyl)oxiranylmethyl]-2H-[1,2,4]triazole-3-thiol were dissolved in 5 ml of THF, and 0.12 ml of triethylamine was added at room temperature. The mixture was stirred briefly, and 62 mg of acetyl chloride were then added. The mixture was stirred at room temperature overnight, another 0.2 ml of triethylamine and 120 mg of acetyl chloride were then added and the mixture was again stirred overnight. Water was then added, and the reaction mixture was extracted repeatedly with ethyl acetate. The organic phase was washed with water, dried and concentrated. The crude product was purified by column chromatography using cyclohexane/ethyl acetate. This gave 270 mg of the desired product (81%, melting point 160° C.).

### EXAMPLE 15

### Synthesis of S-{2-[3-(2-chlorophenyl)-2-(2,4-difluorophenyl)oxiranylmethyl]-2H-[1,2,4]triazole-3-yl}rel-(2S,3R)thio carbonate

300 mg of 2-[rel-(2S,3R)-3-(2-chlorophenyl)-2-(2,4-difluorophenyl)oxiranylmethyl]-2H-[1,2,4]triazole-3-thiol were dissolved in 5 ml of THF, and 21 mg of sodium hydride were added at room temperature. The mixture was stirred briefly, and 75 mg of methyl chloroformate were then added. The mixture was stirred at room temperature overnight, water was added and the mixture was extracted repeatedly with ethyl acetate. The organic phase was washed with water, dried and concentrated. The crude product was purified by column chromatography using cyclohexane/ethyl acetate. This gave 100 mg of the desired product (29%).

HPLC-MS: 3.974 min (438)

### EXAMPLE 16

### Synthesis of sodium rel-(2S,3R)-2-[3-(2-chlorophenyl)-2-(2,4-difluorophenyl)oxiranylmethyl]-2H-[1,2,4]triazole-3-thiolate

300 mg of 2-[rel-(2 5, 3R)-3-(2-chlorophenyl)-2-(2,4-difluorophenyl)oxiranylmethyl]-2H-[1,2,4]triazole-3-thiol were dissolved in 5 ml of THF, and 21 mg of sodium hydride were added at room temperature. The mixture was stirred at room temperature overnight and then concentrated under reduced pressure. This gave 320 mg of the desired product (100%, melting point 188° C.).

### EXAMPLE 17

### Synthesis of 1,2-bis[1-rel-(2S,3R)-3-(2-chlorophenyl)-2-(2,4-difluorophenyl)oxiran-2-ylmethyl-1H-1,2,4-triazole-5-yl]disulfane

A mixture of 1-[rel-(2S,3R)-3-(2-chlorophenyl)-2-(2,4-difluorophenyl)oxiranylmethyl]-1H-[1,2,4]triazole-5(4H)thione (110 mg, 0.29 mmol) and iodine (81 mg, 0.32 mmol) in dry tetrahydrofuran (THF, 5 ml) was stirred at room temperature for 4 hours. The residue was purified by preparative thin-layer chromatography. This gave the target compound (45 mg, 41%) as a white solid (melting point 75-76° C.).

### EXAMPLE 18

### Synthesis of 1,2-bis[1-rel-(2S,3R)-2-(3,4-difluorophenyl)-3-o-tolyloxiran-2-ylmethyl-1H-1,2,4-triazole-5-yl]disulfane

A mixture of 1-[(2S,3R)-2-(3,4-difluorophenyl)-3-o-tolyloxiran-2-ylmethyl]-1H-1,2,4-triazole-5(4H)thione (150 mg, 0.42 mmol) and iodine (64 mg, 0.25 mmol) in dry tetrahydrofuran (THF, 5 ml) was stirred at room temperature for 4 hours. The residue was purified by preparative thin-layer chromatography. This gave the target compound (90 mg, 60%) as a white solid (melting point 64-67° C.).

### Biological Experiments

### Greenhouse

### Preparation of Active Compound

The active compounds were prepared separately or jointly as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below. Alternatively, the active compounds were used as a commercial finished formulation and diluted with water to the concentration of active compounds stated.

### USE EXAMPLE 1

### Protective Activity Against Puccinia recondita on Wheat (Brown Rust of Wheat) (Puccrt P1)

Leaves of potted wheat seedlings were sprayed to a runoff point using an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 24° C. for 24 hours. During this time, the spores germinated and the germ tubes penetrated into the leaf tissue. The next day, the test plants were returned to the greenhouse and cultivated at temperatures between 20 and 24° C. and 65 to 70% relative atmospheric humidity for a further 7 days. The extent of the rust fungus development on the leaves was then determined visually.

### USE EXAMPLE 2

### Activity Against Gray Mold on Bell Pepper Leaves Caused by Botrytis cinerea, 1 Day Protective Application (Botrci P1)

Bell pepper seedlings were, after 2-3 leaves were well developed, sprayed to runoff point with an aqueous suspension having the active compound concentration stated below. The next day, the treated plants were inoculated with a spore suspension of Botrytis cinerea in a 2% strength biomalt solution. The test plants were then placed in a dark climatized chamber at 22 to 24° C. and high atmospheric humidity. After 5 days, the extent of the fungal infection on the leaves could be determined visually in %.

### USE EXAMPLE 3

### Curative Activity Against Soybean Rust Caused by Phakopsora pachyrhizi (Phakpa K1)

Leaves of potted soybean seedlings were inoculated with a spore suspension of soybean rust (Phakopsora pachyrhizi). The pots were then placed in a chamber with high atmospheric humidity (90 to 95%) and at 23 to 27° C. for 24 hours. During this time, the spores germinated and the germ tubes penetrated into the leaf tissue. The infected plants were then sprayed to runoff point with the active compound solution described above at the active compound concentration stated below. After the spray coating had dried on, the test plants were cultivated in a greenhouse at temperatures between 23 and 27° C. and 60 to 80% relative atmospheric humidity for 14 days. The extent of the rust fungus development on the leaves was then determined visually in % infection.

| | | Infection in | Infection in | Infection in | Infection in |
|---|---|---|---|---|---|
| | | % at | % at | % at | % at |
| Compound | | 600 ppm | 600 ppm | 600 ppm | 300 ppm |
| No. | Structure | for Puccrt P1 | for Botrci P1 | for Phakpa K1 | for Puccrt P1 |
| 1 | 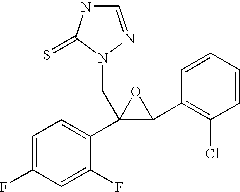 | 0 | 7 | | |
| 14 | 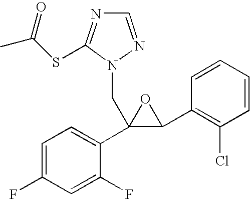 | 0 | 3 | | |
| 10 | 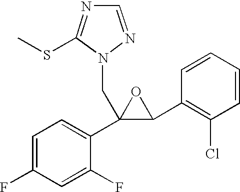 | | | 0 | |
| 3 | 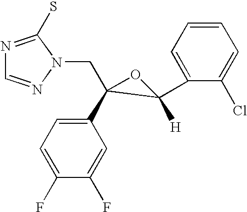 | 0 | | | |
| 9 | 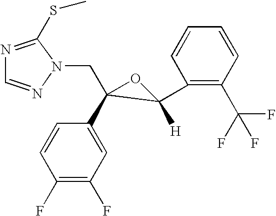 | | | 0 | |
| 8 | 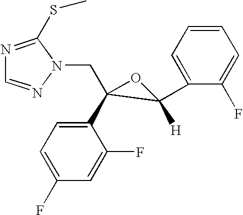 | | | 1 | |
| 5 | 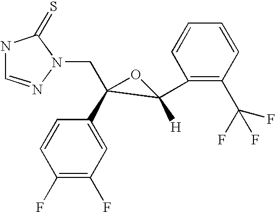 | | | | 10 |
| Untreated | | 90 | 90 | 90 | 90 |

### Microtest

The active compounds were formulated separately or jointly as a stock solution having a concentration of 10 000 ppm in DMSO.

### USE EXAMPLE NO. 4

### Activity Against the Septoria Leaf Spot Pathogen Septoria tritici in the Microtiter Test (Septtr)

The stock solution was pipetted into a microtiter plate (MTP) and diluted with water to the stated active compound concentration. An aqueous malt-based spore suspension of Septoria tritici was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| | | Growth in % |
|---|---|---|
| Compound | | at 31 ppm |
| No. | Structure | for Septtr |
| 12 | 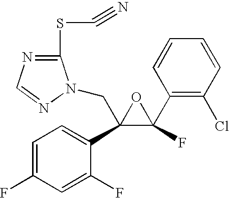 | 1 |
| 13 | 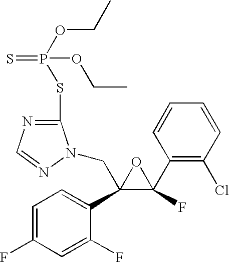 | 4 |

### USE EXAMPLE NO. 5

### Activity Against the Early Blight Pathogen Alternaria solani in the Microtiter Test (Alteso)

The stock solution was pipetted into a microtiter plate (MTP) and diluted with water to the stated active compound concentration. An aqueous malt-based spore suspension of Alternaria solani was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| | | Growth in % |
|---|---|---|
| Compound | | at 31 ppm |
| No. | Structure | for Alteso |
| 12 | 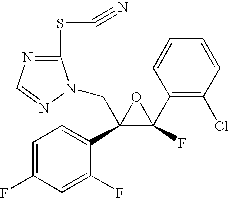 | 43 |

### USE EXAMPLE NO. 6

### Activity Against the Rice Blast Pathogen Pyricularia oryzae in the Microtiter Test (Pyrior)

The stock solution was pipetted into a microtiter plate (MTP) and diluted with water to the stated active compound concentration. An aqueous malt-based spore suspension of Pyricularia oryzae was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| | | Growth in % |
|---|---|---|
| Compound | | at 31 ppm |
| No. | Structure | for Pyrior |
| 4 | 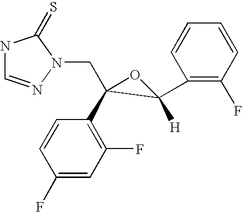 | 7 |

### USE EXAMPLE NO. 7

### Activity Against the Leaf and Gloom Blotch Pathogen Leptosphaeria nodorum in the Microtiter Test (Leptno)

The stock solution was pipetted into a microtiter plate (MTP) and diluted with water to the stated active compound concentration. An aqueous malt-based spore suspension of Leptosphaeria nodorum was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| Compound | | Growth in % at |
|---|---|---|
| No. | Structure | 31 ppm for Leptno |
| 16 | 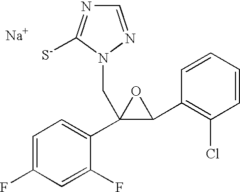 | 4 |

### Comparative Experiments

| | | Growth in % at | Growth in % at |
|---|---|---|---|
| | | 31 ppm for | 31 ppm for |
| Compound No. | Structure | Pyrior | Leptno |
| 15 | 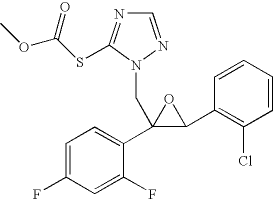 | 64 | |
| WO9742178, p. 55 (4th compound) | 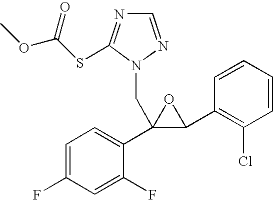 | 94 | |
| 3 | 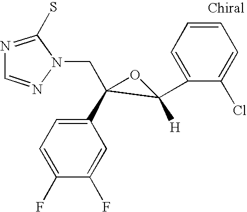 | | 60 |
| WO9638440, p. 7 (2nd compound) | 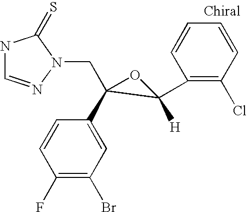 | | 100 |
| 4 | 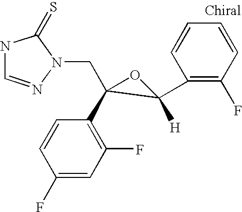 | 7 | |
| WO9638440, p. 9 (2nd compound) | 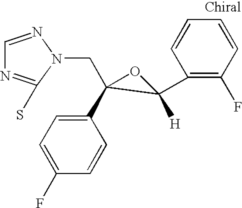 | 30 | |

1-31. (canceled)

32. An azolylmethyloxirane of the formula I 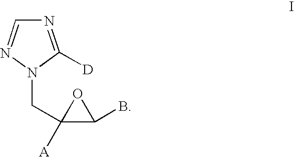
in which
A is phenyl which is substituted by an F and contains a further substituent L different from Br, where the phenyl may additionally contain one or two substituents L;
B is phenyl which is unsubstituted or substituted by one, two, three or four identical or different substituents L, where L is as defined below:
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C_{g}-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O),A¹, C(═O)A², C(═S)A², NA³A⁴, phenyl, phenyloxy or a five- or six-membered saturated, partially unsaturated or aromatic heterocycle which contains one, two, three or four heteroatoms selected from the group consisting of O, N and S; where
n is 0, 1 or 2;
A¹ is hydrogen, hydroxy, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
where the aliphatic and/or alicyclic and/or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino;
D is S—R, where
R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
R³ is C₁-C₈-alkyl, C₁-C₈-haloalkyl, C₁-C₈-alkoxy, C₁-C₈-haloalkoxy or NA³A⁴; and
R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
is a group DI 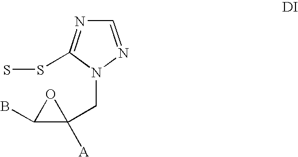
where A and B are as defined above;
is a group DII 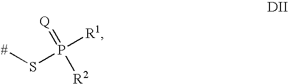
where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxyC₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where
M is an alkali metal cation, an equivalent of an alkaline earth metal cation, an equivalent of a copper, zinc, iron or nickel cation or an ammonium cation of the formula (E) 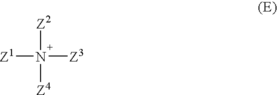
in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and an agriculturally acceptable salt thereof.

33. The compound according to claim 32, wherein A is phenyl which is substituted by F and a substituent selected from the group consisting of F, Cl, C₁-C₄-alkyl, C₁-C₄-haloalkyl, C₁-C₄-alkoxy, C₁-C₄-haloalkoxy, C₁-C₄-haloalkylthio and nitro and does not contain any further substituent or any further substituent L.

34. The compound according to claim 33, wherein A is phenyl which is substituted by F and a substituent L selected from the group consisting of F, Cl, CH₃, OCH₃, CF₃, CHF₂, OCF₃ and OCHF₂ and does not contain any further substituent.

35. The compound according to claim 33, wherein A is phenyl which is substituted by exactly two F or is phenyl which is substituted by one F and exactly one further substituent L selected from the group consisting of Cl, C₁-C₄-alkyl, C₁-C₄-haloalkyl and C₁-C₄-alkoxy and B is in each case phenyl which contains one, two or three substituents L selected from the group consisting of halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio and C₁-C₄-alkylthio.

36. The compound according to claim 33, wherein A is phenyl which is substituted by F and a substituent L selected from the group consisting of F, Cl, CH₃, OCH₃, CF₃, CHF₂, OCF₃ and OCHF₂ and contains a further substituent L.

37. The compound according to claim 36, wherein A is phenyl which is substituted by exactly three F and B is phenyl which is substituted by one, two or three substituents L selected from the group consisting of halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio and C₁-C₄-alkylthio.

38. The compound according to claim 32, where B is phenyl which contains exactly one substituent L.

39. The compound according to claim 32, where B is phenyl which contains two or three independently selected substituents L.

40. The compound according to claim 39, where at least one substituent L is located in the ortho-position to the point of attachment of the phenyl ring to the oxirane ring.

41. The compound according to claim 32, where L is in each case independently selected from the group consisting of halogen, C₁-C₄-alkyl, C₁-C₄-haloalkyl, C₁-C₄-alkoxy, C₁-C₄-haloalkoxy and C₁-C₄-haloalkylthio.

42. The compound according to claim 41, where L is in each case independently selected from the group consisting of F, Cl, CH₃, C₂H₅, CF₃, OCH₃, OC₂H₅, OCF₃, OCHF₂ and SCF₃.

43. The compound according to claim 32, where D is SH or S—C₁-C₄-alkyl.

44. The compound according to claim 32, where D is C(═O)R³ and R³ is C₁-C₄-alkyl.

45. A composition comprising a compound of the formula I according to claim 32, and/or a salt thereof.

46. The composition according to claim 45, further comprising at least one solid or liquid carrier.

47. The composition according to claim 46, further comprising at least one further fungicidally, insecticidally and/or herbicidally active compound.

48. A seed comprising at least one compound of the formula I according to claim 32, and/or an agriculturally acceptable salt thereof.

49. A method for controlling phytopathogenic fungi which comprises treating the fungi or the materials, plants, the soil or seed to be protected against fungal attack with an effective amount of a compound of the formula I according to claim 32 or an agriculturally acceptable salt thereof.

50. A medicament comprising at least one compound of the formula I according to claim 32 and/or a pharmaceutically acceptable salt thereof.

51. A process for preparing an antimycotic which comprises using at least one compound of the formula I according to claim 32 and/or a pharmaceutically acceptable salt thereof.

52. A process for preparing compounds of the formula I according to claim 32, in which D is SR where R=hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a1) reacting a compound IIIb 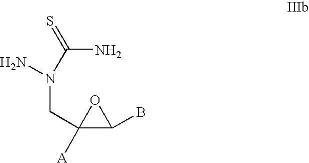
with formic acid to give compounds I-1; 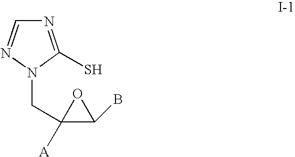
in which D is SH,
and, if desired to obtain a compound of formula I in which D is SR where R is C₁-C₈-haloalkyl, C₂-C₈-alkenyl, C₂-C₈-haloalkenyl, C₂-C₈-alkynyl or C₂-C₈-haloalkynyl:
(bp reacting compounds I-1 with R—X where X is halogen or trifluoro(C₁-C₈)alkylsulfonate.

53. A process for preparing compounds of the formula IIIb: 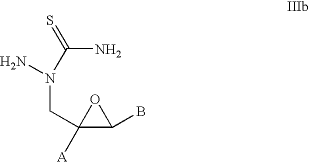
wherein A and B are as defined in claim 32;
comprising
(a2) reacting a compound of the formula Ma 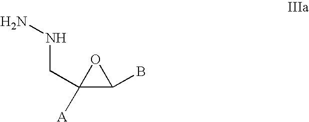
with a thiocyanate YSCN in which Y is an alkali metal or ammonium.

54. A process for preparing compounds I according to claim 32, in which D is SR where R=hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a3) oxidizing a compound of the formula IIIc 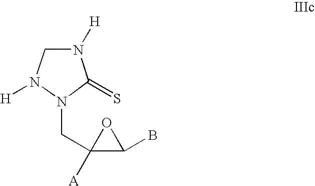
to give a compound I-1; 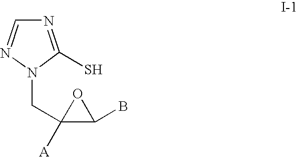
in which D is SH,
and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl:
(b3) reacting compounds I-1 with R—X where X is halogen or trifluoro(C₁-C₈)alkylsulfonate.

55. A process for preparing compounds I according to claim 32, in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a4) reacting a compound IIId 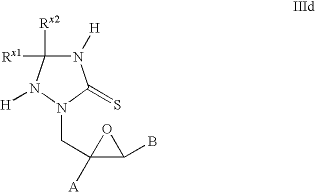
where R^{x1} ═C₁-C₄-alkyl or phenyl, R^{x2}=hydrogen or C₁-C₄-alkyl, or R^{x1} and R^{x2} together form a —(CH₂)₅— chain with formic acid to obtain compounds I-1; 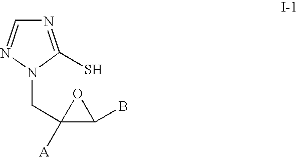
in which D is SH,
and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl:
(b4) reacting compounds I-1 with R—X where X is halogen or trifluoro(C₁-C₈)alkylsulfonate.

56. A compound of formula IIIa 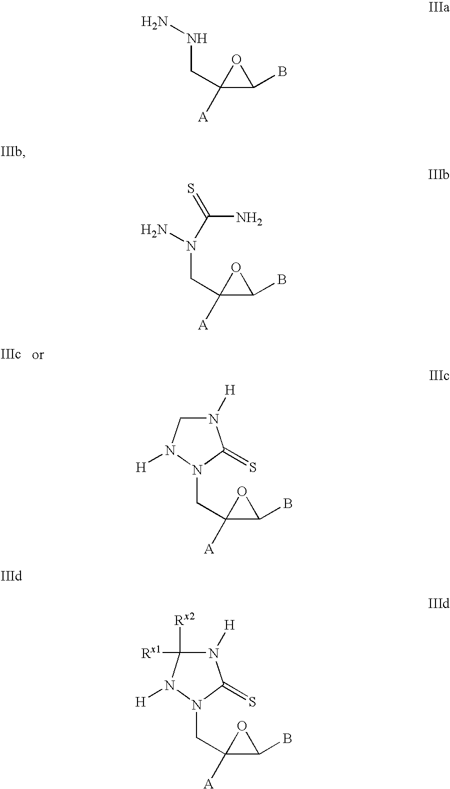
wherein A and B are as defined in claim 32; and R^{x1} is C₁-C₄-alkyl or phenyl, R^{x2} is hydrogen or C₁-C₄-alkyl, or R^{x1} and R^{x2} together form a —(CH₂)₅— chain,
or an agriculturally acceptable salt thereof.

57. A process for preparing compounds I according to claim 32, in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a5) reacting of a compound of the formula II 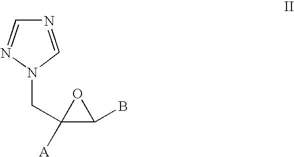
with a strong base and sulfur powder to give a compound I-1; 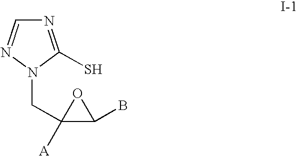
in which D is SH,
and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b5) reacting compounds I-1 with R—X where X is halogen or tri-fluoro(C₁-C₈)alkylsulfonate.

58. A process for preparing compounds of the formula II, 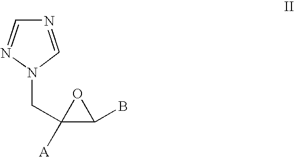
wherein A and B are as defined in claim 32;
which comprises
(a6) reacting a compound of the formula III 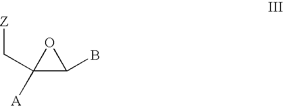
in which Z is a leaving group, with 1,2,4-triazole and a base.

59. The compound of the formula III 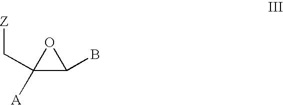
in which Z is a leaving group and A and B are as defined in claim 32, except for the compounds:
anti-2-(2,4-difluorophenyl)-2-(chloromethyl)-3-(4-chlorophenyl)oxirane,
anti-2-(2,4-difluorophenyl)-2-(chloromethyl)-3-(3-chlorophenyl)oxirane,
anti-2-(2,4-difluorophenyl)-2-(chloromethyl)-3-(3,4-dichlorophenyl)oxirane,
anti-2-(2,4-difluorophenyl)-2-(chloromethyl)-3-(2-fluorophenyl)oxirane,
anti-2-(2,4-difluorophenyl)-2-(chloromethyl)-3-(3,4-difluorophenyl)oxirane,
anti-2-(2,4-difluorophenyl)-2-(chloromethyl)-3-(4-methylphenyl)oxirane,
anti-2-(2,4-difluorophenyl)-2-(chloromethyl)-3-(3,5-dichlorophenyl)oxirane,
anti-2-(2,4-difluorophenyl)-2-(chloromethyl)-3-(3-methylphenyl)oxirane,
anti-2-(2,4-difluorophenyl)-2-(chloromethyl)-3-(3,5-dimethylphenyl)oxirane,
anti-2-(2,4-difluorophenyl)-2-(chloromethyl)-3-(3,5-Difluorophenyl)oxirane,
anti-2-(2,4-difluorophenyl)-2-(chloromethyl)-3-(2-trifluoromethylphenyl)oxirane,
anti-2-(2,4-difluorophenyl)-2-(chloromethyl)-3-(2-chlorophenyl)oxirane,
anti-2-(2,5-difluorophenyl)-2-(chloromethyl)-3-(2-chlorophenyl)oxirane,
anti-2-(2,5-difluorophenyl)-2-(chloromethyl)-3-(4-chlorophenyl)oxirane,
anti-2-(2,5-difluorophenyl)-2-(chloromethyl)-3-(2-fluorophenyl)oxirane,
anti-2-(2,5-difluorophenyl)-2-(chloromethyl)-3-(2-methylphenyl)oxirane,
anti-2-(2,5-difluorophenyl)-2-(chloromethyl)-3-(3-chlorophenyl)oxirane,
anti-2-(3,4-difluorophenyl)-2-(chloromethyl)-3-(2-chlorophenyl)oxirane,
anti-2-(3,4-difluorophenyl)-2-(chloromethyl)-3-(4-fluorophenyl)oxirane,
anti-2-(3,4-difluorophenyl)-2-(chloromethyl)-3-(2-methylphenyl)oxirane,
anti-2-(3,4-difluorophenyl)-2-(chloromethyl)-3-(2-fluorophenyl)oxirane,
anti-2-(3,5-difluorophenyl)-2-(chloromethyl)-3-(2-chlorophenyl)oxirane,
anti-2-(3,5-difluorophenyl)-2-(chloromethyl)-3-(2-methylphenyl)oxirane,
anti-2-(3,5-difluorophenyl)-2-(chloromethyl)-3-(4-chlorophenyl)oxirane,
anti-2-(2,4,5-trifluorophenyl)-2-(chloromethyl)-3-(4-fluorophenyl)oxirane,
anti-2-(2,4,5-trifluorophenyl)-2-(chloromethyl)-3-(2-chlorophenyl)oxirane,
anti-2-(2,4,5-trifluorophenyl)-2-(chloromethyl)-3-(2-methylphenyl)oxirane,
anti-2-(3,4,5-trifluorophenyl)-2-(chloromethyl)-3-(4-fluorophenyl)oxirane,
anti-2-(3,4,5-trifluorophenyl)-2-(chloromethyl)-3-(2-methylphenyl)oxirane,
anti-2-(3,4,5-trifluorophenyl)-2-(chloromethyl)-3-(2-chlorophenyl)oxirane,
anti-2-(3,4,5-trifluorophenyl)-2-(chloromethyl)-3-(2-fluorophenyl)oxirane,
anti-2-(4-chloro-2-fluorophenyl)-2-(chloromethyl)-3-(2-chlorophenyl)oxirane,
anti-2-(3-fluoro-4-methoxyphenyl)-2-(chloromethyl)-3-(2-chlorophenyl)oxirane,
anti-2-(2-chloro-4-fluorophenyl)-2-(chloromethyl)-3-(2-fluorophenyl)oxirane,
anti-2-(2-chloro-4-fluorophenyl)-2-(chloromethyl)-3-(4-fluorophenyl)oxirane,
anti-2-(4-chloro-2-fluorophenyl)-2-(chloromethyl)-3-(4-chlorophenyl)oxirane,
anti-2-(4-chloro-2-fluorophenyl)-2-(chloromethyl)-3-(4-methylphenyl)oxirane,
anti-2-(4-chloro-2-fluorophenyl)-2-(chloromethyl)-3-(2-chlorophenyl)oxirane,
anti-2-(2-fluoro-4-chlorophenyl)-2-(chloromethyl)-3-(3-chlorophenyl)oxirane,
anti-2-(2-fluoro-4-chlorophenyl)-2-(chloromethyl)-3-(2-fluorophenyl)oxirane,
anti-2-(2-fluoro-4-methoxyphenyl)-2-(chloromethyl)-3-(2-chlorophenyl)oxirane,
anti-2-(2-fluoro-4-methoxyphenyl)-2-(chloromethyl)-3-(2-methylphenyl)oxirane,
anti-2-(4-fluoro-2-methoxyphenyl)-2-(chloromethyl)-3-(2-methylphenyl)oxirane,
anti-2-(4-fluoro-2-methoxyphenyl)-2-(chloromethyl)-3-(2-fluorophenyl)oxirane,
anti-2-(4-fluoro-2-methoxyphenyl)-2-(chloromethyl)-3-(2-chlorophenyl)oxirane,
cis-2-(4-methylphenylsulfonyloxymethyl)-2-(2,4-difluorophenyl)-3-(2-trifluoromethylphenyl)oxirane,
cis-2-(4-methylphenylsulfonyloxymethyl)-2-(2,4-difluorophenyl)-3-(4-trifluoromethylphenyl)oxirane,
cis-2-(methylsulfonyloxymethyl)-2-(2,4-difluorophenyl)-3-(2-methylphenyl)oxirane,
2-hydroxymethyl-2-(2,4-difluorophenyl)-3-(2-trifluoromethylphenyl)oxirane, and
2-hydroxymethyl-2-(2,4-difluorophenyl)-3-(4-trifluoromethylphenyl)oxirane.

60. The compound III according to claim 59, where B is not orthotrifluoromethylphenyl, para-trifluoromethylphenyl or ortho-methylphenyl when A is 2,4-difluorophenyl.
